(19) United States
(12) Patent Application Publication
(10) Pub. No.: US 2024/0082327 A1
(43) Pub. Date: Mar. 14, 2024

(54) RETROVIRAL VECTORS

(71) Applicant: IP2IPO Innovations Limited, London (GB)

(72) Inventors: Deborah R. GILL, Oxford (GB); Stephen C. HYDE, Oxford (GB)

(21) Appl. No.: 18/456,354

(22) Filed: Aug. 25, 2023

(30) Foreign Application Priority Data
Aug. 26, 2022 (GB) 2212472.1

(51) Int. Cl. A61K 35/76; C12N 7/02; C12N 9/22; C12N 9/24; C12N 9/76; C12N 15/86

(52) U.S. Cl. CPC A61K 35/76; C12N 7/02; C12N 9/22; C12N 9/24; C12N 9/6427; C12N 15/86; C12N 2760/18822; C12N 2760/18832; C12N 2760/18843; C12N 2760/18851; C12Y 302/01018

(57) ABSTRACT

The present invention relates to retroviral vectors, particularly lentiviral vectors, comprising a modified retroviral RNA sequence that is codon-substituted and comprises a reduced number of retroviral open-reading frames, and wherein the retroviral vector is pseudotyped with hemagglutinin-neuraminidase (HN) and fusion (F) proteins from a respiratory paramyxovirus, methods of making the same and uses thereof.

### CROSS-REFERENCE TO RELATED APPLICATIONS

This application claims the benefit of priority to United Kingdom Patent Application No. GB 2212472.1, filed Aug. 26, 2022, hereby incorporated by reference in its entirety.

### SEQUENCE LISTING

The instant application contains a Sequence Listing which has been submitted in XML format and is hereby incorporated by reference in its entirety. Said XML copy, created on Aug. 25, 2023, is named “MSIP.P0030US Sequence Listing” and is 210 kilobytes in size.

### FIELD OF THE DISCLOSURE

The present invention relates to retroviral vectors, particularly lentiviral vectors, comprising a modified retroviral RNA sequence that is codon-substituted and comprises a reduced number of retroviral open-reading frames, and wherein the retroviral vector is pseudotyped with hemagglutinin-neuraminidase (HN) and fusion (F) proteins from a respiratory paramyxovirus, methods of making the same and uses thereof.

### BACKGROUND TO THE INVENTION

Retroviruses are a family of RNA viruses (Retroviridae) that encode the enzyme reverse transcriptase. Lentiviruses are a genus of the Retroviridae family, and are characterised by a long incubation period. Retroviruses, and lentiviruses in particular, can deliver a significant amount of viral RNA into the DNA of the host cell and have the unique ability among retroviruses of being able to infect non-dividing cells, so they are one of the most efficient methods of a gene delivery vector.

Pseudotyping is the process of producing viruses or viral vectors in combination with foreign viral envelope proteins. As such, the foreign viral envelope proteins can be used to alter host tropism or an increased/decreased stability of the virus particles. For example, pseudotyping allows one to specify the character of the envelope proteins. A frequently used protein to pseudotype retroviral and lentiviral vectors is the glycoprotein G of the Vesicular stomatitis virus (VSV), short VSV-G.

Lentiviral vectors, especially those derived from HIV-1, are widely studied and frequently used vectors. The evolution of the lentiviral vectors backbone and the ability of viruses to deliver recombinant DNA molecules (transgenes) into target cells have led to their use in many applications. Two possible applications of viral vectors include restoration of functional genes in genetic therapy and in vitro recombinant protein production.

When designing retroviral/lentiviral vectors suitable for use as gene delivery vectors, one key driver is to make the vector as safe as possible for patients. A second key driver is the need to produce sufficient quantities of the vector not just to treat an individual patient, but to allow wider clinical access to the therapy for all patients who could benefit from the therapy. These two drivers can find themselves in conflict, as modifications which improve vector safety are often associated with decreased yield during vector production.

One example of a clinical setting which would benefit from gene transfer to the airway epithelium is treatment of Cystic Fibrosis (CF). CF is a fatal genetic disorder caused by mutations in the CF transmembrane conductance regulator (CFTR) gene, which acts as a chloride channel in airway epithelial cells. CF is characterised by recurrent chest infections, increased airway secretions, and eventually respiratory failure. In the UK, the current median age at death is ^{˜}25 years. For most genotypes, there are no treatments targeting the basic defect; current treatments for symptomatic relief require hours of self-administered therapy daily. Gene therapy, unlike small molecule drugs, is independent of CFTR mutational class and is thus applicable to all affected CF individuals. However, to date there are no viral vectors approved for clinical use in the treatment of CF, and the same applies to other diseases, particularly many other respiratory tract diseases.

In addition to patient safety and yield issues, there are other difficulties conventionally associated with gene transfer to the airway epithelium.

Gene transfer efficiency to the airway epithelium is generally poor, at least in part because the respective receptors for many viral vectors appear to be predominantly localised to the basolateral surface of the airway epithelium. As such, prior to the inventors' research, the use of lentiviral pseudotypes required disruption of epithelial integrity to transduce the airways, for example by the use of detergents such as lysophosphatidylcholine or ethylene glycol bis(2-aminoethyl ether)-N,N,N′N′-tetraacetic acid, has been linked to an increased risk of sepsis. In addition, conventional gene transfer vectors struggle to penetrate the respiratory tract mucus layer, which also reduces gene transfer efficiency. The ability to administer conventional viral vectors repeatedly, mandatory for the life-long treatment of a self-renewing epithelium, is limited, because of patients' adaptive immune responses, which prevent successful repeat administration.

Administration of the vectors for clinical application is another pertinent factor. Therefore, viral stability through use of clinically relevant devices (e.g. bronchoscope and nebuliser) must be maintained for treatment efficacy.

There is accordingly a need for a gene therapy vector that is able to circumvent one or more of the problems described above. In particular, it is an object of the invention to provide a method for producing a pseudotyped retroviral or lentiviral (e.g. SIV) vector, and the means for carrying out said method, wherein the resulting vector is safe and adapted for improved gene transfer efficiency across the airway epithelium, and is produced at clinically relevant scale.

### SUMMARY OF THE INVENTION

The present inventors have previously developed a lentiviral vector, which has been pseudotyped with hemagglutinin-neuraminidase (HN) and fusion (F) proteins from a respiratory paramyxovirus, comprising a promoter and a transgene. Typically, the backbone of the vector is from a simian immunodeficiency virus (SIV), such as SIV1 or African green monkey SIV (SIV-AGM). Preferably the backbone of a viral vector of the invention is from SIV-AGM. The HN and F proteins function, respectively, to attach to sialic acids and mediate cell fusion for vector entry to target cells. The present inventors discovered that this specifically F/HN-pseudotyped lentiviral vector can efficiently transduce airway epithelium, resulting in transgene expression sustained for periods beyond the proposed lifespan of airway epithelial cells. Importantly, the present inventors also found that re-administration does not result in a loss of efficacy. These features make the vectors of the present invention attractive candidates for treating diseases via their use in expressing therapeutic proteins: (i) within the cells of the respiratory tract; (ii) secreted into the lumen of the respiratory tract; and (iii) secreted into the circulatory system.

However, there were potential safety concerns with this lentiviral vector. In particular, the lentiviral vector includes a significant number of retroviral (i.e., non-transgene) open reading frames (ORFs). There is a theoretical risk that said retroviral ORFs may be expressed following administration to a patient. Expression of retroviral ORFS represents a safety risk to the patient, particularly if said patient were to have an immune response against the expressed retroviral sequences.

Further, a significant degree of sequence homology between the retroviral vector and the GagPol plasmid used in the production creates a further theoretical risk that a replication competent lentivirus (RCL) could be generated either during manufacture, or in clinical use following administration to a patient. This represents an additional safety risk to the patient. The risk of generating replication competent viral particles is an issue for other retroviral/lentiviral vectors as well.

Whilst it would be desirable to mitigate these risks, it is not straightforward to do so, or at least not without eliciting other unacceptable disadvantages. On the one hand, modifications to reduce the number of ORFs, particularly the reduction of the number of ORFs 5′ to the promoter transgene, risks affecting the expression of the downstream transgene. Furthermore, other modifications to the retroviral genome, for example, codon substitutions with the aim of introducing STOP codons to reduce retroviral ORF length can also have deleterious effects, for example on vector yield and/or transgene expression. In addition, it is known in the art that modifications aimed at reducing the risk of RCL, such as codon-optimisation of the manufacturing gag-pol genes typically negatively impacting the titre or yield of the vector. Given the large titres of vector required to treat even a single patient, such a reduction in yield has the potential to render its production commercially unviable.

Described herein, the present inventors have designed and produced a retroviral vector, particularly a SIV vector, comprising a retroviral RNA sequence that has been modified to reduce the number of retroviral ORFs and to introduce specific codon-substitution modifications. The modified retroviral vectors of the invention comprising these newly described retroviral RNA sequences mitigate one or more of the above risks, providing a clinically advantageous product. Furthermore, the inventors have demonstrated that benefits can surprisingly be obtained without the expected disadvantages, such as reduced transgene expression and/or reduction in vector yield. Whilst such modifications had previously been considered in the context of the proviral DNA, the present application is the first to elucidate these modifications within the retroviral/lentiviral RNA sequence itself, rather than within the manufacturing platform. Further, the present application is the first to demonstrate the benefits conferred by particular modifications to the retroviral/lentiviral RNA sequence, and to show that not only does this extend to beneficial effects on vector yield, but also on transgene expression and integration of the retroviral/lentiviral RNA sequence into the host/target cell.

In particular, the inventors identified potential SIV ORFs within the SIV RNA sequence. The SIV RNA sequence was modified to remove one or more SIV ORFs. In particular, the inventors removed one or more SIV ORFs located 5′ to the transgene promoter, one or more SIV ORFs encoding polypeptides greater than or equal to 100 amino acids in length, one or more ORFs that were comprised (at least in part) in a partial RRE sequence and/or one or more ORFs that were comprised (at least in part) in a partial Gag sequence. Removal of the SIV ORFs was achieved by removing the start codon (ATG) of the selected SIV ORFs. To determine which SIV ORFs (and combinations thereof) could be removed without affecting the expression of the downstream transgene, the inventors produced a number of different SIV vectors. Each SIV vector was assessed to quantify vector yield, and transgene expression of the modified SIV vector with the corresponding unmodified vector.

The aforementioned modifications (both codon substitutions and modifications to reduce the number of SIV ORFs) were demonstrated not negatively impact transgene expression by the SIV vector pseudotyped with hemagglutinin-neuraminidase (HN) and fusion (F) proteins from a respiratory paramyxovirus, and can even result in increased transgene expression by the vector. This is surprising, given that it generally accepted that such modifications, whilst addressing potential safety issues, can give rise to detrimental effects on transgene expression.

In addition, the aforementioned mutations (both codon substitutions and modifications to reduce the number of SIV ORFs) did not have negative impact on integration of SIV vector pseudotyped with hemagglutinin-neuraminidase (HN) and fusion (F) proteins from a respiratory paramyxovirus into a host/target cell, and can even result in increased integration. Again, this is surprising, given that it generally accepted that such modifications, whilst addressing potential safety issues, can give rise to detrimental effects on vector integration.

Furthermore, the aforementioned mutations (both codon substitutions and modifications to reduce the number of SIV ORFs) did not have negative impact on the yield of SIV vector pseudotyped with hemagglutinin-neuraminidase (HN) and fusion (F) proteins from a respiratory paramyxovirus, and can even result in increased titre of the vector. Again, this is surprising, given that it generally accepted that such modifications, whilst addressing potential safety issues, can give rise to detrimental effects on vector yield.

Accordingly, the present invention provides a retroviral vector comprising a modified retroviral RNA sequence that is (i) codon-substituted and (ii) comprises a reduced number of retroviral open reading frames (ORFs) compared with the non-modified retroviral RNA sequence from which the modified retroviral RNA sequence is derived; and wherein: (a) the retroviral RNA sequence comprises a promoter and a transgene; and (b) the retroviral vector is pseudotyped with hemagglutinin-neuraminidase (HN) and fusion (F) proteins from a respiratory paramyxovirus.

Also disclosed is a method for the production of a retroviral, particularly a lentiviral vector, such as SIV, comprising a retroviral RNA sequence that is codon-substituted and comprises a reduced number of retroviral ORFs compared with the non-modified plasmid genome vector from which the modified retroviral genome RNA sequence is derived, and wherein (a) the retroviral RNA sequence comprises a promoter and a transgene, and (b) the retroviral vector is pseudotyped with hemagglutinin-neuraminidase (HN) and fusion (F) proteins from a respiratory paramyxovirus which, when administered to a patient, has a reduced risk of immune response, without negatively affecting transgene expression.

The modified retroviral genome RNA sequence may lack: (a) one or more retroviral ORFs 5′ of the promoter; (b) one or more retroviral ORF encoding a polypeptide of ≥100 amino acids in length; (c) one or more retroviral ORF comprised (at least in part) in a partial RRE sequence; and/or (d) one or more retroviral ORF comprised (at least in part) in a partial Gag sequence.

The respiratory paramyxovirus may be a Sendai virus.

The promoter may be selected the group consisting of a hybrid human CMV enhancer/EF1a (hCEF) promoter, a cytomegalovirus (CMV) promoter, and elongation factor 1a (EF1a) promoter. Preferably the vector may comprise a hybrid human CMV enhancer/EF1a (hCEF) promoter.

The transgene may be selected from: (a) CFTR, ABCA3, DNAH5, DNAH11, DNAI1, and DNAI2; or (b) a secreted therapeutic protein, optionally Alpha-1 Antitrypsin (A1AT), Factor VIII, Surfactant Protein B (SFTPB), Factor VII, Factor IX, Factor X, Factor XI, von Willebrand Factor, Granulocyte-Macrophage Colony-Stimulating Factor (GM-CSF) and a monoclonal antibody against an infectious agent. Preferably the transgene may encode: (a) CFTR; (b) A1AT; or (c) FVIII.

The promoter may be a hCEF promoter and the transgene may encode CFTR. The promoter may be a hCEF promoter and the transgene may encode A1AT. The promoter may be a hCEF or CMV promoter and the transgene may encode FVIII.

The retroviral vector may be a lentiviral vector; optionally wherein a lentiviral vector selected from the group consisting of a SIV vector, a Human immunodeficiency virus (HIV) vector, a Feline immunodeficiency virus (FIV) vector, an Equine infectious anaemia virus (EIAV) vector, and a Visna/maedi virus vector. Preferably the retroviral vector may be an SIV vector.

The modified retroviral RNA sequence may be (i) less than 9,000 bases in length and/or (ii) comprise or consist of a nucleic acid sequence having at least 80%, 90%, 91%, 92%, 93%, 94%, 95%, 96%, 97%, 98%, 99%, 99.5%, 99.9%, or up to 100% identity to SEQ ID NO: 1. Preferably the modified retroviral RNA sequence may be (i) less than 9,000 bases in length and (ii) comprise or consist of a nucleic acid sequence having at least 80%, 90%, 91%, 92%, 93%, 94%, 95%, 96%, 97%, 98%, 99%, 99.5%, 99.9%, or up to 100% identity to SEQ ID NO: 1. More preferably, the modified retroviral RNA sequence may comprise or consist of a nucleic acid sequence of SEQ ID NO: 1, still more preferably the modified retroviral RNA sequence may consist of a nucleic acid sequence of SEQ ID NO: 1.

The retroviral vector may further comprise one or more of: (a) a p17 protein comprising or consisting of an amino acid sequence having at least 80%, 90%, 91%, 92%, 93%, 94%, 95%, 96%, 97%, 98%, 99%, 99.5%, 99.9%, or up to 100% sequence identity to SEQ ID NO: 2; (b) a p24 protein comprising or consisting of an amino acid sequence having at least 80%, 90%, 91%, 92%, 93%, 94%, 95%, 96%, 97%, 98%, 99%, 99.5%, 99.9%, or up to 100% sequence identity to SEQ ID NO: 3; (c) a p8 protein comprising or consisting of an amino acid sequence having at least 80%, 90%, 91%, 92%, 93%, 94%, 95%, 96%, 97%, 98%, 99%, 99.5%, 99.9%, or up to 100% sequence identity to SEQ ID NO: 4; (d) a protease comprising or consisting of an amino acid sequence having at least 80%, 90%, 91%, 92%, 93%, 94%, 95%, 96%, 97%, 98%, 99%, 99.5%, 99.9%, or up to 100% sequence identity to SEQ ID NO: 5; (e) a p51 protein comprising or consisting of an amino acid sequence having at least 80%, 90%, 91%, 92%, 93%, 94%, 95%, 96%, 97%, 98%, 99%, 99.5%, 99.9%, or up to 100% sequence identity to SEQ ID NO: 6; (f) a p15 protein comprising or consisting of an amino acid sequence having at least 80%, 90%, 91%, 92%, 93%, 94%, 95%, 96%, 97%, 98%, 99%, 99.5%, 99.9%, or up to 100% sequence identity to SEQ ID NO: 7; and/or (g) a p31 protein comprising or consisting of an amino acid sequence having at least 80%, 90%, 91%, 92%, 93%, 94%, 95%, 96%, 97%, 98%, 99%, 99.5%, 99.9%, or up to 100% sequence identity to SEQ ID NO: 8. Optionally the vector may comprise each of (a) to (g).

The retroviral vector may further comprise one or more of: (a) a Gag protein comprising or consisting of an amino acid sequence having at least 80%, 90%, 91%, 92%, 93%, 94%, 95%, 96%, 97%, 98%, 99%, 99.5%, 99.9%, or up to 100% sequence identity to SEQ ID NO: 9; and or (b) a Pol protein comprising or consisting of an amino acid sequence having at least 80%, 90%, 91%, 92%, 93%, 94%, 95%, 96%, 97%, 98%, 99%, 99.5%, 99.9%, or up to 100% sequence identity to SEQ ID NO: 10.

The invention also provides a SIV vector pseudotyped with Sendai virus hemagglutinin-neuraminidase (HN) and fusion (F) proteins, wherein: (a) said vector comprises a modified retroviral RNA sequence which comprises or consists of a nucleic acid sequence of SEQ ID NO: 1, preferably wherein the modified retroviral RNA sequence consists of a nucleic acid sequence of SEQ ID NO: 1; and (b) the F protein comprises a first subunit which comprises or consists of an amino acid sequence of SEQ ID NO: 14 and a second subunit which comprises or consists of an amino acid sequence of SEQ ID NO: 15. Said vector may further comprise one or more of: (a) a p17 protein comprising or consisting of an amino acid sequence of SEQ ID NO: 2; (b) a p24 protein comprising or consisting of an amino acid sequence of SEQ ID NO: 3; (c) p8 protein comprising or consisting of an amino acid sequence of SEQ ID NO: 4; (d) a protease comprising or consisting of an amino acid sequence of SEQ ID NO: 5; (e) a p51 protein comprising or consisting of an amino acid sequence of SEQ ID NO: 6; (f) a p15 protein comprising or consisting of an amino acid sequence of SEQ ID NO: 7; (g) a p31 protein comprising or consisting of an amino acid sequence of SEQ ID NO: 8; (h) a Gag protein comprising or consisting of an amino acid sequence of SEQ ID NO: 9; and/or (i) a Pol protein comprising or consisting of an amino acid sequence of SEQ ID NO: 10; wherein optionally the vector comprises each of (a) to (g).

Also disclosed is a method for the production of a retroviral, particularly a lentiviral vector, such as SIV, comprising a retroviral RNA sequence that is codon-substituted and comprises a reduced number of retroviral ORFs compared with the non-modified plasmid genome vector from which the modified retroviral genome RNA sequence is derived, and wherein (a) the retroviral RNA sequence comprises a promoter and a transgene, and (b) the retroviral vector is pseudotyped with hemagglutinin-neuraminidase (HN) and fusion (F) proteins from a respiratory paramyxovirus, wherein the method has a reduced risk of RCL, without negatively affecting, or even increasing vector titre, vector integration and/or transgene expression. Thus, the methods of the invention provide for safer vectors produced at commercially desirable yields.

Accordingly the invention also provides a method of producing a retroviral vector which is codon-substituted and comprises a reduced number of ORFs compared with the non-modified retroviral RNA sequence from which the modified retroviral RNA sequence is derived and wherein the retroviral RNA sequence comprises a promoter and a transgene and which is pseudotyped with hemagglutinin-neuraminidase (HN) and fusion (F) proteins from a respiratory paramyxovirus. The method of the invention may comprise or consist of the following steps: (a) growing cells in suspension; (b) transfecting the cells with one or more plasmids; (c) adding a nuclease; (d) harvesting the lentivirus; (e) adding trypsin (or an enzyme with the same cleavage specificity); and (d) purification.

Steps (a)-(f) of the method may be carried out sequentially. The cells may be HEK293 cells (such as HEK293F or HEK293T cells) or 293T/17 cells. The addition of the nuclease may be at the pre-harvest stage. The addition of trypsin (or enzyme with the same cleavage specificity) may be at the post-harvest stage. The purification step may comprise one or more chromatography step.

The invention further provides a retroviral vector which is codon-substituted and comprises a reduced number of ORFs compared with the non-modified retroviral RNA sequence from which the modified retroviral RNA sequence is derived and wherein the retroviral RNA sequence comprises a promoter and a transgene and which is pseudotyped with hemagglutinin-neuraminidase (HN) and fusion (F) proteins from a respiratory paramyxovirus which is obtainable by a method of the invention.

The invention also provides a composition comprising a retroviral vector and a pharmaceutically acceptable excipient or diluent, wherein said retroviral vector comprises a modified retroviral RNA sequence which is codon-substituted and comprises a reduced number of ORFs compared with the non-modified retroviral RNA sequence from which the modified retroviral RNA sequence is derived and wherein the retroviral RNA sequence comprises a promoter and a transgene and the retroviral vector is pseudotyped with hemagglutinin-neuraminidase (HN) and fusion (F) proteins from a respiratory paramyxovirus. Said composition may be formulated for administration to the lungs; optionally wherein the administration is by intratracheal or intranasal instillation, aerosol delivery, intravenous injection, direct injection into the lungs.

The invention also provides a retroviral vector for use in a method of treatment, wherein the retroviral vector comprises a modified retroviral RNA sequence which is codon-substituted and comprises a reduced number of ORFs compared with the non-modified retroviral RNA sequence from which the modified retroviral RNA sequence is derived and wherein the retroviral RNA sequence comprises a promoter and a transgene and the retroviral vector is pseudotyped with hemagglutinin-neuraminidase (HN) and fusion (F) proteins from a respiratory paramyxovirus. The invention also provides a method of treating a disease comprising administering a retroviral vector to a subject in need thereof, wherein the retroviral vector comprises a modified retroviral RNA sequence which is codon-substituted and comprises a reduced number of ORFs compared with the non-modified retroviral RNA sequence from which the modified retroviral RNA sequence is derived and wherein the retroviral RNA sequence comprises a promoter and a transgene and the retroviral vector is pseudotyped with hemagglutinin-neuraminidase (HN) and fusion (F) proteins from a respiratory paramyxovirus. The disease to be treated may be a lung disease, preferably cystic fibrosis.

### BRIEF DESCRIPTION OF THE DRAWINGS

FIG. 1: FIGS. 1A-F show schematic drawings of exemplary plasmids used for production of the vectors of the invention. FIG. 1G shows an unmodified vector genome plasmid.

FIG. 2: FIG. 2 shows a schematic drawings of an exemplary pDNA1 plasmid used for production of the A1AT vectors of the invention.

FIG. 3: FIGS. 3A-D show schematic drawings of exemplary pDNA1 plasmids used for production of the FVIII vectors of the invention.

FIG. 4: FIG. 4 shows the [[The]] fourteen ATG start codons present in the Gag-RRE region of the pGM326 genome plasmid that could result in ORFs of longer than 10 amino-acids. Arrows depict the ORFs that could result from each of the labelled start codons. The circled ATGs are those that have a strong kozak and are in frame with Gag or Env.

FIG. 5: FIG. 5 shows the SIV-CFTR Titre (TU/mL) of LV generated using the Ambr®15 bioreactor system, assessed by A549 FACS Assay. VRC=Vector Reference Control

FIG. 6: FIG. 6 shows the SIV-CFTR titre (TU/mL) of LV generated using the Ambr®15 bioreactor system, assessed by HEK293T 3-Day Integration Assay. Transparent bars indicate values below the lower limit of quantification. VRC=Vector Reference Control. DNA extracted from cells that had been harvested at 3 days was size-selection purified to remove non-integrated DNA and qPCR analysis conducted.

FIG. 7: FIG. 7 shows the A549 cells expressing CFTR protein as a percentage of the live, single cell population analysed by FACS. VRC=Vector Reference Control; samples were diluted 1:20

FIG. 8: FIG. 8 shows the Western blotting (using anti-PIV1 antibody ab20791 at a dilution of 1:5000) shows cleavage of Fct4 by trypsin-like enzyme TrypLE.

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition. It should be understood that this invention is not limited to the particular methodology, protocols, and reagents, etc., described herein and as such can vary.

This disclosure is not limited by the exemplary methods and materials disclosed herein, and any methods and materials similar or equivalent to those described herein can be used in the practice or testing of embodiments of this disclosure. The terminology used herein is for the purpose of describing particular embodiments only, and is not intended to limit the scope of the present invention, which is defined solely by the claims.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

As used herein, the articles “a” and “an” may refer to one or to more than one (e.g. to at least one) of the grammatical object of the article. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

As used herein, the terms “vector”, “retroviral vector” and “retroviral F/HN vector” are used interchangeably to mean a retroviral vector comprising a retroviral RNA sequence and pseudotyped with hemagglutinin-neuraminidase (HN) and fusion (F) proteins from a respiratory paramyxovirus, unless otherwise stated. The terms “lentiviral vector” and “lentiviral F/HN vector” are used interchangeably to mean a lentiviral vector pseudotyped with hemagglutinin-neuraminidase (HN) and fusion (F) proteins from a respiratory paramyxovirus, unless otherwise stated. All disclosure herein in relation to retroviral vectors of the invention applies equally and without reservation to lentiviral vectors of the invention and to SIV vectors that are pseudotyped with hemagglutinin-neuraminidase (HN) and fusion (F) proteins from a respiratory paramyxovirus (also referred to herein as SIV F/HN or SIV-FHN).

As defined herein, the term “retroviral RNA sequence” refers to the nucleic acid molecule that is contained within a retroviral vector. A retroviral RNA sequence comprises long terminal repeat (LTR) elements, nucleic acid sequences necessary for incorporation of the retroviral RNA sequence into retroviral particles, and the transgene expression cassette. The transgene expression cassette is comprised of a suitable enhancer/promoter element, the transgene cDNA and a posttranscriptional regulatory element. The retroviral RNA sequence essentially starts with a 5′ LTR R sequence and essentially ends with a 3′ LTR R sequence. The 5′ region retroviral RNA sequence typically comprises or consists of a retroviral LTR R sequence followed by a retroviral LTR U5 sequence (in 5′ to 3′ order). The 3′ region retroviral RNA sequence typically comprises or consists of a retroviral LTR U3 sequence followed by a retroviral LTR R sequence (in 5′ to 3′ order).

The terms “DNA provirus” or “DNA provirus sequence” and “DNA proviral sequence” refer interchangeably to the DNA sequence which is integrated into the genome of cells transduced with the retrovirus. The DNA provirus sequence contains additional regions of nucleic acid that are not found within the retroviral RNA sequence, including a 5′ LTR U3 sequence and a 3′ LTR U5 sequence. Therefore, the sequences of the DNA provirus and the retroviral RNA sequence are not identical, but rather the sequence of the retroviral RNA sequence is shorter than the proviral DNA sequence from which it is derived. The precise 5′ and 3′ limits of the retroviral RNA sequence compared with the proviral DNA sequence from which it is derived cannot readily and reliably be determined by simple analysis of the proviral DNA sequence.

The retroviral vectors of the invention comprise codon-substituted retroviral RNA sequences. One of ordinary skill in the art will appreciate that codon substitution is a technique to impart advantageous properties on the resulting retroviral RNA sequence, for example, to reduce retroviral ORF length, and/or maximise protein expression. For example, codon substitution includes methods to reduce the length of retroviral ORFs and hence reduce the length of any encoded retroviral (poly)peptides, and/or to increase the translational efficiency of an encoding gene. Translational efficiency may be increased by modification of the nucleic acid sequence. Codon substitution is routine in the art, and it is within the routine practice of one of ordinary skill to devise a codon-substituted version of a given nucleic acid sequence. However, what is not straightforward is predicting the effect of codon substitution on other parameters. By way of non-limiting example, as described herein, conventional wisdom teaches that under normal manufacturing conditions, codon-substitution can decrease vector yield and/or transgene expression.

In addition to codon substitution, the retroviral RNA sequences of the invention additionally comprise modifications to reduce the number of retroviral open reading frames (ORFs). One of ordinary skill in the art appreciates that an open reading frame is a span of DNA or RNA sequence between a start and a stop codon. ORFs can be readily identified using standard techniques known in the art, such as by using software tools such as ORFfinder (ORffinder Home—NCBI (nih.gov)) from the NIH. Standard methods for testing the effect of ORFs on, e.g. vector yield and/or transgene expression are also within the routine skill of one of ordinary skill in the art and exemplary methods are described herein. A retroviral ORF is an ORF that is present in the (unmodified) retroviral RNA sequence that could potentially be expressed in a patient to give rise to a retroviral protein. Partially or fully overlapping ORFs often occur on the same nucleic acid strand. Further, competing ORFs are commonly present on different nucleic acid strands. Following administration of a retroviral vector, expression of one or more retroviral open reading frames (ORFs) to produce a retroviral protein may theoretically trigger an immune response. Specifically, in this context, the terms “ORF reduction”, “ORF elimination” and “ORF disruption” refer interchangeably to the removal of open reading frames, i.e. decreasing the number of ORFs that are translated to express a retroviral protein, peptide or polypeptide sequence. This can be achieved by any appropriate technique, for example, by the deletion of the start codon (otherwise known as an initiation codon) of said ORF. Alternatively, the nucleotides in said start codon may be substituted, or one or more additional nucleotides added to disrupt the start codon. One of ordinary skill in the art will further appreciate that the start codon in a retroviral RNA sequence is AUG. The start codon in the DNA sequence of the corresponding provirus is ATG.

STOP codons signal the termination of translation. One of ordinary skill in the art will appreciate that the standard STOP codons in a retroviral RNA sequence may be selected from UAG, UAA and UGA. Standard STOP codons in the DNA sequence of the corresponding provirus are TAG, TAA and TGA.

The retroviral vectors of the invention may additionally comprise codon-optimised retroviral RNA sequences. One of ordinary skill in the art will appreciate that codon optimisation is a technique to maximise protein expression. For example, codon optimisation can increase the translational efficiency of an encoding gene. Translational efficiency may be increased by modification of the nucleic acid sequence. Codon optimisation is routine in the art, and it is within the routine practice of one of ordinary skill to devise a codon-optimised version of a given nucleic acid sequence. However, what is not straightforward is predicting the effect of codon optimisation on other parameters. By way of non-limiting example, as described herein, conventional wisdom teaches that under normal manufacturing conditions, codon-optimisation of the gag-pol genes typically decreases vector yield.

As used herein, the terms “titre” and “yield” are used interchangeably to mean the amount of lentiviral (e.g. SIV) vector produced by a method of the invention. Titre is the primary benchmark characterising manufacturing efficiency, with higher titres generally indicating that more retroviral/lentiviral (e.g. SIV) vector is manufactured (e.g. using the same amount of reagents). Titre or yield may relate to the number of vector genomes that have integrated into the genome of a target cell (integration titre), which is a measure of “active” virus particles, i.e. the number of particles capable of transducing a cell. Transducing units (TU/mL also referred to as TTU/mL) is a biological readout of the number of host cells that get transduced under certain tissue culture/virus dilutions conditions, and is a measure of the number of “active” virus particles. The total number of (active+inactive) virus particles may also be determined using any appropriate means, such as by measuring either how much Gag is present in the test solution or how many copies of viral RNA are in the test solution. Assumptions are then made that a lentivirus particle contains either 2000 Gag molecules or 2 viral RNA molecules. Once total particle number and a transducing titre/TU have been measured, a particle:infectivity ratio calculated. Amino acids are referred to herein using the name of the amino acid, the three-letter abbreviation or the single letter abbreviation.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogues, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogues of the foregoing.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other suitable nucleic acid molecules are RNA, including siRNA, shRNA, and antisense oligonucleotides. The terms “transgene” and “gene” are also used interchangeably and both terms encompass fragments or variants thereof encoding the target protein.

The transgenes of the present invention include nucleic acid sequences that have been removed from their naturally occurring environment, recombinant or cloned DNA isolates, and chemically synthesized analogues or analogues biologically synthesized by heterologous systems.

Minor variations in the amino acid sequences of the invention are contemplated as being encompassed by the present invention, providing that the variations in the amino acid sequence(s) maintain at least 60%, at least 70%, more preferably at least 80%, at least 85%, at least 90%, at least 95%, and most preferably at least 97% or at least 99% sequence identity to the amino acid sequence of the invention or a fragment thereof as defined anywhere herein. The term homology is used herein to mean identity. As such, the sequence of a variant or analogue sequence of an amino acid sequence of the invention may differ on the basis of substitution (typically conservative substitution) deletion or insertion. Proteins comprising such variations are referred to herein as variants.

Proteins of the invention may include variants in which amino acid residues from one species are substituted for the corresponding residue in another species, either at the conserved or non-conserved positions. Variants of protein molecules disclosed herein may be produced and used in the present invention. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6] quantitative activity-property relationships of proteins can be derived using well-known mathematical techniques, such as statistical regression, pattern recognition and classification [see for example Norman et al. Applied Regression Analysis. Wiley-Interscience; 3rd edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User's Perspective (Oxford Statistical Science Series, No 22 (Paper)). Oxford University Press; (December 2000), ISBN: 0198507089; Witten, Ian H. et al Data Mining: Practical Machine Learning Tools and Techniques with Java Implementations. Morgan Kaufmann; (Oct. 11, 1999), ISBN:1558605525; Denison David G. T. (Editor) et al Bayesian Methods for Nonlinear Classification and Regression (Wiley Series in Probability and Statistics). John Wiley & Sons; (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of proteins can be derived from empirical and theoretical models (for example, analysis of likely contact residues or calculated physicochemical property) of proteins sequence, functional and three-dimensional structures and these properties can be considered individually and in combination.

Amino acids are referred to herein using the name of the amino acid, the three-letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Amino acid residues at non-conserved positions may be substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in a protein of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, lie, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

“Insertions” or “deletions” are typically in the range of about 1, 2, or 3 amino acids. The variation allowed may be experimentally determined by systematically introducing insertions or deletions of amino acids in a protein using recombinant DNA techniques and assaying the resulting recombinant variants for activity. This does not require more than routine experiments for a skilled person.

A “fragment” of a polypeptide comprises at least 50%, at least 60%, at least 70%, at least 80%, at least 90%, at least 95%, at least 97% or more of the original polypeptide.

The polynucleotides of the present invention may be prepared by any means known in the art. For example, large amounts of the polynucleotides may be produced by replication in a suitable host cell. The natural or synthetic DNA fragments coding for a desired fragment will be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured insect, mammalian, plant or other eukaryotic cell lines.

The polynucleotides of the present invention may also be produced by chemical synthesis, e.g. by the phosphoramidite method or the tri-ester method, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

When applied to a nucleic acid sequence, the term “isolated” in the context of the present invention denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

In view of the degeneracy of the genetic code, considerable sequence variation is possible among the polynucleotides of the present invention. Degenerate codons encompassing all possible codons for a given amino acid are set forth below:

| Amino Acid | Codons | Degenerate Codon |
|---|---|---|
| Cys | TGC TGT | TGY |
| Ser | AGC AGT TCA TCC TCG TCT | WSN |
| Thr | ACA ACC ACG ACT | ACN |
| Pro | CCA CCC CCG CCT | CCN |
| Ala | GCA GCC GCG GCT | GCN |
| Gly | GGA GGC GGG GGT | GGN |
| Asn | AAC AAT | AAY |
| Asp | GAC GAT | GAY |
| Glu | GAA GAG | GAR |
| Gln | CAA CAG | CAR |
| His | CAC CAT | CAY |
| Arg | AGA AGG CGA CGC CGG CGT | MGN |
| Lys | AAA AAG | AAR |
| Met | ATG | ATG |
| Ile | ATA ATC ATT | ATH |
| Leu | CTA CTC CTG CTT TTA TTG | YTN |
| Val | GTA GTC GTG GTT | GTN |
| Phe | TTC TTT | TTY |
| Tyr | TAC TAT | TAY |
| Trp | TGG | TGG |
| Ter | TAA TAG TGA | TRR |
| Asn/Asp | | RAY |
| Glu/Gln | | SAR |
| Any | | NNN |

One of ordinary skill in the art will appreciate that flexibility exists when determining a degenerate codon, representative of all possible codons encoding each amino acid. For example, some polynucleotides encompassed by the degenerate sequence may encode variant amino acid sequences, but one of ordinary skill in the art can easily identify such variant sequences by reference to the amino acid sequences of the present invention.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 85, 90, 91, 92, 93, 94, 95, 96, 97, 98, 99 or more % of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridizing under stringent (e.g. highly stringent) hybridization conditions. Nucleic acid sequence hybridization will be affected by such conditions as salt concentration (e.g. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters is much more important than any single parameter.

Methods of determining nucleic acid percentage sequence identity are known in the art. By way of example, when assessing nucleic acid sequence identity, a sequence having a defined number of contiguous nucleotides may be aligned with a nucleic acid sequence (having the same number of contiguous nucleotides) from the corresponding portion of a nucleic acid sequence of the present invention. Tools known in the art for determining nucleic acid percentage sequence identity include Nucleotide BLAST (as described below).

One of ordinary skill in the art appreciates that different species exhibit “preferential codon usage”. As used herein, the term “preferential codon usage” refers to codons that are most frequently used in cells of a certain species, thus favouring one or a few representatives of the possible codons encoding each amino acid. For example, the amino acid threonine (Thr) may be encoded by ACA, ACC, ACG, or ACT, but in mammalian host cells ACC is the most commonly used codon; in other species, different codons may be preferential. Preferential codons for a particular host cell species can be introduced into the polynucleotides of the present invention by a variety of methods known in the art. Introduction of preferential codon sequences into recombinant DNA can, for example, enhance production of the protein by making protein translation more efficient within a particular cell type or species. Thus, according to the invention, in addition to the gag-pol genes any nucleic acid sequence may be codon-optimised for expression in a host or target cell. In particular, the vector genome (or corresponding plasmid), the REV gene (or corresponding plasmid), the fusion protein (F) gene (or correspond plasmid) and/or the hemagglutinin-neuraminidase (HN) gene (or corresponding plasmid, or any combination thereof may be codon-optimised.

A “fragment” of a polynucleotide of interest comprises a series of consecutive nucleotides from the sequence of said full-length polynucleotide. By way of example, a “fragment” of a polynucleotide of interest may comprise (or consist of) at least 30 consecutive nucleotides from the sequence of said polynucleotide (e.g. at least 35, 50, 75, 100, 150, 200, 250, 300, 350, 400, 450, 500, 550, 600, 650, 700, 750, 800 850, 900, 950 or 1000 consecutive nucleic acid residues of said polynucleotide). A fragment may include at least one antigenic determinant and/or may encode at least one antigenic epitope of the corresponding polypeptide of interest. Typically, a fragment as defined herein retains the same function as the full-length polynucleotide.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” encompasses a complete inhibition or reduction as compared to a reference level. “Complete inhibition” is a 100% inhibition (i.e. abrogation) as compared to a reference level.

The terms “increased”, “increase”, “enhance”, or “activate” are all used herein to mean an increase by a statically significant amount. The terms “increased”, “increase”, “enhance”, or “activate” can mean an increase of at least 25%, at least 50% as compared to a reference level, for example an increase of at least about 50%, or at least about 75%, or at least about 80%, or at least about 90%, or at least about 100%, or at least about 150%, or at least about 200%, or at least about 250% or more compared with a reference level, or at least about a 1.5-fold, or at least about a 2-fold, or at least about a 2.5-fold, or at least about a 3-fold, or at least about a 4-fold, or at least about a 5-fold or at least about a 10-fold increase, or any increase between 1.5-fold and 10-fold or greater as compared to a reference level. In the context of a yield or titre, an “increase” is an observable or statistically significant increase in such level.

The terms “individual”, “subject”, and “patient”, are used interchangeably herein to refer to a mammalian subject for whom diagnosis, prognosis, disease monitoring, treatment, therapy, and/or therapy optimisation is desired. The mammal can be (without limitation) a human, non-human primate, mouse, rat, dog, cat, horse, or cow. In a preferred embodiment, the individual, subject, or patient is a human. An “individual” may be an adult, juvenile or infant. An “individual” may be male or female.

A “subject in need” of treatment for a particular condition can be an individual having that condition, diagnosed as having that condition, or at risk of developing that condition.

A subject can be one who has been previously diagnosed with or identified as suffering from or having a condition in need of treatment or one or more complications or symptoms related to such a condition, and optionally, have already undergone treatment for a condition as defined herein or the one or more complications or symptoms related to said condition. Alternatively, a subject can also be one who has not been previously diagnosed as having a condition as defined herein or one or more or symptoms or complications related to said condition. For example, a subject can be one who exhibits one or more risk factors for a condition, or one or more or symptoms or complications related to said condition or a subject who does not exhibit risk factors.

As used herein, the term “healthy individual” refers to an individual or group of individuals who are in a healthy state, e.g. individuals who have not shown any symptoms of the disease, have not been diagnosed with the disease and/or are not likely to develop the disease e.g. cystic fibrosis (CF) or any other disease described herein). Preferably said healthy individual(s) is not on medication affecting CF and has not been diagnosed with any other disease. The one or more healthy individuals may have a similar sex, age, and/or body mass index (BMI) as compared with the test individual. Application of standard statistical methods used in medicine permits determination of normal levels of expression in healthy individuals, and significant deviations from such normal levels.

Herein the terms “control” and “reference population” are used interchangeably.

The term “pharmaceutically acceptable” as used herein means approved by a regulatory agency of the Federal or a state government, or listed in the U.S. Pharmacopeia, European Pharmacopeia or other generally recognized pharmacopeia

The publications discussed herein are provided solely for their disclosure prior to the filing date of the present application. Nothing herein is to be construed as an admission that such publications constitute prior art to the claims appended hereto.

Disclosure related to the various methods of the invention are intended to be applied equally to other methods, therapeutic uses or methods, the data storage medium or device, the computer program product, and vice versa.

### Retroviral and Lentiviral Vectors

The invention relates to a retroviral/lentiviral (e.g. SIV) vector. The term “retrovirus” refers to any member of the Retroviridae family of RNA viruses that encode the enzyme reverse transcriptase. The term “lentivirus” refers to a family of retroviruses. Examples of retroviruses suitable for use in the present invention include gamma retroviruses such as murine leukaemia virus (MLV) and feline leukaemia virus (FLV). Examples of lentiviruses suitable for use in the present invention include Simian immunodeficiency virus (SIV), Human immunodeficiency virus (HIV), Feline immunodeficiency virus (FIV), Equine infectious anaemia virus (EIAV), and Visna/maedi virus. Preferably the invention relates to lentiviral vectors and the production thereof. A particularly preferred lentiviral vector is an SIV vector (including all strains and subtypes), such as a SIV-AGM (originally isolated from African green monkeys, Cercopithecus aethiops). Alternatively the invention relates to HIV vectors.

The retroviral/lentiviral (e.g. SIV) vectors of the invention are typically pseudotyped with hemagglutinin-neuraminidase (HN) and fusion (F) proteins from a respiratory paramyxovirus. Preferably the respiratory paramyxovirus is a Sendai virus (murine parainfluenza virus type 1).

The F protein may be a truncated F protein, typically one in which the cytoplasmic domain is truncated. Preferably the truncated F protein is Fct4, in which 38 amino acids have been truncated from the C-terminus of the F protein, with 4 amino acids of the F protein cytoplasmic domain being retained. Thus, the F protein may comprise or consist of an Fct4 amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% identity to SEQ ID NO: 12 or 13. Preferably the F protein may comprise or consist of an Fct4 amino acid sequence having at least 90%, at least 95%, or at least 99% identity to SEQ ID NO: 12 or 13.

The full length F protein, or C-terminally truncated form thereof (e.g. Fct4) is typically fusion inactive. The fusion inactive form of the F protein may be cleaved to produce two subunits, a first subunit, (also known as F₂) and a second subunit (also known as F₁).

The first subunit of the F protein may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% identity to SEQ ID NO: 14. Preferably the first subunit may be a subunit which may comprises or consists of an amino acid sequence having at least 90%, at least 95%, or at least 99% identity to SEQ ID NO: 14. SEQ ID NO: 14 is the first subunit of Fct4.

Alternatively or in addition, preferably in addition, the second subunit of the F protein may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% identity to SEQ ID NO: 15. Preferably the second subunit may be a subunit which may comprises or consists of an amino acid sequence having at least 90%, at least 95%, or at least 99% identity to SEQ ID NO: 15. SEQ ID NO: 15 is the second subunit of Fct4.

The F protein (e.g. Fct4) may comprise an N-terminal signal peptide. Alternatively, the F protein may lack such a signal peptide. The F protein signal peptide may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% identity to SEQ ID NO: 16. This signal peptide may be cleaved to form the mature F protein. The signal peptide of Fct4 is SEQ ID NO: 16, which forms amino acid residues 1-25 of SEQ ID NO: 13. Thus, the mature form of Fct4 may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% identity to amino acid residues 26-527 of SEQ ID NO: 13.

Within exemplary F protein plasmid (pDNA3a), pGM301, there is a potential alternative start codon upstream to the start codon where translation initiates to produce the Fct4 of SEQ ID NO: 12 and 13. However, according to the present invention, the F protein of the retroviral/lentiviral (e.g. SIV) vectors of the invention, does not comprise an additional amino acid sequence N-terminal to the methionine of position 1 in SEQ ID NO: 13. In particular, the F protein of the retroviral/lentiviral (e.g. SIV) vectors of the invention, typically does not comprise one or more amino acids corresponding to those encoded by bases 1645-1734 of pGM301 (SEQ ID NO: 23), which are translated as MFMPSSFSYSSWATCWLLCCLIILAKNSIA (SEQ ID NO: 46), N-terminal to the methionine of position 1 in SEQ ID NO: 13.

The HN protein may be a truncated and/or chimeric HN protein, typically one in which the cytoplasmic domain is truncated or substituted. Preferably, the HN protein is a chimeric HN protein in which (i) the cytoplasmic domain of the HN is replaced by the cytoplasmic domain of the transmembrane (TMP) protein; or (ii) the cytoplasmic domain of the TMP is added to the cytoplasmic domain of the HN protein. The HN protein may be as described in Kobayashi et al. (J. Virol. (2003) 77(4):2607-2614), which is herein incorporated by reference in its entirety.

The F/HN pseudotyping is particularly efficient at targeting cells in the airway epithelium, and as such, for therapeutic applications it is typically delivered to cells of the respiratory tract, including the cells of the airway epithelium. Accordingly, the retroviral/lentiviral (e.g. SIV) vectors of the invention are particularly suited for treatment of diseases or disorders of the airways, respiratory tract, or lung. Typically, the retroviral/lentiviral (e.g. SIV) vectors may be used for the treatment of a genetic respiratory disease.

The retroviral/lentiviral (e.g. SIV) vectors of the present invention may be pseudotyped with proteins from another virus, provided that the combination of the modified retroviral/lentiviral (e.g. SIV) RNA sequence and/or the use of codon-optimised gag-pol genes (e.g. from SIV) does not negatively impact the manufactured titre of the vector (or even results in an increased titre of the vector) and/or transgene expression (or even results in increased transgene expression). Non-limiting examples of other proteins that may be used to pseudotype retroviral/lentiviral (e.g. SIV) vectors of the present invention include G glycoprotein from Vesicular Stomatitis Virus (G-VSV) and severe acute respiratory syndrome coronavirus 2 (SARS-CoV-2) spike protein or modified forms thereof; such as those described in UK Patent Application Nos. 2118685.3 and 2105278.2, each of which is herein incorporated by reference in its entirety.

The retroviral/lentiviral (e.g. SIV) vector of the invention further comprises Gag, Pol and/or GagPol. Typically the Gag, Pol and/or GagPol is from the desired retroviral/lentiviral (e.g. SIV) vector. By way of non-limiting example, if the retroviral vector of the invention is SIV, then typically the Gag, Pol and/or GagPol are from SIV.

The Gag, Pol and/or GagPol sequences may be codon-optimised. The inventors have previously shown that the manufactured titre of a retroviral vector comprising codon-optimised Gag protein, Pol protein and/or GagPol polyprotein from SIV is unexpectedly not negatively impacted (see International Application No. PCT/GB2022/050524, which is herein incorporated by reference in its entirety). In fact, the inventors have previously shown that the manufactured titre of a retroviral vector pseudotyped with hemagglutinin-neuraminidase (HN) and fusion (F) proteins from a respiratory paramyxovirus and comprising codon-optimised Gag, Pol and/or GagPol from SIV can even be increased. This benefit of maintained/improved retroviral/lentiviral (e.g. SIV) vector yield can be combined with the benefit of the present invention in terms of providing retroviral/lentiviral (e.g. SIV) vectors with maintained/increased transgene expression and/or maintained/increased retroviral/lentiviral (e.g. SIV) RNA sequence integration, whilst addressing the potential safety risks and improving the safety profile of the retroviral/lentiviral (e.g. SIV) vectors as described herein.

In the context of Gag, Pol and/or GagPol, codon optimisation is a technique to maximise protein expression by increasing the translational efficiency of the encoding gene. Translational efficiency is increased by modification of the nucleic acid sequence. Codon optimisation is routine in the art, and it is within the routine practice of one of ordinary skill to devise a codon-optimised version of a given nucleic acid sequence. However, what is not straightforward is predicting the effect of codon optimisation on other parameters. For example, as described herein, conventional wisdom teaches that under normal manufacturing conditions (when the vector genome plasmid, rather than the gag-pol genes, is limiting), codon-optimisation of the gag-pol genes typically decreases vector yield.

The retroviral/lentiviral (e.g. SIV) vectors of the invention may comprise a codon-optimised Gag protein, a codon-optimised Pol protein, a codon-optimised GagPol polyprotein, or a combination thereof. Accordingly, the invention provides a retroviral/lentiviral (e.g. SIV) vector comprising a codon-optimised Gag protein comprising or consisting of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 9. Preferably, the invention provides a retroviral vector comprising a codon-optimised Gag protein comprising or consisting of an amino acid sequence having at least 90%, at least 95%, or at least 99% identity to SEQ ID NO: 9. The invention provides a retroviral vector comprising a codon-optimised Pol protein comprising or consisting of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 10. Preferably, the invention provides a retroviral vector comprising a codon-optimised Pol protein comprising or consisting of an amino acid sequence having a at least 90%, at least 95%, or at least 99% sequence identity to SEQ ID NO: 10.

GagPol is expressed as polyprotein which is processed to produce a number of smaller proteins within viral particles. The extent of processing, and hence the presence and/or concentration of GagPol or any of the constituent proteins within a retroviral/lentiviral (e.g. SIV) vector of the invention may vary with time.

Accordingly, a retroviral/lentiviral (e.g. SIV) vector of the invention may comprise one or more of a p17 protein, a p27 protein, a p8 protein, a protease, a p51 protein, a p15 protein and a p31 protein. One or more of these proteins may be present in combination with Gag, Pol and/or GagPol. Preferably, the invention provides a retroviral vector comprising a p17 protein, a p27 protein, a p8 protein, a protease, a p51 protein, a p15 protein and a p31 protein. Again, these proteins may be present in combination with Gag, Pol and/or GagPol.

The p17 protein may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 2. Preferably, the p17 protein comprises or consists of an amino acid sequence having at least 90%, at least 95%, or at least 99% sequence identity to SEQ ID NO:2.

The p24 protein may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 3. Preferably, the p24 protein comprises or consists of an amino acid sequence having at least 90%, at least 95%, or at least 99% sequence identity to SEQ ID NO: 3.

The p8 protein may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 4. Preferably, the p8 protein comprises or consists of an amino acid sequence having at least 90%, at least 95%, or at least 99% sequence identity to SEQ ID NO: 4.

The protease may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 5. Preferably, the protease comprises or consists of an amino acid sequence having at least 90%, at least 95%, or at least 99% sequence identity to SEQ ID NO: 5.

The p51 protein may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 6. Preferably, the p51 protein comprises or consists of an amino acid sequence having at least 90%, at least 95%, or at least 99% sequence identity to SEQ ID NO: 6.

The p15 protein may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 7. Preferably, the p15 protein comprises or consists of an amino acid sequence having at least 90%, at least 95%, or at least 99% sequence identity to SEQ ID NO: 7.

The p31 protein may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 8. Preferably, the p31 protein comprises or consists of an amino acid sequence having at least 90%, at least 95%, or at least 99% sequence identity to SEQ ID NO: 8.

Retroviral/lentiviral (e.g. SIV) vectors of the invention may comprise a p17 protein comprising or consisting of an amino acid sequence having at least 80%, 90%, 91%, 92%, 93%, 94%, 95%, 96%, 97%, 98%, 99%, 99.5%, 99.9%, or up to 100% sequence identity to SEQ ID NO: 2 (as described above), a p24 protein comprising or consisting of an amino acid sequence having at least 80%, 90%, 91%, 92%, 93%, 94%, 95%, 96%, 97%, 98%, 99%, 99.5%, 99.9%, or up to 100% sequence identity to SEQ ID NO: 3 (as described above), a p8 protein comprising or consisting of an amino acid sequence having at least 80%, 90%, 91%, 92%, 93%, 94%, 95%, 96%, 97%, 98%, 99%, 99.5%, 99.9%, or up to 100% sequence identity to SEQ ID NO: 4 (as described above), a protease comprising or consisting of an amino acid sequence having at least 80%, 90%, 91%, 92%, 93%, 94%, 95%, 96%, 97%, 98%, 99%, 99.5%, 99.9%, or up to 100% sequence identity to SEQ ID NO: 5 (as described above), a p51 protein comprising or consisting of an amino acid sequence having at least 80%, 90%, 91%, 92%, 93%, 94%, 95%, 96%, 97%, 98%, 99%, 99.5%, 99.9%, or up to 100% sequence identity to SEQ ID NO: 6 (as described above), a p15 protein comprising or consisting of an amino acid sequence having at least 80%, 90%, 91%, 92%, 93%, 94%, 95%, 96%, 97%, 98%, 99%, 99.5%, 99.9%, or up to 100% sequence identity to SEQ ID NO: 7 (as described above), and a p31 protein comprising or consisting of an amino acid sequence having at least 80%, 90%, 91%, 92%, 93%, 94%, 95%, 96%, 97%, 98%, 99%, 99.5%, 99.9%, or up to 100% sequence identity to SEQ ID NO: 8 (as described above).

A retroviral/lentiviral (e.g. SIV) vector according to the invention may be integrase-competent (IC). Alternatively, the retroviral/lentiviral (e.g. SIV) vector may be integrase-deficient (ID).

Retroviral/lentiviral (e.g. SIV) vectors, such as those of the invention, can integrate into the genome of transduced cells and lead to long-lasting expression, making them suitable for transduction of stem/progenitor cells. In the lung, several cell types with regenerative capacity have been identified as responsible for maintaining specific cell lineages in the conducting airways and alveoli. These include basal cells and submucosal gland duct cells in the upper airways, club cells and neuroendocrine cells in the bronchiolar airways, bronchioalveolar stem cells in the terminal bronchioles and type II pneumocytes in the alveoli. Therefore, and without being bound by theory, it is believed that said retroviral/lentiviral (e.g. SIV) vectors bring about long term gene expression of the transgene of interest by introducing the transgene into one or more long-lived airway epithelial cells or cell types, such as basal cells and submucosal gland duct cells in the upper airways, club cells and neuroendocrine cells in the bronchiolar airways, bronchioalveolar stem cells in the terminal bronchioles and type II pneumocytes in the alveoli. As demonstrated herein, the integration of retroviral/lentiviral (e.g. SIV) vectors with modified retroviral/lentiviral (e.g. SIV) RNA sequences of the invention into target cell genomes is unexpectedly not negatively impacted, and in fact may even be increased.

Accordingly, the retroviral/lentiviral (e.g. SIV) vectors of the invention may transduce one or more cells or cell lines with regenerative potential within the lung (including the airways and respiratory tract) to achieve long term gene expression. For example, the retroviral/lentiviral (e.g. SIV) vectors may transduce basal cells, such as those in the upper airways/respiratory tract. Basal cells have a central role in processes of epithelial maintenance and repair following injury. In addition, basal cells are widely distributed along the human respiratory epithelium, with a relative distribution ranging from 30% (larger airways) to 6% (smaller airways).

The retroviral/lentiviral (e.g. SIV) vectors of the invention may be used to transduce isolated and expanded stem/progenitor cells ex vivo prior administration to a patient. Preferably, the retroviral/lentiviral (e.g. SIV) vectors of the invention are used to transduce cells within the lung (or airways/respiratory tract) in vivo.

The retroviral/lentiviral (e.g. SIV) vectors of the invention demonstrate remarkable resistance to shear forces with only modest reduction in transduction ability when passaged through clinically-relevant delivery devices such as bronchoscopes, spray bottles and nebulisers.

The retroviral/lentiviral (e.g. SIV) vectors of the present invention enable high levels of transgene expression, resulting in high levels (therapeutic levels) of expression of a therapeutic protein. The retroviral/lentiviral (e.g. SIV) vectors of the present invention typically provide high expression levels of a transgene when administered to a patient. The terms high expression and therapeutic expression are used interchangeably herein. Expression may be measured by any appropriate method (qualitative or quantitative, preferably quantitative), and concentrations given in any appropriate unit of measurement, for example ng/ml or nM.

Expression of a transgene of interest may be given relative to the expression of the corresponding endogenous (defective) gene in a patient. Expression may be measured in terms of mRNA or protein expression. The expression of the transgene of the invention, such as a functional CFTR gene, may be quantified relative to the endogenous gene, such as the endogenous (dysfunctional) CFTR genes in terms of mRNA copies per cell or any other appropriate unit.

Expression levels of a transgene and/or the encoded therapeutic protein of the invention may be measured in the lung tissue, epithelial lining fluid and/or serum/plasma as appropriate. A high and/or therapeutic expression level may therefore refer to the concentration in the lung, epithelial lining fluid and/or serum/plasma.

The retroviral/lentiviral (e.g. SIV) vectors of the invention exhibit efficient airway cell uptake, enhanced transgene expression, and suffer no loss of efficacy upon repeated administration. Accordingly, the retroviral/lentiviral (e.g. SIV) vectors of the invention are capable of producing long-lasting, repeatable, high-level expression in airway cells without inducing an undue immune response.

The retroviral/lentiviral (e.g. SIV) vectors of the present invention enable long-term transgene expression, resulting in long-term expression of a therapeutic protein. As described herein, the phrases “long-term expression”, “sustained expression”, “long-lasting expression” and “persistent expression” are used interchangeably. Long-term expression according to the present invention means expression of a therapeutic gene and/or protein, preferably at therapeutic levels, for at least 45 days, at least 60 days, at least 90 days, at least 120 days, at least 180 days, at least 250 days, at least 360 days, at least 450 days, at least 730 days or more. Preferably long-term expression means expression for at least 90 days, at least 120 days, at least 180 days, at least 250 days, at least 360 days, at least 450 days, at least 720 days or more, more preferably at least 360 days, at least 450 days, at least 720 days or more. This long-term expression may be achieved by repeated doses or by a single dose.

Repeated doses may be administered twice-daily, daily, twice-weekly, weekly, monthly, every two months, every three months, every four months, every six months, yearly, every two years, or more. Dosing may be continued for as long as required, for example, for at least six months, at least one year, two years, three years, four years, five years, ten years, fifteen years, twenty years, or more, up to for the lifetime of the patient to be treated.

Preferably, the invention relates to F/HN retroviral/lentiviral vectors comprising a promoter and a transgene, particularly SIV F/HN vectors.

### Retroviral and Lentiviral RNA Sequences

Each retroviral vector particle comprises a retroviral RNA sequence. The retroviral RNA sequence comprises the LTR elements, sequences necessary for incorporation into particles, along with the transgene expression cassette. By way of non-limiting example, the retroviral RNA sequence may comprise or consist of retroviral LTR elements (typically R and U5 (read 5′ to 3′) at the 5′ end of the sequence, and U3 and R (read 5′ to 3′) at the 3′ end of the sequence), retroviral sequences necessary for incorporation into retroviral particles, along with the transgene expression cassette. The transgene expression cassette is typically comprised of a suitable enhancer/promoter element, the transgene cDNA and a posttranscriptional regulatory element. Particularly preferred is a retroviral RNA sequence which comprises SIV LTR elements, sequences necessary for incorporation into particles, along with the transgene expression cassette. By way of non-limiting example, a SIV RNA sequence may comprise or consist of SIV LTR elements (typically R and U5 (read 5′ to 3′) at the 5′ end of the sequence, and U3 and R (read 5′ to 3′) at the 3′ end of the sequence), SIV sequences necessary for incorporation into retroviral particles, along with the transgene expression cassette.

A retroviral or lentiviral RNA sequence of the invention is modified compared with the unmodified retroviral or lentiviral RNA sequence from which it is derived. Modification of the retroviral or lentiviral RNA sequence may provide advantageous properties compared with the retroviral or lentiviral RNA sequence from which it is derived. Non-limiting examples of such advantageous properties include maintained/increased transgene expression, maintained/increased retroviral/lentiviral (e.g. SIV) RNA sequence integration into a target/host cell genome, maintained/increased vector yield and/or improved patient safety compared with the unmodified retroviral or lentiviral RNA sequence from which it is derived.

The modified retroviral or lentiviral RNA sequence of the invention may be codon-substituted and/or comprise a reduced number of retroviral or lentiviral ORFs compared with the retroviral or lentiviral RNA sequence from which it is derived. For example, a modified retroviral or lentiviral RNA sequence of the invention may comprise a reduced number of retroviral or lentiviral ORFs compared with the retroviral or lentiviral RNA sequence from which it is derived. Typically the modified retroviral or lentiviral RNA sequence of the invention is codon-substituted and comprises reduced number of retroviral or lentiviral ORFs compared with the retroviral or lentiviral RNA sequence from which it is derived.

Codon-substitution of the retroviral or lentiviral RNA sequence may comprise, for example, the introduction of STOP codons and/or the introduction and/or removal of restriction enzyme cleavage sites. At least 1, at least 2, at least 3, at least 4, at least 5 or more codons may be substituted in a modified retroviral or lentiviral genome of the invention. For each codon that is substituted, the nature of the modification may independently be selected from for example, the introduction of STOP codons and/or the introduction and/or removal of restriction enzyme cleavage sites. Standard techniques for codon-substituting the retroviral or lentiviral RNA sequence in this way are known in the art. Preferably the modified retroviral/lentiviral (e.g. SIV) RNA sequence includes one or more codon-substitution to introduce a STOP codon. The introduction of a STOP codon may comprise the introduction of a frameshift.

The introduction of STOP codons can result in the early termination of translation, resulting in ORFs of reduced length compared to the corresponding unmodified ORF in which a STOP sequence has not been introduced. Thus, according to the invention a retroviral or lentiviral RNA sequence is typically modified to introduce one or more STOP codon and thus reduce the length of one or more ORF. For example, the length of one or more ORF may be reduced by the introduction of a UAG, UAA or UGA codon in the retroviral RNA sequence (or TAG, TAA or TGA codon in the pro-retroviral DNA sequence). As described herein, STOP codons may be removed by deletion or substitution of nucleotides within the retroviral RNA sequence or corresponding pro-retroviral DNA sequence to result in a STOP codon, or by the addition of one or more (e.g. 1, 2 or 3) nucleotides to introduce a STOP codon. Preferably the retroviral or lentiviral RNA sequence is modified to reduce the length of one or more retroviral or lentiviral ORF. Reducing the length of one or more retroviral or lentiviral ORF has the potential to improve the safety of the retroviral or lentiviral vector when administered to a subject. Thus, a retroviral or lentiviral vector of the invention comprising a modified retroviral or lentiviral RNA sequence may have an improved safety profile compared with a retroviral or lentiviral vector comprising the non-modified retroviral or lentiviral RNA sequence from which the modified retroviral or lentiviral RNA sequence is derived. By way of non-limiting example, reducing the length of one or more retroviral or lentiviral ORF reduces the risk of an immune response being triggered by expression of the longer polypeptide that is encoded by the corresponding unmodified one or more retroviral or lentiviral ORF. In addition, as demonstrated herein, the length of one or more retroviral or lentiviral ORF can be reduced without negatively affecting the expression of the downstream transgene, integration of the retroviral or lentiviral vector and/or the yield of the retroviral or lentiviral vector. Reduction of the length of one or more retroviral or lentiviral ORF may increase the expression of the downstream transgene, retroviral or lentiviral vector integration and/or the yield of the retroviral or lentiviral vector.

As exemplified herein, such modifications may comprise or consist of modifying the retroviral or lentiviral RNA sequence to introduce STOP codons to reduce the length of one or more viral, particularly retroviral/lentiviral (e.g. SIV) ORF in said sequence compared with the non-modified retroviral or lentiviral RNA sequence from which the modified retroviral or lentiviral RNA sequence is derived. Modification of the retroviral or lentiviral RNA sequence may be achieved by modification of the vector genome plasmid (i.e. pDNA1) as described herein that is used to produce the modified retroviral or lentiviral vector of the invention. Thus, a modified vector genome plasmid (i.e. pDNA1) may comprise one or more ORF, particularly one or more retroviral/lentiviral (e.g. SIV) ORF of reduced length compared with a corresponding non-modified plasmid genome vector (i.e., pDNA1).

By way of non-limiting example, a modified retroviral or lentiviral (e.g. SIV) RNA sequence of the invention may be modified to introduce at least 1, at least 2, at least 3, at least 4, at least 5 or more STOP codons, each of which typically reduces the length of a retroviral or lentiviral (e.g. SIV) ORF. Typically, the length of the one or more retroviral or lentiviral (e.g. SIV) ORF is reduced compared with the corresponding retroviral or lentiviral (e.g. SIV) ORF in the non-modified retroviral or lentiviral (e.g. SIV) RNA sequence from which the modified retroviral or lentiviral (e.g. SIV) RNA sequence is derived. Thus, the vector genome plasmid used to produce the modified retroviral or lentiviral (e.g. SIV) vector of the invention may comprise one or more ORF, particularly one or more retroviral/lentiviral (e.g. SIV) ORF of reduced length compared with a corresponding non-modified plasmid genome vector (i.e., pDNA1).

The retroviral or lentiviral (e.g. SIV) RNA sequence may be modified to reduce the length of one or more retroviral or lentiviral (e.g. SIV) ORFs 5′ (also referred to as upstream) of the transgene and/or the transgene promoter. One or more retroviral or lentiviral (e.g. SIV) ORFs from 5′ of the transgene and/or the transgene promoter may be reduced in length. By way of non-limiting example, at least 1, at least 2, at least 3, at least 4, at least 5 or more retroviral or lentiviral (e.g. SIV) ORFs from 5′ of the transgene and/or the transgene promoter may be reduced in length. Preferably, one or two retroviral or lentiviral (e.g. SIV) ORFs 5′ of the transgene promoter are reduced in length. The length of one or more upstream ORF may be reduced compared with length of the corresponding ORF in the non-modified retroviral or lentiviral (e.g. SIV) RNA sequence from which the modified retroviral or lentiviral (e.g. SIV) RNA sequence is derived. Thus, the vector genome plasmid used to produce the modified retroviral or lentiviral (e.g. SIV) vector of the invention may comprise one or more upstream ORF, particularly one or more upstream retroviral/lentiviral (e.g. SIV) ORF of reduced length compared with a corresponding non-modified plasmid genome vector (i.e., pDNA1).

Introduction of a STOP codon may reduce the length of the polypeptide encoded by a retroviral or lentiviral (e.g. SIV) ORFs by at least 5 amino acids, at least 10 amino acids, at least 20 amino acids, at least 40 amino acids or more.

Alternatively or in addition, each STOP codon introduced may reduce the length of the one or more retroviral or lentiviral (e.g. SIV) ORFs that encodes a polypeptide of at least 10 amino acids in length, such as at least 50 amino acids in length, at least 100 amino acids in length, at least 200 amino acids in length or more, compared with the length of the unmodified ORF prior to introduction of the STOP codon. For example, introduction of a STOP codon may reduce the length of the one or more retroviral or lentiviral (e.g. SIV) ORFs that encodes a polypeptide of at least 230 amino acids in length.

Thus, by way of non-limiting example, introduction of a STOP codon may reduce the length of the polypeptide encoded by a retroviral or lentiviral (e.g. SIV) ORFs, wherein (i) the polypeptide encoded by the (unmodified ORF) is at least 230 amino acids in length; and (ii) the length of the polypeptide encoded by said ORF is reduced by at least 40 amino acids or more.

The introduction of an individual STOP codon may reduce the length of more than one ORF, particularly one or more retroviral/lentiviral ORF. In particular, introduction of an individual STOP codon may reduce the length of 2, or 3 ORFs, particularly 2 or 3 retroviral/lentiviral ORFs, with a reduction in length of 2 ORFs being preferred.

Other codon-substitutions include the removal and/or replacement of one or more restriction enzyme site. Such codon-substitutions may be useful in the production of retroviral/lentiviral vectors of the invention.

Preferred codon-substitutions may comprise or consist of replacement of a frameshift mutation and a STOP codon into the Env ORF of the retroviral/lentiviral RNA sequence. Such substitutions typically reduce the length of the Env ORF and prevent readthrough of from the Env ORF into the cPPT sequence. As exemplified, one such preferred codon-substitution comprises the replacement of a motif corresponding to residues 2347-2352 of SEQ ID NO: 25 with the motif corresponding to residues 2354-2360 of SEQ ID NO: 19. This reduces the length of the polypeptide encoded by the Env ORF from 235 amino acids to 192 amino acids, and also reduces the length of the polypeptide encoded by an additional retroviral/lentiviral ORF from 19 amino acids to 9 amino acids. The motif corresponding to residues 2354-2360 of SEQ ID NO: 19 is found at residues 1601-1607 of SEQ ID NO: 1.

Another preferred codon-substitution that may be used alternatively or in addition to the codon-substitution of the preceding paragraph is the introduction of a SbfI restriction site, which may optionally replace an EcoR1 restriction site within the retroviral/lentiviral RNA sequence. As exemplified, one such preferred codon-substitution comprises the replacement of a motif corresponding to residues 1734-1739 of SEQ ID NO: 25 with the motif corresponding to residues 1738-1746 of SEQ ID NO: 19. The motif corresponding to residues 1738-1746 of SEQ ID NO: 19 is found at residues 985-993 of SEQ ID NO: 1.

Particularly preferred are codon-substitutions which comprise or consist of the combination of (a) replacement of a frameshift mutation and a STOP codon into the Env ORF of the retroviral/lentiviral RNA sequence; and (b) introduction of a SbfI restriction site, which may optionally replace an EcoR1 restriction site within the retroviral/lentiviral RNA sequence. As exemplified, particularly preferred codon-substitutions comprise or consist of (a) the replacement of a motif corresponding to residues 2347-2352 of SEQ ID NO: 25 with the motif corresponding to residues 2354-2360 of SEQ ID NO: 25; and (b) the replacement of a motif corresponding to residues 1734-1739 of SEQ ID NO: 25 with the motif corresponding to residues 1738-1746 of SEQ ID NO: 25.

The retroviral or lentiviral RNA sequence is typically modified to reduce the number of ORFs. For example, the number of ORFs may be reduced by removing AUG codons in the retroviral RNA sequence (or ATG codons in the pro-retroviral DNA sequence). As described herein, start codons may be removed by deletion or substitution of nucleotides within the start codon, or by the addition of one or more (e.g. 1, 2 or 3) nucleotides to disrupt the start codon. Preferably the retroviral or lentiviral RNA sequence is modified to reduce the number of retroviral or lentiviral ORFs. Removal of one or more retroviral or lentiviral ORFs has the potential to improve the safety of the retroviral or lentiviral vector when administered to a subject. Thus, a retroviral or lentiviral vector of the invention comprising a modified retroviral or lentiviral RNA sequence may have an improved safety profile compared with a retroviral or lentiviral vector comprising the non-modified retroviral or lentiviral RNA sequence from which the modified retroviral or lentiviral RNA sequence is derived. By way of non-limiting example, removal of one or more retroviral or lentiviral ORFs reduces the risk of an immune response being triggered by expression of said one or more retroviral or lentiviral ORFs. In addition, as demonstrated herein, one or more retroviral or lentiviral ORF can be removed without negatively affecting the expression of the downstream transgene, integration of the retroviral or lentiviral vector and/or the yield of the retroviral or lentiviral vector. Removal of one or more retroviral or lentiviral ORF may increase the expression of the downstream transgene, integration of the retroviral or lentiviral vector and/or the yield of the retroviral or lentiviral vector.

As exemplified herein, such modifications may comprise or consist of modifying the retroviral or lentiviral RNA sequence to remove viral, particularly retroviral/lentiviral (e.g. SIV), ORFs from said sequence compared with the non-modified retroviral or lentiviral RNA sequence from which the modified retroviral or lentiviral RNA sequence is derived. Modification of the retroviral or lentiviral RNA sequence may be achieved by modification of the vector genome plasmid (i.e. pDNA1) as described herein that is used to produce the modified retroviral or lentiviral vector of the invention. Thus, a modified vector genome plasmid (i.e. pDNA1) may comprise a reduced number of viral, particularly retroviral/lentiviral (e.g. SIV) ORFs compared with a corresponding non-modified plasmid genome vector (i.e., pDNA1). Thus, a modified retroviral or lentiviral vector of the invention comprises a reduced number of non-transgene ORFs on its retroviral or lentiviral RNA sequence.

By way of non-limiting example, a modified retroviral or lentiviral (e.g. SIV) RNA sequence of the invention may be modified to remove at least 1, at least 2, at least 3, at least 4, at least 5, at least 6, at least 7, at least 8, at least 9, at least 10, or more retroviral or lentiviral (e.g. SIV) ORFs, typically at least 6 or at least 7 retroviral or lentiviral (e.g. SIV) ORFs, preferably 6 or 7 retroviral or lentiviral (e.g. SIV) ORFs. Typically, the number of retroviral or lentiviral (e.g. SIV) ORFs is reduced compared with the non-modified retroviral or lentiviral (e.g. SIV) RNA sequence from which the modified retroviral or lentiviral (e.g. SIV)RNA sequence is derived. Thus, the vector genome plasmid used to produce the modified retroviral or lentiviral (e.g. SIV) vector of the invention may have a reduced number of retroviral or lentiviral (e.g. SIV) ORFs compared with the corresponding non-modified vector genome plasmid.

The retroviral or lentiviral (e.g. SIV) RNA sequence may be modified to reduce the number of retroviral or lentiviral (e.g. SIV) ORFs 5′ (also referred to as upstream) of the transgene and/or the transgene promoter. One or more retroviral or lentiviral (e.g. SIV) ORFs from 5′ of the transgene and/or the transgene promoter may be removed. By way of non-limiting example, at least 1, at least 2, at least 3, at least 4, at least 5, at least 6, at least 7, at least 8, at least 9, at least 10 or more retroviral or lentiviral (e.g. SIV) ORFs from 5′ of the transgene and/or the transgene promoter may be removed, typically at least 6 or at least 7 retroviral or lentiviral (e.g. SIV) ORFs, preferably 6 or 7 retroviral or lentiviral (e.g. SIV) ORFs. Preferably, one or more retroviral or lentiviral (e.g. SIV) ORFs is removed from 5′ of the transgene promoter. The number of upstream ORFs may be reduced compared with the non-modified retroviral or lentiviral (e.g. SIV) RNA sequence from which the modified retroviral or lentiviral (e.g. SIV) RNA sequence is derived. Thus, the vector genome plasmid used to produce the modified retroviral or lentiviral (e.g. SIV) vector of the invention may have a reduced number of upstream retroviral or lentiviral (e.g. SIV) ORFs compared with the corresponding non-modified vector genome plasmid.

Alternatively, or additionally, the one or more retroviral or lentiviral (e.g. SIV) ORFs removed according to the invention may each independently encode a polypeptide of greater than or equal to 10 amino acids in length, greater than or equal to 20 amino acids in length, greater than or equal to 30 amino acids in length, greater than or equal to 40 amino acids in length, greater than or equal to 50 amino acids in length, greater than or equal to 60 amino acids in length, greater than or equal to 70 amino acids in length, greater than or equal to 80 amino acids in length, greater than or equal to 90 amino acids in length, greater than or equal to 100 amino acids in length, greater than or equal to 110 amino acids in length, greater than or equal to 120 amino acids in length, greater than or equal to 130 amino acids in length, greater than or equal to 140 amino acids in length or greater than or equal to 150 amino acids in length. Typically, the one or more retroviral or lentiviral (e.g. SIV) ORFs removed according to the invention may each independently encode a polypeptide of greater than or equal to 100 amino acids in length. Preferably, at least one retroviral or lentiviral (e.g. SIV) ORFs encoding a polypeptide of greater than or equal to 100 amino acids in length may be removed from the modified retroviral or lentiviral (e.g. SIV) RNA sequence compared with the non-modified retroviral or lentiviral (e.g. SIV) RNA sequence from which the modified retroviral or lentiviral (e.g. SIV) RNA sequence is derived. Thus, the vector genome plasmid used to produce the modified retroviral or lentiviral (e.g. SIV) vector of the invention may have one or more retroviral or lentiviral (e.g. SIV) ORFs encoding a polypeptide of greater than or equal to 100 amino acids in length removed compared with the non-modified plasmid genome vector from which the modified retroviral RNA sequence is derived.

Thus, a retroviral or lentiviral (e.g. SIV) RNA sequence of the invention may lack any ORFs (other than the transgene) encoding a polypeptide greater than or equal to 200 amino acids in length, greater than or equal to 190 amino acids in length, greater than or equal to 180 amino acids in length, greater than or equal to 170 amino acids in length, or greater than or equal to 160 amino acids in length compared with the non-modified retroviral or lentiviral (e.g. SIV) RNA sequence from which the modified retroviral or lentiviral (e.g. SIV) RNA sequence is derived. Thus, the vector genome plasmid used to produce the modified retroviral or lentiviral (e.g. SIV) vector of the invention may have lack any ORFs (other than the transgene) encoding a polypeptide greater than or equal to 200 amino acids in length as described above compared with the non-modified plasmid genome vector from which the modified retroviral RNA sequence is derived.

A retroviral or lentiviral (e.g. SIV) RNA sequence of the invention may lack any ORFs encoding a polypeptide greater than or equal to 180 amino acids in length, greater than or equal to 100 amino acids in length, greater than or equal to 90 amino acids in length, greater than or equal to 80 amino acids in length, or greater than or equal to 70 amino acids in length within the partial Gag region compared with the non-modified retroviral or lentiviral (e.g. SIV) RNA sequence from which the modified retroviral or lentiviral (e.g. SIV) RNA sequence is derived. Thus, the vector genome plasmid used to produce the modified retroviral or lentiviral (e.g. SIV) vector of the invention may have lack any ORFs (other than the transgene) encoding a polypeptide greater than or equal to 180 amino acids in length in the partial Gag region as described above compared with the non-modified plasmid genome vector from which the modified retroviral RNA sequence is derived.

A retroviral or lentiviral (e.g. SIV) RNA sequence of the invention may lack any ORFs encoding a polypeptide greater than or equal to 200 amino acids in length, greater than or equal to 170 amino acids in length, or greater than or equal to 160 amino acids in length within the partial RRE region compared with the non-modified retroviral or lentiviral (e.g. SIV) RNA sequence from which the modified retroviral or lentiviral (e.g. SIV) RNA sequence is derived. Thus, the vector genome plasmid used to produce the modified retroviral or lentiviral (e.g. SIV) vector of the invention may have lack any ORFs (other than the transgene) encoding a polypeptide of greater than or equal to 160 amino acids in length in the partial RRE region as described above compared with the non-modified plasmid genome vector from which the modified retroviral RNA sequence is derived.

Alternatively, or additionally, the one or more retroviral or lentiviral (e.g. SIV) ORF to be removed may be comprised (at least in part) in an RRE sequence. Preferably, the one or more retroviral or lentiviral (e.g. SIV) ORF is comprised (at least in part) in a partial RRE sequence. Accordingly, the retroviral or lentiviral (e.g. SIV) RNA sequence may be modified to reduce the number of ORFs comprised (at least in part) in a partial RRE sequence, compared with the non-modified retroviral or lentiviral (e.g. SIV) RNA sequence from which the modified retroviral or lentiviral (e.g. SIV) RNA sequence is derived. Thus, the vector genome plasmid used to produce the modified retroviral or lentiviral (e.g. SIV) vector of the invention may have a reduced number of ORFs comprised (at least in part) in a partial RRE sequence compared with the non-modified plasmid genome vector from which the modified retroviral RNA sequence is derived.

Alternatively, or additionally, the one or more retroviral or lentiviral (e.g. SIV) ORF may be comprised (at least in part) in a partial Gag sequence. Accordingly, the retroviral or lentiviral (e.g. SIV) RNA sequence may be modified to reduce the number of ORFs comprised (at least in part) in a partial Gag sequence, compared with the non-modified retroviral or lentiviral (e.g. SIV) RNA sequence from which the modified retroviral or lentiviral (e.g. SIV) RNA sequence is derived. Thus, the vector genome plasmid used to produce the modified retroviral or lentiviral (e.g. SIV) vector of the invention may have a reduced number of ORFs comprised (at least in part) in a partial Gag sequence compared with the non-modified plasmid genome vector from which the modified retroviral RNA sequence is derived.

References herein to an ORF that is comprised in a region of the retroviral/lentiviral (e.g. SIV) sequence, e.g. comprised in a partial Gag sequence or partial RRE sequence also apply equally and without reservation to ORFs that are partially comprised in said region of the retroviral/lentiviral (e.g. SIV) sequence, e.g. comprised in a partial Gag sequence or partial RRE sequence, unless expressly stated to the contrary. An ORF to be removed may run through different regions of the retroviral/lentiviral (e.g. SIV) sequence, and so be comprised by two or more regions of the retroviral/lentiviral (e.g. SIV) sequence. For example, an ORF to be removed may run through a partial Gag sequence into a partial RRE sequence.

Typically, the removal of the one or more retroviral or lentiviral (e.g. SIV) ORFs does not negatively affect the expression of the downstream transgene, compared to a non-modified retroviral or lentiviral (e.g. SIV) RNA sequence. The removal of the one or more retroviral or lentiviral (e.g. SIV) ORFs may increase the expression of the downstream transgene, compared with a non-modified retroviral or lentiviral (e.g. SIV) RNA sequence. The non-modified retroviral RNA sequence may be produced from the aforementioned non-modified plasmid genome vector.

Whilst a modified retroviral RNA or lentiviral (e.g. SIV) sequence may comprise no ORFs (particularly no retroviral or lentiviral (e.g. SIV) ORFs) other than the transgene, this is not essential. Rather, a modified retroviral or lentiviral (e.g. SIV) RNA sequence may still comprise ORFs (including retroviral or lentiviral (e.g. SIV)) other than the transgene, but may comprise a reduced number of non-transgene ORFs compared with the non-modified retroviral or lentiviral (e.g. SIV) RNA sequence from which the modified retroviral or lentiviral (e.g. SIV) RNA sequence is derived. Alternatively or in addition, the length of the remaining non-transgene ORFs may be reduced compared with the non-modified retroviral or lentiviral (e.g. SIV) RNA sequence from which the modified retroviral or lentiviral (e.g. SIV) RNA sequence is derived. Thus, the vector genome plasmid used to produce the modified retroviral or lentiviral (e.g. SIV) vector of the invention may have a reduced number of non-transgene ORFs compared with the unmodified plasmid genome (pDNA1) from which it is derived. Alternatively or in addition, the remaining non-transgene ORFs within the vector genome plasmid used to produce the modified retroviral or lentiviral (e.g. SIV) vector of the invention may be reduced in length compared with the non-modified retroviral or lentiviral (e.g. SIV) RNA sequence from which the modified retroviral or lentiviral (e.g. SIV) RNA sequence is derived.

Preferred modifications to reduce the number of ORFs, particularly retroviral/lentiviral (e.g. SIV) ORFs, may comprise or consist of one or more of: (i) insertion of a nucleic acid (e.g. a U in the retroviral/lentiviral RNA sequence or a T in the corresponding proviral DNA sequence) to disrupt a start codon; (ii) substitution of an A by a U in the retroviral/lentiviral RNA sequence (or an A by a T in the corresponding proviral DNA sequence) to disrupt a start codon; and/or (iii) substitution of a U by an A in the retroviral/lentiviral RNA sequence (or a T by an A in the corresponding proviral DNA sequence) to disrupt a start codon.

As exemplified, such preferred modifications to reduce the number of ORFs, particularly retroviral/lentiviral (e.g. SIV) ORFs, include: (i) introduction of a U in the retroviral/lentiviral RNA sequence or a T in the corresponding proviral DNA sequence immediately 3′ to residue 1183 of SEQ ID NO: 25 (such an insertion corresponds to residue 1184 of SEQ ID NO: 19, and residue 431 of SEQ ID NO: 1); (ii) introduction of a U in the retroviral/lentiviral RNA sequence or a T in the corresponding proviral DNA sequence immediately 3′ to residue 1287 of SEQ ID NO: 25 (such an insertion corresponds to residue 1289 of SEQ ID NO: 19, and residue 536 of SEQ ID NO: 1); (iii) introduction of a U in the retroviral/lentiviral RNA sequence or a T in the corresponding proviral DNA sequence immediately 3′ to residue 1303 of SEQ ID NO: 25 (such an insertion corresponds to residue 1306 of SEQ ID NO: 19, and residue 553 of SEQ ID NO: 1); (iv) introduction of a U in the retroviral/lentiviral RNA sequence or a T in the corresponding proviral DNA sequence immediately 3′ to residue 1625 of SEQ ID NO: 25 (such an insertion corresponds to residue 1629 of SEQ ID NO: 19, and residue 876 of SEQ ID NO: 1); (v) substitution of an A by a U in the retroviral/lentiviral RNA sequence or substitution of an A by a T in the corresponding proviral DNA sequence at residue 1787 of SEQ ID NO: 25 (corresponding to residue 1794 of SEQ ID NO: 19, and residue 1041 of SEQ ID NO: 1); (vi) substitution of a U by an A in the retroviral/lentiviral RNA sequence or a T by an A in the corresponding proviral DNA sequence at residue 2064 of SEQ ID NO: 25 (corresponding to residue 2071 of SEQ ID NO: 19, and residue 1318 of SEQ ID NO: 1); and/or (vii) substitution of a U by an A in the retroviral/lentiviral RNA sequence or a T by an A in the corresponding proviral DNA sequence at residue 2238 of SEQ ID NO: 25 (corresponding to residue 2245 of SEQ ID NO: 19, and residue 1492 of SEQ ID NO: 1).

Particularly preferred modifications to reduce the number of ORFs, particularly retroviral/lentiviral (e.g. SIV) ORFs, are modifications which comprise or consist of the combination of (i) insertion of a nucleic acid (e.g. a U in the retroviral/lentiviral RNA sequence or a T in the corresponding proviral DNA sequence) to disrupt one or more start codon (e.g. 2, 3 or 4, preferably 4, start codons); (ii) substitution of an A by a U in the retroviral/lentiviral RNA sequence (or an A by a T in the corresponding proviral DNA sequence) to disrupt one or more start codon; and/or (iii) substitution of a U by an A in the retroviral/lentiviral RNA sequence (or a T by an A in the corresponding proviral DNA sequence) to disrupt one or more start codon (e.g. 2, 3, or 4, preferably 2, start codons). As exemplified, particularly preferred modifications to remove one or more retroviral/lentiviral (e.g. SIV) ORF comprise or consist of (i) introduction of a U in the retroviral/lentiviral RNA sequence or a T in the corresponding proviral DNA sequence immediately 3′ to residue 1183 of SEQ ID NO: 25 (such an insertion corresponds to residue 1184 of SEQ ID NO: 19, and residue 431 of SEQ ID NO: 1); (ii) introduction of a U in the retroviral/lentiviral RNA sequence or a T in the corresponding proviral DNA sequence immediately 3′ to residue 1287 of SEQ ID NO: 25 (such an insertion corresponds to residue 1289 of SEQ ID NO: 19, and residue 536 of SEQ ID NO: 1); (iii) introduction of a U in the retroviral/lentiviral RNA sequence or a T in the corresponding proviral DNA sequence immediately 3′ to residue 1303 of SEQ ID NO: 25 (such an insertion corresponds to residue 1306 of SEQ ID NO: 19, and residue 553 of SEQ ID NO: 1); (iv) introduction of a U in the retroviral/lentiviral RNA sequence or a T in the corresponding proviral DNA sequence immediately 3′ to residue 1625 of SEQ ID NO: 25 (such an insertion corresponds to residue 1629 of SEQ ID NO: 19, and residue 876 of SEQ ID NO: 1); (v) substitution of an A by a U in the retroviral/lentiviral RNA sequence or substitution of an A by a T in the corresponding proviral DNA sequence at residue 1787 of SEQ ID NO: 25 (corresponding to residue 1794 of SEQ ID NO: 19, and residue 1041 of SEQ ID NO: 1); (vi) substitution of a U by an A in the retroviral/lentiviral RNA sequence or a T by an A in the corresponding proviral DNA sequence at residue 2064 of SEQ ID NO: 25 (corresponding to residue 2071 of SEQ ID NO: 19, and residue 1318 of SEQ ID NO: 1); and (vii) substitution of a U by an A in the retroviral/lentiviral RNA sequence or a T by an A in the corresponding proviral DNA sequence at residue 2238 of SEQ ID NO: 25 (corresponding to residue 2245 of SEQ ID NO: 19, and residue 1492 of SEQ ID NO: 1).

As a specific non-limiting example, the modifications to a modified retroviral or lentiviral (e.g. SIV) RNA sequence may remove retroviral or lentiviral (e.g. SIV) ORFs comprised (at least in part) within the partial Gag region of the retroviral or lentiviral (e.g. SIV) RNA sequence, and/or may reduce the size of one or more retroviral or lentiviral (e.g. SIV) ORFs within said region. Preferably, a modified retroviral or lentiviral (e.g. SIV) RNA sequence of the invention has been modified such that it does not contain any retroviral or lentiviral (e.g. SIV) ORFs encoding polypeptides of greater than 100 amino acids, typically greater than 70 amino acids within the partial Gag region. Preferably, a modified retroviral or lentiviral (e.g. SIV) RNA sequence of the invention has been modified such that it does not contain any retroviral or lentiviral (e.g. SIV) ORFs encoding polypeptides of greater than 200 amino acids, typically greater than 160 amino acids within the partial RRE region. Particularly preferred is a modified retroviral or lentiviral (e.g. SIV) RNA sequence of the invention that has been modified such that it does not contain (i) any retroviral or lentiviral (e.g. SIV) ORFs encoding polypeptides of greater than 100 amino acids, typically greater than 70 amino acids within the partial Gag region; and (ii) any retroviral or lentiviral (e.g. SIV) ORFs encoding polypeptides of greater than 200 amino acids, typically greater than 160 amino acids within the partial RRE region. The invention provides a retroviral or lentiviral (e.g. SIV) vector comprising said modified retroviral or lentiviral (e.g. SIV) RNA sequence.

Any modification or combination thereof to reduce the number of ORFs, particularly retroviral or lentiviral (e.g. SIV) ORFs within a retroviral or lentiviral (e.g. SIV) RNA sequence of the invention may be used in combination with any codon-substitution modification or combination thereof as described herein.

Thus, the invention provides a modified retroviral or lentiviral (e.g. SIV) RNA sequence that: (a) does not contain (i) any retroviral or lentiviral (e.g. SIV) ORFs encoding polypeptides of greater than 100 amino acids, typically greater than 70 amino acids within the partial Gag region; (ii) any retroviral or lentiviral (e.g. SIV) ORFs encoding polypeptides of greater than 200 amino acids, typically greater than 160 amino acids within the partial RRE region; and (b) the codon-substitutions comprise or consist of the combination of (i) replacement of a frameshift mutation and a STOP codon into the Env ORF of the retroviral/lentiviral RNA sequence; and (ii) introduction of a SbfI restriction site, which may optionally replace an EcoR1 restriction site within the retroviral/lentiviral RNA sequence, particularly the individual examples described herein. The invention provides a retroviral or lentiviral (e.g. SIV) vector comprising said modified retroviral or lentiviral (e.g. SIV) RNA sequence.

Any codon-substitution or combination thereof may be used in combination with any modification to reduce the number of ORFs, particularly retroviral/lentiviral (e.g. SIV) ORFs, or combination thereof. Preferred are retroviral/lentiviral (e.g. SIV) RNA sequences wherein (a) the codon-substitutions comprise or consist of the combination of (i) replacement of a frameshift mutation and a STOP codon into the Env ORF of the retroviral/lentiviral RNA sequence; and (ii) introduction of a SbfI restriction site, which may optionally replace an EcoR1 restriction site within the retroviral/lentiviral RNA sequence; and (b) the modifications to reduce the number of ORFs, particularly retroviral/lentiviral (e.g. SIV) ORFs, comprise or consist of the combination of (i) insertion of a nucleic acid (e.g. a U in the retroviral/lentiviral RNA sequence or a T in the corresponding proviral DNA sequence) to disrupt one or more start codon (e.g. 2, 3 or 4, preferably 4, start codons); (ii) substitution of an A by a U in the retroviral/lentiviral RNA sequence (or an A by a T in the corresponding proviral DNA sequence) to disrupt one or more start codon; and (iii) substitution of a U by an A in the retroviral/lentiviral RNA sequence (or a T by an A in the corresponding proviral DNA sequence) to disrupt one or more start codon (e.g. 2, 3, or 4, preferably 2, start codons).

Particularly preferred are retroviral/lentiviral (e.g. SIV) RNA sequences wherein (a) the codon-substitutions comprise or consist of the combination of (i) the replacement of a motif corresponding to residues 2347-2352 of SEQ ID NO: 25 with the motif corresponding to residues 2354-2360 of SEQ ID NO: 25; and (ii) the replacement of a motif corresponding to residues 1734-1739 of SEQ ID NO: 25 with the motif corresponding to residues 1738-1746 of SEQ ID NO: 25; and (b) the modifications to reduce the number of ORFs, particularly retroviral/lentiviral (e.g. SIV) ORFs, comprise or consist of the combination of (i) introduction of a U in the retroviral/lentiviral RNA sequence or a T in the corresponding proviral DNA sequence immediately 3′ to residue 1183 of SEQ ID NO: 25 (such an insertion corresponds to residue 1184 of SEQ ID NO: 19, and residue 431 of SEQ ID NO: 1); (ii) introduction of a U in the retroviral/lentiviral RNA sequence or a T in the corresponding proviral DNA sequence immediately 3′ to residue 1287 of SEQ ID NO: 25 (such an insertion corresponds to residue 1289 of SEQ ID NO: 19, and residue 536 of SEQ ID NO: 1); (iii) introduction of a U in the retroviral/lentiviral RNA sequence or a T in the corresponding proviral DNA sequence immediately 3′ to residue 1303 of SEQ ID NO: 25 (such an insertion corresponds to residue 1306 of SEQ ID NO: 19, and residue 553 of SEQ ID NO: 1); (iv) introduction of a U in the retroviral/lentiviral RNA sequence or a T in the corresponding proviral DNA sequence immediately 3′ to residue 1625 of SEQ ID NO: 25 (such an insertion corresponds to residue 1629 of SEQ ID NO: 19, and residue 876 of SEQ ID NO: 1); (v) substitution of an A by a U in the retroviral/lentiviral RNA sequence or substitution of an A by a T in the corresponding proviral DNA sequence at residue 1787 of SEQ ID NO: 25 (corresponding to residue 1794 of SEQ ID NO: 19, and residue 1041 of SEQ ID NO: 1); (vi) substitution of a U by an A in the retroviral/lentiviral RNA sequence or a T by an A in the corresponding proviral DNA sequence at residue 2064 of SEQ ID NO: 25 (corresponding to residue 2071 of SEQ ID NO: 19, and residue 1318 of SEQ ID NO: 1); and (vii) substitution of a U by an A in the retroviral/lentiviral RNA sequence or a T by an A in the corresponding proviral DNA sequence at residue 2238 of SEQ ID NO: 25 (corresponding to residue 2245 of SEQ ID NO: 19, and residue 1492 of SEQ ID NO: 1).

Of particular preference, the invention provides a SIV vector pseudotyped with Sendai virus hemagglutinin-neuraminidase (HN) and fusion (F) proteins, wherein: (a) said vector comprises a modified retroviral RNA sequence which comprises or consists of a nucleic acid sequence of SEQ ID NO: 1, preferably wherein the modified retroviral RNA sequence consists of a nucleic acid sequence of SEQ ID NO: 1; and (b) the F protein comprises a first subunit which comprises or consists of an amino acid sequence of SEQ ID NO: 14 and a second subunit which comprises or consists of an amino acid sequence of SEQ ID NO: 15. Said vector may further comprise one or more of: (a) a p17 protein comprising or consisting of an amino acid sequence of SEQ ID NO: 2; (b) a p24 protein comprising or consisting of an amino acid sequence of SEQ ID NO: 3; (c) p8 protein comprising or consisting of an amino acid sequence of SEQ ID NO: 4; (d) a protease comprising or consisting of an amino acid sequence of SEQ ID NO: 5; (e) a p51 protein comprising or consisting of an amino acid sequence of SEQ ID NO: 6; (f) a p15 protein comprising or consisting of an amino acid sequence of SEQ ID NO: 7; (g) a p31 protein comprising or consisting of an amino acid sequence of SEQ ID NO: 8; (h) a Gag protein comprising or consisting of an amino acid sequence of SEQ ID NO: 9; and/or (i) a Pol protein comprising or consisting of an amino acid sequence of SEQ ID NO: 10. Optionally said vector comprises each of (a) to (g), and may further comprise one or both of (h) and (i).

A retroviral/lentiviral (e.g. SIV) RNA sequence of the invention may comprise one or more further modifications in addition to the codon-substitutions and/or modifications to reduce retroviral/lentiviral (e.g. SIV) ORFs as described herein. By way of non-limiting example, the retroviral/lentiviral (e.g. SIV) RNA sequence may be CpG-depleted (or CpG-fee) to facilitate gene expression. Standard techniques for modifying the transgene sequence in this way are known in the art.

As exemplified herein, retroviral/lentiviral (e.g. SIV) vectors comprising a modified retroviral/lentiviral (e.g. SIV) RNA sequence of the invention have at least maintained, and potentially increased transgene expression; and/or at least maintained, and potentially increased integration of the retroviral/lentiviral (e.g. SIV) RNA sequence into target cells. Retroviral/lentiviral (e.g. SIV) vectors comprising a modified retroviral/lentiviral (e.g. SIV) RNA sequence of the invention also typically have at least maintained, and potentially increased vector yield compared with retroviral/lentiviral (e.g. SIV) vector comprising the non-modified retroviral/lentiviral (e.g. SIV) RNA sequence from which the modified retroviral/lentiviral (e.g. SIV) RNA sequence is derived. This effect on vector yield may be further increased by the use of codon-optimised GagPol, as described herein.

The retroviral/lentiviral (e.g. SIV) vector comprises a promoter operably linked to a transgene, enabling expression of the transgene. Typically the promoter is a hybrid human CMV enhancer/EF1a (hCEF) promoter. This hCEF promoter may lack the intron corresponding to nucleotides 570-709 and the exon corresponding to nucleotides 728-733 of the hCEF promoter. A preferred example of an hCEF promoter sequence of the invention is provided by SEQ ID NO: 26. The promoter may be a CMV promoter. An example of a CMV promoter sequence is provided by SEQ ID NO: 27. The promoter may be a human elongation factor 1a (EF1a) promoter. An example of a EF1a promoter is provided by SEQ ID NO: 28. Other promoters for transgene expression are known in the art and their suitability for the retroviral/lentiviral (e.g. SIV) vectors of the invention determined using routine techniques known in the art. Non-limiting examples of other promoters include UbC and UCOE. As described herein, the promoter may be modified to further regulate expression of the transgene of the invention.

The promoter included in the retroviral/lentiviral (e.g. SIV) vector of the invention may be specifically selected and/or modified to further refine regulation of expression of the therapeutic gene. Again, suitable promoters and standard techniques for their modification are known in the art. As a non-limiting example, a number of suitable (CpG-free) promoters suitable for use in the present invention are described in Pringle et al. (J. Mol. Med. Berl. 2012, 90(12): 1487-96), which is herein incorporated by reference in its entirety. Preferably, the retroviral/lentiviral vectors (particularly SIV F/HN vectors) of the invention comprise a hCEF promoter having low or no CpG dinucleotide content. The hCEF promoter may have all CG dinucleotides replaced with any one of AG, TG or GT. Thus, the hCEF promoter may be CpG-free. A preferred example of a CpG-free hCEF promoter sequence of the invention is provided by SEQ ID NO: 26. The absence of CpG dinucleotides typically further improves the performance of retroviral/lentiviral (e.g. SIV) vectors of the invention and in particular in situations where it is not desired to induce an immune response against an expressed antigen or an inflammatory response against the delivered expression construct. The elimination of CpG dinucleotides reduces the occurrence of flu-like symptoms and inflammation which may result from administration of constructs, particularly when administered to the airways.

The retroviral/lentiviral (e.g. SIV) vector of the invention may be modified to allow shut down of gene expression. Standard techniques for modifying the vector in this way are known in the art. As a non-limiting example, Tet-responsive promoters are widely used.

A retroviral/lentiviral (e.g. SIV) vector of the invention may comprise a transgene that encodes a polypeptide or protein that is therapeutic for the treatment of such diseases, particularly a disease or disorder of the airways, respiratory tract, or lung.

Accordingly, a retroviral/lentiviral (e.g. SIV) vector of the invention may comprise a transgene encoding a protein selected from: (i) a secreted therapeutic protein, optionally Alpha-1 Antitrypsin (A1AT), Factor VIII, Surfactant Protein B (SFTPB), Factor VII, Factor IX, Factor X, Factor XI, von Willebrand Factor, Granulocyte-Macrophage Colony-Stimulating Factor (GM-CSF) and a monoclonal antibody against an infectious agent; or (ii) CFTR, ABCA3, DNAH5, DNAH11, DNAI1, and DNA12. Other examples of transgenes that may be comprised in a retroviral/lentiviral (e.g. SIV) vector of the invention include genes related to or associated with other surfactant deficiencies.

The transgene included in the vector of the invention may be modified to facilitate expression. For example, the transgene sequence may be in CpG-depleted (or CpG-fee) form and/or further modified to facilitate gene expression. Standard techniques for modifying the transgene sequence in this way are known in the art.

Preferably, the transgene encodes a CFTR. An example of a CFTR cDNA is provided by SEQ ID NO: 29. Variants thereof (as described therein) are also included, particularly variants with at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% identity to SEQ ID NO: 29. Preferably the CFTR transgene has at least 90%, at least 95%, or at least 99% identity to SEQ ID NO: 29.

The transgene may encode an A1AT. An example of an A1AT transgene is provided by SEQ ID NO: 30, or by the complementary sequence of SEQ ID NO: 31. SEQ ID NO: 30 is a codon-optimised CpG depleted A1AT transgene previously designed by the present inventors to enhance translation in human cells. Such optimisation has been shown to enhance gene expression by up to 15-fold. Variants of same sequence (as defined herein) which possess the same technical effect of enhancing translation compared with the unmodified (wild-type) A1AT gene sequence are also encompassed by the present invention. The polypeptide encoded by said A1AT transgene, may be exemplified by the polypeptide of SEQ ID NO: 32. Variants thereof (as described therein) are also included, particularly variants with at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% identity to SEQ ID NO: 30, 31 or 32. Preferably the A1AT variants have at least 90%, at least 95%, or at least 99% identity to SEQ ID NO: 30, 31 or 32.

The transgene may encode a FVIII. Examples of a FVIII transgene are provided by SEQ ID NOs: 33 and 34, or by the respective complementary sequences of SEQ ID NO: 35 and 36. The polypeptide encoded by the FVIII transgene, may be exemplified by the polypeptide of SEQ ID NO: 37 or 38. Variants thereof (as described therein) are also included, particularly variants with at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% identity to any one of SEQ ID NOs: 33 to 38. Preferably the FVIII variants have at least 90%, at least 95%, or at least 99% identity to any one of SEQ ID NOs: 33 to 38.

The transgene of the invention may be any one or more of DNAH5, DNAH11, DNA/1, and DNA/2, or other known related gene.

When the respiratory tract epithelium is targeted for delivery of the retroviral/lentiviral (e.g. SIV) vector, the transgene may encode A1AT, SFTPB, or GM-CSF. The transgene may encode a monoclonal antibody (mAb) against an infectious agent. The transgene may encode anti-TNF alpha. The transgene may encode a therapeutic protein implicated in an inflammatory, immune or metabolic condition.

A retroviral/lentiviral (e.g. SIV) vector of the invention may be delivered to the cells of the respiratory tract to allow production of proteins to be secreted into circulatory system. In such embodiments, the transgene may encode for Factor VII, Factor VIII, Factor IX, Factor X, Factor XI and/or von Willebrand's factor. Such a vector may be used in the treatment of diseases, particularly cardiovascular diseases and blood disorders, preferably blood clotting deficiencies such as haemophilia. Again, the transgene may encode an mAb against an infectious agent or a protein implicated in an inflammatory, immune or metabolic condition, such as, lysosomal storage disease.

The retroviral/lentiviral (e.g. SIV) vector of the invention may have no intron positioned between the promoter and the transgene. Similarly, there may be no intron between the promoter and the transgene in the vector genome (pDNA1) plasmid (for example, pGM830 as described herein, with the sequence of SEQ ID NO: 20).

In some preferred embodiments, the retroviral/lentiviral (e.g. SIV) vector comprises a hCEF promoter and a CFTR transgene, including those described herein. Optionally said retroviral/lentiviral (e.g. SIV) vector may have no intron positioned between the promoter and the transgene. Such a retroviral/lentiviral (e.g. SIV) vector may be produced by the method described herein, using a genome plasmid carrying the CFTR transgene and a promoter.

In some preferred embodiments, the retroviral/lentiviral (e.g. SIV) vector comprises a hCEF promoter and an A1AT transgene, including those described herein. Optionally said retroviral/lentiviral (e.g. SIV) vector may have no intron positioned between the promoter and the transgene. Such a retroviral/lentiviral (e.g. SIV) vector may be produced by the method described herein, using a genome plasmid carrying the A1AT transgene and a promoter.

In some preferred embodiments, the retroviral/lentiviral (e.g. SIV) vector comprises a hCEF or CMW promoter and an FVIII transgene, including those described herein. Optionally said retroviral/lentiviral (e.g. SIV) vector may have no intron positioned between the promoter and the transgene. Such a retroviral/lentiviral (e.g. SIV) vector may be produced by the method described herein, using a genome plasmid carrying the FVIII transgene and a promoter.

The retroviral/lentiviral (e.g. SIV) vector as described herein comprises a transgene. The transgene comprises a nucleic acid sequence encoding a gene product, e.g., a protein, particularly a therapeutic protein.

For example, in one embodiment, the nucleic acid sequence encoding a CFTR, A1AT or FVIII comprises (or consists of) a nucleic acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to the CFTR, A1AT or FVIII nucleic acid sequence respectively, examples of which are described herein. In a further embodiment, the nucleic acid sequence encoding CFTR, A1AT or FVIII comprises (or consists of) a nucleic acid sequence having at least 95% (such as at least 95, 96, 97, 98, 99 or 100%) sequence identity to the CFTR, A1AT or FVIII nucleic acid sequence respectively, examples of which are described herein. In one embodiment, the nucleic acid sequence encoding CFTR is provided by SEQ ID NO: 29, the nucleic acid sequence encoding A1AT is provided by SEQ ID NO: 30, or by the complementary sequence of SEQ ID NO: 31 and/or the nucleic acid sequence encoding FVIII is provided by SEQ ID NO: 33 and 34, or by the respective complementary sequences of SEQ ID NO: 35 and 36, or variants thereof.

The amino acid sequence of the CFTR, A1AT or FVIII transgene may comprise (or consist of) an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100%, preferably at least 90%, at least 95%, or at least 99% identity sequence identity to the functional CFTR, A1AT or FVIII polypeptide sequence respectively.

The retroviral/lentiviral (e.g. SIV) vectors of the invention may comprise a central polypurine tract (cPPT) and/or the Woodchuck hepatitis virus posttranscriptional regulatory elements (WPRE). An exemplary WPRE sequence is provided by SEQ ID NO: 39.

As described herein, the retroviral/lentiviral (e.g. SIV) RNA sequence is derived from the proviral DNA sequence. The proviral DNA sequence is itself provided during the manufacturing process by the vector genome plasmid, pDNA1. However, the retroviral/lentiviral (e.g. SIV) RNA sequence is not identical to the proviral DNA sequence (and hence not identical to the vector genome plasmid, pDNA1). Rather, the retroviral/lentiviral (e.g. SIV) RNA sequence is shorter in length than the corresponding proviral DNA sequence, and the precise limits or boundaries of the retroviral/lentiviral (e.g. SIV) RNA sequence are typically not readily determined. In other words, it is generally not possible to identify a precise retroviral/lentiviral (e.g. SIV) RNA sequence (with the 5′ and 3′ specifically identified) merely from the primary sequence of the proviral DNA sequence (and hence the vector genome plasmid, pDNA1, sequence).

The retroviral/lentiviral (e.g. SIV) vector typically comprises a modified retroviral/lentiviral (e.g. SIV) RNA sequence that is less than 10,000 bases in length, less than 9,000 bases in length, or less than 8,000 bases in length. Preferably, the retroviral/lentiviral (e.g. SIV) vector comprises a modified retroviral/lentiviral (e.g. SIV) RNA sequence that is less than 9,000 bases in length.

The retroviral/lentiviral (e.g. SIV) vector may comprise a modified retroviral/lentiviral (e.g. SIV) RNA sequence that comprises or consists of a nucleic acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% identity to SEQ ID NO: 1. The modified retroviral/lentiviral (e.g. SIV) RNA sequence may comprise or consist of a nucleic acid sequence having at least 90%, at least 95%, or at least 99% identity to SEQ ID NO: 1. The modified retroviral/lentiviral (e.g. SIV) RNA sequence may comprise or consist of a nucleic acid sequence having at least 99% identity to SEQ ID NO: 1. The modified retroviral sequence may comprise or consist of a nucleic acid sequence of SEQ ID NO: 1.

The invention provides a retroviral/lentiviral (e.g. SIV) vector that comprises a retroviral/lentiviral (e.g. SIV) RNA sequence that consists of a nucleic acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% identity to SEQ ID NO: 1. The modified retroviral/lentiviral (e.g. SIV) RNA sequence may consist of a nucleic acid sequence having at least 90%, at least 95%, or at least 99% identity to SEQ ID NO: 1. The modified retroviral/lentiviral (e.g. SIV) RNA sequence may consist of a nucleic acid sequence having at least 99% identity to SEQ ID NO: 1. The invention provides a retroviral/lentiviral (e.g. SIV) vector that comprises a retroviral/lentiviral (e.g. SIV) RNA sequence that consists of a nucleic acid sequence of SEQ ID NO: 1.

The retroviral/lentiviral (e.g. SIV) vector may comprise a modified retroviral/lentiviral (e.g. SIV) RNA sequence that is (a) less than 10,000 bases in length, less than 9,000 bases in length, or less than 8,000 bases in length; and (b) comprises or consists of a nucleic acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% identity to SEQ ID NO: 1.

The retroviral/lentiviral (e.g. SIV) vector may comprise a modified retroviral/lentiviral (e.g. SIV) RNA sequence that is (a) less than 10,000 bases in length, less than 9,000 bases in length, or less than 8,000 bases in length; and (b) comprises or consists of a nucleic acid sequence having at least 90%, at least 91%, at least 92%, at least 93%, at least 94%, at least 95%, at least 96%, at least 97%, at least 98%, at least 99%, at least 99.5%, at least 99.9%, or more, up to 100% identity to SEQ ID NO: 1.

The retroviral/lentiviral (e.g. SIV) vector may comprise a modified retroviral/lentiviral (e.g. SIV) RNA sequence that is (a) less than 10,000 bases in length, less than 9,000 bases in length, or less than 8,000 bases in length; and (b) comprises or consists of a nucleic acid sequence having at least 99%, at least 99.5%, at least 99.9%, or more, up to 100% identity to SEQ ID NO: 1.

The retroviral/lentiviral (e.g. SIV) vector may comprise a modified retroviral/lentiviral (e.g. SIV) RNA sequence that is (a) less than 10,000 bases in length, less than 9,000 bases in length, or less than 8,000 bases in length; and (b) consists of a nucleic acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% identity to SEQ ID NO: 1.

The retroviral/lentiviral (e.g. SIV) vector may comprise a modified retroviral/lentiviral (e.g. SIV) RNA sequence that is (a) less than 10,000 bases in length, less than 9,000 bases in length, or less than 8,000 bases in length; and (b) consists of a nucleic acid sequence having at least 90%, at least 91%, at least 92%, at least 93%, at least 94%, at least 95%, at least 96%, at least 97%, at least 98%, at least 99%, at least 99.5%, at least 99.9%, or more, up to 100% identity to SEQ ID NO: 1.

The retroviral/lentiviral (e.g. SIV) vector may comprise a modified retroviral/lentiviral (e.g. SIV) RNA sequence that is (a) less than 10,000 bases in length, less than 9,000 bases in length, or less than 8,000 bases in length; and (b) consists of a nucleic acid sequence having at least 99%, at least 99.5%, at least 99.9%, or more, up to 100% identity to SEQ ID NO: 1.

The retroviral/lentiviral (e.g. SIV) vector may comprise a modified retroviral/lentiviral (e.g. SIV) RNA sequence that is (a) less than 9,000 bases in length, or less than 8,000 bases in length; and (b) comprises or consists of a nucleic acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% identity to SEQ ID NO: 1.

The retroviral/lentiviral (e.g. SIV) vector may comprise a modified retroviral/lentiviral (e.g. SIV) RNA sequence that is (a) less than 9,000 bases in length, or less than 8,000 bases in length; and (b) comprises or consists of a nucleic acid sequence having at least 90%, at least 91%, at least 92%, at least 93%, at least 94%, at least 95%, at least 96%, at least 97%, at least 98%, at least 99%, at least 99.5%, at least 99.9%, or more, up to 100% identity to SEQ ID NO: 1.

The retroviral/lentiviral (e.g. SIV) vector may comprise a modified retroviral/lentiviral (e.g. SIV) RNA sequence that is (a) less than 9,000 bases in length, or less than 8,000 bases in length; and (b) comprises or consists of a nucleic acid sequence having at least 99%, at least 99.5%, at least 99.9%, or more, up to 100% identity to SEQ ID NO: 1.

The retroviral/lentiviral (e.g. SIV) vector may comprise a modified retroviral/lentiviral (e.g. SIV) RNA sequence that is (a) less than 9,000 bases in length, or less than 8,000 bases in length; and (b) consists of a nucleic acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% identity to SEQ ID NO: 1.

The retroviral/lentiviral (e.g. SIV) vector may comprise a modified retroviral/lentiviral (e.g. SIV) RNA sequence that is (a) less than 9,000 bases in length, or less than 8,000 bases in length; and (b) consists of a nucleic acid sequence having at least 90%, at least 91%, at least 92%, at least 93%, at least 94%, at least 95%, at least 96%, at least 97%, at least 98%, at least 99%, at least 99.5%, at least 99.9%, or more, up to 100% identity to SEQ ID NO: 1.

The retroviral/lentiviral (e.g. SIV) vector may comprise a modified retroviral/lentiviral (e.g. SIV) RNA sequence that is (a) less than 9,000 bases in length, or less than 8,000 bases in length; and (b) consists of a nucleic acid sequence having at least 99%, at least 99.5%, at least 99.9%, or more, up to 100% identity to SEQ ID NO: 1.

Preferably, the retroviral/lentiviral (e.g. SIV) vector comprises a modified retroviral/lentiviral (e.g. SIV) RNA sequence that is (a) less than 9,000 bases in length; and (b) comprises or consists of a nucleic acid sequence having at least 80%, 90%, 91%, 92%, 93%, 94%, 95%, 96%, 97%, 98%, 99%, 99.5%, 99.9%, or up to 100% identity to SEQ ID NO: 1. More preferably, the retroviral/lentiviral (e.g. SIV) vector comprises a modified retroviral/lentiviral (e.g. SIV) RNA sequence that is (a) less than 9,000 bases in length; and (b) comprises or consists of a nucleic acid sequence having at least 99% identity to SEQ ID NO: 1. Still more preferably, the retroviral/lentiviral (e.g. SIV) vector comprises a modified retroviral/lentiviral (e.g. SIV) RNA sequence that is (a) less than 9,000 bases in length; and (b) consists of a nucleic acid sequence having at least 99% identity to SEQ ID NO: 1. Still more preferably, the retroviral/lentiviral (e.g. SIV) vector comprises a modified retroviral/lentiviral (e.g. SIV) RNA sequence that is (a) less than 9,000 bases in length; and (b) comprises or consists of a nucleic acid sequence of SEQ ID NO: 1. Still more preferably, the retroviral/lentiviral (e.g. SIV) vector comprises a modified retroviral/lentiviral (e.g. SIV) RNA sequence that is (a) less than 9,000 bases in length; and (b) consists of a nucleic acid sequence of SEQ ID NO: 1.

The 5′ and/or 3′ limits of a modified retroviral/lentiviral (e.g. SIV) RNA sequence may each independently allow for some degree of flexibility, such that the 5′ end of the modified retroviral/lentiviral (e.g. SIV) RNA sequence may not correspond to the first nucleotide of SEQ ID NO: 1, and/or the 3′ end of the modified retroviral/lentiviral (e.g. SIV) RNA sequence may not correspond to the last nucleotide of SEQ ID NO: 1.

Accordingly, a modified retroviral/lentiviral (e.g. SIV) RNA sequence may comprise up to an additional 200 nucleotides, up to an additional 150 nucleotides, up to an additional 100 nucleotides, up to an additional 75 nucleotides, up to an additional 50 nucleotides, up to an additional 25 nucleotides, up to an additional 10 nucleotides, up to an additional 5, nucleotides at the 5′ and/or 3′ end, e.g. compared with SEQ ID NO: 1. The modified retroviral/lentiviral (e.g. SIV) RNA sequence may comprise an additional 25, 24, 23, 22, 21, 20, 19, 18, 17, 16, 15, 14, 13, 12, 11, 10, 9, 8, 7, 6, 5, 4, 3, 2 or 1 nucleotides at the 5′ and/or 3′ end, e.g. compared with SEQ ID NO: 1. The presence of additional nucleotides and the number thereof at the 5′ end of the modified retroviral/lentiviral (e.g. SIV) RNA sequence is independent from the presence of additional nucleotides and the number thereof at the 3′ end of the modified retroviral/lentiviral (e.g. SIV) RNA sequence. By way of non-limiting example, a modified retroviral/lentiviral (e.g. SIV) RNA sequence may comprise up to an additional 3 nucleotides at the 5′ and up to an additional 200 nucleotides at the 3′ end, e.g. compared with SEQ ID NO: 1. By way of a further non-limiting example, a modified retroviral/lentiviral (e.g. SIV) RNA sequence may comprise no additional nucleotides at the 5′ and an additional 42 nucleotides at the 3′ end, e.g. compared with SEQ ID NO: 1. Preferably, a modified retroviral/lentiviral (e.g. SIV) RNA sequence does not comprise any additional nucleotides at the 5′ end, but may comprise up to an additional 200 nucleotides at the 3′ end (as described above), e.g. compared with SEQ ID NO: 1.

A modified retroviral/lentiviral (e.g. SIV) RNA sequence may comprise up to 200 nucleotides less, up to 150 nucleotides less, up to 100 nucleotides less, up to 75 nucleotides less, up to 50 nucleotides less, up to 25 nucleotides less, up to 10 nucleotides less, up to 5 nucleotides less at the 5′ and/or 3′ end, e.g. compared with SEQ ID NO: 1. The modified retroviral/lentiviral (e.g. SIV) RNA sequence may comprise 25, 24, 23, 22, 21, 20, 19, 18, 17, 16, 15, 14, 13, 12, 11, 10, 9, 8, 7, 6, 5, 4, 3, 2 or 1 nucleotides less at the 5′ and/or 3′ end, e.g. compared with SEQ ID NO: 1. The number of deleted thereof at the 5′ end of the modified retroviral/lentiviral (e.g. SIV) RNA sequence is independent from the presence of deleted nucleotides and the number thereof at the 3′ end of the modified retroviral/lentiviral (e.g. SIV) RNA sequence. By way of non-limiting example, a modified retroviral/lentiviral (e.g. SIV) RNA sequence may comprise up to 3 nucleotides less at the 5′, e.g. compared with SEQ ID NO: 1 and up to 200 nucleotides at the 3′ end, e.g. compared with SEQ ID NO: 1. By way of a further non-limiting example, a modified retroviral/lentiviral (e.g. SIV) RNA sequence may comprise no nucleotides less at the 5′, e.g. compared with SEQ ID NO: 1 and 42 nucleotides less at the 3′ end, e.g. compared with SEQ ID NO: 1. Preferably, a modified retroviral/lentiviral (e.g. SIV) RNA sequence does not comprise any nucleotides less at the 5′ end, but may comprise up to 200 nucleotides less at the 3′ end (as described above), e.g. compared with SEQ ID NO: 1.

One end of the modified retroviral/lentiviral (e.g. SIV) RNA sequence may have additional nucleotides, e.g. compared with SEQ ID NO: 1 and the other end may have fewer nucleotides, e.g. compared with SEQ ID NO: 1. Thus, the 5′ end may have additional nucleotides, e.g. compared with SEQ ID NO: 1, and the 3′ end may have fewer nucleotides, e.g. compared with SEQ ID NO: 1. The 3′ end may have additional nucleotides, e.g. compared with SEQ ID NO: 1, and the 5′ end may have fewer nucleotides, e.g. compared with SEQ ID NO: 1. The disclosure herein in relation to the number of additional and/or deleted nucleotides applies equally and without reservation to modified retroviral/lentiviral (e.g. SIV) RNA sequence in which one end has additional nucleotides, e.g. compared with SEQ ID NO: 1 and the other end has fewer nucleotides, e.g. compared with SEQ ID NO: 1. Preferably, a modified retroviral/lentiviral (e.g. SIV) RNA sequence does not comprise any additional/missing nucleotides at the 5′ end, but may comprise additional or fewer nucleotides at the 3′ end (as described above), e.g. compared with SEQ ID NO: 1.

As described herein, retroviral/lentiviral (e.g. SIV) vectors with modified retroviral/lentiviral (e.g. SIV) RNA sequences according to the invention avoid potential safety risks as described herein, whilst: (i) maintaining or even increasing transgene expression; (ii) maintaining or even increasing retroviral/lentiviral (e.g. SIV) RNA sequence integration into a host cell genome; and/or (iii) maintaining or even increasing retroviral/lentiviral (e.g. SIV) vector yield.

Thus, the retroviral/lentiviral (e.g. SIV) vectors comprising a modified retroviral/lentiviral (e.g. SIV) RNA sequence of the invention typically exhibit high levels of transgene expression. Typically a the retroviral/lentiviral (e.g. SIV) vector with a modified retroviral/lentiviral (e.g. SIV) RNA sequence of the invention is at least equivalent in terms of transgene expression compared with retroviral/lentiviral (e.g. SIV) vector which comprises the unmodified retroviral/lentiviral (e.g. SIV) RNA sequence from which the modified retroviral/lentiviral (e.g. SIV) RNA sequence is derived (i.e. the corresponding unmodified retroviral/lentiviral (e.g. SIV) RNA sequence).

As used herein, the term “equivalent transgene expression” may be defined such that the modified retroviral/lentiviral (e.g. SIV) RNA sequence does not significantly decrease transgene expression of the retroviral/lentiviral (e.g. SIV) vector compared with the corresponding unmodified retroviral/lentiviral (e.g. SIV) RNA sequence. By way of non-limiting example, transgene expression by a retroviral/lentiviral (e.g. SIV) vector comprising the modified retroviral/lentiviral (e.g. SIV) RNA sequence into the host/target cell genome may be no more than 2-fold lower, no more than 1.5-fold lower, no more than 1.0-fold lower, no more than 0.5-fold lower, no more than 0.25-fold lower, or less than transgene expression by the retroviral/lentiviral (e.g. SIV) vector comprising the corresponding unmodified retroviral/lentiviral (e.g. SIV) RNA sequence. The term “equivalent transgene expression” may be defined such that transgene expression by a retroviral/lentiviral (e.g. SIV) vector comprising a modified retroviral/lentiviral (e.g. SIV) RNA sequence into the host/target cell genome is statistically unchanged (e.g. p<0.05, p<0.01) compared with transgene expression by the retroviral/lentiviral (e.g. SIV) vector comprising the corresponding unmodified retroviral/lentiviral (e.g. SIV) RNA sequence.

Preferably, transgene expression by a retroviral/lentiviral (e.g. SIV) vector comprising a modified retroviral/lentiviral (e.g. SIV) RNA sequence vector into the host/target cell genome is increased compared with transgene expression by the retroviral/lentiviral (e.g. SIV) vector comprising the corresponding unmodified retroviral/lentiviral (e.g. SIV) RNA sequence. Transgene expression by a retroviral/lentiviral (e.g. SIV) vector comprising the modified retroviral/lentiviral (e.g. SIV) RNA sequence into the host/target cell genome may be at least 1.5-fold, at least 2-fold, or at least 2.5-fold greater than transgene expression by the retroviral/lentiviral (e.g. SIV) vector comprising the corresponding non-modified retroviral/lentiviral (e.g. SIV) RNA sequence.

Alternatively or in addition, the retroviral/lentiviral (e.g. SIV) vectors comprising a modified retroviral/lentiviral (e.g. SIV) RNA sequence of the invention exhibit high levels of vector integration into the host/target cell genome. Typically a retroviral/lentiviral (e.g. SIV) vector with a modified retroviral/lentiviral (e.g. SIV) RNA sequence of the invention is at least equivalent in terms of integration into the host/target cell genome compared with the retroviral/lentiviral (e.g. SIV) vector which comprises the corresponding unmodified retroviral/lentiviral (e.g. SIV) RNA sequence.

As used herein, the term “equivalent integration” may be defined such that the modified retroviral/lentiviral (e.g. SIV) RNA sequence does not significantly decrease the integration of retroviral/lentiviral (e.g. SIV) vector into the host/target cell genome compared with the corresponding unmodified retroviral/lentiviral (e.g. SIV) RNA sequence. By way of non-limiting example, integration of retroviral/lentiviral (e.g. SIV) vector comprising the modified retroviral/lentiviral (e.g. SIV) RNA sequence of the invention into the host/target cell genome may be no more than 2-fold lower, no more than 1.5-fold lower, no more than 1.0-fold lower, no more than 0.5-fold lower, no more than 0.25-fold lower, or less than the integration into the host/target cell genome of retroviral/lentiviral (e.g. SIV) vector comprising the corresponding unmodified retroviral/lentiviral (e.g. SIV) RNA sequence. The term “equivalent integration” may be defined such that integration of retroviral/lentiviral (e.g. SIV) vector comprising a modified retroviral/lentiviral (e.g. SIV) RNA sequence of the invention into the host/target cell genome is statistically unchanged (e.g. p<0.05, p<0.01) compared with integration of retroviral/lentiviral (e.g. SIV) vector comprising the corresponding unmodified retroviral/lentiviral (e.g. SIV) RNA sequence.

Preferably, the integration of retroviral/lentiviral (e.g. SIV) vector comprising a modified retroviral/lentiviral (e.g. SIV) RNA sequence vector of the invention into the host/target cell genome is increased compared with the integration of retroviral/lentiviral (e.g. SIV) vector comprising the corresponding unmodified retroviral/lentiviral (e.g. SIV) RNA sequence. The integration of retroviral/lentiviral (e.g. SIV) vector comprising the modified retroviral/lentiviral (e.g. SIV) RNA sequence of the invention into the host/target cell genome may be at least 1.5-fold, at least 2-fold, or at least 2.5-fold greater than the integration of retroviral/lentiviral (e.g. SIV) vector comprising the corresponding non-modified retroviral/lentiviral (e.g. SIV) RNA sequence.

Alternatively or in addition, the invention provides high titre purified retroviral/lentiviral (e.g. SIV) vectors comprising a modified retroviral/lentiviral (e.g. SIV) RNA sequence. Typically the titre of a retroviral/lentiviral (e.g. SIV) vector with a modified retroviral/lentiviral (e.g. SIV) RNA sequence of the invention is at least equivalent to the titre of a retroviral/lentiviral (e.g. SIV) vector which comprises the corresponding unmodified retroviral/lentiviral (e.g. SIV) RNA sequence.

As used herein, the term “equivalent titre” may be defined such that the modified retroviral/lentiviral (e.g. SIV) RNA sequence does not significantly decrease the titre of retroviral/lentiviral (e.g. SIV) vector compared with the corresponding unmodified retroviral/lentiviral (e.g. SIV) RNA sequence. By way of non-limiting example, a titre of retroviral/lentiviral (e.g. SIV) vector comprising the modified retroviral/lentiviral (e.g. SIV) RNA sequence of the invention may be no more than 2-fold lower, no more than 1.5-fold lower, no more than 1.0-fold lower, no more than 0.5-fold lower, no more than 0.25-fold lower, or less than the titre of retroviral/lentiviral (e.g. SIV) vector comprising the corresponding unmodified retroviral/lentiviral (e.g. SIV) RNA sequence. The term “equivalent titre” may be defined such that titre of retroviral/lentiviral (e.g. SIV) vector comprising a modified retroviral/lentiviral (e.g. SIV) RNA sequence of the invention is statistically unchanged (e.g. p<0.05, p<0.01) compared with the titre of retroviral/lentiviral (e.g. SIV) vector comprising the corresponding unmodified retroviral/lentiviral (e.g. SIV) RNA sequence.

Preferably, the titre of retroviral/lentiviral (e.g. SIV) vector comprising a modified retroviral/lentiviral (e.g. SIV) RNA sequence vector of the invention is increased compared with the titre of retroviral/lentiviral (e.g. SIV) vector comprising the corresponding unmodified retroviral/lentiviral (e.g. SIV) RNA sequence. The titre of retroviral/lentiviral (e.g. SIV) vector comprising the modified retroviral/lentiviral (e.g. SIV) RNA sequence of the invention may be at least 1.5-fold, at least 2-fold, or at least 2.5-fold greater than the titre of retroviral/lentiviral (e.g. SIV) vector comprising the corresponding non-modified retroviral/lentiviral (e.g. SIV) RNA sequence.

The production of high-titre retroviral/lentiviral (e.g. SIV) vectors may impart other desirable properties on the resulting vector products. For example, without being bound by theory, it is believed that production at high titres without the need for intense concentration by methods such as TFF results in a higher quality vector product than corresponding retroviral/lentiviral (e.g. SIV) vectors with unmodified retroviral/lentiviral (e.g. SIV) RNA sequences because the vectors are exposed to less shear forces which can damage the viral particles and their RNA cargo.

Preferably, the retroviral/lentiviral (e.g. SIV) vector comprising a modified retroviral/lentiviral (e.g. SIV) RNA sequence vector of the invention exhibits maintained/increased transgene expression compared with the titre of retroviral/lentiviral (e.g. SIV) vector comprising the corresponding unmodified retroviral/lentiviral (e.g. SIV) RNA sequence. The retroviral/lentiviral (e.g. SIV) vector comprising a modified retroviral/lentiviral (e.g. SIV) RNA sequence vector of the invention exhibits maintained/increased transgene expression and maintained/increased vector integration compared with the titre of retroviral/lentiviral (e.g. SIV) vector comprising the corresponding unmodified retroviral/lentiviral (e.g. SIV) RNA sequence. The retroviral/lentiviral (e.g. SIV) vector comprising a modified retroviral/lentiviral (e.g. SIV) RNA sequence vector of the invention exhibits maintained/increased transgene expression and maintained/increased vector yield/titre compared with the titre of retroviral/lentiviral (e.g. SIV) vector comprising the corresponding unmodified retroviral/lentiviral (e.g. SIV) RNA sequence. More preferably, the retroviral/lentiviral (e.g. SIV) vector comprising a modified retroviral/lentiviral (e.g. SIV) RNA sequence vector of the invention exhibits maintained/increased transgene expression, maintained/increased vector integration and maintained/increased vector yield/titre compared with the titre of retroviral/lentiviral (e.g. SIV) vector comprising the corresponding unmodified retroviral/lentiviral (e.g. SIV) RNA sequence.

The invention also provides host cells comprising a retroviral/lentiviral (e.g. SIV) vector of the invention. Typically a host cell is a mammalian cell, particularly a human cell or cell line. Non-limiting examples of host cells include HEK293 cells (such as HEK293F or HEK293T cells) and 293T/17 cells. Commercial cell lines suitable for the production of virus are also readily available (as described herein).

### Methods of Production

Methods for the production of retroviral/lentiviral (e.g. SIV) vectors of the invention as also described herein.

The present inventors have previously demonstrated that the use of codon-optimised gal-pol genes from SIV does not negatively impact the manufactured titre of a SIV vector pseudotyped with hemagglutinin-neuraminidase (HN) and fusion (F) proteins from a respiratory paramyxovirus, and can even result in an increased titre of the vector. This is described in PCT/GB2022/050524, which is herein incorporated by reference in its entirety.

The present inventors have now shown that retroviral/lentiviral (e.g. SIV) vectors can be produced with modified retroviral/lentiviral (e.g. SIV) RNA sequences which avoid potential safety risks as described herein, whilst: (i) maintaining or even increasing transgene expression; (ii) maintaining or even increasing retroviral/lentiviral (e.g. SIV) RNA sequence integration into a host cell genome; and/or (iii) maintaining or even increasing retroviral/lentiviral (e.g. SIV) vector yield. Furthermore, the vector genome plasmids which are used in the manufacture of the retroviral/lentiviral (e.g. SIV) vectors of the invention can be combined with the use of codon-optimised gag-pol genes as described herein, again whilst maintaining, or even increasing the vector titre.

Accordingly, the present invention provides a method of producing a retroviral/lentiviral (e.g. SIV) vector comprising a modified retroviral/lentiviral (e.g. SIV) RNA sequence as described herein, where said retroviral/lentiviral (e.g. SIV) is pseudotyped with hemagglutinin-neuraminidase (HN) and fusion (F) proteins from a respiratory paramyxovirus, and which comprises a promoter and a transgene. Preferably said retroviral/lentiviral (e.g. SIV) vector is a lentiviral vector, with Simian immunodeficiency virus (SIV) vectors being particularly preferred.

The method of the invention may be a scalable GMP-compatible method.

The method of the invention typically allows the generation of retroviral/lentiviral (e.g. SIV) vectors comprising a modified retroviral/lentiviral (e.g. SIV) RNA sequence with high levels of transgene expression. Typically a method of the invention produces retroviral/lentiviral (e.g. SIV) vector with a modified retroviral/lentiviral (e.g. SIV) RNA sequence as described herein that are at least equivalent in terms of transgene expression compared with retroviral/lentiviral (e.g. SIV) vector which comprises the unmodified retroviral/lentiviral (e.g. SIV) RNA sequence from which the modified retroviral/lentiviral (e.g. SIV) RNA sequence is derived (i.e. the corresponding unmodified retroviral/lentiviral (e.g. SIV) RNA sequence) when produced by the same method.

As used herein, the term “equivalent transgene expression” may be defined such that the modified retroviral/lentiviral (e.g. SIV) RNA sequence does not significantly decrease transgene expression of the retroviral/lentiviral (e.g. SIV) vector compared with the corresponding unmodified retroviral/lentiviral (e.g. SIV) RNA sequence. By way of non-limiting example, transgene expression by a retroviral/lentiviral (e.g. SIV) vector comprising the modified retroviral/lentiviral (e.g. SIV) RNA sequence into the host/target cell genome is no more than 2-fold lower, no more than 1.5-fold lower, no more than 1.0-fold lower, no more than 0.5-fold lower, no more than 0.25-fold lower, or less than transgene expression by the retroviral/lentiviral (e.g. SIV) vector comprising the corresponding unmodified retroviral/lentiviral (e.g. SIV) RNA sequence. The term “equivalent transgene expression” may be defined such that transgene expression by a retroviral/lentiviral (e.g. SIV) vector comprising a modified retroviral/lentiviral (e.g. SIV) RNA sequence into the host/target cell genome is statistically unchanged (e.g. p<0.05, p<0.01) compared with transgene expression by the retroviral/lentiviral (e.g. SIV) vector comprising the corresponding unmodified retroviral/lentiviral (e.g. SIV) RNA sequence produced by the same method.

Preferably, transgene expression by a retroviral/lentiviral (e.g. SIV) vector comprising a modified retroviral/lentiviral (e.g. SIV) RNA sequence vector into the host/target cell genome is increased compared with transgene expression by the retroviral/lentiviral (e.g. SIV) vector comprising the corresponding unmodified retroviral/lentiviral (e.g. SIV) RNA sequence produced by the same method. Transgene expression by a retroviral/lentiviral (e.g. SIV) vector comprising the modified retroviral/lentiviral (e.g. SIV) RNA sequence into the host/target cell genome may be at least 1.5-fold, at least 2-fold, or at least 2.5-fold greater than transgene expression by the retroviral/lentiviral (e.g. SIV) vector comprising the corresponding non-modified retroviral/lentiviral (e.g. SIV) RNA sequence produced by the same method.

The method of the invention typically allows the generation of retroviral/lentiviral (e.g. SIV) vectors comprising a modified retroviral/lentiviral (e.g. SIV) RNA sequence with high levels of vector integration into the host/target cell genome. Typically a method of the invention produces retroviral/lentiviral (e.g. SIV) vector with a modified retroviral/lentiviral (e.g. SIV) RNA sequence as described herein that are at least equivalent in terms of integration into the host/target cell genome compared with retroviral/lentiviral (e.g. SIV) vector which comprises the corresponding unmodified retroviral/lentiviral (e.g. SIV) RNA sequence produced by the same method.

As used herein, the term “equivalent integration” may be defined such that the modified retroviral/lentiviral (e.g. SIV) RNA sequence does not significantly decrease the integration of retroviral/lentiviral (e.g. SIV) vector into the host/target cell genome compared with the corresponding unmodified retroviral/lentiviral (e.g. SIV) RNA sequence. By way of non-limiting example, integration of retroviral/lentiviral (e.g. SIV) vector comprising the modified retroviral/lentiviral (e.g. SIV) RNA sequence into the host/target cell genome is no more than 2-fold lower, no more than 1.5-fold lower, no more than 1.0-fold lower, no more than 0.5-fold lower, no more than 0.25-fold lower, or less than the integration into the host/target cell genome of retroviral/lentiviral (e.g. SIV) vector comprising the corresponding unmodified retroviral/lentiviral (e.g. SIV) RNA sequence. The term “equivalent integration” may be defined such that integration of retroviral/lentiviral (e.g. SIV) vector comprising a modified retroviral/lentiviral (e.g. SIV) RNA sequence into the host/target cell genome is statistically unchanged (e.g. p<0.05, p<0.01) compared with integration of retroviral/lentiviral (e.g. SIV) vector comprising the corresponding unmodified retroviral/lentiviral (e.g. SIV) RNA sequence produced by the same method.

Preferably, the integration of retroviral/lentiviral (e.g. SIV) vector comprising a modified retroviral/lentiviral (e.g. SIV) RNA sequence vector into the host/target cell genome is increased compared with the integration of retroviral/lentiviral (e.g. SIV) vector comprising the corresponding unmodified retroviral/lentiviral (e.g. SIV) RNA sequence produced by the same method. The integration of retroviral/lentiviral (e.g. SIV) vector comprising the modified retroviral/lentiviral (e.g. SIV) RNA sequence into the host/target cell genome may be at least 1.5-fold, at least 2-fold, or at least 2.5-fold greater than the integration of retroviral/lentiviral (e.g. SIV) vector comprising the corresponding non-modified retroviral/lentiviral (e.g. SIV) RNA sequence produced by the same method.

The method of the invention typically allows the generation of high titre purified retroviral/lentiviral (e.g. SIV) vectors comprising a modified retroviral/lentiviral (e.g. SIV) RNA sequence. Typically a method of the invention produces a titre of retroviral/lentiviral (e.g. SIV) vector with a modified retroviral/lentiviral (e.g. SIV) RNA sequence as described herein that is at least equivalent to the titre of a retroviral/lentiviral (e.g. SIV) vector which comprises the corresponding unmodified retroviral/lentiviral (e.g. SIV) RNA sequence when produced by a corresponding method.

As used herein, the term “equivalent titre” may be defined such that the modified retroviral/lentiviral (e.g. SIV) RNA sequence does not significantly decrease the titre of retroviral/lentiviral (e.g. SIV) vector compared with the corresponding unmodified retroviral/lentiviral (e.g. SIV) RNA sequence. By way of non-limiting example, a titre of retroviral/lentiviral (e.g. SIV) vector comprising the modified retroviral/lentiviral (e.g. SIV) RNA sequence that is no more than 2-fold lower, no more than 1.5-fold lower, no more than 1.0-fold lower, no more than 0.5-fold lower, no more than 0.25-fold lower, or less than the titre of retroviral/lentiviral (e.g. SIV) vector comprising the corresponding unmodified retroviral/lentiviral (e.g. SIV) RNA sequence. The term “equivalent titre” may be defined such that titre of retroviral/lentiviral (e.g. SIV) vector comprising a modified retroviral/lentiviral (e.g. SIV) RNA sequence is statistically unchanged (e.g. p<0.05, p<0.01) compared with the titre of retroviral/lentiviral (e.g. SIV) vector comprising the corresponding unmodified retroviral/lentiviral (e.g. SIV) RNA sequence produced by the same method.

Preferably, the titre of retroviral/lentiviral (e.g. SIV) vector comprising a modified retroviral/lentiviral (e.g. SIV) RNA sequence vector is increased compared with the titre of retroviral/lentiviral (e.g. SIV) vector comprising the corresponding unmodified retroviral/lentiviral (e.g. SIV) RNA sequence produced by the same method. The titre of retroviral/lentiviral (e.g. SIV) vector comprising the modified retroviral/lentiviral (e.g. SIV) RNA sequence may be at least 1.5-fold, at least 2-fold, or at least 2.5-fold greater than the titre of retroviral/lentiviral (e.g. SIV) vector comprising the corresponding non-modified retroviral/lentiviral (e.g. SIV) RNA sequence produced by the same method.

The production of retroviral/lentiviral (e.g. SIV) vectors typically employs one or more plasmids which provide the elements needed for the production of the vector: the genome for the retroviral/lentiviral vector, the Gag-Pol, Rev, F and HN. Multiple elements can be provided on a single plasmid. Preferably each element is provided on a separate plasmid, such that there five plasmids, one for each of the vector genome, the Gag-Pol, Rev, F and HN, respectively.

Alternatively, a single plasmid may provide the Gag-Pol and Rev elements, and may be referred to as a packaging plasmid (pDNA2). The remaining elements (genome, F and HN) may be provided by separate plasmids (pDNA1, pDNA3a, pDNA3b respectively), such that four plasmids are used for the production of a retroviral/lentiviral (e.g. SIV) vector according to the invention. In the four plasmid methods, pDNA1, pDNA3a and pDNA3b may be as described herein in the context of the five-plasmid method.

In the preferred five plasmid method of the invention, the vector genome plasmid encodes all the genetic material that is packaged into final retroviral/lentiviral vector, including the transgene. The vector genome plasmid may be designated herein as “pDNA1”, and typically comprises the transgene and the transgene promoter. As described herein, only a portion of the genetic material found in the vector genome plasmid ends up in the virus, and the precise limits and boundaries of this portion cannot be readily deduced based on the primary sequence of the pDNA1. The present invention elucidates for the first time the nucleic acid sequence of a modified RNA sequence of a SIV vector which addresses numerous potential safety risks, whilst providing maintained or even increased (i) transgene expression, (ii) SIV RNA sequence integration, and/or (iii) vector yield.

The other four plasmids are manufacturing plasmids encoding the Gag-Pol, Rev, F and HN proteins. These plasmids may be designated “pDNA2a”, “pDNA2b”, “pDNA3a” and “pDNA3b” respectively.

Typically, the lentivirus is SIV, such as SIV1, preferably SIV-AGM. The F and HN proteins are derived from a respiratory paramyxovirus, preferably a Sendai virus.

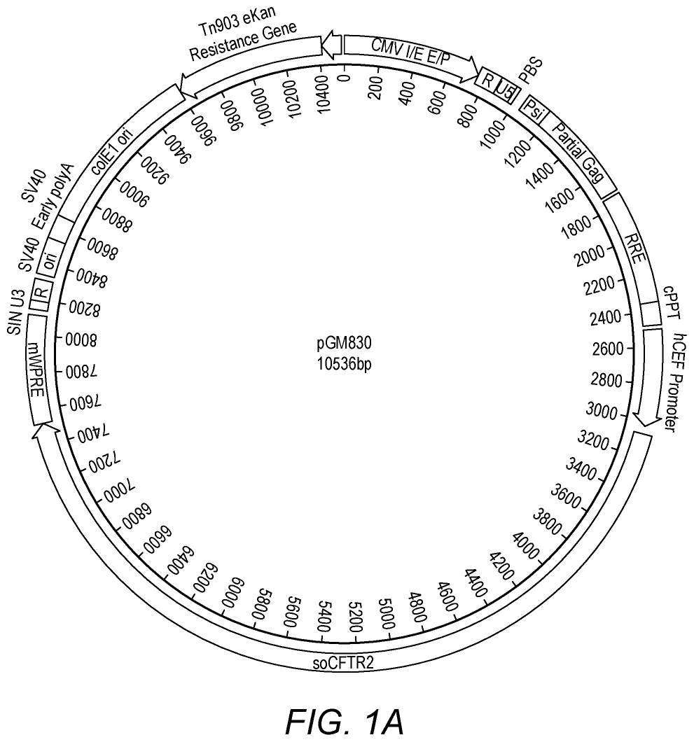

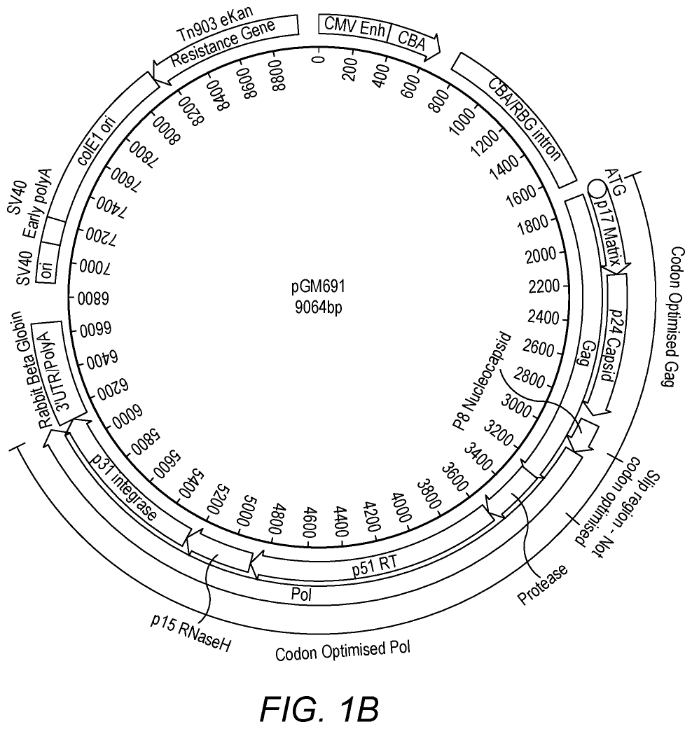

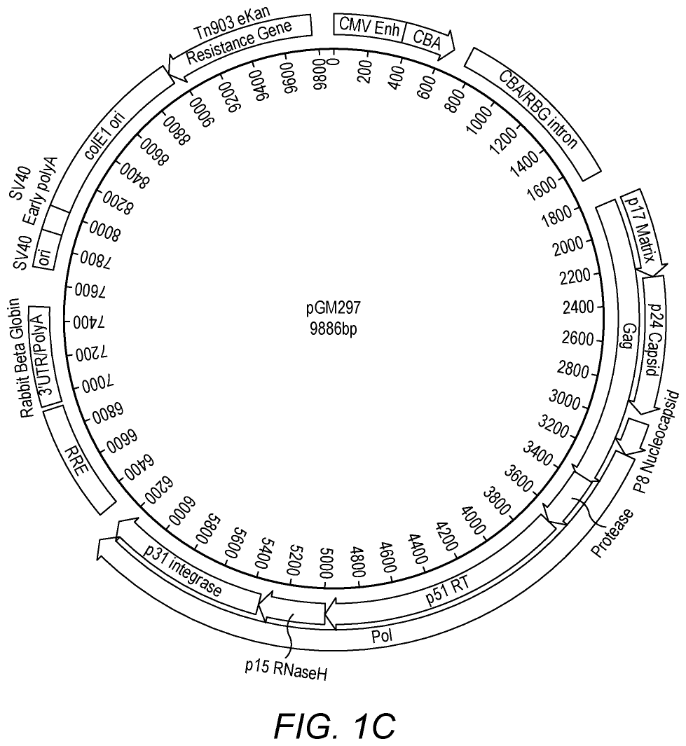

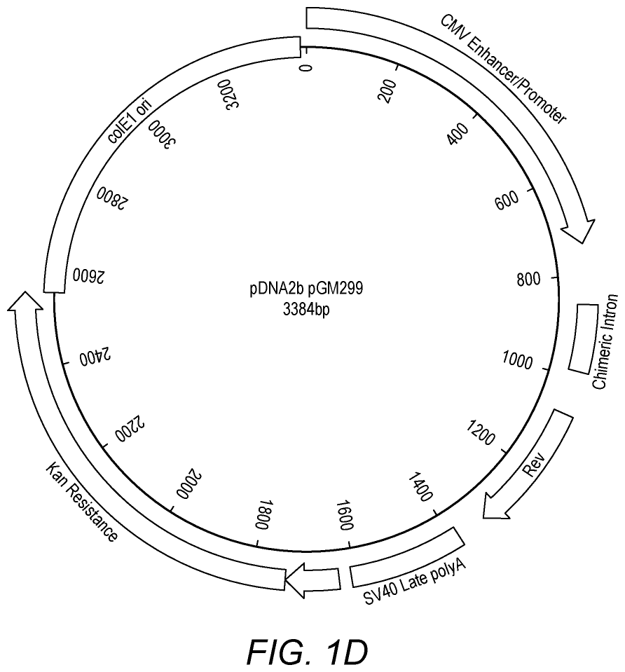

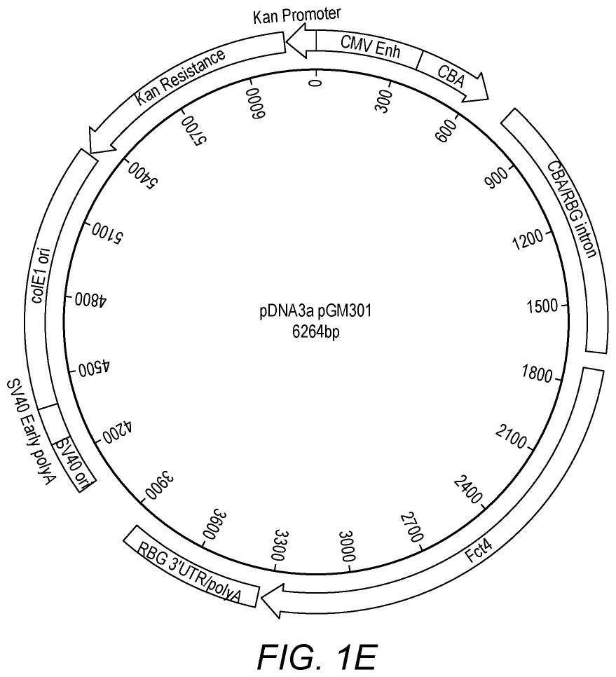

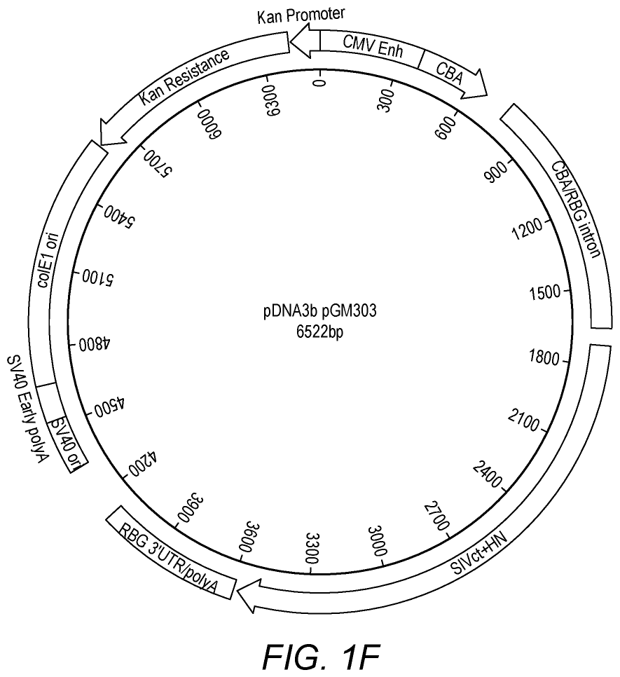

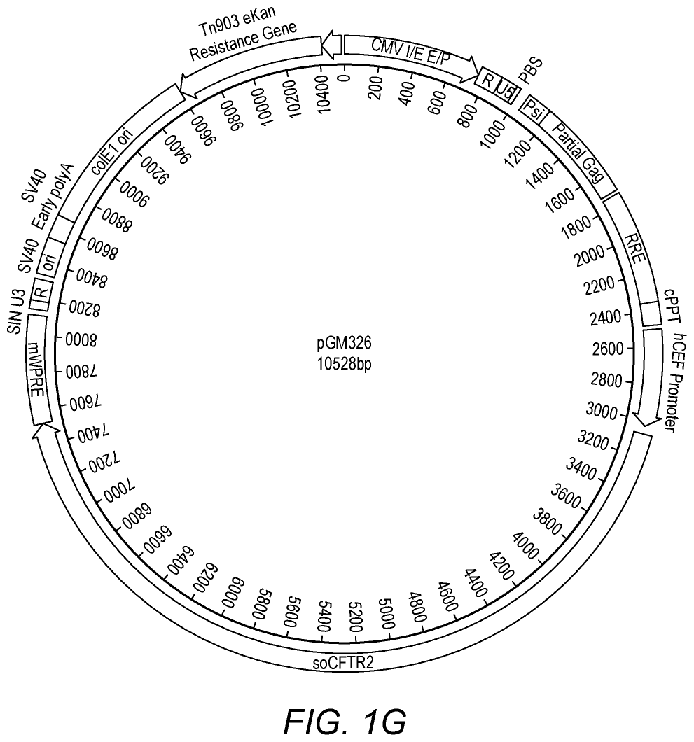

In a specific embodiment relating to CFTR, the five plasmids are characterised by FIGS. 1A-1F, thus pDNA1 is the pGM830 plasmid of FIG. 1A, pDNA2a is the pGM691 plasmid of FIG. 1B or the pGM297 plasmid of FIG. 1C, pDNA2b is the pGM299 plasmid of FIG. 1D, pDNA3a is the pGM301 plasmid of FIG. 1E and pDNA3b is the pGM303 plasmid of FIG. 1F, or variants thereof any of these plasmids (as described herein). pGM326 (as shown in FIG. 1G) is an unmodified of the vector genome plasmid from which pGM830 is derived.

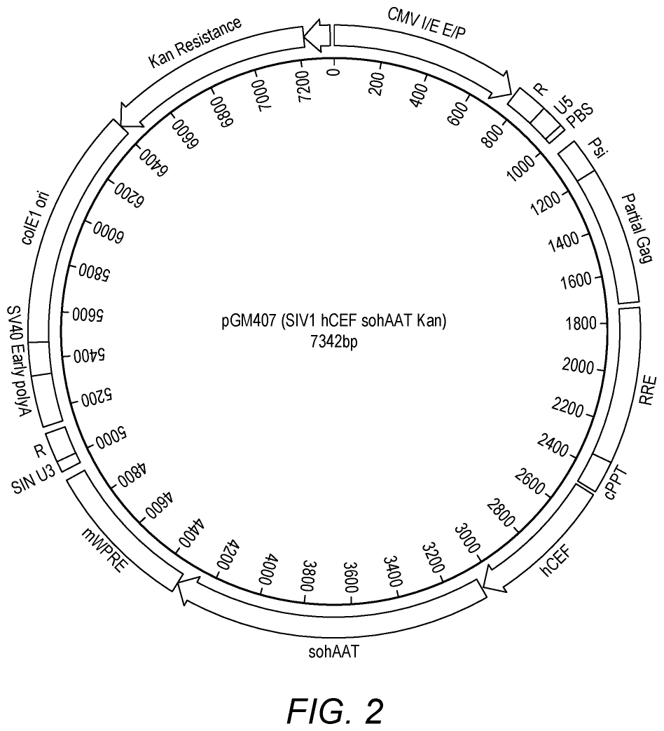

When a method of the invention is used to produce A1AT, the five plasmids may be characterised by FIG. 2 (thus plasmid pDNA1 may be pGM407) and all of FIG. 1B or 1C and 1D-1F (as above for the specific CFTR embodiment), or variants of any of these plasmids (as described herein).

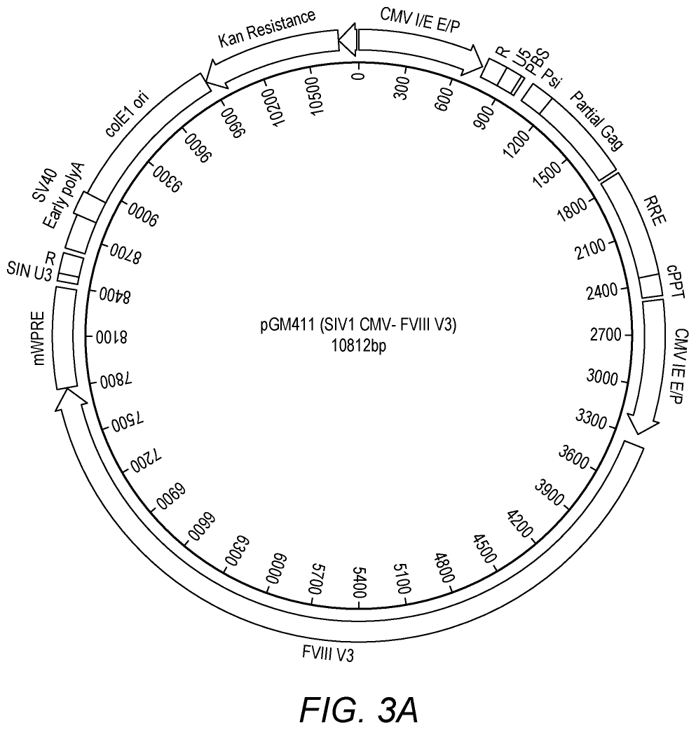

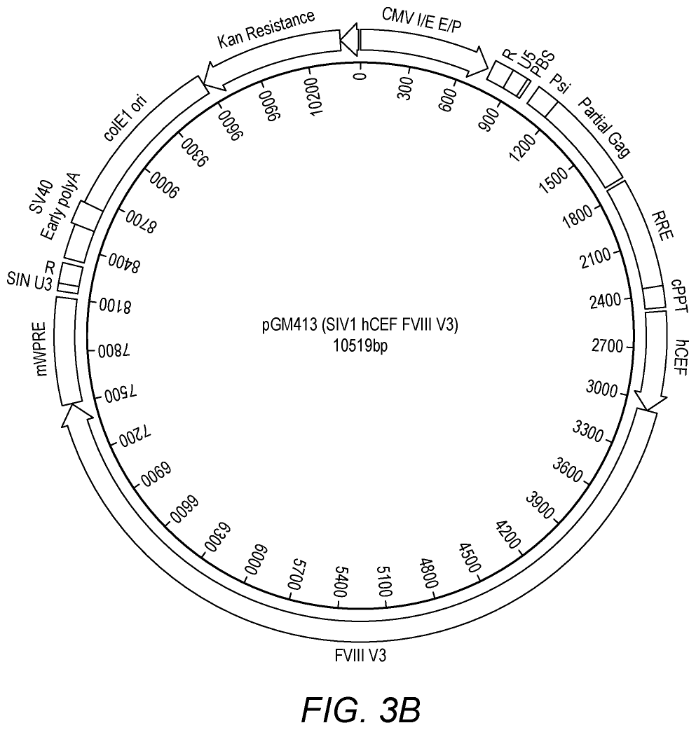

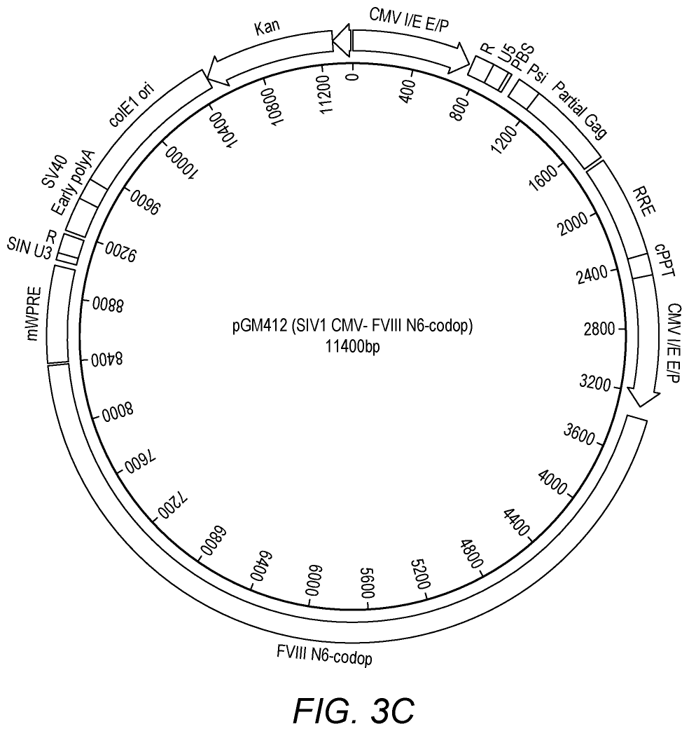

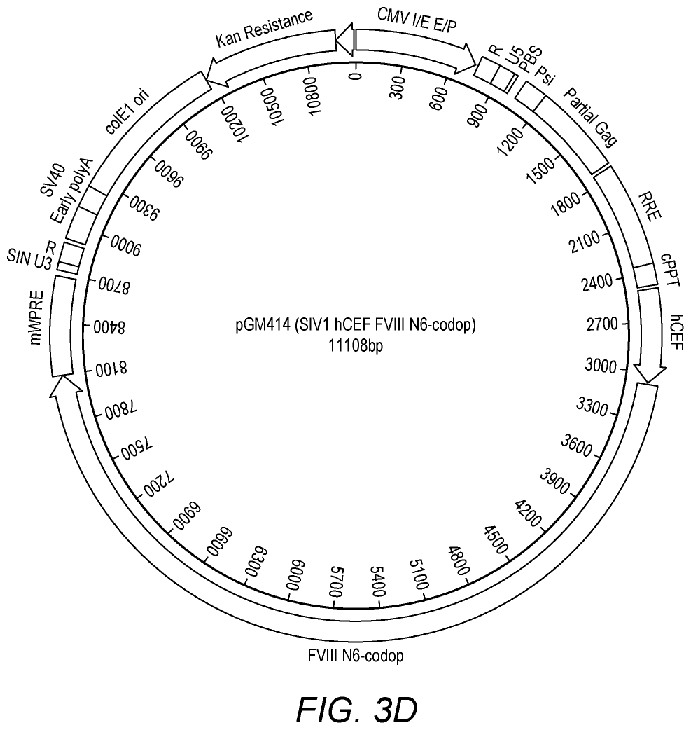

When a method of the invention is used to produce FVIII, the five plasmids may be characterised by one of FIGS. 3A-3D (thus plasmid pDNA1 may be pGM411, pGM412, pGM413 or pGM414) and all of FIG. 1B or 1C and 1D-1F, or variants of any of these plasmids (as described herein).

The plasmid as defined in FIG. 1A is represented by SEQ ID NO: 19; the plasmid as defined in FIG. 1B is represented by SEQ ID NO: 20; the plasmid as defined in FIG. 1C is represented by SEQ ID NO: 21; the plasmid as defined in FIG. 1D is represented by SEQ ID NO: 22; the plasmid as defined in FIG. 1E is represented by SEQ ID NO: 23; the plasmid as defined in FIG. 1F is represented by SEQ ID NO: 24; the plasmid as defined in FIG. 1G is represented by SEQ ID NO: 25; the plasmid as defined in FIG. 2 is represented by SEQ ID NO: 40 and the F/HN-SIV-CMV-HFVIII-V3, F/HN-SIV-hCEF-HFVIII-V3, F/HN-SIV-CMV-HFVIII-N6-co and/or F/HN-SIV-hCEF-HFVIII-N6-co plasmids as defined in FIGS. 3A to 3D are represented by SEQ ID NOs: 41 to 44 respectively. Variants (as defined herein) of these plasmids are also encompassed by the present invention. In particular, variants having at least 90% (such as at least 90, 92, 94, 95, 96, 97, 98, 99, 99.5 or 100%) sequence identity to any one of SEQ ID NOs: 19 to 25 and 40 to 44 are encompassed.

In the five-plasmid method of the invention all five plasmids contribute to the formation of the final retroviral/lentiviral (e.g. SIV) vector, although only the vector genome plasmid provides nucleic acid sequence comprised in the retroviral/lentiviral (e.g. SIV) RNA sequence. During manufacture of the retroviral/lentiviral (e.g. SIV) vector, the vector genome plasmid (pDNA1) provides the enhancer/promoter, Psi, RRE, cPPT, mWPRE, SIN LTR, SV40 polyA (see FIG. 1A), which are important for virus manufacture. Using pGM830 as non-limiting examples of a pDNA1, the CMV enhancer/promoter, SV40 polyA, colE1 Ori and KanR are involved in manufacture of the retroviral/lentiviral (e.g. SIV) vector of the invention (e.g. vGM195 or vGM244), but are not found in the final retroviral/lentiviral (e.g. SIV) vector. The RRE, cPPT (central polypurine tract), hCEF, soCFTR2 (transgene) and mWPRE from pGM326 or pGM830 are found in the final retroviral/lentiviral (e.g. SIV) vector. SIN LTR (long terminal repeats, SIN/IN self-inactivating) and Psi (packaging signal) may be found in the final retroviral/lentiviral (e.g. SIV) vector.

For other retroviral/lentiviral (e.g. SIV) vectors of the invention, corresponding elements from the other vector genome plasmids (pDNA1) are required for manufacture (but not found in the final vector), or are present in the final retroviral/lentiviral (e.g. SIV) vector.

The F and HN proteins from pDNA3a and pDNA3b (preferably Sendai F and HN proteins) are important for infection of target cells with the final retroviral/lentiviral (e.g. SIV) vector, i.e. for entry of a patient's epithelial cells (typically lung or nasal cells as described herein). The products of the pDNA2a and pDNA2b plasmids are important for virus transduction, i.e. for inserting the retroviral/lentiviral (e.g. SIV) DNA into the host's genome. The promoter, regulatory elements (such as WPRE) and transgene are important for transgene expression within the target cell(s).

A method of the invention may comprise or consist of the following steps: (a) growing cells in suspension; (b) transfecting the cells with one or more plasmids; (c) adding a nuclease; (d) harvesting the lentivirus (e.g. SIV); (e) adding trypsin; and (f) purification of the lentivirus (e.g. SIV).

This method may use the four- or five-plasmid system described herein. Thus, for the preferred five-plasmid method, the one or more plasmids may comprise or consist of: a vector genome plasmid pDNA1; a gagpol plasmid (e.g. codon-optimised gagpol plasmid), pDNA2a; a Rev plasmid, pDNA2b; a fusion (F) protein plasmid, pDNA3a; and a hemagglutinin-neuraminidase (HN) plasmid, pDNA3b. The pDNA1 may be pGM830. The pDNA2a may be pGM297 or pGM691, preferably pGM691. The pDNA2b may be pGM299. The pDNA3a may be pGM301. The pDNA3b may be pGM303. Any combination of pDNA1, pDNA2a, pDNA2b, pDNA3a and pDNA3b may be used. Preferably, the pDNA1 is pGM830; the pDNA2a is pGM691; the pDNA2b is pGM299; the pDNA3a is pGM301; and the pDNA3b is pGM303.

Any appropriate ratio of vector genome plasmid:gagpol plasmid:Rev plasmid:F plasmid:HN plasmid may be used to further optimise (increase) the retroviral/lentiviral (e.g. SIV) titre produced. By way of non-limiting example, the ratio of vector genome plasmid:gagpol plasmid:Rev plasmid:F plasmid:HN plasmid may by in the range of 10-40:-4-20:3-12:3-12:3-12, typically 15-20:7-11:4-8:4-8:4-8, such as about 18-22:7-11:4-8:4-8:4-8, 19-21:8-10:5-7:5-7:5-7. Preferably the ratio of vector genome plasmid:gagpol plasmid:Rev plasmid:F plasmid:HN plasmid is about 20:9:6:6:6.

Steps (a)-(f) of the method are typically carried out sequentially, starting at step (a) and continuing through to step (f). The method may include one or more additional step, such as additional purification steps, buffer exchange, concentration of the retroviral/lentiviral (e.g. SIV) vector after purification, and/or formulation of the retroviral/lentiviral (e.g. SIV) vector after purification (or concentration). Each of the steps may comprise one or more sub-steps. For example, harvesting may involve one or more steps or sub-steps, and/or purification may involve one or more steps or sub-steps.

Any appropriate cell type may be transfected with the one or more plasmids (e.g. the five-plasmids described herein) to produce a retroviral/lentiviral (e.g. SIV) vector of the invention. Typically mammalian cells, particularly human cell lines are used. Non-limiting examples of cells suitable for use in the methods of the invention are HEK293 cells (such as HEK293F or HEK293T cells) and 293T/17 cells. Commercial cell lines suitable for the production of virus are also readily available (e.g. Gibco Viral Production Cells—Catalogue Number A35347 from ThermoFisher Scientific).

The cells may be grown in animal-component free media, including serum-free media. The cells may be grown in a media which contains human components. The cells may be grown in a defined media comprising or consisting of synthetically produced components.

Any appropriate transfection means may be used according to the invention. Selection of appropriate transfection means is within the routine practice of one of ordinary skill in the art. By way of non-limiting example, transfection may be carried out by the use of PEIPro™, Lipofectamine2000™ or Lipofectamine3000™.

Any appropriate nuclease may be used according to the invention. Selection of appropriate nuclease is within the routine practice of one of ordinary skill in the art. Typically the nuclease is an endonuclease. By way of non-limiting example, the nuclease may be Benzonase® or Denarase®. The addition of the nuclease may be at the pre-harvest stage or at the post-harvest stage, or between harvesting steps.

The gag-pol genes used in the production of a retroviral/lentiviral (e.g. SIV) vectors of the invention may be codon-optimised. Thus, the gag-pol genes within the pDNA2a plasmid may be codon-optimised. By way of non-limiting example, codon-optimised gag-pol genes may comprise or consist of the nucleic acid sequence of SEQ ID NO: 17, or a variant thereof (as defined herein). In particular, the codon-optimised gag-pol genes of the invention may comprise or consist of a nucleic acid sequence having at least 80%, at least 85%, at least 90%, at least 95%, at least 96%, at least 97%, at least 98%, at least 99% or more sequence identity to SEQ ID NO: 17, preferably at least 95%, identity to SEQ ID NO: 17. The codon-optimised gag-pol genes may consist of the nucleic acid sequence of SEQ ID NO: 17. The preferred pDNA2a, pGM691, comprises the codon-optimised gag-pol genes of SEQ ID NO: 17.

The gag-pol genes (e.g. SIV gag-pol genes), including codon-optimised gag-pol genes are typically operably linked to a promoter to facilitate expression of the gag-pol proteins. Any suitable promoter may be used, including those described herein in the context of promoters for the transgene. Preferably, the promoter is a CAG promoter, as used on the exemplified pGM691 plasmid. An exemplary CAG promoter is set out in SEQ ID NO: 45. The codon-optimised gag-pol genes of SEQ ID NO: 17 comprise a translational slip, and so do not form a single conventional open reading frame.

Codon-optimised gag-pol genes (or nucleic acids comprising or consisting thereof) and plasmids comprising said genes or nucleic acids are advantageous in the production of retroviral/lentiviral (e.g. SIV) vectors using methods of the invention, as they allow for the production of high titre F/HN retroviral/lentiviral (e.g. SIV) vectors. Typically said codon-optimised gag-pol genes (or nucleic acids comprising or consisting thereof) and plasmids comprising said genes or nucleic acids can be used to produces a titre of retroviral/lentiviral (e.g. SIV) vector that is at least equivalent to the titre of retroviral/lentiviral (e.g. SIV) vector produced by a corresponding method which does not use codon-optimised gag-pol genes, as described herein. Thus, the use of codon-optimised gag-pol genes can be combined with a modified retroviral/lentiviral (e.g. SIV) RNA sequence to further maintain/increase vector titre.

Codon-optimised gag-pol genes are further disclosed in PCT/GB2022/050524, which is herein incorporated by reference in its entirety.

The invention also provides a retroviral/lentiviral (e.g. SIV) vector obtainable by a method of the invention.

Typically, the retroviral/lentiviral (e.g. SIV) vector obtainable by a method of the invention is produced at a high-titre, as described herein. Titre may be measured in terms of transducing units, as defined here. As described herein, the methods of the invention typically produce retroviral/lentiviral (e.g. SIV) vectors comprising a modified retroviral/lentiviral (e.g. SIV) RNA sequence at equivalent or higher titres than retroviral/lentiviral (e.g. SIV) vectors comprising the corresponding unmodified retroviral/lentiviral (e.g. SIV) RNA sequence, and/methods which do not use codon-optimised gag-pol genes.

Accordingly, the retroviral/lentiviral (e.g. SIV) vectors of the invention, including those obtainable by a method of the invention may optionally be at a titre of at least about 2.5×10⁶ TU/mL, at least about 3.0×10⁶ TU/mL, at least about 3.1×10⁶ TU/mL, at least about 3.2×10⁶ TU/mL, at least about 3.3×10⁶ TU/mL, at least about 3.4×10⁶ TU/mL, at least about 3.5×10⁶ TU/mL, at least about 3.6×10⁶ TU/mL, at least about 3.7×10⁶ TU/mL, at least about 3.8×10⁶ TU/mL, at least about 3.9×10⁶ TU/mL, at least about 4.0×10⁶ TU/mL or more. Preferably the retroviral/lentiviral (e.g. SIV) vector is produced at a titre of at least about 3.0×10⁶ TU/mL, or at least about 3.5×10⁶ TU/mL.

The production of high-titre retroviral/lentiviral (e.g. SIV) vectors may impart other desirable properties on the resulting vector products. For example, without being bound by theory, it is believed that production at high titres without the need for intense concentration by methods such as TFF results in a higher quality vector product than retroviral/lentiviral (e.g. SIV) vectors produced by corresponding methods without the use of codon-optimised gag-pol genes (and optionally a modified vector genome plasmid), because the vectors are exposed to less shear forces which can damage the viral particles and their RNA cargo.

Typically the gag-pol genes (e.g. codon-optimised gag-pol genes) used are matched to the retroviral/lentiviral vector being produced. By way of non-limiting example, when the lentiviral vector is an HIV vector, the codon-optimised gag-pol genes used are HIV gag-pol genes. By way of non-limiting example, when the lentiviral vector is an SIV vector, the codon-optimised gag-pol genes used are SIV gag-pol genes.

Preferably the codon-optimised gag-pol genes used are SIV gag-pol genes.

As described herein, the retroviral/lentiviral (e.g. SIV) vectors of the invention comprise a modified retroviral/lentiviral (e.g. SIV) RNA sequence, which is typically modified to reduce the number of retroviral/lentiviral (e.g. SIV) ORFs. Accordingly, the vector genome plasmid used in the production of a retroviral/lentiviral (e.g. SIV) vector of the invention may be modified to reduce the number of retroviral/lentiviral (e.g. SIV) ORFs. Any disclosure herein in relation to modification of the retroviral/lentiviral (e.g. SIV) RNA sequence, including modifications to reduce the number of retroviral/lentiviral (e.g. SIV) ORFs within the retroviral/lentiviral (e.g. SIV) RNA sequence, applies equally and without reservation to the vector genome plasmids (pDNA1) described herein, which may be used in the production of retroviral/lentiviral (e.g. SIV) vectors of the invention.

As used herein, the term “trypsin” refers to both trypsin and equivalents thereof. An equivalent enzyme is one with the same or essentially the same cleavage specificity as trypsin. Trypsin cleavage activity may be defined as cleavage C-terminal to arginine or lysine residues, typically exclusively C-terminal to arginine or lysine residues. The trypsin activity may preferably be provided by an animal origin free, recombinant enzyme such as TrypLE Select™. The addition of trypsin may be at the pre-harvest stage or at the post-harvest stage, or between harvesting steps.

Any appropriate purification means may be used to purify the retroviral/lentiviral (e.g. SIV) vector. Non-limiting examples of suitable purification steps include depth/end filtration, tangential flow filtration (TFF) and chromatography. The purification step typically comprises at least on chromatography step. Non-limiting examples of chromatography steps that may be used in accordance with the invention include mixed-mode size exclusion chromatography (SEC) and/or anion exchange chromatography. Elution may be carried out with or without the use of a salt gradient, preferably without.

This method may be used to produce the retroviral/lentiviral (e.g. SIV) vectors of the invention, such as those comprising a CFTR, A1AT and/or FVIII gene as described herein. Alternatively, the retroviral/lentiviral (e.g. SIV) vector of the invention comprises any of the above-mentioned genes, or the genes encoding the above-mentioned proteins.

The method, may use any combination of one or more of the specific plasmid constructs provided by FIGS. 1A-1F, FIG. 2 and/or FIG. 3A-3D is used to provide a retroviral/lentiviral (e.g. SIV) vector of the invention. Particularly the plasmid constructs of FIGS. 1B and 1D-1F are used, preferably in combination with the plasmid of FIG. 1A, FIG. 2 or FIG. 3A-3D, with the plasmid of FIG. 1A being particularly preferred.

The invention also provides a method of increasing retroviral/lentiviral (e.g. SIV) vector titre comprising the use of a modified retroviral/lentiviral (e.g. SIV) RNA sequence as described herein, or a vector genome plasmid from which such a modified retroviral/lentiviral (e.g. SIV) RNA sequence is derived. This method may be combined with the use of codon-optimised gag-pol genes (or nucleic acids comprising or consisting thereof), a plasmid comprising said genes or nucleic acids as described herein to further increase retroviral/lentiviral (e.g. SIV) vector titre. Said method of increasing retroviral/lentiviral (e.g. SIV) vector titre according to the invention may increase titre by at least 1.5-fold, at least 2-fold, or at least 2.5-fold or more compared with a corresponding method which uses the corresponding non-modified retroviral/lentiviral (e.g. SIV) RNA sequence or a vector genome plasmid from which the corresponding non-modified retroviral/lentiviral (e.g. SIV) RNA sequence is derived, and optionally also uses non-codon-optimised versions of the gag-pol genes (or nucleic acids comprising or consisting thereof), or plasmids or host cells comprising said non-codon optimised gag-pol genes or nucleic acids. Alternatively, a method of increasing retroviral/lentiviral (e.g. SIV) titre according to the invention may increase titre by at least about 25%, at least about 50%, at least about 100%, at least about 150%, at least about 200% or more compared with a corresponding method which uses the corresponding non-modified retroviral/lentiviral (e.g. SIV) RNA sequence or a vector genome plasmid from which the corresponding non-modified retroviral/lentiviral (e.g. SIV) RNA sequence is derived, and optionally also uses non-codon-optimised versions of the gag-pol genes (or nucleic acids comprising or consisting thereof), or plasmids comprising said non-codon optimised genes or nucleic acids. Preferably, a method of increasing retroviral/lentiviral (e.g. SIV) vector titre according to the invention may increase titre by (a) by at least 1.5-fold or at least 2-fold; and/or (b) by at least about 25%, more preferably at least about 50%, even more preferably at least about 100%. Typically the corresponding method is identical to the method of the invention except for the use of the corresponding non-modified retroviral/lentiviral (e.g. SIV) RNA sequence or a vector genome plasmid from which the corresponding non-modified retroviral/lentiviral (e.g. SIV) RNA sequence is derived, and optionally the codon-optimised gag-pol genes (or nucleic acids comprising or consisting thereof), a plasmid comprising said genes or nucleic acids. All the disclosure herein in relation to method of producing a retroviral/lentiviral (e.g. SIV) vector applies equally and without reservation to the methods of increasing retroviral/lentiviral (e.g. SIV) titre of the invention.

The invention also provides the use of a modified retroviral/lentiviral (e.g. SIV) RNA sequence of the invention (or vector genome plasmid from which said modified retroviral/lentiviral (e.g. SIV) RNA sequence is derived) to increase the titre of a retroviral/lentiviral (e.g. SIV) vector. This use may be combined with the use of codon-optimised gag-pol genes (or nucleic acids comprising or consisting thereof), a plasmid comprising said genes or nucleic acids as described herein to further increase retroviral/lentiviral (e.g. SIV) vector titre. Said use may increase retroviral/lentiviral (e.g. SIV) vector titre by at least 1.5-fold, at least 2-fold, or at least 2.5-fold or more compared with the use of a modified retroviral/lentiviral (e.g. SIV) RNA sequence of the invention (or vector genome plasmid from which said modified retroviral/lentiviral (e.g. SIV) RNA sequence is derived), and optionally a corresponding non-codon-optimised version of the gag-pol genes (or nucleic acids comprising or consisting thereof), or plasmids comprising said non-codon optimised genes or nucleic acids. Alternatively, said use may increase retroviral/lentiviral (e.g. SIV) titre by at least about 25%, at least about 50%, at least about 100%, at least about 150%, at least about 200% or more compared with the use of a modified retroviral/lentiviral (e.g. SIV) RNA sequence of the invention (or vector genome plasmid from which said modified retroviral/lentiviral (e.g. SIV) RNA sequence is derived), and optionally a corresponding non-codon-optimised version of the gag-pol genes (or nucleic acids comprising or consisting thereof), or plasmids comprising said non-codon optimised genes or nucleic acids. Preferably, said use increases retroviral/lentiviral (e.g. SIV) titre by (a) by at least 1.5-fold or at least 2-fold; and/or (b) at least about 25%, more preferably at least about 50%, even more preferably at least about 100%. Typically the corresponding use is identical to the method of the invention except for the use of the modified retroviral/lentiviral (e.g. SIV) RNA sequence of the invention (or vector genome plasmid from which said modified retroviral/lentiviral (e.g. SIV) RNA sequence is derived), and optionally the codon-optimised gag-pol genes (or nucleic acids comprising or consisting thereof), a plasmid comprising said genes or nucleic acids. All the disclosure herein in relation to method of producing a retroviral/lentiviral (e.g. SIV) vector applies equally and without reservation to the use of a modified retroviral/lentiviral (e.g. SIV) RNA sequence of the invention (or vector genome plasmid from which said modified retroviral/lentiviral (e.g. SIV) RNA sequence is derived) and optionally codon-optimised gag-pol genes (or nucleic acids comprising or consisting thereof), a plasmid comprising said genes or nucleic acids to increase the titre of a retroviral/lentiviral (e.g. SIV) vector according to the invention.

The use of codon-optimised gag-pol genes in combination with a modified retroviral/lentiviral (e.g. SIV) RNA sequence of the invention, or vector genome plasmid from which said modified retroviral/lentiviral (e.g. SIV) RNA sequence is derived, may provide a further advantage, in terms of safety and/or vector titre. Thus, the increased vector yields as described herein may be achieved using a modified retroviral/lentiviral (e.g. SIV) RNA sequence of the invention (or vector genome plasmid from which said modified retroviral/lentiviral (e.g. SIV) RNA sequence is derived) in combination with codon-optimised gag-pol genes. Any and all disclosure herein in relation to increased vector titre in the context of methods using a modified retroviral/lentiviral (e.g. SIV) RNA sequence of the invention (or vector genome plasmid from which said modified retroviral/lentiviral (e.g. SIV) RNA sequence is derived) applies equally and without reservation to methods using a modified retroviral/lentiviral (e.g. SIV) RNA sequence of the invention (or vector genome plasmid from which said modified retroviral/lentiviral (e.g. SIV) RNA sequence is derived) in combination with codon-optimised gag-pol genes, and to vectors produced by such methods.

### Therapeutic Indications

The retroviral/lentiviral (e.g. SIV) vectors of the present invention enable higher and sustained gene expression through efficient gene transfer whilst also reducing the risk of side-effects due to the expression of retroviral ORFs, such as upstream ORFs. The F/HN-pseudotyped retroviral/lentiviral (e.g. SIV) vectors of the invention are capable of: (i) airway transduction without disruption of epithelial integrity; (ii) persistent gene expression; (iii) lack of chronic toxicity; and (iv) efficient repeat administration. Long term/persistent stable gene expression, preferably at a therapeutically-effective level, may be achieved using repeat doses of a vector of the present invention. Alternatively, a single dose may be used to achieve the desired long-term expression.

Thus, advantageously, the retroviral/lentiviral (e.g. SIV) vectors of the present invention can be used in gene therapy. By way of example, the efficient airway cell uptake properties of the retroviral/lentiviral (e.g. SIV) vectors of the invention make them highly suitable for treating respiratory tract diseases. The retroviral/lentiviral (e.g. SIV) vectors of the invention can also be used in methods of gene therapy to promote secretion of therapeutic proteins. By way of further example, the invention provides secretion of therapeutic proteins into the lumen of the respiratory tract or the circulatory system. Thus, administration of a retroviral/lentiviral (e.g. SIV) vector of the invention and its uptake by airway cells may enable the use of the lungs (or nose or airways) as a “factory” to produce a therapeutic protein that is then secreted and enters the general circulation at therapeutic levels, where it can travel to cells/tissues of interest to elicit a therapeutic effect. In contrast to intracellular or membrane proteins, the production of such secreted proteins does not rely on specific disease target cells being transduced, which is a significant advantage and achieves high levels of protein expression. Thus, other diseases which are not respiratory tract diseases, such as cardiovascular diseases and blood disorders, particularly blood clotting deficiencies, can also be treated by the retroviral/lentiviral (e.g. SIV) vectors of the present invention.

Retroviral/lentiviral (e.g. SIV) vectors of the invention can effectively treat a disease by providing a transgene for the correction of the disease. For example, inserting a functional copy of the CFTR gene to ameliorate or prevent lung disease in CF patients, independent of the underlying mutation. Accordingly, retroviral/lentiviral (e.g. SIV) vectors of the invention may be used to treat cystic fibrosis (CF), typically by gene therapy with a CFTR transgene as described herein.

As another example, retroviral/lentiviral (e.g. SIV) vectors of the invention may be used to treat Alpha-1 Antitrypsin (A1AT) deficiency, typically by gene therapy with a A1AT transgene as described herein. A1AT is a secreted anti-protease that is produced mainly in the liver and then trafficked to the lung, with smaller amounts also being produced in the lung itself. The main function of A1AT is to bind and neutralise/inhibit neutrophil elastase. Gene therapy with A1AT according to the present invention is relevant to A1AT deficient patient, as well as in other lung diseases such as CF or chronic obstructive pulmonary disease (COPD), and offers the opportunity to overcome some of the problems encountered by conventional enzyme replacement therapy (in which A1AT isolated from human blood and administered intravenously every week), providing stable, long-lasting expression in the target tissue (lung/nasal epithelium), ease of administration and unlimited availability.

Transduction with a retroviral/lentiviral (e.g. SIV) vector of the invention may lead to secretion of the recombinant protein into the lumen of the lung as well as into the circulation. One benefit of this is that the therapeutic protein reaches the interstitium. A1AT gene therapy may therefore also be beneficial in other disease indications, non-limiting examples of which include type 1 and type 2 diabetes, acute myocardial infarction, ischemic heart disease, rheumatoid arthritis, inflammatory bowel disease, transplant rejection, graft versus host (GvH) disease, multiple sclerosis, liver disease, cirrhosis, vasculitides and infections, such as bacterial and/or viral infections.

A1AT has numerous other anti-inflammatory and tissue-protective effects, for example in pre-clinical models of diabetes, graft versus host disease and inflammatory bowel disease. The production of A1AT in the lung and/or nose following transduction according to the present invention may, therefore, be more widely applicable, including to these indications.

Other examples of diseases that may be treated with gene therapy of a secreted protein according to the present invention include cardiovascular diseases and blood disorders, particularly blood clotting deficiencies such as haemophilia (A, B or C), von Willebrand disease and Factor VII deficiency.

Other examples of diseases or disorders to be treated include Primary Ciliary Dyskinesia (PCD), acute lung injury, Surfactant Protein B (SFTB) deficiency, Pulmonary Alveolar Proteinosis (PAP), Chronic Obstructive Pulmonary Disease (COPD) and/or inflammatory, infectious, immune or metabolic conditions, such as lysosomal storage diseases.

Accordingly, the invention provides a method of treating a disease, the method comprising administering a retroviral/lentiviral (e.g. SIV) vector of the invention to a subject. Typically the retroviral/lentiviral (e.g. SIV) vector is produced using a method of the present invention. Any disease described herein may be treated according to the invention. In particular, the invention provides a method of treating a lung disease using a retroviral/lentiviral (e.g. SIV) vector of the invention. The disease to be treated may be a chronic disease. Preferably, a method of treating CF is provided.

The invention also provides a retroviral/lentiviral (e.g. SIV) vector as described herein for use in a method of treating a disease. Typically the retroviral/lentiviral (e.g. SIV) vector is produced using a method of the present disclosure. Any disease described herein may be treated according to the invention. In particular, the invention provides a retroviral/lentiviral (e.g. SIV) vector of the invention for use in a method of treating a lung disease. The disease to be treated may be a chronic disease. Preferably, a retroviral/lentiviral (e.g. SIV) vector for use in treating CF is provided.

The invention also provides the use of a retroviral/lentiviral (e.g. SIV) vector as described herein in the manufacture of a medicament for use in a method of treating a disease. Typically the retroviral/lentiviral (e.g. SIV) vector is produced using a method of the present disclosure. Any disease described herein may be treated according to the invention. In particular, the invention provides the use of a retroviral/lentiviral (e.g. SIV) vector of the invention for the manufacture of a medicament for use in a method of treating a lung disease. The disease to be treated may be a chronic disease. Preferably, the use of a retroviral/lentiviral (e.g. SIV) vector in the manufacture of a medicament for use in a method of treating CF is provided.

### Formulation and Administration

The retroviral/lentiviral (e.g. SIV) vectors of the invention may be administered in any dosage appropriate for achieving the desired therapeutic effect. Appropriate dosages may be determined by a clinician or other medical practitioner using standard techniques and within the normal course of their work. Non-limiting examples of suitable dosages include 1×10⁸ transduction units (TU), 1×10⁹ TU, 1×10¹⁰ TU, 1×10¹¹ TU or more.

The invention also provides compositions comprising the retroviral/lentiviral (e.g. SIV) vectors described above, and a pharmaceutically-acceptable carrier. Non-limiting examples of pharmaceutically acceptable carriers include water, saline, and phosphate-buffered saline. In some embodiments, however, the composition is in lyophilized form, in which case it may include a stabilizer, such as bovine serum albumin (BSA). In some embodiments, it may be desirable to formulate the composition with a preservative, such as thiomersal or sodium azide, to facilitate long-term storage.

The retroviral/lentiviral (e.g. SIV) vectors of the invention may be administered by any appropriate route. It may be desired to direct the compositions of the present invention (as described above) to the respiratory system of a subject. Efficient transmission of a therapeutic/prophylactic composition or medicament to the site of infection in the respiratory tract may be achieved by oral or intra-nasal administration, for example, as aerosols (e.g. nasal sprays), or by catheters. Typically the retroviral/lentiviral (e.g. SIV) vectors of the invention are stable in clinically relevant nebulisers, inhalers (including metered dose inhalers), catheters and aerosols, etc. Typically, therefore, the retroviral/lentiviral (e.g. SIV) vectors of the invention are formulated for administration to the lungs by any appropriate means, e.g. they may be formulated for intratracheal administration, intranasal administration, aerosol delivery, or direct injection or delivery to the lungs (e.g. delivered by catheter). Other modes of delivery, e.g. intravenous delivery, are also encompassed by the invention.

In some embodiments the nose is a preferred production site for a therapeutic protein using a retroviral/lentiviral (e.g. SIV) vector of the invention for at least one of the following reasons: (i) extracellular barriers such as inflammatory cells and sputum are less pronounced in the nose; (ii) ease of vector administration; (iii) smaller quantities of vector required; and (iv) ethical considerations. Thus, transduction of nasal epithelial cells with a retroviral/lentiviral (e.g. SIV) vector of the invention may result in efficient (high-level) and long-lasting expression of the therapeutic transgene of interest. Accordingly, nasal administration of a retroviral/lentiviral (e.g. SIV) vector of the invention may be preferred.

Formulations for intra-nasal administration may be in the form of nasal droplets or a nasal spray. An intra-nasal formulation may comprise droplets having approximate diameters in the range of 100-5000 μm, such as 500-4000 μm, 1000-3000 μm or 100-1000 μm. Alternatively, in terms of volume, the droplets may be in the range of about 0.001-100 μl, such as 0.1-50 μl or 1.0-25 μl, or such as 0.001-1 μl.

The aerosol formulation may take the form of a powder, suspension or solution. The size of aerosol particles is relevant to the delivery capability of an aerosol. Smaller particles may travel further down the respiratory airway towards the alveoli than would larger particles. In one embodiment, the aerosol particles have a diameter distribution to facilitate delivery along the entire length of the bronchi, bronchioles, and alveoli. Alternatively, the particle size distribution may be selected to target a particular section of the respiratory airway, for example the alveoli. In the case of aerosol delivery of the medicament, the particles may have diameters in the approximate range of 0.1-50 μm, preferably 1-25 μm, more preferably 1-5 μm.

Aerosol particles may be for delivery using a nebulizer (e.g. via the mouth) or nasal spray. An aerosol formulation may optionally contain a propellant and/or surfactant.

The formulation of pharmaceutical aerosols is routine to those skilled in the art, see for example, Sciarra, J. in Remington's Pharmaceutical Sciences (supra). The agents may be formulated as solution aerosols, dispersion or suspension aerosols of dry powders, emulsions or semisolid preparations. The aerosol may be delivered using any propellant system known to those skilled in the art. The aerosols may be applied to the upper respiratory tract, for example by nasal inhalation, or to the lower respiratory tract or to both. The part of the lung that the medicament is delivered to may be determined by the disorder. Compositions comprising a vector of the invention, in particular where intranasal delivery is to be used, may comprise a humectant. This may help reduce or prevent drying of the mucus membrane and to prevent irritation of the membranes. Suitable humectants include, for instance, sorbitol, mineral oil, vegetable oil and glycerol; soothing agents; membrane conditioners; sweeteners; and combinations thereof. The compositions may comprise a surfactant. Suitable surfactants include non-ionic, anionic and cationic surfactants. Examples of surfactants that may be used include, for example, polyoxyethylene derivatives of fatty acid partial esters of sorbitol anhydrides, such as for example, Tween 80, Polyoxyl 40 Stearate, Polyoxy ethylene 50 Stearate, fusieates, bile salts and Octoxynol.

In some cases after an initial administration a subsequent administration of a retroviral/lentiviral (e.g. SIV) vector may be performed. The administration may, for instance, be at least a week, two weeks, a month, two months, six months, a year or more after the initial administration. In some instances, retroviral/lentiviral (e.g. SIV) vector of the invention may be administered at least once a week, once a fortnight, once a month, every two months, every six months, annually or at longer intervals. Preferably, administration is every six months, more preferably annually. The retroviral/lentiviral (e.g. SIV) vectors may, for instance, be administered at intervals dictated by when the effects of the previous administration are decreasing.

Any two or more retroviral/lentiviral (e.g. SIV) vectors of the invention may be administered separately, sequentially or simultaneously. Thus two retroviral/lentiviral (e.g. SIV) vectors or more retroviral/lentiviral (e.g. SIV) vectors, where at least one retroviral/lentiviral (e.g. SIV) vectors is a retroviral/lentiviral (e.g. SIV) vector of the invention, may be administered separately, simultaneously or sequentially and in particular two or more retroviral/lentiviral (e.g. SIV) vectors of the invention may be administered in such a manner. The two may be administered in the same or different compositions. In a preferred instance, the two retroviral/lentiviral (e.g. SIV) vectors may be delivered in the same composition.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position—Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

### Alignment Scores for Determining Sequence Identity

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:
Total number of identical matches
   ______×100
   [length of the longer sequence plus the number of gaps introduced into the longer sequence in order to align the two sequences]

Substantially homologous polypeptides are characterized as having one or more amino acid substitutions, deletions or additions. These changes are preferably of a minor nature, that is conservative amino acid substitutions (as described herein) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

### EXAMPLES

The invention is now described with reference to the Examples below. These are not limiting on the scope of the invention, and a person skilled in the art would be appreciate that suitable equivalents could be used within the scope of the present invention. Thus, the Examples may be considered component parts of the invention, and the individual aspects described therein may be considered as disclosed independently, or in any combination.

### Example 1—Modifying the Vector Genome Plasmid, Including Reducing the Number of Intact SIV ORFs within the Vector Genome Plasmid Maintains, or Even Increases, Vector Yield

The inventors reviewed sequences of the construction plasmids and identified several regions of concern within the original vector genome plasmid pGM326. In particular, the pGM326 partial Gag RRE cPPT hCEF region contains:
77 start codons (ATGs);
32 ORFs ≥10 amino acids in length
2 large ORFs in the 5′ to 3′ direction
   189 amino acids from the most 5′ ATG in vector genome (Gag/RRE fusion), encoding p17 Matrix and part of p24 capsid
   250 amino acids from ATG internal to RRE (RRE/cPPT/hCEF fusion)

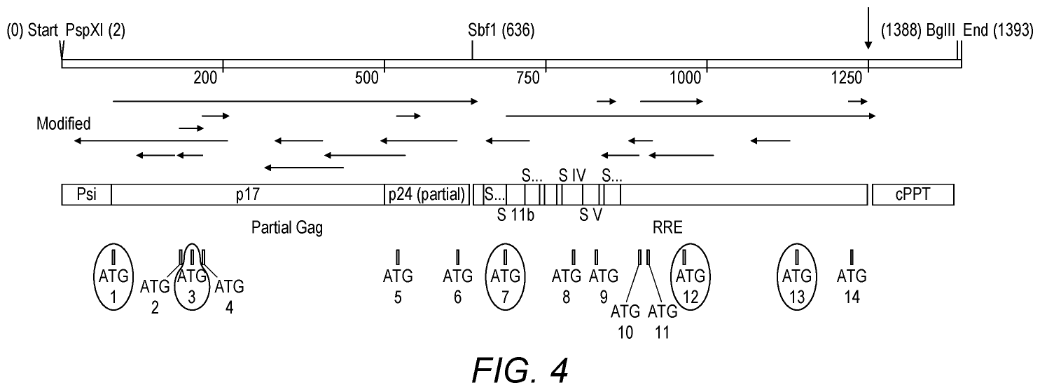

In particular, 14 ATG start codons were identified in the partial Gag/RRE region of the pGM326 genome plasmid that could result in ORFs of longer than 10 amino acids. These are illustrated in FIG. 4. The circled ATGs are those with a strong kozak sequence and that are in-frame with Gag or Env.

As such, the inventors designed a modified version of the pGM326 plasmid with a combination of additional modifications intended to reduce the number of intact SIV ORFs (and in particular to remove these 2 large ORFs) for improved safety. The modifications are made to the 2 large ORFs upstream of the hCEF promoter and CFTR transgene (soCFTR2). The changes made were as follows:

| Approach | Modification(s) | Edited Region | Plasmid |
|---|---|---|---|
| 1 | 4 fsATGs | Partial Gag | pGM826 |
| 2 | 2 fsATGs | Partial Gag | pGM827 |
| 3 | 2 mtATGs | Partial Gag | pGM828 |
| 4 | mtSTOP + 1 mtATGs | Partial Gag | pGM829 |
| 5 | 4 fsATGs + 3 mtATGs | Partial Gag + RRE | pGM830 |
| 6 | mtSTOP + 4 mtATGs | Partial Gag + RRE | pGM831 |

fsATG = frameshift ATG;
mtATG = ATG with point mutations (ATG disrupted);
mtSTOP = mutated ATG −> stop codon (introduced)

Approach 1 made frameshift mutations to ATG codons (fsATG) 1, 2, 3 and 5 in the SIV-CFTR partial-Gag region. Approach 2 made frameshift mutations to ATG codons 1 and 3 in the SIV-CFTR partial-Gag region. Approach 3 made point mutations to ATG codons (mtATG) 1 and 3 in the SIV-CFTR partial-Gag region. Approach 4 made a mutation of the 6th codon of the SIV-CFTR partial-Gag region into a STOP codon, and a point mutation to ATG codon 3 in the partial-Gag region. Approach 5 made frameshift mutations to ATG codons 1, 2, 3 and 5 and point mutations to ATG codons 7, 12 and 13 of the SIV-CFTR partial-Gag/RRE region. Approach 6 made a mutation of the 6th codon of the SIV-CFTR partial-Gag region into a STOP codon, and point mutations to ATG codons 3, 7, 12 and 13 across the SIV-CFTR partial-Gag/RRE region. Approach 5 produced the vector genome plasmid of pGM830 as shown in FIG. 1A, with the sequence of SEQ ID NO: 19.

Each novel vector genome plasmid was assessed for functionality by two rounds of transient lentiviral vector (LV) production, comprising transfection of the plasmid being tested with SIV GagPol, SIV Rev, SeV Fct4 and SIVct+SeV HN plasmids into A459 cells in an Ambr®15 bioreactor system at 12 mL volume. Following LV production, vector product was activated before being filtered through a 0.45 μm filter and stored at −80° C. Post thaw, activated material was diluted 1 in 50 and transduced onto into A459 cells. The resulting LV titre was quantified using CFTR FACS.

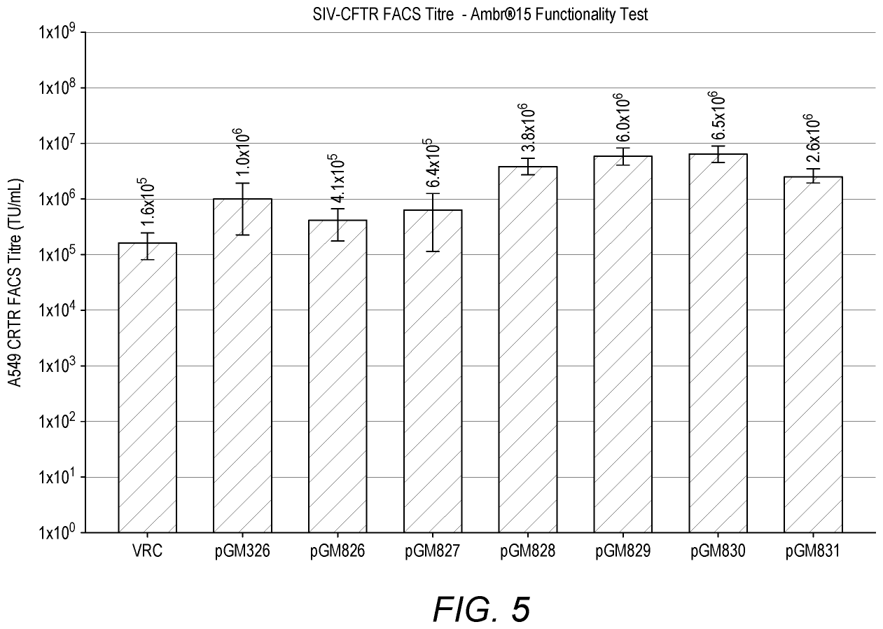

As shown in FIG. 5, several of the modified vector genome plasmids resulted in an observable increase in LV titre compared with the unmodified pGM326 vector genome plasmid. The pGM830 vector genome plasmid gave rise to the highest LV titre (6.5×10⁶ TU/mL), compared with 1.0×10⁶ TU/mL for the unmodified pGM326.

Comparisons of vector titre using either pGM326 and the modified vector genome plasmids in an otherwise identical production protocol demonstrated that the use of modified vector genome plasmids at least gave a comparable titre to pGM326, indicating that an improved safety profile could be achieved without adversely affecting titre.

### Example 2—Modifying the Vector Genome Plasmid, Including Reducing the Number of Intact SIV ORFs within the Vector Genome Plasmid Maintains, or Even Increases, Vector Integration

The LV production of Example 1 was repeated using HEK239T cells.

The resulting LV titre was quantified using a 3-day integration assay. DNA from transduced cells was harvested 3-days post-transduction and non-integrated DNA removed. qPCR was then used to determine and quantify the vector was present/integrated into the host cell DNA.

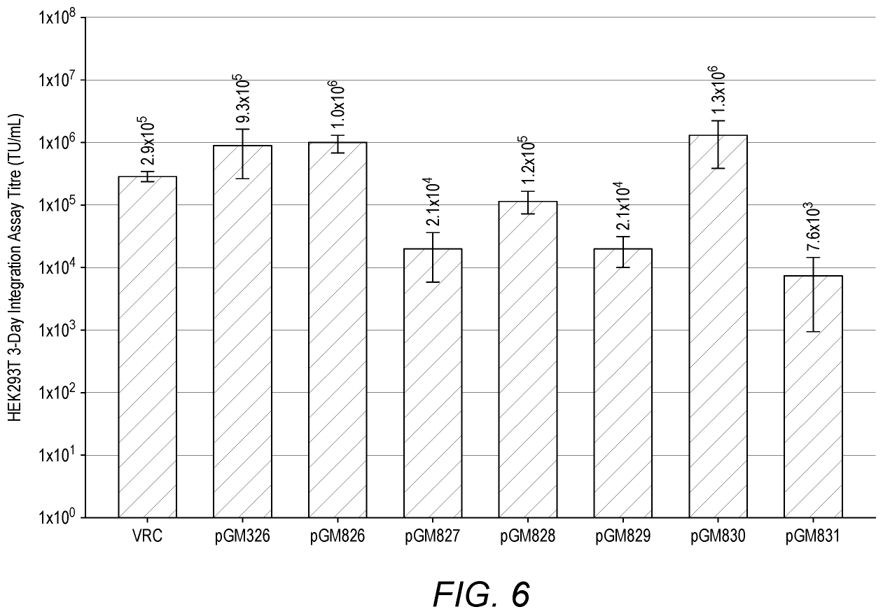

As shown in FIG. 6, the pGM826 and pGM830 modified vector genome plasmids resulted in an observable increase in LV integration compared with the unmodified pGM326 vector genome plasmid. The pGM830 vector genome plasmid gave rise to the highest LV integration (1.3×10⁶ TU/mL), compared with 9.3×10⁵ TU/mL for the unmodified pGM326.

Again, comparisons of vector titre using either pGM326 and the modified vector genome plasmids in an otherwise identical production protocol demonstrated that the use of modified vector genome plasmids at least gave a comparable LV integration to pGM326, indicating that an improved safety profile could be achieved without adversely affecting LV functionality.

### Example 3—Modifying the Vector Genome Plasmid, Including Reducing the Number of Intact SIV ORFs within the Vector Genome Plasmid Maintains, or Even Increases, Transgene Expression

SIV-CFTR generated using pGM326or pGM830 were used to transduce A549 cells in the presence and absence of AZT and Raltegravir. All cells were stained for CFTR expression 3-days post-transduction, and subsequently only cells transduced in the absence of inhibitors were passaged and stained again for CFTR expression 10-Days post-transduction, in order to investigate the extent of pseudotransduction (transduction without proviral DNA integration into the host genome), which could also give rise to CFTR expression.

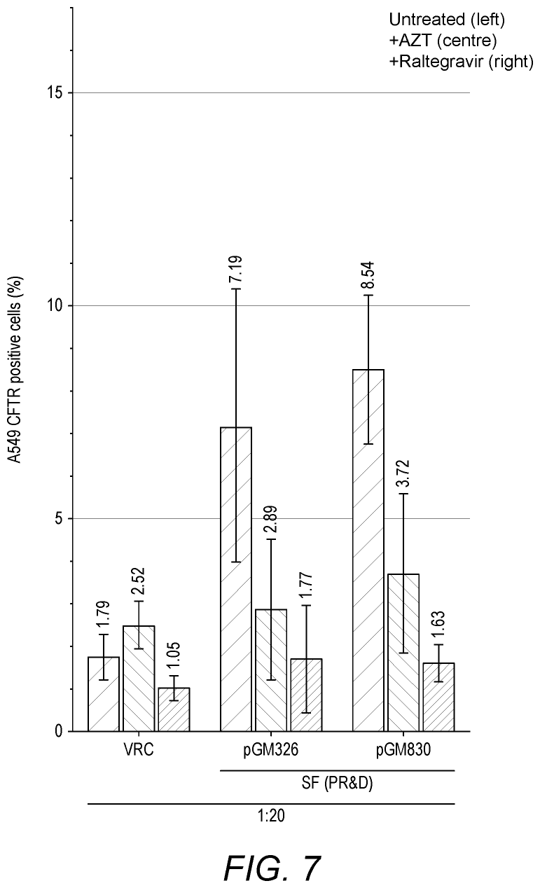

As shown in FIG. 7, when inhibitors of reverse transcription (azidothymidine, AZT) and SIV integration (raltegravir) are used, the number of cells expressing CFTR is almost the same as the negative control, meaning that CFTR expression is a result of LV integration.

Furthermore, FIG. 7 also demonstrates that the % of CFTR positive cells was greater for the LV produced using pGM830, even when AZT was included during transduction, compared with LV produced using pGM326.

Thus, this comparison of CFTR transgene expression using either pGM326 and pGM830 demonstrated that the use of modified vector genome plasmids at least gave comparable transgene expression compared with LV produced using unmodified pGM326, indicating that an improved safety profile could be achieved without adversely affecting LV functionality.

### Example 4—Fct4 is Cleaved by Enzymes with Trypsin-Like Cleavage Specificity to Produce the Fusion Active Form Comprising F₁ and F₂ Fragments

LV produced according to Example 1 was assessed for F protein cleavage following the addition of a trypsin-like enzyme. Activation of F protein occurs by cleavage into 2 subunits, F₁ and F₂. Thus, cleavage of F protein is an accepted proxy for F protein activation and hence fusion capability.

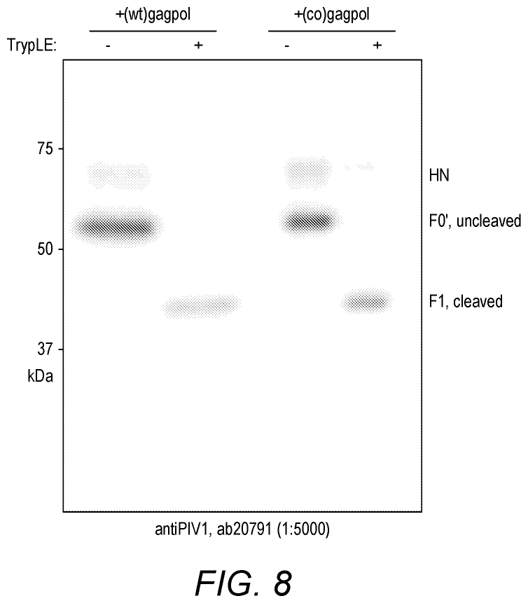

Following incubation of the LV with the trypsin-like enzyme, Western blotting was carried out using an anti-PIV1 antibody ab20791 at a dilution of 1:5000. As shown in FIG. 8, incubation with a trypsin-like enzyme successfully cleaves Fct4, as in the presence of said enzyme, no uncleaved F₀ is detected, but rather only the F₁.

### Sequence Information

### Key to Sequences

SEQ ID NO: 1 modified SIV/CFTR RNA sequence
SEQ ID NO: 2 p17 protein sequence
SEQ ID NO: 3 p24 protein sequence
SEQ ID NO: 4 p8 protein sequence
SEQ ID NO: 5 Protease sequence
SEQ ID NO: 6 p51 protein sequence
SEQ ID NO: 7 p15 protein sequence
SEQ ID NO: 8 p31 protein sequence
SEQ ID NO: 9 Gag protein
SEQ ID NO: 10 Pol protein
SEQ ID NO: 11 (skipped)
SEQ ID NO: 12 Fct4 protein
SEQ ID NO: 13 Fct4 protein (including signal sequence)
SEQ ID NO: 14 Fct4 protein (fragment 1)
SEQ ID NO: 15 Fct4 protein (fragment 2)
SEQ ID NO: 16 Fct4 protein signal sequence
SEQ ID NO: 17 Codon-optimised SIV gag-pol nucleic acid sequence
SEQ ID NO: 18 Wild-type SIV gag-pol nucleic acid sequence
SEQ ID NO: 19 Plasmid as defined in FIG. 2A (pDNA1 pGM830)
SEQ ID NO:20 Plasmid as defined in FIG. 2B (pDNA1 pGM691)
SEQ ID NO: 21 Plasmid as defined in FIG. 2C (pDNA2a pGM297)
SEQ ID NO: 22 Plasmid as defined in FIG. 2D (pDNA2b pGM299)
SEQ ID NO:23 Plasmid as defined in FIG. 2E (pDNA3a pGM301)
SEQ ID NO: 24 Plasmid as defined in FIG. 2F (pDNA3b pGM303)
SEQ ID NO: 25 Plasmid as defined in FIG. 2G (pDNA2a pGM326)
SEQ ID NO: 26 Exemplified hCEF promoter
SEQ ID NO: 27 Exemplified CMV promoter
SEQ ID NO: 28 Exemplified EF1a promoter
SEQ ID NO: 29 Exemplified CFTR transgene (soCFTR2)
SEQ ID NO: 30 Exemplified A1AT transgene
SEQ ID NO: 31 Complementary strand to the exemplified A1AT transgene
SEQ ID NO: 32 Exemplified A1A1 polypeptide
SEQ ID NO: 33 Exemplified FVIII transgene (N6)
SEQ ID NO: 34 Exemplified FVIII transgene (V3)
SEQ ID NO: 35 Complementary strand to the exemplified FVIII transgene (N6)
SEQ ID NO: 36 Complementary strand to the exemplified FVIII transgene (V3)
SEQ ID NO: 37 Exemplified FVIII polypeptide (N6)
SEQ ID NO: 38 Exemplified FVIII polypeptide (V3)
SEQ ID NO: 39 Exemplified WPRE component (mWPRE)
SEQ ID NO: 40 F/HN-SIV-hCEF-soA1AT plasmid as defined in FIG. 3 (pDNA1 pGM407)
SEQ ID NO: 41 F/HN-SIV-CMV-HFVIII-V3 plasmid as defined in FIG. 4A (pDNA1 pGM411)
SEQ ID NO: 42 F/HN-SIV-hCEF-HFVIII-V3 plasmid as defined in FIG. 4B (pDNA1 pGM413)
SEQ ID NO: 43 F/HN-SIV-CMV-HFVIII-N6-co plasmid as defined in FIG. 4C (pDNA1 pGM412)
SEQ ID NO: 44 F/HN-SIV-hCEF-HFVIII-N6-co plasmid as defined in FIG. 4D (pDNA1 pGM414)
SEQ ID NO: 45 Exemplary CAG promoter
SEQ ID NO: 46 Additional amino acid sequence encoded from false transcription start site upstream of that encoding the Fct4 of SEQ ID NO: 13

### Sequences

```
<210> SEQ ID NO: 1
<211> 7553
<223> Modified SIV/CFTR RNA sequence
ucucuuacua ggagaccagc uugagccugg guguucgcug guuagccuaa ccugguuggc 60
caccaggggu aaggacuccu uggcuuagaa agcuaauaaa cuugccugca uuagagcuua 120
ucugagucaa guguccucau ugacgccuca cucucuugaa cgggaaucuu ccuuacuggg 180
uucucucucu gacccaggcg agagaaacuc cagcaguggc gcccgaacag ggacuugagu 240
gagaguguag gcacquacag cugagaaggc gucggacgcg aaggaagcgc ggggugcgac 300
gcgaccaaga aggagacuug gugaguaggc uucucgagug ccgggaaaaa gcucgagccu 360
aguuagagga cuaggagagg ccguagccgu aacuacucug ggcaaguagg gcaggcggug 420
gguacgcaau ugggggcggc uaccucagca cuaaauagga gacaauuaga ccaauuugag 480
aaaauacgac uucgcccgaa cggaaagaaa aaguaccaaa uuaaacauuu aauauugggc 540
aggcaaggag auuggagcgc uucggccucc augagagguu guuggagaca gaggaggggu 600
guaaaagaau cauagaaguc cucuaccccc uagaaccaac aggaucggag ggcuuaaaaa 660
gucuguucaa ucuugugugc gugcuauauu gcuugcacaa ggaacagaaa gugaaagaca 720
cagaggaagc aguagcaaca guaagacaac acugccaucu aguggaaaaa gaaaaaagug 780
caacagagac aucuagugga caaaagaaaa augacaaggg aauagcagcg ccaccuggug 840
gcagucagaa uuuuccagcg caacaacaag gaaauugccu ggguacaugu acccuuguca 900
ccgcgcaccu uaaaugcgug gguaaaagca guagaggaga aaaaauuugg agcagaaaua 960
guacccaugu uucaagcccu aucgccugca ggccguuugu gcuaggguuc uuaggcuucu 1020
ugggggcugc uggaacugca uugggagcag cggcgacagc ccugacgguc cagucucagc 1080
auuugcuugc ugggauacug cagcagcaga agaaucugcu ggcggcugug gaggcucaac 1140
agcagauguu gaagcugacc auuuggggug uuaaaaaccu caaugcccgc gucacagccc 1200
uugagaagua ccuagaggau caggcacgac uaaacuccug ggggugcgca uggaaacaag 1260
uaugucauac cacaguggag uggcccugga caaaucggac uccggauugg caaaauaaga 1320
cuugguugga gugggaaaga caaauagcug auuuggaaag caacauuacg agacaauuag 1380
ugaaggcuag agaacaagag gaaaagaauc uagaugccua ucagaaguua acuaguuggu 1440
cagauuucug gucuugguuc gauuucucaa aauggcuuaa cauuuuaaaa aagggauuuu 1500
uaguaauagu aggaauaaua ggguuaagau uacuuuacac aguauaugga uguauaguga 1560
ggguuaggca gggauauguu ccucuaucuc cacagaucca uauaaagcgg caauuuuaaa 1620
agaaagggag gaauaggggg acagacuuca gcagagagac uaauuaauau aauaacaaca 1680
caauuagaaa uacaacauuu acaaaccaaa auucaaaaaa uuuuaaauuu uagagccgcg 1740
gagaucuguu acauaacuua ugguaaaugg ccugccuggc ugacugccca augaccccug 1800
cccaaugaug ucaauaauga uguauguucc cauguaaugc caauagggac uuuccauuga 1860
ugucaauggg uggaguauuu augguaacug cccacuuggc aguacaucaa guguaucaua 1920
ugccaaguau gcccccuauu gaugucaaug augguaaaug gccugccugg cauuaugccc 1980
aguacaugac cuuaugggac uuuccuacuu ggcaguacau cuauguauua gucauugcua 2040
uuaccauggg aauucacuag uggagaagag caugcuugag ggcugagugc cccucagugg 2100
gcagagagca cauggcccac agucccugag aaguuggggg gagggguggg caauugaacu 2160
ggugccuaga gaaggugggg cuuggguaaa cugggaaagu gauguggugu acuggcucca 2220
ccuuuuuccc cagggugggg gagaaccaua uauaagugca guagucucug ugaacauuca 2280
agcuucugcc uucucccucc ugugaguuug cuagccacca ugcagagaag cccucuggag 2340
aaggccucug uggugagcaa gcuguucuuc agcuggacca ggcccauccu gaggaagggc 2400
uacaggcaga gacuggagcu gucugacauc uaccagaucc ccucugugga cucugcugac 2460
aaccugucug agaagcugga gagggagugg gauagagagc uggccagcaa gaagaacccc 2520
aagcugauca augcccugag gagaugcuuc uucuggagau ucauguucua uggcaucuuc 2580
cuguaccugg gggaagugac caaggcugug cagccucugc ugcugggcag aaucauugcc 2640
agcuaugacc cugacaacaa ggaggagagg agcauugcca ucuaccuggg cauuggccug 2700
ugccugcugu ucauugugag gacccugcug cugcacccug ccaucuuugg ccugcaccac 2760
auuggcaugc agaugaggau ugccauguuc agccugaucu acaagaaaac ccugaagcug 2820
uccagcagag ugcuggacaa gaucagcauu ggccagcugg ugagccugcu gagcaacaac 2880
cugaacaagu uugaugaggg ccuggcccug gcccacuuug uguggauugc cccucugcag 2940
guggcccugc ugaugggccu gauuugggag cugcugcagg ccucugccuu uuguggccug 3000
ggcuuccuga uugugcuggc ccuguuucag gcuggccugg gcaggaugau gaugaaguac 3060
agggaccaga gggcaggcaa gaucagugag aggcugguga ucaccucuga gaugauugag 3120
aacauccagu cugugaaggc cuacuguugg gaggaagcua uggagaagau gauugaaaac 3180
cugaggcaga cagagcugaa gcugaccagg aaggcugccu augugagaua cuucaacagc 3240
ucugccuucu ucuucucugg cuucunugug guguuccugu cugugcugcc cuaugcccug 3300
aucaagggga ucauccugag aaagauuuuc accaccauca gcuucugcau ugugcugagg 3360
auggcuguga ccagacaguu ccccugggcu gugcagaccu gguaugacag ccugggggcc 3420
aucaacaaga uccaggacuu ccugcagaag caggaguaca agacccugga guacaaccug 3480
accaccacag aaguggugau ggagaaugug acagccuucu gggaggaggg cuuuggggag 3540
cuguuugaga aggccaagca gaacaacaac aacagaaaga ccagcaaugg ggaugacucc 3600
cuguucuucu ccaacuucuc ccugcugggc acaccugugc ugaaggacau caacuucaag 3660
auugagaggg ggcagcugcu ggcuguggcu ggaucuacag gggcuggcaa gaccagccug 3720
cugaugauga ucauggggga gcuggagccu ucugagggca agaucaagca cucuggcagg 3780
aucagcuuuu gcagccaguu cagcuggauc augccuggca ccaucaagga gaacaucauc 3840
uuuggaguga gcuaugauga guacagauac aggaguguga ucaaggccug ccagcuggag 3900
gaggacauca gcaaguuugc ugagaaggac aacauugugc ugggggaggg aggcauuaca 3960
cugucugggg gccagagagc cagaaucagc cuggccaggg cuguguacaa ggaugcugac 4020
cuguaccugc uggacucccc cuuuggcuac cuggaugugc ugacagagaa ggagauuuuu 4080
gagagcugug ugugcaagcu gauggccaac aagaccagaa uccuggugac cagcaagaug 4140
gagcaccuga agaaggcuga caagauccug auccugcaug agggcagcag cuacuucuau 4200
gggaccuucu cugagcugca gaaccugcag ccugacuuca gcucuaagcu gaugggcugu 4260
gacagcuuug accaguucuc ugcugagagg aggaacagca uccugacaga gacccugcac 4320
agauucagcc uggagggaga ugccccugug agcuggacag agaccaagaa gcagagcuuc 4380
aagcagacag gggaguuugg ggagaagagg aagaacucca uccugaaccc caucaacagc 4440
aucaggaagu ucagcauugu gcagaaaacc ccccugcaga ugaauggcau ugaggaagau 4500
ucugaugagc cccuggagag gagacugagc cuggugccug auucugagca gggagaggcc 4560
auccugccua ggaucucugu gaucagcaca ggcccuacac ugcaggccag aaggaggcag 4620
ucugugcuga accugaugac ccacucugug aaccagggcc agaacaucca caggaaaacc 4680
acagccucca ccaggaaagu gagccuggcc ccucaggcca aucugacaga gcuggacauc 4740
uacagcagga ggcugucuca ggagacaggc cuggagauuu cugaggagau caaugaggag 4800
gaccugaaag agugcuucuu ugaugacaug gagagcaucc cugcugugac caccuggaac 4860
accuaccuga gauacaucac agugcacaag agccugaucu uugugcugau cuggugccug 4920
gugaucuucc uggcugaagu ggcugccucu cugguggugc uguggcugcu gggaaacacc 4980
ccacugcagg acaagggcaa cagcacccac agcaggaaca acagcuaugc ugugaucauc 5040
accuccaccu ccagcuacua uguguucuac aucuaugugg gaguggcuga uacccugcug 5100
gcuaugggcu ucuuuagagg ccugccccug gugcacacac ugaucacagu gagcaagauc 5160
cuccaccaca agaugcugca cucugugcug caggcuccua ugagcacccu gaauacccug 5220
aaggcugggg gcauccugaa cagauucucc aaggauauug ccauccugga ugaccugcug 5280
ccucucacca ucuuugacuu cauccagcug cugcugauug ugauuggggc cauugcugug 5340
guggcagugc ugcagcccua caucuuugug gccacagugc cugugauugu ggccuucauc 5400
augcugaggg ccuacuuucu gcagaccucc cagcagcuga agcagcugga gucugagggc 5460
agaagcccca ucuucaccca ccuggugaca agccugaagg gccuguggac ccugagagcc 5520
uuuggcaggc agcccuacuu ugagacccug uuccacaagg cccugaaccu gcacacagcc 5580
aacugguucc ucuaccuguc cacccugaga ugguuccaga ugagaauuga gaugaucuuu 5640
gucaucuucu ucauugcugu gaccuucauc agcauucuga ccacaggaga gggagagggc 5700
agagugggca uuauccugac ccuggccaug aacaucauga gcacacugca gugggcagug 5760
aacagcagca uugaugugga cagccugaug aggaguguga gcagaguguu caaguucauu 5820
gauaugccca cagagggcaa gccuaccaag agcaccaagc ccuacaagaa uggccagcug 5880
agcaaaguga ugaucauuga gaacagccau gugaagaagg augauaucug gcccagugga 5940
ggccagauga cagugaagga ccugacagcc aaguacacag aggggggcaa ugcuauccug 6000
gagaacaucu ccuucagcau cuccccuggc cagagagugg gacugcuggg aagaacaggc 6060
ucuggcaagu cuacccugcu gucugccuuc cugaggcugc ugaacacaga gggagagauc 6120
cagauugaug gaguguccug ggacagcauc acacugcagc aguggaggaa ggccuuuggu 6180
gugauccccc agaaaguguu caucuucagu ggcaccuuca ggaagaaccu ggaccccuau 6240
gagcaguggu cugaccagga gauuuggaaa guggcugaug aagugggccu gagaagugug 6300
auugagcagu ucccuggcaa gcuggacuuu guccuggugg augggggcug ugugcugagc 6360
cauggccaca agcagcugau gugccuggcc agaucagugc ugagcaaggc caagauccug 6420
cugcuggaug agccuucugc ccaccuggau ccugugaccu accagaucau caggaggacc 6480
cucaagcagg ccuuugcuga cugcacaguc auccugugug agcacaggau ugaggccaug 6540
cuggagugcc agcaguuccu ggugauugag gagaacaaag ugaggcagua ugacagcauc 6600
cagaagcugc ugaaugagag gagccuguuc aggcaggcca ucagccccuc ugauagagug 6660
aagcuguucc cccacaggaa cagcuccaag ugcaagagca agccccagau ugcugcccug 6720
aaggaggaga cagaggagga agugcaggac accaggcugu gagggcccaa ucaaccucug 6780
gauuacaaaa uuugugaaag auugacuggu auucuuaacu auguugcucc uuuuacgcua 6840
uguggauacg cugcuuuaau gccuuuguau caugcuauug cuucccguau ggcuuucauu 6900
uucuccuccu uguauaaauc cugguugcug ucucuuuaug aggaguugug gcccguuguc 6960
aggcaacgug gcguggugug cacuguguuu gcugacgcaa cccccacugg uuggggcauu 7020
gccaccaccu gucagcuccu uuccgggacu uucgcuuucc cccucccuau ugccacggcg 7080
gaacucaucg ccgccugccu ugcccgcugc uggacagggg cucggcuguu gggcacugac 7140
aauuccgugg uguugucggg gaaaucaucg uccuuuccuu ggcugcucgc cuguguugcc 7200
accuggauuc ugcgcgggac guccuucugc uacgucccuu cggcccucaa uccagcggac 7260
cuuccuuccc gcggccugcu gccggcucug cggccucuuc cgcgucuucg ccuucgcccu 7320
cagacgaguc ggaucucccu uugggccgcc uccccgcaag cuucgcacuu uuuaaaagaa 7380
aagggaggac uggaugggau uuauuacucc gauaggacgc uggcuuguaa cucagucucu 7440
uacuaggaga ccagcuugag ccuggguguu cgcugguuag ccuaaccugg uuggccacca 7500
gggguaagga cuccuuggcu uagaaagcua auaaacuugc cugcauuaga gcu 7553
<210> SEQ ID NO: 2
<211> 140
<223> p17 protein
Gly Ala Ala Thr Ser Ala Leu Asn Arg Arg Gln Leu Asp Gln Phe Glu
1 5 10 15
Lys Ile Arg Leu Arg Pro Asn Gly Lys Lys Lys Tyr Gln Ile Lys His
                        20 25 30
Leu Ile Trp Ala Gly Lys Glu Met Glu Arg Phe Gly Leu His Glu Arg
                35 40 45
Leu Leu Glu Thr Glu Glu Gly Cys Lys Arg Ile Ile Glu Val Leu Tyr
        50 55 60
Pro Leu Glu Pro Thr Gly Ser Glu Gly Leu Lys Ser Leu Phe Asn Leu
65 70 75 80
Val Cys Val Leu Tyr Cys Leu His Lys Glu Gln Lys Val Lys Asp Thr
                                85 90 95
Glu Glu Ala Val Ala Thr Val Arg Gln His Cys His Leu Val Glu Lys
                        100 105 110
Glu Lys Ser Ala Thr Glu Thr Ser Ser Gly Gln Lys Lys Asn Asp Lys
                115 120 125
Gly Ile Ala Ala Pro Pro Gly Gly Ser Gln Asn Phe
        130 135 140
<210> SEQ ID NO: 3
<211> 231
<223> p24 protein
Pro Ala Gln Gln Gln Gly Asn Ala Trp Val His Val Pro Leu Ser Pro
1 5 10 15
Arg Thr Leu Asn Ala Trp Val Lys Ala Val Glu Glu Lys Lys Phe Gly
                        20 25 30
Ala Glu Ile Val Pro Met Phe Gln Ala Leu Ser Glu Gly Cys Thr Pro
                35 40 45
Tyr Asp Ile Asn Gln Met Leu Asn Val Leu Gly Asp His Gln Gly Ala
        50 55 60
Leu Gln Ile Val Lys Glu Ile Ile Asn Glu Glu Ala Ala Gln Trp Asp
65 70 75 80
Val Thr His Pro Leu Pro Ala Gly Pro Leu Pro Ala Gly Gln Leu Arg
                                85 90 95
Asp Pro Arg Gly Ser Asp Ile Ala Gly Thr Thr Ser Ser Val Gln Glu
                        100 105 110
Gln Leu Glu Trp Ile Tyr Thr Ala Asn Pro Arg Val Asp Val Gly Ala
                115 120 125
Ile Tyr Arg Arg Trp Ile Ile Leu Gly Leu Gln Lys Cys Val Lys Met
        130 135 140
Tyr Asn Pro Val Ser Val Leu Asp Ile Arg Gln Gly Pro Lys Glu Pro
145 150 155 160
Phe Lys Asp Tyr Val Asp Arg Phe Tyr Lys Ala Ile Arg Ala Glu Gln
                                165 170 175
Ala Ser Gly Glu Val Lys Gln Trp Met Thr Glu Ser Leu Leu Ile Gln
                        180 185 190
Asn Ala Asn Pro Asp Cys Lys Val Ile Leu Lys Gly Leu Gly Met His
                195 200 205
Pro Thr Leu Glu Glu Met Leu Thr Ala Cys Gln Gly Val Gly Gly Pro
        210 215 220
Ser Tyr Lys Ala Lys Val Met
225 230
<210> SEQ ID NO: 4
<211> 54
<223> p8 protein
Val Gln Gln Gly Gly Pro Lys Arg Gln Arg Pro Pro Leu Arg Cys Tyr
1 5 10 15
Asn Cys Gly Lys Phe Gly His Met Gln Arg Gln Cys Pro Glu Pro Arg
                        20 25 30
Lys Thr Lys Cys Leu Lys Cys Gly Lys Leu Gly His Leu Ala Lys Asp
                35 40 45
Cys Arg Gly Gln Val Asn
        50
<210> SEQ ID NO: 5
<211> 101
<223> protease
Phe Glu Leu Pro Leu Trp Arg Arg Pro Ile Lys Thr Val Tyr Ile Glu
1 5 10 15
Gly Val Pro Ile Lys Ala Leu Leu Asp Thr Gly Ala Asp Asp Thr Ile
                        20 25 30
Ile Lys Glu Asn Asp Leu Gln Leu Ser Gly Pro Trp Arg Pro Lys Ile
                35 40 45
Ile Gly Gly Ile Gly Gly Gly Leu Asn Val Lys Glu Tyr Asn Asp Arg
        50 55 60
Glu Val Lys Ile Glu Asp Lys Ile Leu Arg Gly Thr Ile Leu Leu Gly
65 70 75 80
Ala Thr Pro Ile Asn Ile Ile Gly Arg Asn Leu Leu Ala Pro Ala Gly
                                85 90 95
Ala Arg Leu Val Met
                        100
<210> SEQ ID NO: 6
<211> 441
<223> p51 protein
Gly Gln Leu Ser Glu Lys Ile Pro Val Thr Pro Val Lys Leu Lys Glu
1 5 10 15
Gly Ala Arg Gly Pro Cys Val Arg Gln Trp Pro Leu Ser Lys Glu Lys
                        20 25 30
Ile Glu Ala Leu Gln Glu Ile Cys Ser Gln Leu Glu Gln Glu Gly Lys
                35 40 45
Ile Ser Arg Val Gly Gly Glu Asn Ala Tyr Asn Thr Pro Ile Phe Cys
        50 55 60
Ile Lys Lys Lys Asp Lys Ser Gln Trp Arg Met Leu Val Asp Phe Arg
65 70 75 80
Glu Leu Asn Lys Ala Thr Gln Asp Phe Phe Glu Val Gln Leu Gly Ile
                                85 90 95
Pro His Pro Ala Gly Leu Arg Lys Met Arg Gln Ile Thr Val Leu Asp
                        100 105 110
Val Gly Asp Ala Tyr Tyr Ser Ile Pro Leu Asp Pro Asn Phe Arg Lys
                115 120 125
Tyr Thr Ala Phe Thr Ile Pro Thr Val Asn Asn Gln Gly Pro Gly Ile
        130 135 140
Arg Tyr Gln Phe Asn Cys Leu Pro Gln Gly Trp Lys Gly Ser Pro Thr
145 150 155 160
Ile Phe Gln Asn Thr Ala Ala Ser Ile Leu Glu Glu Ile Lys Arg Asn
                                165 170 175
Leu Pro Ala Leu Thr Ile Val Gln Tyr Met Asp Asp Leu Trp Val Gly
                        180 185 190
Ser Gln Glu Asn Glu His Thr His Asp Lys Leu Val Glu Gln Leu Arg
                195 200 205
Thr Lys Leu Gln Ala Trp Gly Leu Glu Thr Pro Glu Lys Lys Val Gln
        210 215 220
Lys Glu Pro Pro Tyr Glu Trp Met Gly Tyr Lys Leu Trp Pro His Lys
225 230 235 240
Trp Glu Leu Ser Arg Ile Gln Leu Glu Glu Lys Asp Glu Trp Thr Val
                                245 250 255
Asn Asp Ile Gln Lys Leu Val Gly Lys Leu Asn Trp Ala Ala Gln Leu
                        260 265 270
Tyr Pro Gly Leu Arg Thr Lys Asn Ile Cys Lys Leu Ile Arg Gly Lys
                275 280 285
Lys Asn Leu Leu Glu Leu Val Thr Trp Thr Pro Glu Ala Glu Ala Glu
        290 295 300
Tyr Ala Glu Asn Ala Glu Ile Leu Lys Thr Glu Gln Glu Gly Thr Tyr
305 310 315 320
Tyr Lys Pro Gly Ile Pro Ile Arg Ala Ala Val Gln Lys Leu Glu Gly
                                325 330 335
Gly Gln Trp Ser Tyr Gln Phe Lys Gln Glu Gly Gln Val Leu Lys Val
                        340 345 350
Gly Lys Tyr Thr Lys Gln Lys Asn Thr His Thr Asn Glu Leu Arg Thr
                355 360 365
Leu Ala Gly Leu Val Gln Lys Ile Cys Lys Glu Ala Leu Val Ile Trp
        370 375 380
Gly Ile Leu Pro Val Leu Glu Leu Pro Ile Glu Arg Glu Val Trp Glu
385 390 395 400
Gln Trp Trp Ala Asp Tyr Trp Gln Val Ser Trp Ile Pro Glu Trp Asp
                                405 410 415
Phe Val Ser Thr Pro Pro Leu Leu Lys Leu Trp Tyr Thr Leu Thr Lys
                        420 425 430
Glu Pro Ile Pro Lys Glu Asp Val Tyr
                435 440
<210> SEQ ID NO: 7
<211> 120
<223> p15 protein
Tyr Val Asp Gly Ala Cys Asn Arg Asn Ser Lys Glu Gly Lys Ala Gly
1 5 10 15
Tyr Ile Ser Gln Tyr Gly Lys Gln Arg Val Glu Thr Leu Glu Asn Thr
                        20 25 30
Thr Asn Gln Gln Ala Glu Leu Thr Ala Ile Lys Met Ala Leu Glu Asp
                35 40 45
Ser Gly Pro Asn Val Asn Ile Val Thr Asp Ser Gln Tyr Ala Met Gly
        50 55 60
Ile Leu Thr Ala Gln Pro Thr Gln Ser Asp Ser Pro Leu Val Glu Gln
65 70 75 80
Ile Ile Ala Leu Met Ile Gln Lys Gln Gln Ile Tyr Leu Gln Trp Val
                                85 90 95
Pro Ala His Lys Gly Ile Gly Gly Asn Glu Glu Ile Asp Lys Leu Val
                        100 105 110
Ser Lys Gly Ile Arg Arg Val Leu
                120 115
<210> SEQ ID NO: 8
<211> 291
<223> p31 protein
Phe Leu Glu Lys Ile Glu Glu Ala Gln Glu Glu His Glu Arg Tyr His
1 5 10 15
Asn Asn Trp Lys Asn Leu Ala Asp Thr Tyr Gly Leu Pro Gln Ile Val
                        20 25 30
Ala Lys Glu Ile Val Ala Met Cys Pro Lys Cys Gln Ile Lys Gly Glu
                35 40 45
Pro Val His Gly Gln Val Asp Ala Ser Pro Gly Thr Trp Gln Met Asp
        50 55 60
Cys Thr His Leu Glu Gly Lys Val Val Ile Val Ala Val His Val Ala
65 70 75 80
Ser Gly Phe Ile Glu Ala Glu Val Ile Pro Arg Glu Thr Gly Lys Glu
                                85 90 95
Thr Ala Lys Phe Leu Leu Lys Ile Leu Ser Arg Trp Pro Ile Thr Gln
                        100 105 110
Leu His Thr Asp Asn Gly Pro Asn Phe Thr Ser Gln Glu Val Ala Ala
                115 120 125
Ile Cys Trp Trp Gly Lys Ile Glu His Thr Thr Gly Ile Pro Tyr Asn
        130 135 140
Pro Gln Ser Gln Gly Ser Ile Glu Ser Met Asn Lys Gln Leu Lys Glu
145 150 155 160
Ile Ile Gly Lys Ile Arg Asp Asp Cys Gln Tyr Thr Glu Thr Ala Val
                                165 170 175
Leu Met Ala Cys His Ile His Asn Phe Lys Arg Lys Gly Gly Ile Gly
                        180 185 190
Gly Gln Thr Ser Ala Glu Arg Leu Ile Asn Ile Ile Thr Thr Gln Leu
                195 200 205
Glu Ile Gln His Leu Gln Thr Lys Ile Gln Lys Ile Leu Asn Phe Arg
        210 215 220
Val Tyr Tyr Arg Glu Gly Arg Asp Pro Val Trp Lys Gly Pro Ala Gln
225 230 235 240
Leu Ile Trp Lys Gly Glu Gly Ala Val Val Leu Lys Asp Gly Ser Asp
                                245 250 255
Leu Lys Val Val Pro Arg Arg Lys Ala Lys Ile Ile Lys Asp Tyr Glu
                        260 265 270
Pro Lys Gln Arg Val Gly Asn Glu Gly Asp Val Glu Gly Thr Arg Gly
                275 280 285
Ser Asp Asn
        290
<210> SEQ ID NO: 9
<211> 519
<223> Gag protein
Met Gly Ala Ala Thr Ser Ala Leu Asn Arg Arg Gln Leu Asp Gln Phe
1 5 10 15
Glu Lys Ile Arg Leu Arg Pro Asn Gly Lys Lys Lys Tyr Gln Ile Lys
                        20 25 30
His Leu Ile Trp Ala Gly Lys Glu Met Glu Arg Phe Gly Leu His Glu
                35 40 45
Arg Leu Leu Glu Thr Glu Glu Gly Cys Lys Arg Ile Ile Glu Val Leu
        50 55 60
Tyr Pro Leu Glu Pro Thr Gly Ser Glu Gly Leu Lys Ser Leu Phe Asn
65 70 75 80
Leu Val Cys Val Leu Tyr Cys Leu His Lys Glu Gln Lys Val Lys Asp
                                85 90 95
Thr Glu Glu Ala Val Ala Thr Val Arg Gln His Cys His Leu Val Glu
                        100 105 110
Lys Glu Lys Ser Ala Thr Glu Thr Ser Ser Gly Gln Lys Lys Asn Asp
                115 120 125
Lys Gly Ile Ala Ala Pro Pro Gly Gly Ser Gln Asn Phe Pro Ala Gln
        130 135 140
Gln Gln Gly Asn Ala Trp Val His Val Pro Leu Ser Pro Arg Thr Leu
145 150 155 160
Asn Ala Trp Val Lys Ala Val Glu Glu Lys Lys Phe Gly Ala Glu Ile
                                165 170 175
Val Pro Met Phe Gln Ala Leu Ser Glu Gly Cys Thr Pro Tyr Asp Ile
                        180 185 190
Asn Gln Met Leu Asn Val Leu Gly Asp His Gln Gly Ala Leu Gln Ile
                195 200 205
Val Lys Glu Ile Ile Asn Glu Glu Ala Ala Gln Trp Asp Val Thr His
        210 215 220
Pro Leu Pro Ala Gly Pro Leu Pro Ala Gly Gln Leu Arg Asp Pro Arg
225 230 235 240
Gly Ser Asp Ile Ala Gly Thr Thr Ser Ser Val Gln Glu Gln Leu Glu
                                245 250 255
Trp Ile Tyr Thr Ala Asn Pro Arg Val Asp Val Gly Ala Ile Tyr Arg
                        260 265 270
Arg Trp Ile Ile Leu Gly Leu Gln Lys Cys Val Lys Met Tyr Asn Pro
                275 280 285
Val Ser Val Leu Asp Ile Arg Gln Gly Pro Lys Glu Pro Phe Lys Asp
        290 295 300
Tyr Val Asp Arg Phe Tyr Lys Ala Ile Arg Ala Glu Gln Ala Ser Gly
305 310 315 320
Glu Val Lys Gln Trp Met Thr Glu Ser Leu Leu Ile Gln Asn Ala Asn
                                325 330 335
Pro Asp Cys Lys Val Ile Leu Lys Gly Leu Gly Met His Pro Thr Leu
                        340 345 350
Glu Glu Met Leu Thr Ala Cys Gln Gly Val Gly Gly Pro Ser Tyr Lys
                355 360 365
Ala Lys Val Met Ala Glu Met Met Gln Thr Met Gln Asn Gln Asn Met
        370 375 380
Val Gln Gln Gly Gly Pro Lys Arg Gln Arg Pro Pro Leu Arg Cys Tyr
385 390 395 400
Asn Cys Gly Lys Phe Gly His Met Gln Arg Gln Cys Pro Glu Pro Arg
                                405 410 415
Lys Thr Lys Cys Leu Lys Cys Gly Lys Leu Gly His Leu Ala Lys Asp
                        420 425 430
Cys Arg Gly Gln Val Asn Phe Leu Gly Tyr Gly Arg Trp Met Gly Ala
                435 440 445
Lys Pro Arg Asn Phe Pro Ala Ala Thr Leu Gly Ala Glu Pro Ser Ala
        450 455 460
Pro Pro Pro Pro Ser Gly Thr Thr Pro Tyr Asp Pro Ala Lys Lys Leu
465 470 475 480
Leu Gln Gln Tyr Ala Glu Lys Gly Lys Gln Leu Arg Glu Gln Lys Arg
                                485 490 495
Asn Pro Pro Ala Met Asn Pro Asp Trp Thr Glu Gly Tyr Ser Leu Asn
                        500 505 510
Ser Leu Phe Gly Glu Asp Gln
                515
<210> SEQ ID NO: 10
<211> 1044
<223> Pol protein
Met Ser Lys Val Trp Lys Ile Gly Thr Pro Ser Lys Arg Leu Gln Gly
1 5 10 15
Thr Gly Glu Phe Phe Arg Val Trp Thr Val Asp Gly Gly Lys Thr Glu
                        20 25 30
Lys Phe Ser Arg Arg Tyr Ser Trp Ser Gly Thr Glu Cys Ala Ser Ser
                35 40 45
Thr Glu Arg His His Pro Ile Arg Pro Ser Lys Glu Ala Pro Ala Ala
        50 55 60
Ile Cys Arg Glu Arg Glu Thr Thr Glu Gly Ala Lys Glu Glu Ser Thr
65 70 75 80
Gly Asn Glu Ser Gly Leu Asp Arg Gly Ile Phe Phe Glu Leu Pro Leu
                                85 90 95
Trp Arg Arg Pro Ile Lys Thr Val Tyr Ile Glu Gly Val Pro Ile Lys
                        100 105 110
Ala Leu Leu Asp Thr Gly Ala Asp Asp Thr Ile Ile Lys Glu Asn Asp
                115 120 125
Leu Gln Leu Ser Gly Pro Trp Arg Pro Lys Ile Ile Gly Gly Ile Gly
        130 135 140
Gly Gly Leu Asn Val Lys Glu Tyr Asn Asp Arg Glu Val Lys Ile Glu
145 150 155 160
Asp Lys Ile Leu Arg Gly Thr Ile Leu Leu Gly Ala Thr Pro Ile Asn
                                165 170 175
Ile Ile Gly Arg Asn Leu Leu Ala Pro Ala Gly Ala Arg Leu Val Met
                        180 185 190
Gly Gln Leu Ser Glu Lys Ile Pro Val Thr Pro Val Lys Leu Lys Glu
                195 200 205
Gly Ala Arg Gly Pro Cys Val Arg Gln Trp Pro Leu Ser Lys Glu Lys
        210 215 220
Ile Glu Ala Leu Gln Glu Ile Cys Ser Gln Leu Glu Gln Glu Gly Lys
225 230 235 240
Ile Ser Arg Val Gly Gly Glu Asn Ala Tyr Asn Thr Pro Ile Phe Cys
                                245 250 255
Ile Lys Lys Lys Asp Lys Ser Gln Trp Arg Met Leu Val Asp Phe Arg
                        260 265 270
Glu Leu Asn Lys Ala Thr Gln Asp Phe Phe Glu Val Gln Leu Gly Ile
                275 280 285
Pro His Pro Ala Gly Leu Arg Lys Met Arg Gln Ile Thr Val Leu Asp
        290 295 300
Val Gly Asp Ala Tyr Tyr Ser Ile Pro Leu Asp Pro Asn Phe Arg Lys
305 310 315 320
Tyr Thr Ala Phe Thr Ile Pro Thr Val Asn Asn Gln Gly Pro Gly Ile
                                325 330 335
Arg Tyr Gln Phe Asn Cys Leu Pro Gln Gly Trp Lys Gly Ser Pro Thr
                        340 345 350
Ile Phe Gln Asn Thr Ala Ala Ser Ile Leu Glu Glu Ile Lys Arg Asn
                355 360 365
Leu Pro Ala Leu Thr Ile Val Gln Tyr Met Asp Asp Leu Trp Val Gly
        370 375 380
Ser Gln Glu Asn Glu His Thr His Asp Lys Leu Val Glu Gln Leu Arg
385 390 395 400
Thr Lys Leu Gln Ala Trp Gly Leu Glu Thr Pro Glu Lys Lys Val Gln
                                405 410 415
Lys Glu Pro Pro Tyr Glu Trp Met Gly Tyr Lys Leu Trp Pro His Lys
                        420 425 430
Trp Glu Leu Ser Arg Ile Gln Leu Glu Glu Lys Asp Glu Trp Thr Val
                435 440 445
Asn Asp Ile Gln Lys Leu Val Gly Lys Leu Asn Trp Ala Ala Gln Leu
        450 455 460
Tyr Pro Gly Leu Arg Thr Lys Asn Ile Cys Lys Leu Ile Arg Gly Lys
465 470 475 480
Lys Asn Leu Leu Glu Leu Val Thr Trp Thr Pro Glu Ala Glu Ala Glu
                                485 490 495
Tyr Ala Glu Asn Ala Glu Ile Leu Lys Thr Glu Gln Glu Gly Thr Tyr
                        500 505 510
Tyr Lys Pro Gly Ile Pro Ile Arg Ala Ala Val Gln Lys Leu Glu Gly
                  515 520 525
Gly Gln Trp Ser Tyr Gln Phe Lys Gln Glu Gly Gln Val Leu Lys Val
        530 535 540
Gly Lys Tyr Thr Lys Gln Lys Asn Thr His Thr Asn Glu Leu Arg Thr
545 550 555 560
Leu Ala Gly Leu Val Gln Lys Ile Cys Lys Glu Ala Leu Val Ile Trp
                                565 570 575
Gly Ile Leu Pro Val Leu Glu Leu Pro Ile Glu Arg Glu Val Trp Glu
                        580 585 590
Gln Trp Trp Ala Asp Tyr Trp Gln Val Ser Trp Ile Pro Glu Trp Asp
                595 600 605
Phe Val Ser Thr Pro Pro Leu Leu Lys Leu Trp Tyr Thr Leu Thr Lys
        610 615 620
Glu Pro Ile Pro Lys Glu Asp Val Tyr Tyr Val Asp Gly Ala Cys Asn
625 630 635 640
Arg Asn Ser Lys Glu Gly Lys Ala Gly Tyr Ile Ser Gln Tyr Gly Lys
                                645 650 655
Gln Arg Val Glu Thr Leu Glu Asn Thr Thr Asn Gln Gln Ala Glu Leu
                        660 665 670
Thr Ala Ile Lys Met Ala Leu Glu Asp Ser Gly Pro Asn Val Asn Ile
                675 680 685
Val Thr Asp Ser Gln Tyr Ala Met Gly Ile Leu Thr Ala Gln Pro Thr
        690 695 700
Gln Ser Asp Ser Pro Leu Val Glu Gln Ile Ile Ala Leu Met Ile Gln
705 710 715 720
Lys Gln Gln Ile Tyr Leu Gln Trp Val Pro Ala His Lys Gly Ile Gly
                                725 730 735
Gly Asn Glu Glu Ile Asp Lys Leu Val Ser Lys Gly Ile Arg Arg Val
                        740 745 750
Leu Phe Leu Glu Lys Ile Glu Glu Ala Gln Glu Glu His Glu Arg Tyr
                755 760 765
His Asn Asn Trp Lys Asn Leu Ala Asp Thr Tyr Gly Leu Pro Gln Ile
        770 775 780
Val Ala Lys Glu Ile Val Ala Met Cys Pro Lys Cys Gln Ile Lys Gly
785 790 795 800
Glu Pro Val His Gly Gln Val Asp Ala Ser Pro Gly Thr Trp Gln Met
                                805 810 815
Asp Cys Thr His Leu Glu Gly Lys Val Val Ile Val Ala Val His Val
                        820 825 830
Ala Ser Gly Phe Ile Glu Ala Glu Val Ile Pro Arg Glu Thr Gly Lys
                835 840 845
Glu Thr Ala Lys Phe Leu Leu Lys Ile Leu Ser Arg Trp Pro Ile Thr
        850 855 860
Gln Leu His Thr Asp Asn Gly Pro Asn Phe Thr Ser Gln Glu Val Ala
865 870 875 880
Ala Ile Cys Trp Trp Gly Lys Ile Glu His Thr Thr Gly Ile Pro Tyr
                                885 890 895
Asn Pro Gln Ser Gln Gly Ser Ile Glu Ser Met Asn Lys Gln Leu Lys
                        900 905 910
Glu Ile Ile Gly Lys Ile Arg Asp Asp Cys Gln Tyr Thr Glu Thr Ala
                915 920 925
Val Leu Met Ala Cys His Ile His Asn Phe Lys Arg Lys Gly Gly Ile
        930 935 940
Gly Gly Gln Thr Ser Ala Glu Arg Leu Ile Asn Ile Ile Thr Thr Gln
945 950 955 960
Leu Glu Ile Gln His Leu Gln Thr Lys Ile Gln Lys Ile Leu Asn Phe
                                965 970 975
Arg Val Tyr Tyr Arg Glu Gly Arg Asp Pro Val Trp Lys Gly Pro Ala
                        980 985 990
Gln Leu Ile Trp Lys Gly Glu Gly Ala Val Val Leu Lys Asp Gly Ser
                995 1000 1005
Asp Leu Lys Val Val Pro Arg Arg Lys Ala Lys Ile Ile Lys Asp
        1010 1015 1020
Tyr Glu Pro Lys Gln Arg Val Gly Asn Glu Gly Asp Val Glu Gly
        1025 1030 1035
Thr Arg Gly Ser Asp Asn
        1040
<210> SEQ ID NO: 11
<211> 0
<212> 000
<223> 000
<210> SEQ ID NO: 12
<211> 502
<223> Fct4 protein
Gln Ile Pro Arg Asp Arg Leu Ser Asn Ile Gly Val Ile Val Asp Glu
1 5 10 15
Gly Lys Ser Leu Lys Ile Ala Gly Ser His Glu Ser Arg Tyr Ile Val
                        20 25 30
Leu Ser Leu Val Pro Gly Val Asp Phe Glu Asn Gly Cys Gly Thr Ala
                35 40 45
Gln Val Ile Gln Tyr Lys Ser Leu Leu Asn Arg Leu Leu Ile Pro Leu
        50 55 60
Arg Asp Ala Leu Asp Leu Gln Glu Ala Leu Ile Thr Val Thr Asn Asp
65 70 75 80
Thr Thr Gln Asn Ala Gly Ala Pro Gln Ser Arg Phe Phe Gly Ala Val
                                85 90 95
Ile Gly Thr Ile Ala Leu Gly Val Ala Thr Ser Ala Gln Ile Thr Ala
                        100 105 110
Gly Ile Ala Leu Ala Glu Ala Arg Glu Ala Lys Arg Asp Ile Ala Leu
                115 120 125
Ile Lys Glu Ser Met Thr Lys Thr His Lys Ser Ile Glu Leu Leu Gln
        130 135 140
Asn Ala Val Gly Glu Gln Ile Leu Ala Leu Lys Thr Leu Gln Asp Phe
145 150 155 160
Val Asn Asp Glu Ile Lys Pro Ala Ile Ser Glu Leu Gly Cys Glu Thr
                                165 170 175
Ala Ala Leu Arg Leu Gly Ile Lys Leu Thr Gln His Tyr Ser Glu Leu
                        180 185 190
Leu Thr Ala Phe Gly Ser Asn Phe Gly Thr Ile Gly Glu Lys Ser Leu
                195 200 205
Thr Leu Gln Ala Leu Ser Ser Leu Tyr Ser Ala Asn Ile Thr Glu Ile
        210 215 220
Met Thr Thr Ile Arg Thr Gly Gln Ser Asn Ile Tyr Asp Val Ile Tyr
225 230 235 240
Thr Glu Gln Ile Lys Gly Thr Val Ile Asp Val Asp Leu Glu Arg Tyr
                                245 250 255
Met Val Thr Leu Ser Val Lys Ile Pro Ile Leu Ser Glu Val Pro Gly
                        260 265 270
Val Leu Ile His Lys Ala Ser Ser Ile Ser Tyr Asn Ile Asp Gly Glu
                275 280 285
Glu Trp Tyr Val Thr Val Pro Ser His Ile Leu Ser Arg Ala Ser Phe
        290 295 300
Leu Gly Gly Ala Asp Ile Thr Asp Cys Val Glu Ser Arg Leu Thr Tyr
305 310 315 320
Ile Cys Pro Arg Asp Pro Ala Gln Leu Ile Pro Asp Ser Gln Gln Lys
                                325 330 335
Cys Ile Leu Gly Asp Thr Thr Arg Cys Pro Val Thr Lys Val Val Asp
                        340 345 350
Ser Leu Ile Pro Lys Phe Ala Phe Val Asn Gly Gly Val Val Ala Asn
                355 360 365
Cys Ile Ala Ser Thr Cys Thr Cys Gly Thr Gly Arg Arg Pro Ile Ser
        370 375 380
Gln Asp Arg Ser Lys Gly Val Val Phe Leu Thr His Asp Asn Cys Gly
385 390 395 400
Leu Ile Gly Val Asn Gly Val Glu Leu Tyr Ala Asn Arg Arg Gly His
                                405 410 415
Asp Ala Thr Trp Gly Val Gln Asn Leu Thr Val Gly Pro Ala Ile Ala
                        420 425 430
Ile Arg Pro Val Asp Ile Ser Leu Asn Leu Ala Asp Ala Thr Asn Phe
                435 440 445
Leu Gln Asp Ser Lys Ala Glu Leu Glu Lys Ala Arg Lys Ile Leu Ser
        450 455 460
Glu Val Gly Arg Trp Tyr Asn Ser Arg Glu Thr Val Ile Thr Ile Ile
465 470 475 480
Val Val Met Val Val Ile Leu Val Val Ile Ile Val Ile Ile Ile Val
                                485 490 495
Leu Tyr Arg Leu Arg Arg
                        500
<210> SEQ ID NO: 13
<211> 527
<223> Fct 4 (including signal sequence)
Met Ala Thr Tyr Ile Gln Arg Val Gln Cys Ile Ser Thr Ser Leu Leu
1 5 10 15
Val Val Leu Thr Thr Leu Val Ser Cys Gln Ile Pro Arg Asp Arg Leu
                        20 25 30
Ser Asn Ile Gly Val Ile Val Asp Glu Gly Lys Ser Leu Lys Ile Ala
                35 40 45
Gly Ser His Glu Ser Arg Tyr Ile Val Leu Ser Leu Val Pro Gly Val
        50 55 60
Asp Phe Glu Asn Gly Cys Gly Thr Ala Gln Val Ile Gln Tyr Lys Ser
65 70 75 80
Leu Leu Asn Arg Leu Leu Ile Pro Leu Arg Asp Ala Leu Asp Leu Gln
                                85 90 95
Glu Ala Leu Ile Thr Val Thr Asn Asp Thr Thr Gln Asn Ala Gly Ala
                        100 105 110
Pro Gln Ser Arg Phe Phe Gly Ala Val Ile Gly Thr Ile Ala Leu Gly
                115 120 125
Val Ala Thr Ser Ala Gln Ile Thr Ala Gly Ile Ala Leu Ala Glu Ala
        130 135 140
Arg Glu Ala Lys Arg Asp Ile Ala Leu Ile Lys Glu Ser Met Thr Lys
145 150 155 160
Thr His Lys Ser Ile Glu Leu Leu Gln Asn Ala Val Gly Glu Gln Ile
                                165 170 175
Leu Ala Leu Lys Thr Leu Gln Asp Phe Val Asn Asp Glu Ile Lys Pro
                        180 185 190
Ala Ile Ser Glu Leu Gly Cys Glu Thr Ala Ala Leu Arg Leu Gly Ile
                195 200 205
Lys Leu Thr Gln His Tyr Ser Glu Leu Leu Thr Ala Phe Gly Ser Asn
        210 215 220
Phe Gly Thr Ile Gly Glu Lys Ser Leu Thr Leu Gln Ala Leu Ser Ser
225 230 235 240
Leu Tyr Ser Ala Asn Ile Thr Glu Ile Met Thr Thr Ile Arg Thr Gly
                                245 250 255
Gln Ser Asn Ile Tyr Asp Val Ile Tyr Thr Glu Gln Ile Lys Gly Thr
                        260 265 270
Val Ile Asp Val Asp Leu Glu Arg Tyr Met Val Thr Leu Ser Val Lys
                275 280 285
Ile Pro Ile Leu Ser Glu Val Pro Gly Val Leu Ile His Lys Ala Ser
        290 295 300
Ser Ile Ser Tyr Asn Ile Asp Gly Glu Glu Trp Tyr Val Thr Val Pro
305 310 315 320
Ser His Ile Leu Ser Arg Ala Ser Phe Leu Gly Gly Ala Asp Ile Thr
                                325 330 335
Asp Cys Val Glu Ser Arg Leu Thr Tyr Ile Cys Pro Arg Asp Pro Ala
                        340 345 350
Gln Leu Ile Pro Asp Ser Gln Gln Lys Cys Ile Leu Gly Asp Thr Thr
                355 360 365
Arg Cys Pro Val Thr Lys Val Val Asp Ser Leu Ile Pro Lys Phe Ala
        370 375 380
Phe Val Asn Gly Gly Val Val Ala Asn Cys Ile Ala Ser Thr Cys Thr
385 390 395 400
Cys Gly Thr Gly Arg Arg Pro Ile Ser Gln Asp Arg Ser Lys Gly Val
                                405 410 415
Val Phe Leu Thr His Asp Asn Cys Gly Leu Ile Gly Val Asn Gly Val
                        420 425 430
Glu Leu Tyr Ala Asn Arg Arg Gly His Asp Ala Thr Trp Gly Val Gln
                435 440 445
Asn Leu Thr Val Gly Pro Ala Ile Ala Ile Arg Pro Val Asp Ile Ser
        450 455 460
Leu Asn Leu Ala Asp Ala Thr Asn Phe Leu Gln Asp Ser Lys Ala Glu
465 470 475 480
Leu Glu Lys Ala Arg Lys Ile Leu Ser Glu Val Gly Arg Trp Tyr Asn
                                485 490 495
Ser Arg Glu Thr Val Ile Thr Ile Ile Val Val Met Val Val Ile Leu
                        500 505 510
Val Val Ile Ile Val Ile Ile Ile Val Leu Tyr Arg Leu Arg Arg
                  515 520 525
<210> SEQ ID NO: 14
<211>411
<223> Fct4 (fragment 1)
Phe Phe Gly Ala Val Ile Gly Thr Ile Ala Leu Gly Val Ala Thr Ser
1 5 10 15
Ala Gln Ile Thr Ala Gly Ile Ala Leu Ala Glu Ala Arg Glu Ala Lys
                        20 25 30
Arg Asp Ile Ala Leu Ile Lys Glu Ser Met Thr Lys Thr His Lys Ser
                35 40 45
Ile Glu Leu Leu Gln Asn Ala Val Gly Glu Gln Ile Leu Ala Leu Lys
        50 55 60
Thr Leu Gln Asp Phe Val Asn Asp Glu Ile Lys Pro Ala Ile Ser Glu
65 70 75 80
Leu Gly Cys Glu Thr Ala Ala Leu Arg Leu Gly Ile Lys Leu Thr Gln
                                85 90 95
His Tyr Ser Glu Leu Leu Thr Ala Phe Gly Ser Asn Phe Gly Thr Ile
                        100 105 110
Gly Glu Lys Ser Leu Thr Leu Gln Ala Leu Ser Ser Leu Tyr Ser Ala
                115 120 125
Asn Ile Thr Glu Ile Met Thr Thr Ile Arg Thr Gly Gln Ser Asn Ile
        130 135 140
Tyr Asp Val Ile Tyr Thr Glu Gln Ile Lys Gly Thr Val Ile Asp Val
145 150 155 160
Asp Leu Glu Arg Tyr Met Val Thr Leu Ser Val Lys Ile Pro Ile Leu
                                165 170 175
Ser Glu Val Pro Gly Val Leu Ile His Lys Ala Ser Ser Ile Ser Tyr
                        180 185 190
Asn Ile Asp Gly Glu Glu Trp Tyr Val Thr Val Pro Ser His Ile Leu
                195 200 205
Ser Arg Ala Ser Phe Leu Gly Gly Ala Asp Ile Thr Asp Cys Val Glu
        210 215 220
Ser Arg Leu Thr Tyr Ile Cys Pro Arg Asp Pro Ala Gln Leu Ile Pro
225 230 235 240
Asp Ser Gln Gln Lys Cys Ile Leu Gly Asp Thr Thr Arg Cys Pro Val
                                245 250 255
Thr Lys Val Val Asp Ser Leu Ile Pro Lys Phe Ala Phe Val Asn Gly
                        260 265 270
Gly Val Val Ala Asn Cys Ile Ala Ser Thr Cys Thr Cys Gly Thr Gly
                275 280 285
Arg Arg Pro Ile Ser Gln Asp Arg Ser Lys Gly Val Val Phe Leu Thr
        290 295 300
His Asp Asn Cys Gly Leu Ile Gly Val Asn Gly Val Glu Leu Tyr Ala
305 310 315 320
Asn Arg Arg Gly His Asp Ala Thr Trp Gly Val Gln Asn Leu Thr Val
                                325 330 335
Gly Pro Ala Ile Ala Ile Arg Pro Val Asp Ile Ser Leu Asn Leu Ala
                        340 345 350
Asp Ala Thr Asn Phe Leu Gln Asp Ser Lys Ala Glu Leu Glu Lys Ala
                355 360 365
Arg Lys Ile Leu Ser Glu Val Gly Arg Trp Tyr Asn Ser Arg Glu Thr
        370 375 380
Val Ile Thr Ile Ile Val Val Met Val Val Ile Leu Val Val Ile Ile
385 390 395 400
Val Ile Ile Ile Val Leu Tyr Arg Leu Arg Arg
                                405 410
<210> SEQ ID NO: 15
<211> 91
<223> Fct4 (fragment 2)
Gln Ile Pro Arg Asp Arg Leu Ser Asn Ile Gly Val Ile Val Asp Glu
1 5 10 15
Gly Lys Ser Leu Lys Ile Ala Gly Ser His Glu Ser Arg Tyr Ile Val
                        20 25 30
Leu Ser Leu Val Pro Gly Val Asp Phe Glu Asn Gly Cys Gly Thr Ala
                35 40 45
Gln Val Ile Gln Tyr Lys Ser Leu Leu Asn Arg Leu Leu Ile Pro Leu
        50 55 60
Arg Asp Ala Leu Asp Leu Gln Glu Ala Leu Ile Thr Val Thr Asn Asp
65 70 75 80
Thr Thr Gln Asn Ala Gly Ala Pro Gln Ser Arg
                                85 90
<210> SEQ ID NO: 16
<211> <223> 25
Fct4 signal peptide
MATYIQRVQC ISTSLLVVLT TLVSC 25
<210> SEQ ID NO: 17
<211> 4391
<223> codon-optimised SIV gal-pol nucleic acid sequence (from pGM691)
atgggagctg ccacatctgc cctgaataga cggcagctgg accagttcga gaagatcaga 60
ctgcggccca acggcaagaa gaagtaccag atcaagcacc tgatctgggc cggcaaagag 120
atggaaagat tcggcctgca cgagcggctg ctggaaaccg aggaaggctg caagagaatt 180
atcgaggtgc tgtaccctct ggaacctacc ggctctgagg gcctgaagtc cctgttcaat 240
ctcgtgtgcg tgctgtactg cctgcacaaa gaacagaaag tgaaggacac cgaagaggcc 300
gtggccacag ttagacagca ctgccacctg gtggaaaaag agaagtccgc cacagagaca 360
agcagcggcc agaagaagaa cgacaaggga attgctgccc ctcctggcgg cagccagaat 420
tttcctgctc agcagcaggg aaacgcctgg gtgcacgttc cactgagccc tagaacactg 480
aatgcctggg tcaaagccgt ggaagagaag aagtttggcg ccgagatcgt gcccatgttc 540
caggctctgt ctgagggctg caccccttac gacatcaacc agatgctgaa cgtgctggga 600
gatcaccagg gcgctctgca gatcgtgaaa gagatcatca acgaagaggc tgcccagtgg 660
gacgtgacac atccattgcc tgctggacct ctgccagccg gacaactgag agatcctaga 720
ggctctgata tcgccggcac caccagctct gtgcaagagc agctggaatg gatctacacc 780
gccaatccta gagtggacgt gggcgccatc tacagaagat ggatcatcct gggcctgcag 840
aaatgcgtga agatgtacaa ccccgtgtcc gtgctggaca tcagacaggg acccaaagag 900
cccttcaagg actacgtgga ccggttctat aaggccatta gagccgagca ggccagcggc 960
gaagtgaagc agtggatgac agagagcctg ctgatccaga acgccaatcc agactgcaaa 1020
gtgatcctga aaggcctggg catgcacccc acactggaag agatgctgac agcctgtcaa 1080
ggcgttggcg gcccttctta caaagccaaa gtgatggccg agatgatgca gaccatgcag 1140
aaccagaaca tggtgcagca aggcggccct aagagacaga ggcctcctct gagatgctac 1200
aactgcggca agttcggcca catgcagaga cagtgtcctg agcctaggaa aacaaaatgt 1260
ctaaagtgtg gaaaattggg acacctagca aaagactgca ggggacaggt gaatttttta 1320
gggtatggac ggtggatggg ggcaaaaccg agaaattttc ccgccgctac tcttggagcg 1380
gaaccgagtg cgcctcctcc accgageggc accaccccat acgacccagc aaagaagctc 1440
ctgcagcaat atgcagagaa agggaaacaa ctgagggagc aaaagaggaa tccaccggca 1500
atgaatccgg attggaccga gggatattct ttgaactccc tctttggaga agaccaataa 1560
agaccgtgta catcgagggc gtgcccatca aggctctgct ggatacaggc gccgacgaca 1620
ccatcatcaa agagaacgac ctgcagctga gcggcccttg gaggcctaag atcattggag 1680
gaatcggcgg aggcctgaac gtcaaagagt acaacgaccg ggaagtgaag atcgaggaca 1740
agatcctgag gggcacaatc ctgctgggcg ccacacctat caacatcatc ggcagaaatc 1800
tgctggcccc tgccggcgct agactggtta tgggacagct ctctgagaag atccccgtga 1860
cacccgtgaa gctgaaagaa ggcgctagag gaccttgtgt gcgacagtgg cctctgagca 1920
aagagaagat tgaggccctg caagaaatct gtagccagct ggaacaagag ggcaagatca 1980
gcagagttgg cggcgagaac gcctacaata cccctatctt ctgcatcaag aaaaaggaca 2040
agagccagtg gcggatgctg gtggacttta gagagctgaa caaggctacc caggacttct 2100
tcgaggtgca gctgggaatt cctcatcctg ccggcctgcg gaagatgaga cagatcacag 2160
tgctggatgt gggcgacgcc tactacagca tccctctgga ccccaacttc agaaagtaca 2220
ccgccttcac aatccccacc gtgaacaatc aaggccctgg catcagatac cagttcaact 2280
gcctgcctca aggctggaag ggcagcccca ccatttttca gaataccgcc gccagcatcc 2340
tggaagaaat caagagaaac ctgcctgctc tgaccatcgt gcagtacatg gacgatctgt 2400
gggtcggaag ccaagagaat gagcacaccc acgacaagct ggtggaacag ctgagaacaa 2460
agctgcaggc ctggggcctc gaaacccctg agaagaaggt gcagaaagaa cctccttacg 2520
agtggatggg ctacaagctg tggcctcaca agtgggagct gagccggatt cagctcgaag 2580
agaaggacga gtggaccgtg aacgacatcc agaaactcgt gggcaagctg aattgggcag 2640
cccagctgta tcccggcctg aggaccaaga acatctgcaa gctgatccgg ggaaagaaga 2700
acctgctgga actggtcaca tggacacctg aggccgaggc cgaatatgcc gagaatgccg 2760
aaatcctgaa aaccgagcaa gaggggacct actacaagcc tggcattcca atcagagctg 2820
ccgtgcagaa actggaaggc ggccagtggt cctaccagtt taagcaagaa ggccaggtcc 2880
tgaaagtggg caagtacacc aagcagaaga acacccacac caacgagctg aggacactgg 2940
ctggcctggt ccagaaaatc tgcaaagagg ccctggtcat ttggggcatc ctgcctgttc 3000
tggaactgcc cattgagcgg gaagtgtggg aacagtggtg ggccgattac tggcaagtgt 3060
cttggatccc cgagtgggac ttcgtgtcta cccctcctct gctgaaactg tggtacaccc 3120
tgacaaaaga gcccattcct aaagaggacg tctactacgt tgacggcgcc tgcaaccgga 3180
actccaaaga aggcaaggcc ggctacatca gccagtacgg caagcagaga gtggaaaccc 3240
tggaaaacac caccaaccag caggccgagc tgaccgccat taagatggcc ctggaagata 3300
gcggccccaa tgtgaacatc gtgaccgact ctcagtacgc catgggaatc ctgacagccc 3360
agcctacaca gagcgatagc cctctggttg agcagatcat tgccctgatg attcagaagc 3420
agcaaatcta cctgcagtgg gtgcccgctc acaaaggcat cggcggaaac gaagagatcg 3480
ataagctggt gtccaaggga atcagacggg tgctgttcct ggaaaagatt gaagaggccc 3540
aagaggaaca cgagcgctac cacaacaact ggaagaatct ggccgacacc tacggactgc 3600
cccagatcgt ggccaaagaa atcgtggcta tgtgccccaa gtgtcagatc aagggcgaac 3660
ctgtgcacgg ccaagtggat gcttctcctg gcacatggca gatggactgt acccacctgg 3720
aaggcaaagt ggtcatcgtg gctgtgcacg tggcctccgg ctttattgag gccgaagtga 3780
tccccagaga gacaggcaaa gaaaccgcca agttcctgct gaagatcctg tccagatggc 3840
ccatcacaca gctgcacacc gacaacggcc ctaacttcac atctcaagag gtggccgcca 3900
tctgttggtg gggaaagatt gagcacacaa ccggcattcc ctacaatcca cagagccagg 3960
gcagcatcga gtccatgaac aagcagctca aagagattat cggcaagatc cgggacgact 4020
gccagtacac agaaacagcc gtgctgatgg cctgtcacat ccacaacttc aagcggaaag 4080
gcggcatcgg aggacagaca tctgccgaga gactgatcaa tatcatcacc actcagctgg 4140
aaatccagca cctccagacc aagatccaga agattctgaa cttccgggtg tactaccgcg 4200
agggcagaga tcctgtttgg aaaggcccag cacagctgat ctggaaaggc gaaggtgccg 4260
tggtgctgaa ggatggctct gatctgaagg tggtgcccag acggaaggcc aagattatca 4320
aggattacga gcccaaacag cgcgtgggca atgaaggcga cgttgagggc acaagaggca 4380
gcgacaattg a 4391
<210> SEQ ID NO: 18
<211> 4391
<213> Wild-type Simian immunodeficiency virus gagpol
atgggggcgg ctacctcagc actaaatagg agacaattag accaatttga gaaaatacga 60
cttcgcccga acggaaagaa aaagtaccaa attaaacatt taatatgggc aggcaaggag 120
atggagcgct tcggcctcca tgagaggttg ttggagacag aggaggggtg taaaagaatc 180
atagaagtcc tctaccccct agaaccaaca ggatcggagg gcttaaaaag tctgttcaat 240
cttgtgtgcg tactatattg cttgcacaag gaacagaaag tgaaagacac agaggaagca 300
gtagcaacag taagacaaca ctgccatcta gtggaaaaag aaaaaagtgc aacagagaca 360
tctagtggac aaaagaaaaa tgacaaggga atagcagcgc cacctggtgg cagtcagaat 420
tttccagcgc aacaacaagg aaatgcctgg gtacatgtac ccttgtcacc gcgcacctta 480
aatgcgtggg taaaagcagt agaggagaaa aaatttggag cagaaatagt acccatgttt 540
caagccctat cagaaggctg cacaccctat gacattaatc agatgcttaa tgtgctagga 600
gatcatcaag gggcattaca aatagtgaaa gagatcatta atgaagaagc agcccagtgg 660
gatgtaacac acccactacc cgcaggaccc ctaccagcag gacagctcag ggaccctcgc 720
ggctcagata tagcagggac caccagctca gtacaagaac agttagaatg gatctatact 780
gctaaccccc gggtagatgt aggtgccatc taccggagat ggattattct aggacttcaa 840
aagtgtgtca aaatgtacaa cccagtatca gtcctagaca ttaggcaggg acctaaagag 900
cccttcaagg attatgtgga cagattttac aaggcaatta gagcagaaca agcctcaggg 960
gaagtgaaac aatggatgac agaatcatta ctcattcaaa atgctaatcc agattgtaag 1020
gtcatcctga agggcctagg aatgcacccc acccttgaag aaatgttaac ggcttgtcag 1080
ggggtaggag gcccaagcta caaagcaaaa gtaatggcag aaatgatgca gaccatgcaa 1140
aatcaaaaca tggtgcagca gggaggtcca aaaagacaaa gacccccact aagatgttat 1200
aattgtggaa aatttggcca tatgcaaaga caatgtccgg aaccaaggaa aacaaaatgt 1260
ctaaagtgtg gaaaattggg acacctagca aaagactgca ggggacaggt gaatttttta 1320
gggtatggac ggtggatggg ggcaaaaccg agaaattttc ccgccgctac tcttggagcg 1380
gaaccgagtg cgcctcctcc accgageggc accaccccat acgacccagc aaagaagctc 1440
ctgcagcaat atgcagagaa agggaaacaa ctgagggagc aaaagaggaa tccaccggca 1500
atgaatccgg attggaccga gggatattct ttgaactccc tctttggaga agaccaataa 1560
agacagtgta tatagaaggg gtccccatta aggcactgct agacacaggg gcagatgaca 1620
ccataattaa agaaaatgat ttacaattat caggtccatg gagacccaaa attatagggg 1680
gcataggagg aggccttaat gtaaaagaat ataacgacag ggaagtaaaa atagaagata 1740
aaattttgag aggaacaata ttgttaggag caactcccat taatataata ggtagaaatt 1800
tgctggcccc ggcaggtgcc cggttagtaa tgggacaatt atcagaaaaa attcctgtca 1860
cacctgtcaa attgaaggaa ggggctcggg gaccctgtgt aagacaatgg cctctctcta 1920
aagagaagat tgaagcttta caggaaatat gttoccaatt agagcaggaa ggaaaaatca 1980
gtagagtagg aggagaaaat gcatacaata ccccaatatt ttgcataaag aagaaggaca 2040
aatcccagtg gaggatgcta gtagacttta gagagttaaa taaggcaacc caagatttct 2100
ttgaagtgca attagggata ccccacccag caggattaag aaagatgaga cagataacag 2160
ttttagatgt aggagacgcc tattattcca taccattgga tccaaatttt aggaaatata 2220
ctgcttttac tattcccaca gtgaataatc agggacccgg gattaggtat caattcaact 2280
gtctcccgca agggtggaaa ggatctccta caatcttcca aaatacagca gcatccattt 2340
tggaggagat aaaaagaaac ttgccagcac taaccattgt acaatacatg gatgatttat 2400
gggtaggttc tcaagaaaat gaacacaccc atgacaaatt agtagaacag ttaagaacaa 2460
aattacaagc ctggggctta gaaaccccag aaaagaaggt gcaaaaagaa ccaccttatg 2520
agtggatggg atacaaactt tggcctcaca aatgggaact aagcagaata caactggagg 2580
aaaaagatga atggactgtc aatgacatcc agaagttagt tgggaaacta aattgggcag 2640
cacaattgta tccaggtctt aggaccaaga atatatgcaa gttaattaga ggaaagaaaa 2700
atctgttaga gctagtgact tggacacctg aggcagaagc tgaatatgca gaaaatgcag 2760
agattcttaa aacagaacag gaaggaacct attacaaacc aggaatacct attagggcag 2820
cagtacagaa attggaagga ggacagtgga gttaccaatt caaacaagaa ggacaagtct 2880
tgaaagtagg aaaatacacc aagcaaaaga acacccatac aaatgaactt cgcacattag 2940
ctggtttagt gcagaagatt tgcaaagaag ctctagttat ttgggggata ttaccagttc 3000
tagaactccc gatagaaaga gaggtatggg aacaatggtg ggcggattac tggcaggtaa 3060
gctggattcc cgaatgggat tttgtcagca ccccaccttt gctcaaacta tggtacacat 3120
taacaaaaga acccataccc aaggaggacg tttactatgt agatggagca tgcaacagaa 3180
attcaaaaga aggaaaagca ggatacatct cacaatacgg aaaacagaga gtagaaacat 3240
tagaaaacac taccaatcag caagcagaat taacagctat aaaaatggct ttggaagaca 3300
gtgggcctaa tgtgaacata gtaacagact ctcaatatgc aatgggaatt ttgacagcac 3360
aacccacaca aagtgattca ccattagtag agcaaattat agccttaatg atacaaaagc 3420
aacaaatata tttgcagtgg gtaccagcac ataaaggaat aggaggaaat gaggagatag 3480
ataaattagt gagtaaaggc attagaagag ttttattctt agaaaaaata gaagaagctc 3540
aagaagagca tgaaagatat cataataatt ggaaaaacct agcagataca tatgggcttc 3600
cacaaatagt agcaaaagag atagtggcca tgtgtccaaa atgtcagata aagggagaac 3660
cagtgcatgg acaagtggat gcctcacctg gaacatggca gatggattgt actcatctag 3720
aaggaaaagt agtcatagtt gcggtccatg tagccagtgg attcatagaa gcagaagtca 3780
tacctaggga aacaggaaaa gaaacggcaa agtttctatt aaaaatactg agtagatggc 3840
ctataacaca gttacacaca gacaatgggc ctaactttac ctcccaagaa gtggcagcaa 3900
tatgttggtg gggaaaaatt gaacatacaa caggtatacc atataacccc caatctcaag 3960
gatcaataga aagcatgaac aaacaattaa aagagataat tgggaaaata agagatgatt 4020
gccaatatac agagacagca gtactgatgg cttgccatat tcacaatttt aaaagaaagg 4080
gaggaatagg gggacagact tcagcagaga gactaattaa tataataaca acacaattag 4140
aaatacaaca tttacaaacc aaaattcaaa aaattttaaa ttttagagtc tactacagag 4200
aagggagaga ccctgtgtgg aaaggaccag cacaattaat ctggaaaggg gaaggagcag 4260
tggtcctcaa ggacggaagt gacctaaagg ttgtaccaag aaggaaagct aaaattatta 4320
aggattatga acccaaacaa agagtgggta atgagggtga cgtggaaggt accaggggat 4380
ctgataacta a 4391
<210> SEQ ID NO: 19
<211> 10536
<223> pGM830
ggtacctcaa tattggccat tagccatatt attcattggt tatatagcat aaatcaatat 60
tggctattgg ccattgcata cgttgtatct atatcataat atgtacattt atattggctc 120
atgtccaata tgaccgccat gttggcattg attattgact agttattaat agtaatcaat 180
tacggggtca ttagttcata gcccatatat ggagttccgc gttacataac ttacggtaaa 240
tggcccgcct ggctgaccgc ccaacgaccc ccgcccattg acgtcaataa tgacgtatgt 300
tcccatagta acgccaatag ggactttcca ttgacgtcaa tgggtggagt atttacggta 360
aactgcccac ttggcagtac atcaagtgta tcatatgcca agtccgcccc ctattgacgt 420
caatgacggt aaatggcccg cctggcatta tgcccagtac atgaccttac gggactttcc 480
tacttggcag tacatctacg tattagtcat cgctattacc atggtgatgc ggttttggca 540
gtacaccaat gggcgtggat agcggtttga ctcacgggga tttccaagtc tccaccccat 600
tgacgtcaat gggagtttgt tttggcacca aaatcaacgg gactttccaa aatgtcgtaa 660
caactgcgat cgcccgcccc gttgacgcaa atgggcggta ggcgtgtacg gtgggaggtc 720
tatataagca gagctcgctg gcttgtaact cagtctctta ctaggagacc agcttgagcc 780
tgggtgttcg ctggttagcc taacctggtt ggccaccagg ggtaaggact ccttggctta 840
gaaagctaat aaacttgcct gcattagagc ttatctgagt caagtgtcct cattgacgcc 900
tcactctctt gaacgggaat cttccttact gggttctctc tctgacccag gcgagagaaa 960
ctccagcagt ggcgcccgaa cagggacttg agtgagagtg taggcacgta cagctgagaa 1020
ggcgtcggac gcgaaggaag cgcggggtgc gacgcgacca agaaggagac ttggtgagta 1080
ggcttctcga gtgccgggaa aaagctcgag cctagttaga ggactaggag aggccgtagc 1140
cgtaactact ctgggcaagt agggcaggcg gtgggtacgc aattgggggc ggctacctca 1200
gcactaaata ggagacaatt agaccaattt gagaaaatac gacttcgccc gaacggaaag 1260
aaaaagtacc aaattaaaca tttaatattg ggcaggcaag gagattggag cgcttcggcc 1320
tccatgagag gttgttggag acagaggagg ggtgtaaaag aatcatagaa gtcctctacc 1380
ccctagaacc aacaggatcg gagggcttaa aaagtctgtt caatcttgtg tgcgtgctat 1440
attgcttgca caaggaacag aaagtgaaag acacagagga agcagtagca acagtaagac 1500
aacactgcca tctagtggaa aaagaaaaaa gtgcaacaga gacatctagt ggacaaaaga 1560
aaaatgacaa gggaatagca gcgccacctg gtggcagtca gaattttcca gcgcaacaac 1620
aaggaaattg cctgggtaca tgtacccttg tcaccgcgca ccttaaatgc gtgggtaaaa 1680
gcagtagagg agaaaaaatt tggagcagaa atagtaccca tgtttcaagc cctatcgcct 1740
gcaggccgtt tgtgctaggg ttcttaggct tcttgggggc tgctggaact gcattgggag 1800
cagcggcgac agccctgacg gtccagtctc agcatttgct tgctgggata ctgcagcagc 1860
agaagaatct gctggcggct gtggaggctc aacagcagat gttgaagctg accatttggg 1920
gtgttaaaaa cctcaatgcc cgcgtcacag cccttgagaa gtacctagag gatcaggcac 1980
gactaaactc ctgggggtgc gcatggaaac aagtatgtca taccacagtg gagtggccct 2040
ggacaaatcg gactccggat tggcaaaata agacttggtt ggagtgggaa agacaaatag 2100
ctgatttgga aagcaacatt acgagacaat tagtgaaggc tagagaacaa gaggaaaaga 2160
atctagatgc ctatcagaag ttaactagtt ggtcagattt ctggtcttgg ttcgatttct 2220
caaaatggct taacatttta aaaaagggat ttttagtaat agtaggaata atagggttaa 2280
gattacttta cacagtatat ggatgtatag tgagggttag gcagggatat gttcctctat 2340
ctccacagat ccatataaag cggcaatttt aaaagaaagg gaggaatagg gggacagact 2400
tcagcagaga gactaattaa tataataaca acacaattag aaatacaaca tttacaaacc 2460
aaaattcaaa aaattttaaa ttttagagcc gcggagatct gttacataac ttatggtaaa 2520
tggcctgcct ggctgactgc ccaatgaccc ctgcccaatg atgtcaataa tgatgtatgt 2580
tcccatgtaa tgccaatagg gactttccat tgatgtcaat gggtggagta tttatggtaa 2640
ctgcccactt ggcagtacat caagtgtatc atatgccaag tatgccccct attgatgtca 2700
atgatggtaa atggcctgcc tggcattatg cccagtacat gaccttatgg gactttccta 2760
cttggcagta catctatgta ttagtcattg ctattaccat gggaattcac tagtggagaa 2820
gagcatgctt gagggctgag tgcccctcag tgggcagaga gcacatggcc cacagtccct 2880
gagaagttgg ggggaggggt gggcaattga actggtgcct agagaaggtg gggcttgggt 2940
aaactgggaa agtgatgtgg tgtactggct ccaccttttt ccccagggtg ggggagaacc 3000
atatataagt gcagtagtct ctgtgaacat tcaagcttct gccttctccc tcctgtgagt 3060
ttgctagcca ccatgcagag aagccctctg gagaaggcct ctgtggtgag caagctgttc 3120
ttcagctgga ccaggcccat cctgaggaag ggctacaggc agagactgga gctgtctgac 3180
atctaccaga tcccctctgt ggactctgct gacaacctgt ctgagaagct ggagagggag 3240
tgggatagag agctggccag caagaagaac cccaagctga tcaatgccct gaggagatgc 3300
ttcttctgga gattcatgtt ctatggcatc ttcctgtacc tgggggaagt gaccaaggct 3360
gtgcagcctc tgctgctggg cagaatcatt gccagctatg accctgacaa caaggaggag 3420
aggagcattg ccatctacct gggcattggc ctgtgcctgc tgttcattgt gaggaccctg 3480
ctgctgcacc ctgccatctt tggcctgcac cacattggca tgcagatgag gattgccatg 3540
ttcagcctga tctacaagaa aaccctgaag ctgtccagca gagtgctgga caagatcagc 3600
attggccagc tggtgagcct gctgagcaac aacctgaaca agtttgatga gggcctggcc 3660
ctggcccact ttgtgtggat tgcccctctg caggtggccc tgctgatggg cctgatttgg 3720
gagctgctgc aggcctctgc cttttgtggc ctgggcttcc tgattgtgct ggccctgttt 3780
caggctggcc tgggcaggat gatgatgaag tacagggacc agagggcagg caagatcagt 3840
gagaggctgg tgatcacctc tgagatgatt gagaacatcc agtctgtgaa ggcctactgt 3900
tgggaggaag ctatggagaa gatgattgaa aacctgaggc agacagagct gaagctgacc 3960
aggaaggctg cctatgtgag atacttcaac agctctgcct tcttcttctc tggcttcttt 4020
gtggtgttcc tgtctgtgct gccctatgcc ctgatcaagg ggatcatcct gagaaagatt 4080
ttcaccacca tcagcttctg cattgtgctg aggatggctg tgaccagaca gttcccctgg 4140
gctgtgcaga cctggtatga cagcctgggg gccatcaaca agatccagga cttcctgcag 4200
aagcaggagt acaagaccct ggagtacaac ctgaccacca cagaagtggt gatggagaat 4260
gtgacagcct tctgggagga gggctttggg gagctgtttg agaaggccaa gcagaacaac 4320
aacaacagaa agaccagcaa tggggatgac tccctgttct tctccaactt ctccctgctg 4380
ggcacacctg tgctgaagga catcaacttc aagattgaga gggggcagct gctggctgtg 4440
gctggatcta caggggctgg caagaccagc ctgctgatga tgatcatggg ggagctggag 4500
ccttctgagg gcaagatcaa gcactctggc aggatcagct tttgcagcca gttcagctgg 4560
atcatgcctg gcaccatcaa ggagaacatc atctttggag tgagctatga tgagtacaga 4620
tacaggagtg tgatcaaggc ctgccagctg gaggaggaca tcagcaagtt tgctgagaag 4680
gacaacattg tgctggggga gggaggcatt acactgtctg ggggccagag agccagaatc 4740
agcctggcca gggctgtgta caaggatgct gacctgtacc tgctggactc cccctttggc 4800
tacctggatg tgctgacaga gaaggagatt tttgagagct gtgtgtgcaa gctgatggcc 4860
aacaagacca gaatcctggt gaccagcaag atggagcacc tgaagaaggc tgacaagatc 4920
ctgatcctgc atgagggcag cagctacttc tatgggacct tctctgagct gcagaacctg 4980
cagcctgact tcagctctaa gctgatgggc tgtgacagct ttgaccagtt ctctgctgag 5040
aggaggaaca gcatcctgac agagaccctg cacagattca gcctggaggg agatgcccct 5100
gtgagctgga cagagaccaa gaagcagagc ttcaagcaga caggggagtt tggggagaag 5160
aggaagaact ccatcctgaa ccccatcaac agcatcagga agttcagcat tgtgcagaaa 5220
acccccctgc agatgaatgg cattgaggaa gattctgatg agcccctgga gaggagactg 5280
agcctggtgc ctgattctga gcagggagag gccatcctgc ctaggatctc tgtgatcagc 5340
acaggcccta cactgcaggc cagaaggagg cagtctgtgc tgaacctgat gacccactct 5400
gtgaaccagg gccagaacat ccacaggaaa accacagcct ccaccaggaa agtgagcctg 5460
gcccctcagg ccaatctgac agagctggac atctacagca ggaggctgtc tcaggagaca 5520
ggcctggaga tttctgagga gatcaatgag gaggacctga aagagtgctt ctttgatgac 5580
atggagagca tccctgctgt gaccacctgg aacacctacc tgagatacat cacagtgcac 5640
aagagcctga tctttgtgct gatctggtgc ctggtgatct tcctggctga agtggctgcc 5700
tctctggtgg tgctgtggct gctgggaaac accccactgc aggacaaggg caacagcacc 5760
cacagcagga acaacagcta tgctgtgatc atcacctcca cctccagcta ctatgtgttc 5820
tacatctatg tgggagtggc tgataccctg ctggctatgg gcttctttag aggcctgccc 5880
ctggtgcaca cactgatcac agtgagcaag atcctccacc acaagatgct gcactctgtg 5940
ctgcaggctc ctatgagcac cctgaatacc ctgaaggctg ggggcatcct gaacagattc 6000
tccaaggata ttgccatcct ggatgacctg ctgcctctca ccatctttga cttcatccag 6060
ctgctgctga ttgtgattgg ggccattgct gtggtggcag tgctgcagcc ctacatcttt 6120
gtggccacag tgcctgtgat tgtggccttc atcatgctga gggcctactt tctgcagacc 6180
tcccagcagc tgaagcagct ggagtctgag ggcagaagcc ccatcttcac ccacctggtg 6240
acaagcctga agggcctgtg gaccctgaga gcctttggca ggcagcccta ctttgagacc 6300
ctgttccaca aggccctgaa cctgcacaca gccaactggt tcctctacct gtccaccctg 6360
agatggttcc agatgagaat tgagatgatc tttgtcatct tcttcattgc tgtgaccttc 6420
atcagcattc tgaccacagg agagggagag ggcagagtgg gcattatcct gaccctggcc 6480
atgaacatca tgagcacact gcagtgggca gtgaacagca gcattgatgt ggacagcctg 6540
atgaggagtg tgagcagagt gttcaagttc attgatatgc ccacagaggg caagcctacc 6600
aagagcacca agccctacaa gaatggccag ctgagcaaag tgatgatcat tgagaacagc 6660
catgtgaaga aggatgatat ctggcccagt ggaggccaga tgacagtgaa ggacctgaca 6720
gccaagtaca cagagggggg caatgctatc ctggagaaca tctccttcag catctcccct 6780
ggccagagag tgggactgct gggaagaaca ggctctggca agtctaccct gctgtctgcc 6840
ttcctgaggc tgctgaacac agagggagag atccagattg atggagtgtc ctgggacagc 6900
atcacactgc agcagtggag gaaggccttt ggtgtgatcc cccagaaagt gttcatcttc 6960
agtggcacct tcaggaagaa cctggacccc tatgagcagt ggtctgacca ggagatttgg 7020
aaagtggctg atgaagtggg cctgagaagt gtgattgagc agttccctgg caagctggac 7080
tttgtcctgg tggatggggg ctgtgtgctg agccatggcc acaagcagct gatgtgcctg 7140
gccagatcag tgctgagcaa ggccaagatc ctgctgctgg atgagccttc tgcccacctg 7200
gatcctgtga cctaccagat catcaggagg accctcaagc aggcctttgc tgactgcaca 7260
gtcatcctgt gtgagcacag gattgaggcc atgctggagt gccagcagtt cctggtgatt 7320
gaggagaaca aagtgaggca gtatgacagc atccagaagc tgctgaatga gaggagcctg 7380
ttcaggcagg ccatcagccc ctctgataga gtgaagctgt tcccccacag gaacagctcc 7440
aagtgcaaga gcaagcccca gattgctgcc ctgaaggagg agacagagga ggaagtgcag 7500
gacaccaggc tgtgagggcc caatcaacct ctggattaca aaatttgtga aagattgact 7560
ggtattctta actatgttgc tccttttacg ctatgtggat acgctgcttt aatgcctttg 7620
tatcatgcta ttgcttcccg tatggctttc attttctcct ccttgtataa atcctggttg 7680
ctgtctcttt atgaggagtt gtggcccgtt gtcaggcaac gtggcgtggt gtgcactgtg 7740
tttgctgacg caacccccac tggttggggc attgccacca cctgtcaget cctttccggg 7800
actttcgctt tccccctccc tattgccacg gcggaactca tcgccgcctg ccttgcccgc 7860
tgctggacag gggctcggct gttgggcact gacaattccg tggtgttgtc ggggaaatca 7920
tcgtcctttc cttggctgct cgcctgtgtt gccacctgga ttctgcgcgg gacgtccttc 7980
tgctacgtcc cttcggccct caatccagcg gaccttcctt cccgcggcct gctgccggct 8040
ctgcggcctc ttccgcgtct tcgccttcgc cctcagacga gtcggatctc cctttgggcc 8100
gcctccccgc aagcttcgca ctttttaaaa gaaaagggag gactggatgg gatttattac 8160
tccgatagga cgctggcttg taactcagtc tcttactagg agaccagctt gagcctgggt 8220
gttcgctggt tagcctaacc tggttggcca ccaggggtaa ggactccttg gcttagaaag 8280
ctaataaact tgcctgcatt agagctctta cgcgtcccgg gctcgagatc cgcatctcaa 8340
ttagtcagca accatagtcc cgcccctaac tccgcccatc ccgcccctaa ctccgcccag 8400
ttccgcccat tctccgcccc atggctgact aatttttttt atttatgcag aggccgaggc 8460
cgcctcggcc tctgagctat tccagaagta gtgaggaggc ttttttggag gcctaggctt 8520
ttgcaaaaag ctaacttgtt tattgcagct tataatggtt acaaataaag caatagcatc 8580
acaaatttca caaataaagc atttttttca ctgcattcta gttgtggttt gtccaaactc 8640
atcaatgtat cttatcatgt ctgtccgctt cctcgctcac tgactcgctg cgctcggtcg 8700
ttcggctgcg gcgagcggta tcagctcact caaaggcggt aatacggtta tccacagaat 8760
caggggataa cgcaggaaag aacatgtgag caaaaggcca gcaaaaggcc aggaaccgta 8820
aaaaggccgc gttgctggcg tttttccata ggctccgccc ccctgacgag catcacaaaa 8880
atcgacgctc aagtcagagg tggcgaaacc cgacaggact ataaagatac caggcgtttc 8940
cccctggaag ctccctcgtg cgctctcctg ttccgaccct gccgcttacc ggatacctgt 9000
ccgcctttct cccttcggga agcgtggcgc tttctcatag ctcacgctgt aggtatctca 9060
gttcggtgta ggtcgttcgc tccaagctgg gctgtgtgca cgaacccccc gttcagcccg 9120
accgctgcgc cttatccggt aactatcgtc ttgagtccaa cccggtaaga cacgacttat 9180
cgccactggc agcagccact ggtaacagga ttagcagagc gaggtatgta ggcggtgcta 9240
cagagttctt gaagtggtgg cctaactacg gctacactag aagaacagta tttggtatct 9300
gcgctctgct gaagccagtt accttcggaa aaagagttgg tagctcttga tccggcaaac 9360
aaaccaccgc tggtagcggt ggtttttttg tttgcaagca gcagattacg cgcagaaaaa 9420
aaggatctca agaagatcct ttgatctttt ctacggggtc tgacgctcag tggaacgaaa 9480
actcacgtta agggattttg gtcatgagat tatcaaaaag gatcttcacc tagatccttt 9540
taaattaaaa atgaagtttt aaatcaatct aaagtatata tgagtaaact tggtctgaca 9600
gttagaaaaa ctcatcgagc atcaaatgaa actgcaattt attcatatca ggattatcaa 9660
taccatattt ttgaaaaagc cgtttctgta atgaaggaga aaactcaccg aggcagttcc 9720
ataggatggc aagatcctgg tatcggtctg cgattccgac tcgtccaaca tcaatacaac 9780
ctattaattt cccctcgtca aaaataaggt tatcaagtga gaaatcacca tgagtgacga 9840
ctgaatccgg tgagaatggc aacagcttat gcatttcttt ccagacttgt tcaacaggcc 9900
agccattacg ctcgtcatca aaatcactcg catcaaccaa accgttattc attcgtgatt 9960
gcgcctgagc gagacgaaat acgcgatcgc tgttaaaagg acaattacaa acaggaatcg 10020
aatgcaaccg gcgcaggaac actgccagcg catcaacaat attttcacct gaatcaggat 10080
attcttctaa tacctggaat gctgtttttc cggggatcgc agtggtgagt aaccatgcat 10140
catcaggagt acggataaaa tgcttgatgg tcggaagagg cataaattcc gtcagccagt 10200
ttagtctgac catctcatct gtaacatcat tggcaacgct acctttgcca tgtttcagaa 10260
acaactctgg cgcatcgggc ttcccataca atcgatagat tgtcgcacct gattgcccga 10320
cattatcgcg agcccattta tacccatata aatcagcatc catgttggaa tttaatcgcg 10380
gcctagagca agacgtttcc cgttgaatat ggctcataac accccttgta ttactgttta 10440
tgtaagcaga cagttttatt gttcatgatg atatattttt atcttgtgca atgtaacatc 10500
agagattttg agacacaaca attggtcgac ggatcc 10536
<210> SEQ ID NO: 20
<211> 9064
<223> pGM691
attgattatt gactagttat taatagtaat caattacggg gtcattagtt catagcccat 60
atatggagtt ccgcgttaca taacttacgg taaatggccc gcctggctga ccgcccaacg 120
acccccgccc attgacgtca ataatgacgt atgttcccat agtaacgcca atagggactt 180
tccattgacg tcaatgggtg gagtatttac ggtaaactgc ccacttggca gtacatcaag 240
tgtatcatat gccaagtacg ccccctattg acgtcaatga cggtaaatgg cccgcctggc 300
attatgccca gtacatgacc ttatgggact ttcctacttg gcagtacatc tacgtattag 360
tcatcgctat taccatggtc gaggtgagcc ccacgttctg cttcactctc cccatctccc 420
ccccctcccc acccccaatt ttgtatttat ttatttttta attattttgt gcagcgatgg 480
gggcgggggg gggggggggg cgcgcgccag gcggggcggg gcggggcgag gggcggggcg 540
gggcgaggcg gagaggtgcg gcggcagcca atcagagcgg cgcgctccga aagtttcctt 600
ttatggcgag gcggcggcgg cggcggccct ataaaaagcg aagcgcgcgg cgggcgggag 660
tcgctgcgcg ctgccttcgc cccgtgcccc gctccgccgc cgcctcgcgc cgcccgcccc 720
ggctctgact gaccgcgtta ctcccacagg tgagcgggcg ggacggccct tctcctccgg 780
gctgtaatta gcgcttggtt taatgacggc ttgtttcttt tctgtggctg cgtgaaagcc 840
ttgaggggct ccgggagggc cctttgtgcg gggggagcgg ctcggggggt gcgtgcgtgt 900
gtgtgtgcgt ggggagcgcc gcgtgcggct ccgcgctgcc cggcggctgt gagcgctgcg 960
ggcgcggcgc ggggctttgt gcgctccgca gtgtgcgcga ggggagcgcg gccgggggcg 1020
gtgccccgcg gtgcgggggg ggctgcgagg ggaacaaagg ctgcgtgcgg ggtgtgtgcg 1080
tgggggggtg agcagggggt gtgggcgcgt cggtcgggct gcaacccccc ctgcaccccc 1140
ctccccgagt tgctgagcac ggcccggctt cgggtgcggg gctccgtacg gggcgtggcg 1200
cggggctcgc cgtgccgggc ggggggtggc ggcaggtggg ggtgccgggc ggggcggggc 1260
cgcctcgggc cggggagggc tcgggggagg ggcgcggcgg cccccggagc gccggcggct 1320
gtcgaggcgc ggcgagccgc agccattgcc ttttatggta atcgtgcgag agggcgcagg 1380
gacttccttt gtcccaaatc tgtgcggagc cgaaatctgg gaggcgccgc cgcaccccct 1440
ctagcgggcg cggggcgaag cggtgcggcg ccggcaggaa ggaaatgggc ggggagggcc 1500
ttcgtgcgtc gccgcgccgc cgtccccttc tccctctcca gcctcggggc tgtccgcggg 1560
gggacggctg ccttcggggg ggacggggca gggcggggtt cggcttctgg cgtgtgaccg 1620
gcggctctag agcctctgct aaccatgttc atgccttctt ctttttccta cagctcctgg 1680
gcaacgtgct ggttattgtg ctgtctcatc attttggcaa agaattgctc gagccaccat 1740
gggagctgcc acatctgccc tgaatagacg gcagctggac cagttcgaga agatcagact 1800
gcggcccaac ggcaagaaga agtaccagat caagcacctg atctgggccg gcaaagagat 1860
ggaaagattc ggcctgcacg agcggctgct ggaaaccgag gaaggctgca agagaattat 1920
cgaggtgctg taccctctgg aacctaccgg ctctgagggc ctgaagtccc tgttcaatct 1980
cgtgtgcgtg ctgtactgcc tgcacaaaga acagaaagtg aaggacaccg aagaggccgt 2040
ggccacagtt agacagcact gccacctggt ggaaaaagag aagtccgcca cagagacaag 2100
cagcggccag aagaagaacg acaagggaat tgctgcccct cctggcggca gccagaattt 2160
tcctgctcag cagcagggaa acgcctgggt gcacgttcca ctgagcccta gaacactgaa 2220
tgcctgggtc aaagccgtgg aagagaagaa gtttggcgcc gagatcgtgc ccatgttcca 2280
ggctctgtct gagggctgca ccccttacga catcaaccag atgctgaacg tgctgggaga 2340
tcaccagggc gctctgcaga tcgtgaaaga gatcatcaac gaagaggctg cccagtggga 2400
cgtgacacat ccattgcctg ctggacctct gccagccgga caactgagag atcctagagg 2460
ctctgatatc gccggcacca ccagctctgt gcaagagcag ctggaatgga tctacaccgc 2520
caatcctaga gtggacgtgg gcgccatcta cagaagatgg atcatcctgg gcctgcagaa 2580
atgcgtgaag atgtacaacc ccgtgtccgt gctggacatc agacagggac ccaaagagcc 2640
cttcaaggac tacgtggacc ggttctataa ggccattaga gccgagcagg ccagcggcga 2700
agtgaagcag tggatgacag agagcctgct gatccagaac gccaatccag actgcaaagt 2760
gatcctgaaa ggcctgggca tgcaccccac actggaagag atgctgacag cctgtcaagg 2820
cgttggcggc ccttcttaca aagccaaagt gatggccgag atgatgcaga ccatgcagaa 2880
ccagaacatg gtgcagcaag gcggccctaa gagacagagg cctcctctga gatgctacaa 2940
ctgcggcaag ttcggccaca tgcagagaca gtgtcctgag cctaggaaaa caaaatgtct 3000
aaagtgtgga aaattgggac acctagcaaa agactgcagg ggacaggtga attttttagg 3060
gtatggacgg tggatggggg caaaaccgag aaattttccc gccgctactc ttggagcgga 3120
accgagtgcg cctcctccac cgagcggcac caccccatac gacccagcaa agaagctcct 3180
gcagcaatat gcagagaaag ggaaacaact gagggagcaa aagaggaatc caccggcaat 3240
gaatccggat tggaccgagg gatattcttt gaactccctc tttggagaag accaataaag 3300
accgtgtaca tcgagggcgt gcccatcaag gctctgctgg atacaggcgc cgacgacacc 3360
atcatcaaag agaacgacct gcagctgagc ggcccttgga ggcctaagat cattggagga 3420
atcggcggag gcctgaacgt caaagagtac aacgaccggg aagtgaagat cgaggacaag 3480
atcctgaggg gcacaatcct gctgggcgcc acacctatca acatcatcgg cagaaatctg 3540
ctggcccctg ccggcgctag actggttatg ggacagctct ctgagaagat ccccgtgaca 3600
cccgtgaagc tgaaagaagg cgctagagga ccttgtgtgc gacagtggcc tctgagcaaa 3660
gagaagattg aggccctgca agaaatctgt agccagctgg aacaagaggg caagatcagc 3720
agagttggcg gcgagaacgc ctacaatacc cctatcttct gcatcaagaa aaaggacaag 3780
agccagtggc ggatgctggt ggactttaga gagctgaaca aggctaccca ggacttcttc 3840
gaggtgcagc tgggaattcc tcatcctgcc ggcctgcgga agatgagaca gatcacagtg 3900
ctggatgtgg gcgacgccta ctacagcatc cctctggacc ccaacttcag aaagtacacc 3960
gccttcacaa tccccaccgt gaacaatcaa ggccctggca tcagatacca gttcaactgc 4020
ctgcctcaag gctggaaggg cagccccacc atttttcaga ataccgccgc cagcatcctg 4080
gaagaaatca agagaaacct gcctgctctg accatcgtgc agtacatgga cgatctgtgg 4140
gtcggaagcc aagagaatga gcacacccac gacaagctgg tggaacagct gagaacaaag 4200
ctgcaggcct ggggcctcga aacccctgag aagaaggtgc agaaagaacc tccttacgag 4260
tggatgggct acaagctgtg gcctcacaag tgggagctga gccggattca gctcgaagag 4320
aaggacgagt ggaccgtgaa cgacatccag aaactcgtgg gcaagctgaa ttgggcagcc 4380
cagctgtatc ccggcctgag gaccaagaac atctgcaagc tgatccgggg aaagaagaac 4440
ctgctggaac tggtcacatg gacacctgag gccgaggccg aatatgccga gaatgccgaa 4500
atcctgaaaa ccgagcaaga ggggacctac tacaagcctg gcattccaat cagagctgcc 4560
gtgcagaaac tggaaggcgg ccagtggtcc taccagttta agcaagaagg ccaggtcctg 4620
aaagtgggca agtacaccaa gcagaagaac acccacacca acgagctgag gacactggct 4680
ggcctggtcc agaaaatctg caaagaggcc ctggtcattt ggggcatcct gcctgttctg 4740
gaactgccca ttgagcggga agtgtgggaa cagtggtggg ccgattactg gcaagtgtct 4800
tggatccccg agtgggactt cgtgtctacc cctcctctgc tgaaactgtg gtacaccctg 4860
acaaaagagc ccattcctaa agaggacgtc tactacgttg acggcgcctg caaccggaac 4920
tccaaagaag gcaaggccgg ctacatcagc cagtacggca agcagagagt ggaaaccctg 4980
gaaaacacca ccaaccagca ggccgagctg accgccatta agatggccct ggaagatagc 5040
ggccccaatg tgaacatcgt gaccgactct cagtacgcca tgggaatcct gacagcccag 5100
cctacacaga gcgatagccc tctggttgag cagatcattg ccctgatgat tcagaagcag 5160
caaatctacc tgcagtgggt gcccgctcac aaaggcatcg gcggaaacga agagatcgat 5220
aagctggtgt ccaagggaat cagacgggtg ctgttcctgg aaaagattga agaggcccaa 5280
gaggaacacg agcgctacca caacaactgg aagaatctgg ccgacaccta cggactgccc 5340
cagatcgtgg ccaaagaaat cgtggctatg tgccccaagt gtcagatcaa gggcgaacct 5400
gtgcacggcc aagtggatgc ttctcctggc acatggcaga tggactgtac ccacctggaa 5460
ggcaaagtgg tcatcgtggc tgtgcacgtg gcctccggct ttattgaggc cgaagtgatc 5520
cccagagaga caggcaaaga aaccgccaag ttcctgctga agatcctgtc cagatggccc 5580
atcacacagc tgcacaccga caacggccct aacttcacat ctcaagaggt ggccgccatc 5640
tgttggtggg gaaagattga gcacacaacc ggcattccct acaatccaca gagccagggc 5700
agcatcgagt ccatgaacaa gcagctcaaa gagattatcg gcaagatccg ggacgactgc 5760
cagtacacag aaacagccgt gctgatggcc tgtcacatcc acaacttcaa gcggaaaggc 5820
ggcatcggag gacagacatc tgccgagaga ctgatcaata tcatcaccac tcagctggaa 5880
atccagcacc tccagaccaa gatccagaag attctgaact tccgggtgta ctaccgcgag 5940
ggcagagatc ctgtttggaa aggcccagca cagctgatct ggaaaggcga aggtgccgtg 6000
gtgctgaagg atggctctga tctgaaggtg gtgcccagac ggaaggccaa gattatcaag 6060
gattacgagc ccaaacagcg cgtgggcaat gaaggcgacg ttgagggcac aagaggcagc 6120
gacaattgaa attcactcct caggtgcagg ctgcctatca gaaggtggtg gctggtgtgg 6180
ccaatgccct ggctcacaaa taccactgag atctttttcc ctctgccaaa aattatgggg 6240
acatcatgaa gccccttgag catctgactt ctggctaata aaggaaattt attttcattg 6300
caatagtgtg ttggaatttt ttgtgtctct cactcggaag gacatatggg agggcaaatc 6360
atttaaaaca tcagaatgag tatttggttt agagtttggc aacatatgcc catatgctgg 6420
ctgccatgaa caaaggttgg ctataaagag gtcatcagta tatgaaacag ccccctgctg 6480
tccattcctt attccataga aaagccttga cttgaggtta gatttttttt atattttgtt 6540
ttgtgttatt tttttcttta acatccctaa aattttcctt acatgtttta ctagccagat 6600
ttttcctcct ctcctgacta ctcccagtca tagctgtccc tcttctctta tggagatccc 6660
tcgacctgca gcccaagctt ggcgtaatca tggtcatagc tgtttcctgt gtgaaattgt 6720
tatccgctca caattccaca caacatacga gccggaagca taaagtgtaa agcctggggt 6780
gcctaatgag tgagctaact cacattaatt gcgttgcgct cactgcccgc tttccagtcg 6840
ggaaacctgt cgtgccagcg gatccgcatc tcaattagtc agcaaccata gtcccgcccc 6900
taactccgcc catcccgccc ctaactccgc ccagttccgc ccattctccg ccccatggct 6960
gactaatttt ttttatttat gcagaggccg aggccgcctc ggcctctgag ctattccaga 7020
agtagtgagg aggctttttt ggaggcctag gcttttgcaa aaagctaact tgtttattgc 7080
agcttataat ggttacaaat aaagcaatag catcacaaat ttcacaaata aagcattttt 7140
ttcactgcat tctagttgtg gtttgtccaa actcatcaat gtatcttatc atgtctgtcc 7200
gcttcctcgc tcactgactc gctgcgctcg gtcgttcggc tgcggcgagc ggtatcagct 7260
cactcaaagg cggtaatacg gttatccaca gaatcagggg ataacgcagg aaagaacatg 7320
tgagcaaaag gccagcaaaa ggccaggaac cgtaaaaagg ccgcgttgct ggcgtttttc 7380
cataggctcc gcccccctga cgagcatcac aaaaatcgac gctcaagtca gaggtggcga 7440
aacccgacag gactataaag ataccaggcg tttccccctg gaagctccct cgtgcgctct 7500
cctgttccga ccctgccgct taccggatac ctgtccgcct ttctcccttc gggaagcgtg 7560
gcgctttctc atagctcacg ctgtaggtat ctcagttcgg tgtaggtcgt tcgctccaag 7620
ctgggctgtg tgcacgaacc ccccgttcag cccgaccgct gcgccttatc cggtaactat 7680
cgtcttgagt ccaacccggt aagacacgac ttatcgccac tggcagcagc cactggtaac 7740
aggattagca gagcgaggta tgtaggcggt gctacagagt tcttgaagtg gtggcctaac 7800
tacggctaca ctagaagaac agtatttggt atctgcgctc tgctgaagcc agttaccttc 7860
ggaaaaagag ttggtagctc ttgatccggc aaacaaacca ccgctggtag cggtggtttt 7920
tttgtttgca agcagcagat tacgcgcaga aaaaaaggat ctcaagaaga tcctttgatc 7980
ttttctacgg ggtctgacgc tcagtggaac gaaaactcac gttaagggat tttggtcatg 8040
agattatcaa aaaggatctt cacctagatc cttttaaatt aaaaatgaag ttttaaatca 8100
atctaaagta tatatgagta aacttggtct gacagttaga aaaactcatc gagcatcaaa 8160
tgaaactgca atttattcat atcaggatta tcaataccat atttttgaaa aagccgtttc 8220
tgtaatgaag gagaaaactc accgaggcag ttccatagga tggcaagatc ctggtatcgg 8280
tctgcgattc cgactcgtcc aacatcaata caacctatta atttcccctc gtcaaaaata 8340
aggttatcaa gtgagaaatc accatgagtg acgactgaat ccggtgagaa tggcaacagc 8400
ttatgcattt ctttccagac ttgttcaaca ggccagccat tacgctcgtc atcaaaatca 8460
ctcgcatcaa ccaaaccgtt attcattcgt gattgcgcct gagcgagacg aaatacgcga 8520
tcgctgttaa aaggacaatt acaaacagga atcgaatgca accggcgcag gaacactgcc 8580
agcgcatcaa caatattttc acctgaatca ggatattctt ctaatacctg gaatgctgtt 8640
tttccgggga tcgcagtggt gagtaaccat gcatcatcag gagtacggat aaaatgcttg 8700
atggtcggaa gaggcataaa ttccgtcagc cagtttagtc tgaccatctc atctgtaaca 8760
tcattggcaa cgctaccttt gccatgtttc agaaacaact ctggcgcatc gggcttccca 8820
tacaatcgat agattgtcgc acctgattgc ccgacattat cgcgagccca tttataccca 8880
tataaatcag catccatgtt ggaatttaat cgcggcctag agcaagacgt ttcccgttga 8940
atatggctca taacacccct tgtattactg tttatgtaag cagacagttt tattgttcat 9000
gatgatatat ttttatcttg tgcaatgtaa catcagagat tttgagacac aacaattggt 9060
cgac 9064
<210> SEQ ID NO: 21
<211> 9886
<223> pGM297
attgattatt gactagttat taatagtaat caattacggg gtcattagtt catagcccat 60
atatggagtt ccgcgttaca taacttacgg taaatggccc gcctggctga ccgcccaacg 120
acccccgccc attgacgtca ataatgacgt atgttcccat agtaacgcca atagggactt 180
tccattgacg tcaatgggtg gagtatttac ggtaaactgc ccacttggca gtacatcaag 240
tgtatcatat gccaagtacg ccccctattg acgtcaatga cggtaaatgg cccgcctggc 300
attatgccca gtacatgacc ttatgggact ttcctacttg gcagtacatc tacgtattag 360
tcatcgctat taccatggtc gaggtgagcc ccacgttctg cttcactctc cccatctccc 420
ccccctcccc acccccaatt ttgtatttat ttatttttta attattttgt gcagcgatgg 480
gggcgggggg gggggggggg cgcgcgccag gcggggcggg gcggggcgag gggcggggcg 540
gggcgaggcg gagaggtgcg gcggcagcca atcagagcgg cgcgctccga aagtttcctt 600
ttatggcgag gcggcggcgg cggcggccct ataaaaagcg aagcgcgcgg cgggcgggag 660
tcgctgcgcg ctgccttcgc cccgtgcccc gctccgccgc cgcctcgcgc cgcccgcccc 720
ggctctgact gaccgcgtta ctcccacagg tgagcgggcg ggacggccct tctcctccgg 780
gctgtaatta gcgcttggtt taatgacggc ttgtttcttt tctgtggctg cgtgaaagcc 840
ttgaggggct ccgggagggc cctttgtgcg gggggagcgg ctcggggggt gcgtgcgtgt 900
gtgtgtgcgt ggggagcgcc gcgtgcggct ccgcgctgcc cggcggctgt gagcgctgcg 960
ggcgcggcgc ggggctttgt gcgctccgca gtgtgcgcga ggggagcgcg gccgggggcg 1020
gtgccccgcg gtgcgggggg ggctgcgagg ggaacaaagg ctgcgtgcgg ggtgtgtgcg 1080
tggggggggg agcagggggt gtgggcgcgt cggtcgggct gcaacccccc ctgcaccccc 1140
ctccccgagt tgctgagcac ggcccggctt cgggtgcggg gctccgtacg gggcgtggcg 1200
cggggctcgc cgtgccgggc ggggggtggc ggcaggtggg ggtgccgggc ggggcggggc 1260
cgcctcgggc cggggagggc tcgggggagg ggcgcggcgg cccccggagc gccggcggct 1320
gtcgaggcgc ggcgagccgc agccattgcc ttttatggta atcgtgcgag agggcgcagg 1380
gacttccttt gtcccaaatc tgtgcggagc cgaaatctgg gaggcgccgc cgcaccccct 1440
ctagcgggcg cggggcgaag cggtgcggcg ccggcaggaa ggaaatgggc ggggagggcc 1500
ttcgtgcgtc gccgcgccgc cgtccccttc tccctctcca gcctcggggc tgtccgcggg 1560
gggacggctg ccttcggggg ggacggggca gggcggggtt cggcttctgg cgtgtgaccg 1620
gcggctctag agcctctgct aaccatgttc atgccttctt ctttttccta cagctcctgg 1680
gcaacgtgct ggttattgtg ctgtctcatc attttggcaa agaattgctc gagactagtg 1740
acttggtgag taggcttcga gcctagttag aggactagga gaggccgtag ccgtaactac 1800
tctgggcaag tagggcaggc ggtgggtacg caatgggggc ggctacctca gcactaaata 1860
ggagacaatt agaccaattt gagaaaatac gacttcgccc gaacggaaag aaaaagtacc 1920
aaattaaaca tttaatatgg gcaggcaagg agatggagcg cttcggcctc catgagaggt 1980
tgttggagac agaggagggg tgtaaaagaa tcatagaagt cctctacccc ctagaaccaa 2040
caggatcgga gggcttaaaa agtctgttca atcttgtgtg cgtactatat tgcttgcaca 2100
aggaacagaa agtgaaagac acagaggaag cagtagcaac agtaagacaa cactgccatc 2160
tagtggaaaa agaaaaaagt gcaacagaga catctagtgg acaaaagaaa aatgacaagg 2220
gaatagcagc gccacctggt ggcagtcaga attttccagc gcaacaacaa ggaaatgcct 2280
gggtacatgt acccttgtca ccgcgcacct taaatgcgtg ggtaaaagca gtagaggaga 2340
aaaaatttgg agcagaaata gtacccatgt ttcaagccct atcagaaggc tgcacaccct 2400
atgacattaa tcagatgctt aatgtgctag gagatcatca aggggcatta caaatagtga 2460
aagagatcat taatgaagaa gcagcccagt gggatgtaac acacccacta cccgcaggac 2520
ccctaccagc aggacagctc agggaccctc gcggctcaga tatagcaggg accaccagct 2580
cagtacaaga acagttagaa tggatctata ctgctaaccc ccgggtagat gtaggtgcca 2640
tctaccggag atggattatt ctaggacttc aaaagtgtgt caaaatgtac aacccagtat 2700
cagtcctaga cattaggcag ggacctaaag agcccttcaa ggattatgtg gacagatttt 2760
acaaggcaat tagagcagaa caagcctcag gggaagtgaa acaatggatg acagaatcat 2820
tactcattca aaatgctaat ccagattgta aggtcatcct gaagggccta ggaatgcacc 2880
ccacccttga agaaatgtta acggcttgtc agggggtagg aggcccaagc tacaaagcaa 2940
aagtaatggc agaaatgatg cagaccatgc aaaatcaaaa catggtgcag cagggaggtc 3000
caaaaagaca aagaccccca ctaagatgtt ataattgtgg aaaatttggc catatgcaaa 3060
gacaatgtcc ggaaccaagg aaaacaaaat gtctaaagtg tggaaaattg ggacacctag 3120
caaaagactg caggggacag gtgaattttt tagggtatgg acggtggatg ggggcaaaac 3180
cgagaaattt tcccgccgct actcttggag cggaaccgag tgcgcctcct ccaccgagcg 3240
gcaccacccc atacgaccca gcaaagaagc tcctgcagca atatgcagag aaagggaaac 3300
aactgaggga gcaaaagagg aatccaccgg caatgaatcc ggattggacc gagggatatt 3360
ctttgaactc cctctttgga gaagaccaat aaagacagtg tatatagaag gggtccccat 3420
taaggcactg ctagacacag gggcagatga caccataatt aaagaaaatg atttacaatt 3480
atcaggtcca tggagaccca aaattatagg gggcatagga ggaggcctta atgtaaaaga 3540
atataacgac agggaagtaa aaatagaaga taaaattttg agaggaacaa tattgttagg 3600
agcaactccc attaatataa taggtagaaa tttgctggcc ccggcaggtg cccggttagt 3660
aatgggacaa ttatcagaaa aaattcctgt cacacctgtc aaattgaagg aaggggctcg 3720
gggaccctgt gtaagacaat ggcctctctc taaagagaag attgaagctt tacaggaaat 3780
atgttcccaa ttagagcagg aaggaaaaat cagtagagta ggaggagaaa atgcatacaa 3840
taccccaata ttttgcataa agaagaagga caaatcccag tggaggatgc tagtagactt 3900
tagagagtta aataaggcaa cccaagattt ctttgaagtg caattaggga taccccaccc 3960
agcaggatta agaaagatga gacagataac agttttagat gtaggagacg cctattattc 4020
cataccattg gatccaaatt ttaggaaata tactgctttt actattccca cagtgaataa 4080
tcagggaccc gggattaggt atcaattcaa ctgtctcccg caagggtgga aaggatctcc 4140
tacaatcttc caaaatacag cagcatccat tttggaggag ataaaaagaa acttgccagc 4200
actaaccatt gtacaataca tggatgattt atgggtaggt tctcaagaaa atgaacacac 4260
ccatgacaaa ttagtagaac agttaagaac aaaattacaa gcctggggct tagaaacccc 4320
agaaaagaag gtgcaaaaag aaccacctta tgagtggatg ggatacaaac tttggcctca 4380
caaatgggaa ctaagcagaa tacaactgga ggaaaaagat gaatggactg tcaatgacat 4440
ccagaagtta gttgggaaac taaattgggc agcacaattg tatccaggtc ttaggaccaa 4500
gaatatatgc aagttaatta gaggaaagaa aaatctgtta gagctagtga cttggacacc 4560
tgaggcagaa gctgaatatg cagaaaatgc agagattctt aaaacagaac aggaaggaac 4620
ctattacaaa ccaggaatac ctattagggc agcagtacag aaattggaag gaggacagtg 4680
gagttaccaa ttcaaacaag aaggacaagt cttgaaagta ggaaaataca ccaagcaaaa 4740
gaacacccat acaaatgaac ttcgcacatt agctggttta gtgcagaaga tttgcaaaga 4800
agctctagtt atttggggga tattaccagt tctagaactc ccgatagaaa gagaggtatg 4860
ggaacaatgg tgggcggatt actggcaggt aagctggatt cccgaatggg attttgtcag 4920
caccccacct ttgctcaaac tatggtacac attaacaaaa gaacccatac ccaaggagga 4980
cgtttactat gtagatggag catgcaacag aaattcaaaa gaaggaaaag caggatacat 5040
ctcacaatac ggaaaacaga gagtagaaac attagaaaac actaccaatc agcaagcaga 5100
attaacagct ataaaaatgg ctttggaaga cagtgggcct aatgtgaaca tagtaacaga 5160
ctctcaatat gcaatgggaa ttttgacagc acaacccaca caaagtgatt caccattagt 5220
agagcaaatt atagccttaa tgatacaaaa gcaacaaata tatttgcagt gggtaccagc 5280
acataaagga ataggaggaa atgaggagat agataaatta gtgagtaaag gcattagaag 5340
agttttattc ttagaaaaaa tagaagaagc tcaagaagag catgaaagat atcataataa 5400
ttggaaaaac ctagcagata catatgggct tccacaaata gtagcaaaag agatagtggc 5460
catgtgtcca aaatgtcaga taaagggaga accagtgcat ggacaagtgg atgcctcacc 5520
tggaacatgg cagatggatt gtactcatct agaaggaaaa gtagtcatag ttgcggtcca 5580
tgtagccagt ggattcatag aagcagaagt catacctagg gaaacaggaa aagaaacggc 5640
aaagtttcta ttaaaaatac tgagtagatg gcctataaca cagttacaca cagacaatgg 5700
gcctaacttt acctcccaag aagtggcagc aatatgttgg tggggaaaaa ttgaacatac 5760
aacaggtata ccatataacc cccaatctca aggatcaata gaaagcatga acaaacaatt 5820
aaaagagata attgggaaaa taagagatga ttgccaatat acagagacag cagtactgat 5880
ggcttgccat attcacaatt ttaaaagaaa gggaggaata gggggacaga cttcagcaga 5940
gagactaatt aatataataa caacacaatt agaaatacaa catttacaaa ccaaaattca 6000
aaaaatttta aattttagag tctactacag agaagggaga gaccctgtgt ggaaaggacc 6060
agcacaatta atctggaaag gggaaggagc agtggtcctc aaggacggaa gtgacctaaa 6120
ggttgtacca agaaggaaag ctaaaattat taaggattat gaacccaaac aaagagtggg 6180
taatgagggt gacgtggaag gtaccagggg atctgataac taaatggcag ggaatagtca 6240
gatattggat gagacaaaga aatttgaaat ggaactatta tatgcatcag ctggcggccg 6300
cgaattcact agtgattccc gtttgtgcta gggttcttag gcttcttggg ggctgctgga 6360
actgcaatgg gagcageggc gacagccctg acggtccagt ctcagcattt gcttgctggg 6420
atactgcagc agcagaagaa tctgctggcg gctgtggagg ctcaacagca gatgttgaag 6480
ctgaccattt ggggtgttaa aaacctcaat gcccgcgtca cagcccttga gaagtaccta 6540
gaggatcagg cacgactaaa ctcctggggg tgcgcatgga aacaagtatg tcataccaca 6600
gtggagtggc cctggacaaa tcggactccg gattggcaaa atatgacttg gttggagtgg 6660
gaaagacaaa tagctgattt ggaaagcaac attacgagac aattagtgaa ggctagagaa 6720
caagaggaaa agaatctaga tgcctatcag aagttaacta gttggtcaga tttctggtct 6780
tggttcgatt tctcaaaatg gottaacatt ttaaaaatgg gatttttagt aatagtagga 6840
ataatagggt taagattact ttacacagta tatggatgta tagtgagggt taggcaggga 6900
tatgttcctc tatctccaca gatccatatc caatcgaatt cccgcggccg caattcactc 6960
ctcaggtgca ggctgcctat cagaaggtgg tggctggtgt ggccaatgcc ctggctcaca 7020
aataccactg agatcttttt ccctctgcca aaaattatgg ggacatcatg aagccccttg 7080
agcatctgac ttctggctaa taaaggaaat ttattttcat tgcaatagtg tgttggaatt 7140
ttttgtgtct ctcactcgga aggacatatg ggagggcaaa tcatttaaaa catcagaatg 7200
agtatttggt ttagagtttg gcaacatatg cccatatgct ggctgccatg aacaaaggtt 7260
ggctataaag aggtcatcag tatatgaaac agccccctgc tgtccattcc ttattccata 7320
gaaaagcctt gacttgaggt tagatttttt ttatattttg ttttgtgtta tttttttctt 7380
taacatccct aaaattttcc ttacatgttt tactagccag atttttcctc ctctcctgac 7440
tactcccagt catagctgtc cctcttctct tatggagatc cctcgacctg cagcccaagc 7500
ttggcgtaat catggtcata gctgtttcct gtgtgaaatt gttatccgct cacaattcca 7560
cacaacatac gagccggaag cataaagtgt aaagcctggg gtgcctaatg agtgagctaa 7620
ctcacattaa ttgcgttgcg ctcactgccc gctttccagt cgggaaacct gtcgtgccag 7680
cggatccgca tctcaattag tcagcaacca tagtcccgcc cctaactccg cccatcccgc 7740
ccctaactcc gcccagttcc gcccattctc cgccccatgg ctgactaatt ttttttattt 7800
atgcagaggc cgaggccgcc tcggcctctg agctattcca gaagtagtga ggaggctttt 7860
ttggaggcct aggcttttgc aaaaagctaa cttgtttatt gcagcttata atggttacaa 7920
ataaagcaat agcatcacaa atttcacaaa taaagcattt ttttcactgc attctagttg 7980
tggtttgtcc aaactcatca atgtatctta tcatgtctgt ccgcttcctc gctcactgac 8040
tcgctgcgct cggtcgttcg gctgcggcga gcggtatcag ctcactcaaa ggcggtaata 8100
cggttatcca cagaatcagg ggataacgca ggaaagaaca tgtgagcaaa aggccagcaa 8160
aaggccagga accgtaaaaa ggccgcgttg ctggcgtttt tccataggct ccgcccccct 8220
gacgagcatc acaaaaatcg acgctcaagt cagaggtggc gaaacccgac aggactataa 8280
agataccagg cgtttccccc tggaagctcc ctcgtgcgct ctcctgttcc gaccctgccg 8340
cttaccggat acctgtccgc ctttctccct tcgggaagcg tggcgctttc tcatagctca 8400
cgctgtaggt atctcagttc ggtgtaggtc gttcgctcca agctgggctg tgtgcacgaa 8460
ccccccgttc agcccgaccg ctgcgcctta tccggtaact atcgtcttga gtccaacccg 8520
gtaagacacg acttatcgcc actggcagca gccactggta acaggattag cagagcgagg 8580
tatgtaggcg gtgctacaga gttcttgaag tggtggccta actacggcta cactagaaga 8640
acagtatttg gtatctgcgc tctgctgaag ccagttacct tcggaaaaag agttggtagc 8700
tcttgatccg gcaaacaaac caccgctggt agcggtggtt tttttgtttg caagcagcag 8760
attacgcgca gaaaaaaagg atctcaagaa gatcctttga tcttttctac ggggtctgac 8820
gctcagtgga acgaaaactc acgttaaggg attttggtca tgagattatc aaaaaggatc 8880
ttcacctaga tccttttaaa ttaaaaatga agttttaaat caatctaaag tatatatgag 8940
taaacttggt ctgacagtta gaaaaactca tcgagcatca aatgaaactg caatttattc 9000
atatcaggat tatcaatacc atatttttga aaaagccgtt tctgtaatga aggagaaaac 9060
tcaccgaggc agttccatag gatggcaaga tcctggtatc ggtctgcgat tccgactcgt 9120
ccaacatcaa tacaacctat taatttcccc tcgtcaaaaa taaggttatc aagtgagaaa 9180
tcaccatgag tgacgactga atccggtgag aatggcaaca gcttatgcat ttctttccag 9240
acttgttcaa caggccagcc attacgctcg tcatcaaaat cactcgcatc aaccaaaccg 9300
ttattcattc gtgattgcgc ctgagcgaga cgaaatacgc gatcgctgtt aaaaggacaa 9360
ttacaaacag gaatcgaatg caaccggcgc aggaacactg ccagcgcatc aacaatattt 9420
tcacctgaat caggatattc ttctaatacc tggaatgctg tttttccggg gatcgcagtg 9480
gtgagtaacc atgcatcatc aggagtacgg ataaaatgct tgatggtcgg aagaggcata 9540
aattccgtca gccagtttag tctgaccatc tcatctgtaa catcattggc aacgctacct 9600
ttgccatgtt tcagaaacaa ctctggcgca tcgggcttcc catacaatcg atagattgtc 9660
gcacctgatt gcccgacatt atcgcgagcc catttatacc catataaatc agcatccatg 9720
ttggaattta atcgcggcct agagcaagac gtttcccgtt gaatatggct cataacaccc 9780
cttgtattac tgtttatgta agcagacagt tttattgttc atgatgatat atttttatct 9840
tgtgcaatgt aacatcagag attttgagac acaacaattg gtcgac 9886
<210> SEQ ID NO: 22
<211> 3384
<223> pGM299
tcaatattgg ccattagcca tattattcat tggttatata gcataaatca atattggcta 60
ttggccattg catacgttgt atctatatca taatatgtac atttatattg gctcatgtcc 120
aatatgaccg ccatgttggc attgattatt gactagttat taatagtaat caattacggg 180
gtcattagtt catagcccat atatggagtt ccgcgttaca taacttacgg taaatggccc 240
gcctggctga ccgcccaacg acccccgccc attgacgtca ataatgacgt atgttcccat 300
agtaacgcca atagggactt tccattgacg tcaatgggtg gagtatttac ggtaaactgc 360
ccacttggca gtacatcaag tgtatcatat gccaagtccg ccccctattg acgtcaatga 420
cggtaaatgg cccgcctggc attatgccca gtacatgacc ttacgggact ttcctacttg 480
gcagtacatc tacgtattag tcatcgctat taccatggtg atgcggtttt ggcagtacac 540
caatgggcgt ggatagcggt ttgactcacg gggatttcca agtctccacc ccattgacgt 600
caatgggagt ttgttttggc accaaaatca acgggacttt ccaaaatgtc gtaataaccc 660
cgccccgttg acgcaaatgg gcggtaggcg tgtacggtgg gaggtctata taagcagagc 720
tcgtttagtg aaccgtcaga tcactagaag ctttattgcg gtagtttatc acagttaaat 780
tgctaacgca gtcagtgctt ctgacacaac agtctcgaac ttaagctgca gaagttggtc 840
gtgaggcact gggcaggtaa gtatcaaggt tacaagacag gtttaaggag accaatagaa 900
actgggcttg tcgagacaga gaagactctt gcgtttctga taggcaccta ttggtcttac 960
tgacatccac tttgcctttc tctccacagg tgtccactcc cagttcaatt acagctctta 1020
aggctagagt acttaatacg actcactata ggctagcctc gagaattcga ttatgcccct 1080
aggaccagaa gaaagaagat tgcttcgctt gatttggctc ctttacagca ccaatccata 1140
tccaccaagt ggggaaggga cggccagaca acgccgacga gccaggagaa ggtggagaca 1200
acagcaggat caaattagag tcttggtaga aagactccaa gagcaggtgt atgcagttga 1260
ccgcctggct gacgaggctc aacacttggc tatacaacag ttgcctgacc ctcctcattc 1320
agcttagaat cactagtgaa ttcacgcgtg gtacctctag agtcgacccg ggcggccgct 1380
tcgagcagac atgataagat acattgatga gtttggacaa accacaacta gaatgcagtg 1440
aaaaaaatgc tttatttgtg aaatttgtga tgctattgct ttatttgtaa ccattataag 1500
ctgcaataaa caagttaaca acaacaattg cattcatttt atgtttcagg ttcaggggga 1560
gatgtgggag gttttttaaa gcaagtaaaa cctctacaaa tgtggtaaaa tcgataagga 1620
tccgtcgacc aattgttgtg tctcaaaatc tctgatgtta cattgcacaa gataaaaata 1680
tatcatcatg aacaataaaa ctgtctgctt acataaacag taatacaagg ggtgttatga 1740
gccatattca acgggaaacg tcttgctcta ggccgcgatt aaattccaac atggatgctg 1800
atttatatgg gtataaatgg gctcgcgata atgtcgggca atcaggtgcg acaatctatc 1860
gattgtatgg gaagcccgat gcgccagagt tgtttctgaa acatggcaaa ggtagcgttg 1920
ccaatgatgt tacagatgag atggtcagac taaactggct gacggaattt atgcctcttc 1980
cgaccatcaa gcattttatc cgtactcctg atgatgcatg gttactcacc actgcgatcc 2040
ccggaaaaac agcattccag gtattagaag aatatcctga ttcaggtgaa aatattgttg 2100
atgcgctggc agtgttcctg cgccggttgc attcgattcc tgtttgtaat tgtcctttta 2160
acagcgatcg cgtatttcgt ctcgctcagg cgcaatcacg aatgaataac ggtttggttg 2220
atgcgagtga ttttgatgac gagcgtaatg gctggcctgt tgaacaagtc tggaaagaaa 2280
tgcataagct gttgccattc tcaccggatt cagtcgtcac tcatggtgat ttctcacttg 2340
ataaccttat ttttgacgag gggaaattaa taggttgtat tgatgttgga cgagtcggaa 2400
tcgcagaccg ataccaggat cttgccatcc tatggaactg cctcggtgag ttttctcctt 2460
cattacagaa acggcttttt caaaaatatg gtattgataa tcctgatatg aataaattgc 2520
agtttcattt gatgctcgat gagtttttct aactgtcaga ccaagtttac tcatatatac 2580
tttagattga tttaaaactt catttttaat ttaaaaggat ctaggtgaag atcctttttg 2640
ataatctcat gaccaaaatc ccttaacgtg agttttcgtt ccactgagcg tcagaccccg 2700
tagaaaagat caaaggatct tcttgagatc ctttttttct gcgcgtaatc tgctgcttgc 2760
aaacaaaaaa accaccgcta ccagcggtgg tttgtttgcc ggatcaagag ctaccaactc 2820
tttttccgaa ggtaactggc ttcagcagag cgcagatacc aaatactgtt cttctagtgt 2880
agccgtagtt aggccaccac ttcaagaact ctgtagcacc gcctacatac ctcgctctgc 2940
taatcctgtt accagtggct gctgccagtg gcgataagtc gtgtcttacc gggttggact 3000
caagacgata gttaccggat aaggcgcagc ggtcgggctg aacggggggt tcgtgcacac 3060
agcccagctt ggagcgaacg acctacaccg aactgagata cctacagcgt gagctatgag 3120
aaagcgccac gcttcccgaa gggagaaagg cggacaggta tccggtaagc ggcagggtcg 3180
gaacaggaga gcgcacgagg gagcttccag ggggaaacgc ctggtatctt tatagtcctg 3240
tcgggtttcg ccacctctga cttgagcgtc gatttttgtg atgctcgtca ggggggcgga 3300
gcctatggaa aaacgccagc aacgcggcct ttttacggtt cctggccttt tgctggcctt 3360
ttgctcacat ggctcgacag atct 3384
<210> SEQ ID NO: 23
<211> 6264
<223> pGM301
attgattatt gactagttat taatagtaat caattacggg gtcattagtt catagcccat 60
atatggagtt ccgcgttaca taacttacgg taaatggccc gcctggctga ccgcccaacg 120
acccccgccc attgacgtca ataatgacgt atgttcccat agtaacgcca atagggactt 180
tccattgacg tcaatgggtg gagtatttac ggtaaactgc ccacttggca gtacatcaag 240
tgtatcatat gccaagtacg ccccctattg acgtcaatga cggtaaatgg cccgcctggc 300
attatgccca gtacatgacc ttatgggact ttcctacttg gcagtacatc tacgtattag 360
tcatcgctat taccatggtc gaggtgagcc ccacgttctg cttcactctc cccatctccc 420
ccccctcccc acccccaatt ttgtatttat ttatttttta attattttgt gcagcgatgg 480
gggcgggggg gggggggggg cgcgcgccag gcggggcggg gcggggcgag gggcggggcg 540
gggcgaggcg gagaggtgcg gcggcagcca atcagagcgg cgcgctccga aagtttcctt 600
ttatggcgag gcggcggcgg cggcggccct ataaaaagcg aagcgcgcgg cgggcgggag 660
tcgctgcgcg ctgccttcgc cccgtgcccc gctccgccgc cgcctcgcgc cgcccgcccc 720
ggctctgact gaccgcgtta ctcccacagg tgagcgggcg ggacggccct tctcctccgg 780
gctgtaatta gcgcttggtt taatgacggc ttgtttcttt tctgtggctg cgtgaaagcc 840
ttgaggggct ccgggagggc cctttgtgcg gggggagcgg ctcggggggt gcgtgcgtgt 900
gtgtgtgcgt ggggagcgcc gcgtgcggct ccgcgctgcc cggcggctgt gagcgctgcg 960
ggcgcggcgc ggggctttgt gcgctccgca gtgtgcgcga ggggagcgcg gccgggggcg 1020
gtgccccgcg gtgcgggggg ggctgcgagg ggaacaaagg ctgcgtgcgg ggtgtgtgcg 1080
tgggggggtg agcagggggt gtgggcgcgt cggtcgggct gcaacccccc ctgcaccccc 1140
ctccccgagt tgctgagcac ggcccggctt cgggtgcggg gctccgtacg gggcgtggcg 1200
cggggctcgc cgtgccgggc ggggggtggc ggcaggtggg ggtgccgggc ggggcggggc 1260
cgcctcgggc cggggagggc tcgggggagg ggcgcggcgg cccccggagc gccggcggct 1320
gtcgaggcgc ggcgagccgc agccattgcc ttttatggta atcgtgcgag agggcgcagg 1380
gacttccttt gtcccaaatc tgtgcggagc cgaaatctgg gaggcgccgc cgcaccccct 1440
ctagcgggcg cggggcgaag cggtgcggcg ccggcaggaa ggaaatgggc ggggagggcc 1500
ttcgtgcgtc gccgcgccgc cgtccccttc tccctctcca gcctcggggc tgtccgcggg 1560
gggacggctg ccttcggggg ggacggggca gggcggggtt cggcttctgg cgtgtgaccg 1620
gcggctctag agcctctgct aaccatgttc atgccttctt ctttttccta cagctcctgg 1680
gcaacgtgct ggttattgtg ctgtctcatc attttggcaa agaattcgat tgccatggca 1740
acatatatcc agagagtaca gtgcatctca acatcactac tggttgttct caccacattg 1800
gtctcgtgtc agattcccag ggataggctc tctaacatag gggtcatagt cgatgaaggg 1860
aaatcactga agatagctgg atcccacgaa tcgaggtaca tagtactgag tctagttccg 1920
ggggtagact ttgagaatgg gtgcggaaca gcccaggtta tccagtacaa gagcctactg 1980
aacaggctgt taatcccatt gagggatgcc ttagatcttc aggaggctct gataactgtc 2040
accaatgata cgacacaaaa tgccggtgct ccccagtoga gattcttcgg tgctgtgatt 2100
ggtactatcg cacttggagt ggcgacatca gcacaaatca ccgcagggat tgcactagcc 2160
gaagcgaggg aggccaaaag agacatagcg ctcatcaaag aatcgatgac aaaaacacac 2220
aagtctatag aactgctgca aaacgctgtg ggggaacaaa ttcttgctct aaagacactc 2280
caggatttcg tgaatgatga gatcaaaccc gcaataagcg aattaggctg tgagactgct 2340
gccttaagac tgggtataaa attgacacag cattactccg agctgttaac tgcgttcggc 2400
tcgaatttcg gaaccatcgg agagaagagc ctcacgctgc aggcgctgtc ttcactttac 2460
tctgctaaca ttactgagat tatgaccaca atcaggacag ggcagtctaa catctatgat 2520
gtcatttata cagaacagat caaaggaacg gtgatagatg tggatctaga gagatacatg 2580
gtcaccctgt ctgtgaagat ccctattctt tctgaagtcc caggtgtgct catacacaag 2640
gcatcatcta tttcttacaa catagacggg gaggaatggt atgtgactgt ccccagccat 2700
atactcagtc gtgcttcttt cttagggggt gcagacataa ccgattgtgt tgagtccaga 2760
ttgacctata tatgccccag ggatcccgca caactgatac ctgacagcca gcaaaagtgt 2820
atcctggggg acacaacaag gtgtcctgtc acaaaagttg tggacagcct tatccccaag 2880
tttgcttttg tgaatggggg cgttgttgct aactgcatag catccacatg tacctgcggg 2940
acaggccgaa gaccaatcag tcaggatcgc tctaaaggtg tagtattcct aacccatgac 3000
aactgtggtc ttataggtgt caatggggta gaattgtatg ctaaccggag agggcacgat 3060
gccacttggg gggtccagaa cttgacagtc ggtcctgcaa ttgctatcag acccgttgat 3120
atttctctca accttgctga tgctacgaat ttcttgcaag actctaaggc tgagcttgag 3180
aaagcacgga aaatcctctc ggaggtaggt agatggtaca actcaagaga gactgtgatt 3240
acgatcatag tagttatggt cgtaatattg gtggtcatta tagtgatcat catcgtgctt 3300
tatagactca gaaggtgaaa tcactagtga attcactcct caggtgcagg ctgcctatca 3360
gaaggtggtg gctggtgtgg ccaatgccct ggctcacaaa taccactgag atctttttcc 3420
ctctgccaaa aattatgggg acatcatgaa gccccttgag catctgactt ctggctaata 3480
aaggaaattt attttcattg caatagtgtg ttggaatttt ttgtgtctct cactcggaag 3540
gacatatggg agggcaaatc atttaaaaca tcagaatgag tatttggttt agagtttggc 3600
aacatatgcc catatgctgg ctgccatgaa caaaggttgg ctataaagag gtcatcagta 3660
tatgaaacag ccccctgctg tccattcctt attccataga aaagccttga cttgaggtta 3720
gatttttttt atattttgtt ttgtgttatt tttttcttta acatccctaa aattttcctt 3780
acatgtttta ctagccagat ttttcctcct ctcctgacta ctcccagtca tagctgtccc 3840
tcttctctta tggagatccc tcgacctgca gcccaagctt ggcgtaatca tggtcatagc 3900
tgtttcctgt gtgaaattgt tatccgctca caattccaca caacatacga gccggaagca 3960
taaagtgtaa agcctggggt gcctaatgag tgagctaact cacattaatt gcgttgcgct 4020
cactgcccgc tttccagtcg ggaaacctgt cgtgccagcg gatccgcatc tcaattagtc 4080
agcaaccata gtcccgcccc taactccgcc catcccgccc ctaactccgc ccagttccgc 4140
ccattctccg ccccatggct gactaatttt ttttatttat gcagaggccg aggccgcctc 4200
ggcctctgag ctattccaga agtagtgagg aggctttttt ggaggcctag gcttttgcaa 4260
aaagctaact tgtttattgc agcttataat ggttacaaat aaagcaatag catcacaaat 4320
ttcacaaata aagcattttt ttcactgcat tctagttgtg gtttgtccaa actcatcaat 4380
gtatcttatc atgtctgtcc gcttcctcgc tcactgactc gctgcgctcg gtcgttcggc 4440
tgcggcgagc ggtatcagct cactcaaagg cggtaatacg gttatccaca gaatcagggg 4500
ataacgcagg aaagaacatg tgagcaaaag gccagcaaaa ggccaggaac cgtaaaaagg 4560
ccgcgttgct ggcgtttttc cataggctcc gcccccctga cgagcatcac aaaaatcgac 4620
gctcaagtca gaggtggcga aacccgacag gactataaag ataccaggcg tttccccctg 4680
gaagctccct cgtgcgctct cctgttccga ccctgccgct taccggatac ctgtccgcct 4740
ttctcccttc gggaagcgtg gcgctttctc atagctcacg ctgtaggtat ctcagttcgg 4800
tgtaggtcgt tcgctccaag ctgggctgtg tgcacgaacc ccccgttcag cccgaccgct 4860
gcgccttatc cggtaactat cgtcttgagt ccaacccggt aagacacgac ttatcgccac 4920
tggcagcagc cactggtaac aggattagca gagcgaggta tgtaggcggt gctacagagt 4980
tcttgaagtg gtggcctaac tacggctaca ctagaagaac agtatttggt atctgcgctc 5040
tgctgaagcc agttaccttc ggaaaaagag ttggtagctc ttgatccggc aaacaaacca 5100
ccgctggtag cggtggtttt tttgtttgca agcagcagat tacgcgcaga aaaaaaggat 5160
ctcaagaaga tcctttgatc ttttctacgg ggtctgacgc tcagtggaac gaaaactcac 5220
gttaagggat tttggtcatg agattatcaa aaaggatctt cacctagatc cttttaaatt 5280
aaaaatgaag ttttaaatca atctaaagta tatatgagta aacttggtct gacagttaga 5340
aaaactcatc gagcatcaaa tgaaactgca atttattcat atcaggatta tcaataccat 5400
atttttgaaa aagccgtttc tgtaatgaag gagaaaactc accgaggcag ttccatagga 5460
tggcaagatc ctggtatcgg tctgcgattc cgactcgtcc aacatcaata caacctatta 5520
atttcccctc gtcaaaaata aggttatcaa gtgagaaatc accatgagtg acgactgaat 5580
ccggtgagaa tggcaacagc ttatgcattt ctttccagac ttgttcaaca ggccagccat 5640
tacgctcgtc atcaaaatca ctcgcatcaa ccaaaccgtt attcattcgt gattgcgcct 5700
gagcgagacg aaatacgcga tcgctgttaa aaggacaatt acaaacagga atcgaatgca 5760
accggcgcag gaacactgcc agcgcatcaa caatattttc acctgaatca ggatattctt 5820
ctaatacctg gaatgctgtt tttccgggga tcgcagtggt gagtaaccat gcatcatcag 5880
gagtacggat aaaatgcttg atggtcggaa gaggcataaa ttccgtcagc cagtttagtc 5940
tgaccatctc atctgtaaca tcattggcaa cgctaccttt gccatgtttc agaaacaact 6000
ctggcgcatc gggcttccca tacaatcgat agattgtcgc acctgattgc ccgacattat 6060
cgcgagccca tttataccca tataaatcag catccatgtt ggaatttaat cgcggcctag 6120
agcaagacgt ttcccgttga atatggctca taacacccct tgtattactg tttatgtaag 6180
cagacagttt tattgttcat gatgatatat ttttatcttg tgcaatgtaa catcagagat 6240
tttgagacac aacaattggt cgac 6264
<210> SEQ ID NO: 24
<211> 6522
<223> pGM303
attgattatt gactagttat taatagtaat caattacggg gtcattagtt catagcccat 60
atatggagtt ccgcgttaca taacttacgg taaatggccc gcctggctga ccgcccaacg 120
acccccgccc attgacgtca ataatgacgt atgttcccat agtaacgcca atagggactt 180
tccattgacg tcaatgggtg gagtatttac ggtaaactgc ccacttggca gtacatcaag 240
tgtatcatat gccaagtacg ccccctattg acgtcaatga cggtaaatgg cccgcctggc 300
attatgccca gtacatgacc ttatgggact ttcctacttg gcagtacatc tacgtattag 360
tcatcgctat taccatggtc gaggtgagcc ccacgttctg cttcactctc cccatctccc 420
ccccctcccc acccccaatt ttgtatttat ttatttttta attattttgt gcagcgatgg 480
gggcgggggg gggggggggg cgcgcgccag gcggggcggg gcggggcgag gggcggggcg 540
gggcgaggcg gagaggtgcg gcggcagcca atcagagcgg cgcgctccga aagtttcctt 600
ttatggcgag gcggcggcgg cggcggccct ataaaaagcg aagcgcgcgg cgggcgggag 660
tcgctgcgcg ctgccttcgc cccgtgcccc gctccgccgc cgcctcgcgc cgcccgcccc 720
ggctctgact gaccgcgtta ctcccacagg tgagcgggcg ggacggccct tctcctccgg 780
gctgtaatta gcgcttggtt taatgacggc ttgtttcttt tctgtggctg cgtgaaagcc 840
ttgaggggct ccgggagggc cctttgtgcg gggggagcgg ctcggggggt gcgtgcgtgt 900
gtgtgtgcgt ggggagcgcc gcgtgcggct ccgcgctgcc cggcggctgt gagcgctgcg 960
ggcgcggcgc ggggctttgt gcgctccgca gtgtgcgcga ggggagcgcg gccgggggcg 1020
gtgccccgcg gtgcgggggg ggctgcgagg ggaacaaagg ctgcgtgcgg ggtgtgtgcg 1080
tgggggggtg agcagggggt gtgggcgcgt cggtcgggct gcaacccccc ctgcaccccc 1140
ctccccgagt tgctgagcac ggcccggctt cgggtgcggg gctccgtacg gggcgtggcg 1200
cggggctcgc cgtgccgggc ggggggtggc ggcaggtggg ggtgccgggc ggggcggggc 1260
cgcctcgggc cggggagggc tcgggggagg ggcgcggcgg cccccggagc gccggcggct 1320
gtcgaggcgc ggcgagccgc agccattgcc ttttatggta atcgtgcgag agggcgcagg 1380
gacttccttt gtcccaaatc tgtgcggagc cgaaatctgg gaggcgccgc cgcaccccct 1440
ctagcgggcg cggggcgaag cggtgcggcg ccggcaggaa ggaaatgggc ggggagggcc 1500
ttcgtgcgtc gccgcgccgc cgtccccttc tccctctcca gcctcggggc tgtccgcggg 1560
gggacggggc agggcggggt tcggcttctg gcgtgtgacc ggcggctcta gagcctctgc 1620
taaccatgtt catgccttct tctttttcct acagctcctg ggcaacgtgc tggttattgt 1680
gctgtctcat cattttggca aagaattcct cgagcatgtg gtctgagtta aaaatcagga 1740
gcaacgacgg aggtgaagga ccagaggacg ccaacgaccc ccggggaaag ggggtgcaac 1800
acatccatat ccagccatct ctacctgttt atggacagag ggttagggat ggtgataggg 1860
gcaaacgtga ctcgtactgg tctacttctc ctagtggtag caccacaaaa ccagcatcag 1920
gttgggagag gtcaagtaaa gccgacacat ggttgctgat tctctcattc acccagtggg 1980
ctttgtcaat tgccacagtg atcatctgta tcataatttc tgctagacaa gggtatagta 2040
tgaaagagta ctcaatgact gtagaggcat tgaacatgag cagcagggag gtgaaagagt 2100
cacttaccag tctaataagg caagaggtta tagcaagggc tgtcaacatt cagagctctg 2160
tgcaaaccgg aatcccagtc ttgttgaaca aaaacagcag ggatgtcatc cagatgattg 2220
ataagtcgtg cagcagacaa gagctcactc agcactgtga gagtacgatc gcagtccacc 2280
atgccgatgg aattgcccca cttgagccac atagtttctg gagatgccct gtcggagaac 2340
cgtatcttag ctcagatcct gaaatctcat tgctgcctgg tccgagcttg ttatctggtt 2400
ctacaacgat ctctggatgt gttaggctcc cttcactctc aattggcgag gcaatctatg 2460
cctattcatc aaatctcatt acacaaggtt gtgctgacat agggaaatca tatcaggtcc 2520
tgcagctagg gtacatatca ctcaattcag atatgttccc tgatcttaac cccgtagtgt 2580
cccacactta tgacatcaac gacaatcgga aatcatgctc tgtggtggca accgggacta 2640
ggggttatca gctttgctcc atgccgactg tagacgaaag aaccgactac tctagtgatg 2700
gtattgagga tctggtcctt gatgtcctgg atctcaaagg gagaactaag tctcaccggt 2760
atcgcaacag cgaggtagat cttgatcacc cgttctctgc actatacccc agtgtaggca 2820
acggcattgc aacagaaggc tcattgatat ttcttgggta tggtggacta accacccctc 2880
tgcagggtga tacaaaatgt aggacccaag gatgccaaca ggtgtcgcaa gacacatgca 2940
atgaggctct gaaaattaca tggctaggag ggaaacaggt ggtcagcgtg atcatccagg 3000
tcaatgacta tctctcagag aggccaaaga taagagtcac aaccattcca atcactcaaa 3060
actatctcgg ggcggaaggt agattattaa aattgggtga tcgggtgtac atctatacaa 3120
gatcatcagg ctggcactct caactgcaga taggagtact tgatgtcagc caccctttga 3180
ctatcaactg gacacctcat gaagccttgt ctagaccagg aaataaagag tgcaattggt 3240
acaataagtg tccgaaggaa tgcatatcag gcgtatacac tgatgcttat ccattgtccc 3300
ctgatgcagc taacgtcgct accgtcacgc tatatgccaa tacatcgcgt gtcaacccaa 3360
caatcatgta ttctaacact actaacatta taaatatgtt aaggataaag gatgttcaat 3420
tagaggctgc atataccacg acatcgtgta tcacgcattt tggtaaaggc tactgctttc 3480
acatcatcga gatcaatcag aagagcctga ataccttaca gccgatgctc tttaagacta 3540
gcatccctaa attatgcaag gccgagtctt aagcggccgc gcatgcgaat tcactcctca 3600
ggtgcaggct gcctatcaga aggtggtggc tggtgtggcc aatgccctgg ctcacaaata 3660
ccactgagat ctttttccct ctgccaaaaa ttatggggac atcatgaagc cccttgagca 3720
tctgacttct ggctaataaa ggaaatttat tttcattgca atagtgtgtt ggaatttttt 3780
gtgtctctca ctcggaagga catatgggag ggcaaatcat ttaaaacatc agaatgagta 3840
tttggtttag agtttggcaa catatgccca tatgctggct gccatgaaca aaggttggct 3900
ataaagaggt catcagtata tgaaacagcc ccctgctgtc tattccttat tccatagaaa 3960
agccttgact tgaggttaga ttttttttat attttgtttt gtgttatttt tttctttaac 4020
atccctaaaa ttttccttac atgttttact agccagattt ttcctcctct cctgactact 4080
cccagtcata gctgtccctc ttctcttatg gagatccctc gacctgcagc ccaagcttgg 4140
cgtaatcatg gtcatagctg tttcctgtgt gaaattgtta tccgctcaca attccacaca 4200
acatacgagc cggaagcata aagtgtaaag cctggggtgc ctaatgagtg agctaactca 4260
cattaattgc gttgcgctca ctgcccgctt tccagtcggg aaacctgtcg tgccagcgga 4320
tccgcatctc aattagtcag caaccatagt cccgccccta actccgccca tcccgcccct 4380
aactccgccc agttccgccc attctccgcc ccatggctga ctaatttttt ttatttatgc 4440
agaggccgag gccgcctcgg cctctgagct attccagaag tagtgaggag gcttttttgg 4500
aggcctaggc ttttgcaaaa agctaacttg tttattgcag cttataatgg ttacaaataa 4560
agcaatagca tcacaaattt cacaaataaa gcattttttt cactgcattc tagttgtggt 4620
ttgtccaaac tcatcaatgt atcttatcat gtctgtccgc ttcctcgctc actgactcgc 4680
tgcgctcggt cgttcggctg cggcgagcgg tatcagctca ctcaaaggcg gtaatacggt 4740
tatccacaga atcaggggat aacgcaggaa agaacatgtg agcaaaaggc cagcaaaagg 4800
ccaggaaccg taaaaaggcc gcgttgctgg cgtttttcca taggctccgc ccccctgacg 4860
agcatcacaa aaatcgacgc tcaagtcaga ggtggcgaaa cccgacagga ctataaagat 4920
accaggcgtt tccccctgga agctccctcg tgcgctctcc tgttccgacc ctgccgctta 4980
ccggatacct gtccgccttt ctcccttcgg gaagcgtggc gctttctcat agctcacgct 5040
gtaggtatct cagttcggtg taggtcgttc gctccaagct gggctgtgtg cacgaacccc 5100
ccgttcagcc cgaccgctgc gccttatccg gtaactatcg tcttgagtcc aacccggtaa 5160
gacacgactt atcgccactg gcagcagcca ctggtaacag gattagcaga gcgaggtatg 5220
taggcggtgc tacagagttc ttgaagtggt ggcctaacta cggctacact agaagaacag 5280
tatttggtat ctgcgctctg ctgaagccag ttaccttcgg aaaaagagtt ggtagctctt 5340
gatccggcaa acaaaccacc gctggtagcg gtggtttttt tgtttgcaag cagcagatta 5400
cgcgcagaaa aaaaggatct caagaagatc ctttgatctt ttctacgggg tctgacgctc 5460
agtggaacga aaactcacgt taagggattt tggtcatgag attatcaaaa aggatcttca 5520
cctagatcct tttaaattaa aaatgaagtt ttaaatcaat ctaaagtata tatgagtaaa 5580
cttggtctga cagttagaaa aactcatcga gcatcaaatg aaactgcaat ttattcatat 5640
caggattatc aataccatat ttttgaaaaa gccgtttctg taatgaagga gaaaactcac 5700
cgaggcagtt ccataggatg gcaagatcct ggtatcggtc tgcgattccg actcgtccaa 5760
catcaataca acctattaat ttcccctcgt caaaaataag gttatcaagt gagaaatcac 5820
catgagtgac gactgaatcc ggtgagaatg gcaacagctt atgcatttct ttccagactt 5880
gttcaacagg ccagccatta cgctcgtcat caaaatcact cgcatcaacc aaaccgttat 5940
tcattcgtga ttgcgcctga gcgagacgaa atacgcgatc gctgttaaaa ggacaattac 6000
aaacaggaat cgaatgcaac cggcgcagga acactgccag cgcatcaaca atattttcac 6060
ctgaatcagg atattcttct aatacctgga atgctgtttt tccggggatc gcagtggtga 6120
gtaaccatgc atcatcagga gtacggataa aatgcttgat ggtcggaaga ggcataaatt 6180
ccgtcagcca gtttagtctg accatctcat ctgtaacatc attggcaacg ctacctttgc 6240
catgtttcag aaacaactct ggcgcatcgg gcttcccata caatcgatag attgtcgcac 6300
ctgattgccc gacattatcg cgagcccatt tatacccata taaatcagca tccatgttgg 6360
aatttaatcg cggcctagag caagacgttt cccgttgaat atggctcata acaccccttg 6420
tattactgtt tatgtaagca gacagtttta ttgttcatga tgatatattt ttatcttgtg 6480
caatgtaaca tcagagattt tgagacacaa caattggtcg ac 6522
<210> SEQ ID NO: 25
<211> 10528
<223> pGM326
ggtacctcaa tattggccat tagccatatt attcattggt tatatagcat aaatcaatat 60
tggctattgg ccattgcata cgttgtatct atatcataat atgtacattt atattggctc 120
atgtccaata tgaccgccat gttggcattg attattgact agttattaat agtaatcaat 180
tacggggtca ttagttcata gcccatatat ggagttccgc gttacataac ttacggtaaa 240
tggcccgcct ggctgaccgc ccaacgaccc ccgcccattg acgtcaataa tgacgtatgt 300
tcccatagta acgccaatag ggactttcca ttgacgtcaa tgggtggagt atttacggta 360
aactgcccac ttggcagtac atcaagtgta tcatatgcca agtccgcccc ctattgacgt 420
caatgacggt aaatggcccg cctggcatta tgcccagtac atgaccttac gggactttcc 480
tacttggcag tacatctacg tattagtcat cgctattacc atggtgatgc ggttttggca 540
gtacaccaat gggcgtggat agcggtttga ctcacgggga tttccaagtc tccaccccat 600
tgacgtcaat gggagtttgt tttggcacca aaatcaacgg gactttccaa aatgtcgtaa 660
caactgcgat cgcccgcccc gttgacgcaa atgggcggta ggcgtgtacg gtgggaggtc 720
tatataagca gagctcgctg gcttgtaact cagtctctta ctaggagacc agcttgagcc 780
tgggtgttcg ctggttagcc taacctggtt ggccaccagg ggtaaggact ccttggctta 840
gaaagctaat aaacttgcct gcattagagc ttatctgagt caagtgtcct cattgacgcc 900
tcactctctt gaacgggaat cttccttact gggttctctc tctgacccag gcgagagaaa 960
ctccagcagt ggcgcccgaa cagggacttg agtgagagtg taggcacgta cagctgagaa 1020
ggcgtcggac gcgaaggaag cgcggggtgc gacgcgacca agaaggagac ttggtgagta 1080
ggcttctcga gtgccgggaa aaagctcgag cctagttaga ggactaggag aggccgtagc 1140
cgtaactact ctgggcaagt agggcaggcg gtgggtacgc aatgggggcg gctacctcag 1200
cactaaatag gagacaatta gaccaatttg agaaaatacg acttcgcccg aacggaaaga 1260
aaaagtacca aattaaacat ttaatatggg caggcaagga gatggagcgc ttcggcctcc 1320
atgagaggtt gttggagaca gaggaggggt gtaaaagaat catagaagtc ctctaccccc 1380
tagaaccaac aggatcggag ggcttaaaaa gtctgttcaa tcttgtgtgc gtgctatatt 1440
gcttgcacaa ggaacagaaa gtgaaagaca cagaggaagc agtagcaaca gtaagacaac 1500
actgccatct agtggaaaaa gaaaaaagtg caacagagac atctagtgga caaaagaaaa 1560
atgacaaggg aatagcagcg ccacctggtg gcagtcagaa ttttccagcg caacaacaag 1620
gaaatgcctg ggtacatgta cccttgtcac cgcgcacctt aaatgcgtgg gtaaaagcag 1680
tagaggagaa aaaatttgga gcagaaatag tacccatgtt tcaagcccta tcgaattccc 1740
gtttgtgcta gggttcttag gcttcttggg ggctgctgga actgcaatgg gagcagcggc 1800
gacagccctg acggtccagt ctcagcattt gcttgctggg atactgcagc agcagaagaa 1860
tctgctggcg gctgtggagg ctcaacagca gatgttgaag ctgaccattt ggggtgttaa 1920
aaacctcaat gcccgcgtca cagcccttga gaagtaccta gaggatcagg cacgactaaa 1980
ctcctggggg tgcgcatgga aacaagtatg tcataccaca gtggagtggc cctggacaaa 2040
tcggactccg gattggcaaa atatgacttg gttggagtgg gaaagacaaa tagctgattt 2100
ggaaagcaac attacgagac aattagtgaa ggctagagaa caagaggaaa agaatctaga 2160
tgcctatcag aagttaacta gttggtcaga tttctggtct tggttcgatt tctcaaaatg 2220
gcttaacatt ttaaaaatgg gatttttagt aatagtagga ataatagggt taagattact 2280
ttacacagta tatggatgta tagtgagggt taggcaggga tatgttcctc tatctccaca 2340
gatccatatc cgcggcaatt ttaaaagaaa gggaggaata gggggacaga cttcagcaga 2400
gagactaatt aatataataa caacacaatt agaaatacaa catttacaaa ccaaaattca 2460
aaaaatttta aattttagag ccgcggagat ctgttacata acttatggta aatggcctgc 2520
ctggctgact gcccaatgac ccctgcccaa tgatgtcaat aatgatgtat gttcccatgt 2580
aatgccaata gggactttcc attgatgtca atgggtggag tatttatggt aactgcccac 2640
ttggcagtac atcaagtgta tcatatgcca agtatgcccc ctattgatgt caatgatggt 2700
aaatggcctg cctggcatta tgcccagtac atgaccttat gggactttcc tacttggcag 2760
tacatctatg tattagtcat tgctattacc atgggaattc actagtggag aagagcatgc 2820
ttgagggctg agtgcccctc agtgggcaga gagcacatgg cccacagtcc ctgagaagtt 2880
ggggggaggg gtgggcaatt gaactggtgc ctagagaagg tggggcttgg gtaaactggg 2940
aaagtgatgt ggtgtactgg ctccaccttt ttccccaggg tgggggagaa ccatatataa 3000
gtgcagtagt ctctgtgaac attcaagctt ctgccttctc cctcctgtga gtttgctagc 3060
caccatgcag agaagccctc tggagaaggc ctctgtggtg agcaagctgt tcttcagctg 3120
gaccaggccc atcctgagga agggctacag gcagagactg gagctgtctg acatctacca 3180
gatcccctct gtggactctg ctgacaacct gtctgagaag ctggagaggg agtgggatag 3240
agagctggcc agcaagaaga accccaagct gatcaatgcc ctgaggagat gcttcttctg 3300
gagattcatg ttctatggca tcttcctgta cctgggggaa gtgaccaagg ctgtgcagcc 3360
tctgctgctg ggcagaatca ttgccagcta tgaccctgac aacaaggagg agaggagcat 3420
tgccatctac ctgggcattg gcctgtgcct gctgttcatt gtgaggaccc tgctgctgca 3480
ccctgccatc tttggcctgc accacattgg catgcagatg aggattgcca tgttcagcct 3540
gatctacaag aaaaccctga agctgtccag cagagtgctg gacaagatca gcattggcca 3600
gctggtgagc ctgctgagca acaacctgaa caagtttgat gagggcctgg ccctggccca 3660
ctttgtgtgg attgcccctc tgcaggtggc cctgctgatg ggcctgattt gggagctgct 3720
gcaggcctct gccttttgtg gcctgggctt cctgattgtg ctggccctgt ttcaggctgg 3780
cctgggcagg atgatgatga agtacaggga ccagagggca ggcaagatca gtgagaggct 3840
ggtgatcacc tctgagatga ttgagaacat ccagtctgtg aaggcctact gttgggagga 3900
agctatggag aagatgattg aaaacctgag gcagacagag ctgaagctga ccaggaaggc 3960
tgcctatgtg agatacttca acagctctgc cttcttcttc tctggcttct ttgtggtgtt 4020
cctgtctgtg ctgccctatg ccctgatcaa ggggatcatc ctgagaaaga ttttcaccac 4080
catcagcttc tgcattgtgc tgaggatggc tgtgaccaga cagttcccct gggctgtgca 4140
gacctggtat gacagcctgg gggccatcaa caagatccag gacttcctgc agaagcagga 4200
gtacaagacc ctggagtaca acctgaccac cacagaagtg gtgatggaga atgtgacagc 4260
cttctgggag gagggctttg gggagctgtt tgagaaggcc aagcagaaca acaacaacag 4320
aaagaccagc aatggggatg actccctgtt cttctccaac ttctccctgc tgggcacacc 4380
tgtgctgaag gacatcaact tcaagattga gagggggcag ctgctggctg tggctggatc 4440
tacaggggct ggcaagacca gcctgctgat gatgatcatg ggggagctgg agccttctga 4500
gggcaagatc aagcactctg gcaggatcag cttttgcagc cagttcagct ggatcatgcc 4560
tggcaccatc aaggagaaca tcatctttgg agtgagctat gatgagtaca gatacaggag 4620
tgtgatcaag gcctgccagc tggaggagga catcagcaag tttgctgaga aggacaacat 4680
tgtgctgggg gagggaggca ttacactgtc tgggggccag agagccagaa tcagcctggc 4740
cagggctgtg tacaaggatg ctgacctgta cctgctggac tccccctttg gctacctgga 4800
tgtgctgaca gagaaggaga tttttgagag ctgtgtgtgc aagctgatgg ccaacaagac 4860
cagaatcctg gtgaccagca agatggagca cctgaagaag gctgacaaga tcctgatcct 4920
gcatgagggc agcagctact tctatgggac cttctctgag ctgcagaacc tgcagcctga 4980
cttcagctct aagctgatgg gctgtgacag ctttgaccag ttctctgctg agaggaggaa 5040
cagcatcctg acagagaccc tgcacagatt cagcctggag ggagatgccc ctgtgagctg 5100
gacagagacc aagaagcaga gcttcaagca gacaggggag tttggggaga agaggaagaa 5160
ctccatcctg aaccccatca acagcatcag gaagttcagc attgtgcaga aaacccccct 5220
gcagatgaat ggcattgagg aagattctga tgagcccctg gagaggagac tgagcctggt 5280
gcctgattct gagcagggag aggccatcct gcctaggatc tctgtgatca gcacaggccc 5340
tacactgcag gccagaagga ggcagtctgt gctgaacctg atgacccact ctgtgaacca 5400
gggccagaac atccacagga aaaccacagc ctccaccagg aaagtgagcc tggcccctca 5460
ggccaatctg acagagctgg acatctacag caggaggctg tctcaggaga caggcctgga 5520
gatttctgag gagatcaatg aggaggacct gaaagagtgc ttctttgatg acatggagag 5580
catccctgct gtgaccacct ggaacaccta cctgagatac atcacagtgc acaagagcct 5640
gatctttgtg ctgatctggt gcctggtgat cttcctggct gaagtggctg cctctctggt 5700
ggtgctgtgg ctgctgggaa acaccccact gcaggacaag ggcaacagca cccacagcag 5760
gaacaacagc tatgctgtga tcatcacctc cacctccagc tactatgtgt tctacatcta 5820
tgtgggagtg gctgataccc tgctggctat gggcttcttt agaggcctgc ccctggtgca 5880
cacactgatc acagtgagca agatcctcca ccacaagatg ctgcactctg tgctgcaggc 5940
tcctatgagc accctgaata ccctgaaggc tgggggcatc ctgaacagat tctccaagga 6000
tattgccatc ctggatgacc tgctgcctct caccatcttt gacttcatcc agctgctgct 6060
gattgtgatt ggggccattg ctgtggtggc agtgctgcag ccctacatct ttgtggccac 6120
agtgcctgtg attgtggcct tcatcatgct gagggcctac tttctgcaga cctcccagca 6180
gctgaagcag ctggagtctg agggcagaag ccccatcttc acccacctgg tgacaagcct 6240
gaagggcctg tggaccctga gagcctttgg caggcagccc tactttgaga ccctgttcca 6300
caaggccctg aacctgcaca cagccaactg gttcctctac ctgtccaccc tgagatggtt 6360
ccagatgaga attgagatga tctttgtcat cttcttcatt gctgtgacct tcatcagcat 6420
tctgaccaca ggagagggag agggcagagt gggcattatc ctgaccctgg ccatgaacat 6480
catgagcaca ctgcagtggg cagtgaacag cagcattgat gtggacagcc tgatgaggag 6540
tgtgagcaga gtgttcaagt tcattgatat gcccacagag ggcaagccta ccaagagcac 6600
caagccctac aagaatggcc agctgagcaa agtgatgatc attgagaaca gccatgtgaa 6660
gaaggatgat atctggccca gtggaggcca gatgacagtg aaggacctga cagccaagta 6720
cacagagggg ggcaatgcta tcctggagaa catctccttc agcatctccc ctggccgag 6780
agtgggactg ctgggaagaa caggctctgg caagtctacc ctgctgtctg ccttcctgag 6840
gctgctgaac acagagggag agatccagat tgatggagtg tcctgggaca gcatcacact 6900
gcagcagtgg aggaaggcct ttggtgtgat cccccagaaa gtgttcatct tcagtggcac 6960
cttcaggaag aacctggacc cctatgagca gtggtctgac caggagattt ggaaagtggc 7020
tgatgaagtg ggcctgagaa gtgtgattga gcagttccct ggcaagctgg actttgtcct 7080
ggtggatggg ggctgtgtgc tgagccatgg ccacaagcag ctgatgtgcc tggccagatc 7140
agtgctgagc aaggccaaga tcctgctgct ggatgagcct tctgcccacc tggatcctgt 7200
gacctaccag atcatcagga ggaccctcaa gcaggccttt gctgactgca cagtcatcct 7260
gtgtgagcac aggattgagg ccatgctgga gtgccagcag ttcctggtga ttgaggagaa 7320
caaagtgagg cagtatgaca gcatccagaa gctgctgaat gagaggagcc tgttcaggca 7380
ggccatcagc ccctctgata gagtgaagct gttcccccac aggaacagct ccaagtgcaa 7440
gagcaagccc cagattgctg ccctgaagga ggagacagag gaggaagtgc aggacaccag 7500
gctgtgaggg cccaatcaac ctctggatta caaaatttgt gaaagattga ctggtattct 7560
taactatgtt gctcctttta cgctatgtgg atacgctgct ttaatgcctt tgtatcatgc 7620
tattgcttcc cgtatggctt tcattttctc ctccttgtat aaatcctggt tgctgtctct 7680
ttatgaggag ttgtggcccg ttgtcaggca acgtggcgtg gtgtgcactg tgtttgctga 7740
cgcaaccccc actggttggg gcattgccac cacctgtcag ctcctttccg ggactttcgc 7800
tttccccctc cctattgcca cggcggaact catcgccgcc tgccttgccc gctgctggac 7860
aggggctcgg ctgttgggca ctgacaattc cgtggtgttg tcggggaaat catcgtcctt 7920
tccttggctg ctcgcctgtg ttgccacctg gattctgcgc gggacgtcct tctgctacgt 7980
cccttcggcc ctcaatccag cggaccttcc ttcccgcggc ctgctgccgg ctctgcggcc 8040
tcttccgcgt cttcgccttc gccctcagac gagtcggatc tccctttggg ccgcctcccc 8100
gcaagcttcg cactttttaa aagaaaaggg aggactggat gggatttatt actccgatag 8160
gacgctggct tgtaactcag tctcttacta ggagaccagc ttgagcctgg gtgttcgctg 8220
gttagcctaa cctggttggc caccaggggt aaggactcct tggcttagaa agctaataaa 8280
cttgcctgca ttagagctct tacgcgtccc gggctcgaga tccgcatctc aattagtcag 8340
caaccatagt cccgccccta actccgccca tcccgcccct aactccgccc agttccgccc 8400
attctccgcc ccatggctga ctaatttttt ttatttatgc agaggccgag gccgcctcgg 8460
cctctgagct attccagaag tagtgaggag gcttttttgg aggcctaggc ttttgcaaaa 8520
agctaacttg tttattgcag cttataatgg ttacaaataa agcaatagca tcacaaattt 8580
cacaaataaa gcattttttt cactgcattc tagttgtggt ttgtccaaac tcatcaatgt 8640
atcttatcat gtctgtccgc ttcctcgctc actgactcgc tgcgctcggt cgttcggctg 8700
cggcgagcgg tatcagctca ctcaaaggcg gtaatacggt tatccacaga atcaggggat 8760
aacgcaggaa agaacatgtg agcaaaaggc cagcaaaagg ccaggaaccg taaaaaggcc 8820
gcgttgctgg cgtttttcca taggctccgc ccccctgacg agcatcacaa aaatcgacgc 8880
tcaagtcaga ggtggcgaaa cccgacagga ctataaagat accaggcgtt tccccctgga 8940
agctccctcg tgcgctctcc tgttccgacc ctgccgctta ccggatacct gtccgccttt 9000
ctcccttcgg gaagcgtggc gctttctcat agctcacgct gtaggtatct cagttcggtg 9060
taggtcgttc gctccaagct gggctgtgtg cacgaacccc ccgttcagcc cgaccgctgc 9120
gccttatccg gtaactatcg tcttgagtcc aacccggtaa gacacgactt atcgccactg 9180
gcagcagcca ctggtaacag gattagcaga gcgaggtatg taggcggtgc tacagagttc 9240
ttgaagtggt ggcctaacta cggctacact agaagaacag tatttggtat ctgcgctctg 9300
ctgaagccag ttaccttcgg aaaaagagtt ggtagctctt gatccggcaa acaaaccacc 9360
gctggtagcg gtggtttttt tgtttgcaag cagcagatta cgcgcagaaa aaaaggatct 9420
caagaagatc ctttgatctt ttctacgggg tctgacgctc agtggaacga aaactcacgt 9480
taagggattt tggtcatgag attatcaaaa aggatcttca cctagatcct tttaaattaa 9540
aaatgaagtt ttaaatcaat ctaaagtata tatgagtaaa cttggtctga cagttagaaa 9600
aactcatcga gcatcaaatg aaactgcaat ttattcatat caggattatc aataccatat 9660
ttttgaaaaa gccgtttctg taatgaagga gaaaactcac cgaggcagtt ccataggatg 9720
gcaagatcct ggtatcggtc tgcgattccg actcgtccaa catcaataca acctattaat 9780
ttcccctcgt caaaaataag gttatcaagt gagaaatcac catgagtgac gactgaatcc 9840
ggtgagaatg gcaacagctt atgcatttct ttccagactt gttcaacagg ccagccatta 9900
cgctcgtcat caaaatcact cgcatcaacc aaaccgttat tcattcgtga ttgcgcctga 9960
gcgagacgaa atacgcgatc gctgttaaaa ggacaattac aaacaggaat cgaatgcaac 10020
cggcgcagga acactgccag cgcatcaaca atattttcac ctgaatcagg atattcttct 10080
aatacctgga atgctgtttt tccggggatc gcagtggtga gtaaccatgc atcatcagga 10140
gtacggataa aatgcttgat ggtcggaaga ggcataaatt ccgtcagcca gtttagtctg 10200
accatctcat ctgtaacatc attggcaacg ctacctttgc catgtttcag aaacaactct 10260
ggcgcatcgg gcttcccata caatcgatag attgtcgcac ctgattgccc gacattatcg 10320
cgagcccatt tatacccata taaatcagca tccatgttgg aatttaatcg cggcctagag 10380
caagacgttt cccgttgaat atggctcata acaccccttg tattactgtt tatgtaagca 10440
gacagtttta ttgttcatga tgatatattt ttatcttgtg caatgtaaca tcagagattt 10500
tgagacacaa caattggtcg acggatcc 10528
<210> SEQ ID NO: 26
<211> 574
<223> hCEF promoter
agatctgtta cataacttat ggtaaatggc ctgcctggct gactgcccaa tgacccctgc 60
ccaatgatgt caataatgat gtatgttccc atgtaatgcc aatagggact ttccattgat 120
gtcaatgggt ggagtattta tggtaactgc ccacttggca gtacatcaag tgtatcatat 180
gccaagtatg ccccctattg atgtcaatga tggtaaatgg cctgcctggc attatgccca 240
gtacatgacc ttatgggact ttcctacttg gcagtacatc tatgtattag tcattgctat 300
taccatggga attcactagt ggagaagagc atgcttgagg gctgagtgcc cctcagtggg 360
cagagagcac atggcccaca gtccctgaga agttgggggg aggggtgggc aattgaactg 420
gtgcctagag aaggtggggc ttgggtaaac tgggaaagtg atgtggtgta ctggctccac 480
ctttttcccc agggtggggg agaaccatat ataagtgcag tagtctctgt gaacattcaa 540
gcttctgcct tctccctcct gtgagtttgc tagc 574
<210> SEQ ID NO: 27
<211> 873
<223> CMV promoter
ccgcggagat ctcaatattg gccattagcc atattattca ttggttatat agcataaatc 60
aatattggct attggccatt gcatacgttg tatctatatc ataatatgta catttatatt 120
ggctcatgtc caatatgacc gccatgttgg cattgattat tgactagtta ttaatagtaa 180
tcaattacgg ggtcattagt tcatagccca tatatggagt tccgcgttac ataacttacg 240
gtaaatggcc cgcctggctg accgcccaac gacccccgcc cattgacgtc aataatgacg 300
tatgttccca tagtaacgcc aatagggact ttccattgac gtcaatgggt ggagtattta 360
cggtaaactg cccacttggc agtacatcaa gtgtatcata tgccaagtcc gccccctatt 420
gacgtcaatg acggtaaatg gcccgcctgg cattatgccc agtacatgac cttacgggac 480
tttcctactt ggcagtacat ctacgtatta gtcatcgcta ttaccatggt gatgcggttt 540
tggcagtaca ccaatgggcg tggatagcgg tttgactcac ggggatttcc aagtctccac 600
cccattgacg tcaatgggag tttgttttgg caccaaaatc aacgggactt tccaaaatgt 660
cgtaataacc ccgccccgtt gacgcaaatg ggcggtaggc gtgtacggtg ggaggtctat 720
ataagcagag ctcgtttagt gaaccgtcag atcactagaa gctttattgc ggtagtttat 780
cacagttaaa ttgctaacgc agtcagtgct tctgacacaa cagtctcgaa cttaagctgc 840
agaagttggt cgtgaggcac tgggcaggct agc 873
<210> SEQ ID NO: 28
<211> 395
<223> EFla promoter
agatccatat ccgcggcaat tttaaaagaa agggaggaat agggggacag acttcagcag 60
agagactaat taatataata acaacacaat tagaaataca acatttacaa accaaaattc 120
aaaaaatttt aaattttaga gccgcggaga tcccgtgagg ctccggtgcc cgtcagtggg 180
cagagcgcac atcgcccaca gtccccgaga agttgggggg aggggtcggc aattgaaccg 240
gtgcctagag aaggtggcgc ggggtaaact gggaaagtga tgtcgtgtac tggctccgcc 300
tttttcccga gggtggggga gaaccgtata taagtgcagt agtcgccgtg aacgttcttt 360
ttcgcaacgg gtttgccgcc agaacacagg ctagc 395
<210> SEQ ID NO: 29
<211> 4459
<223> SOCFTR2
gctagccac atgcagagaa gccctctgga gaaggcctct gtggtgagca agctgttctt 60
cagctggacc aggcccatcc tgaggaaggg ctacaggcag agactggagc tgtctgacat 120
ctaccagatc ccctctgtgg actctgctga caacctgtct gagaagctgg agagggagtg 180
ggatagagag ctggccagca agaagaaccc caagctgatc aatgccctga ggagatgctt 240
cttctggaga ttcatgttct atggcatctt cctgtacctg ggggaagtga ccaaggctgt 300
gcagcctctg ctgctgggca gaatcattgc cagctatgac cctgacaaca aggaggagag 360
gagcattgcc atctacctgg gcattggcct gtgcctgctg ttcattgtga ggaccctgct 420
gctgcaccct gccatctttg gcctgcacca cattggcatg cagatgagga ttgccatgtt 480
cagcctgatc tacaagaaaa ccctgaagct gtccagcaga gtgctggaca agatcagcat 540
tggccagctg gtgagcctgc tgagcaacaa cctgaacaag tttgatgagg gcctggccct 600
ggcccacttt gtgtggattg cccctctgca ggtggccctg ctgatgggcc tgatttggga 660
gctgctgcag gcctctgcct tttgtggcct gggcttcctg attgtgctgg ccctgtttca 720
ggctggcctg ggcaggatga tgatgaagta cagggaccag agggcaggca agatcagtga 780
gaggctggtg atcacctctg agatgattga gaacatccag tctgtgaagg cctactgttg 840
ggaggaagct atggagaaga tgattgaaaa cctgaggcag acagagctga agctgaccag 900
gaaggctgcc tatgtgagat acttcaacag ctctgccttc ttcttctctg gcttctttgt 960
ggtgttcctg tctgtgctgc cctatgccct gatcaagggg atcatcctga gaaagatttt 1020
caccaccatc agcttctgca ttgtgctgag gatggctgtg accagacagt tcccctgggc 1080
tgtgcagacc tggtatgaca gcctgggggc catcaacaag atccaggact tcctgcagaa 1140
gcaggagtac aagaccctgg agtacaacct gaccaccaca gaagtggtga tggagaatgt 1200
gacagccttc tgggaggagg gctttgggga gctgtttgag aaggccaagc agaacaacaa 1260
caacagaaag accagcaatg gggatgactc cctgttcttc tccaacttct ccctgctggg 1320
cacacctgtg ctgaaggaca tcaacttcaa gattgagagg gggcagctgc tggctgtggc 1380
tggatctaca ggggctggca agaccagcct gctgatgatg atcatggggg agctggagcc 1440
ttctgagggc aagatcaagc actctggcag gatcagcttt tgcagccagt tcagctggat 1500
catgcctggc accatcaagg agaacatcat ctttggagtg agctatgatg agtacagata 1560
caggagtgtg atcaaggcct gccagctgga ggaggacatc agcaagtttg ctgagaagga 1620
caacattgtg ctgggggagg gaggcattac actgtctggg ggccagagag ccagaatcag 1680
cctggccagg gctgtgtaca aggatgctga cctgtacctg ctggactccc cctttggcta 1740
cctggatgtg ctgacagaga aggagatttt tgagagctgt gtgtgcaagc tgatggccaa 1800
caagaccaga atcctggtga ccagcaagat ggagcacctg aagaaggctg acaagatcct 1860
gatcctgcat gagggcagca gctacttcta tgggaccttc tctgagctgc agaacctgca 1920
gcctgacttc agctctaagc tgatgggctg tgacagcttt gaccagttct ctgctgagag 1980
gaggaacagc atcctgacag agaccctgca cagattcagc ctggagggag atgcccctgt 2040
gagctggaca gagaccaaga agcagagctt caagcagaca ggggagtttg gggagaagag 2100
gaagaactcc atcctgaacc ccatcaacag catcaggaag ttcagcattg tgcagaaaac 2160
ccccctgcag atgaatggca ttgaggaaga ttctgatgag cccctggaga ggagactgag 2220
cctggtgcct gattctgagc agggagaggc catcctgcct aggatctctg tgatcagcac 2280
aggccctaca ctgcaggcca gaaggaggca gtctgtgctg aacctgatga cccactctgt 2340
gaaccagggc cagaacatcc acaggaaaac cacagcctcc accaggaaag tgagcctggc 2400
ccctcaggcc aatctgacag agctggacat ctacagcagg aggctgtctc aggagacagg 2460
cctggagatt tctgaggaga tcaatgagga ggacctgaaa gagtgcttct ttgatgacat 2520
ggagagcatc cctgctgtga ccacctggaa cacctacctg agatacatca cagtgcacaa 2580
gagcctgatc tttgtgctga tctggtgcct ggtgatcttc ctggctgaag tggctgcctc 2640
tctggtggtg ctgtggctgc tgggaaacac cccactgcag gacaagggca acagcaccca 2700
cagcaggaac aacagctatg ctgtgatcat cacctccacc tccagctact atgtgttcta 2760
catctatgtg ggagtggctg ataccctgct ggctatgggc ttctttagag gcctgcccct 2820
ggtgcacaca ctgatcacag tgagcaagat cctccaccac aagatgctgc actctgtgct 2880
gcaggctcct atgagcaccc tgaataccct gaaggctggg ggcatcctga acagattctc 2940
caaggatatt gccatcctgg atgacctgct gcctctcacc atctttgact tcatccagct 3000
gctgctgatt gtgattgggg ccattgctgt ggtggcagtg ctgcagccct acatctttgt 3060
ggccacagtg cctgtgattg tggccttcat catgctgagg gcctactttc tgcagacctc 3120
ccagcagctg aagcagctgg agtctgaggg cagaagcccc atcttcaccc acctggtgac 3180
aagcctgaag ggcctgtgga ccctgagagc ctttggcagg cagccctact ttgagaccct 3240
gttccacaag gccctgaacc tgcacacagc caactggttc ctctacctgt ccaccctgag 3300
atggttccag atgagaattg agatgatctt tgtcatcttc ttcattgctg tgaccttcat 3360
cagcattctg accacaggag agggagaggg cagagtgggc attatcctga ccctggccat 3420
gaacatcatg agcacactgc agtgggcagt gaacagcagc attgatgtgg acagcctgat 3480
gaggagtgtg agcagagtgt tcaagttcat tgatatgccc acagagggca agcctaccaa 3540
gagcaccaag ccctacaaga atggccagct gagcaaagtg atgatcattg agaacagcca 3600
tgtgaagaag gatgatatct ggcccagtgg aggccagatg acagtgaagg acctgacagc 3660
caagtacaca gaggggggca atgctatcct ggagaacatc tccttcagca tctcccctgg 3720
ccagagagtg ggactgctgg gaagaacagg ctctggcaag tctaccctgc tgtctgcctt 3780
cctgaggctg ctgaacacag agggagagat ccagattgat ggagtgtcct gggacagcat 3840
cacactgcag cagtggagga aggcctttgg tgtgatcccc cagaaagtgt tcatcttcag 3900
tggcaccttc aggaagaacc tggaccccta tgagcagtgg tctgaccagg agatttggaa 3960
agtggctgat gaagtgggcc tgagaagtgt gattgagcag ttccctggca agctggactt 4020
tgtcctggtg gatgggggct gtgtgctgag ccatggccac aagcagctga tgtgcctggc 4080
cagatcagtg ctgagcaagg ccaagatcct gctgctggat gagccttctg cccacctgga 4140
tcctgtgacc taccagatca tcaggaggac cctcaagcag gcctttgctg actgcacagt 4200
catcctgtgt gagcacagga ttgaggccat gctggagtgc cagcagttcc tggtgattga 4260
ggagaacaaa gtgaggcagt atgacagcat ccagaagctg ctgaatgaga ggagcctgtt 4320
caggcaggcc atcagcccct ctgatagagt gaagctgttc ccccacagga acagctccaa 4380
gtgcaagagc aagccccaga ttgctgccct gaaggaggag acagaggagg aagtgcagga 4440
caccaggctg tgagggccc 4459
<210> SEQ ID NO: 30
<211> 1257
<223> sohAAT
atgcccagct ctgtgtcctg gggcattctg ctgctggctg gcctgtgctg tctggtgcct 60
gtgtccctgg ctgaggaccc tcagggggat gctgcccaga aaacagacac ctcccaccat 120
gaccaggacc accccacctt caacaagatc acccccaacc tggcagagtt tgccttcagc 180
ctgtacagac agctggccca ccagagcaac agcaccaaca tctttttcag ccctgtgtcc 240
attgccacag cctttgccat gctgagcctg ggcaccaagg ctgacaccca tgatgagatc 300
ctggaaggcc tgaacttcaa cctgacagag atccctgagg cccagatcca tgagggcttc 360
caggaactgc tgagaaccct gaaccagcca gacagccagc tgcagctgac aacaggcaat 420
gggctgttcc tgtctgaggg cctgaagctg gtggacaagt ttctggaaga tgtgaagaag 480
ctgtaccact ctgaggcctt cacagtgaac tttggggaca cagaagaggc caagaaacag 540
atcaatgact atgtggaaaa gggcacccag ggcaagattg tggaccttgt gaaagagctg 600
gacagggaca ctgtgtttgc ccttgtgaac tacatcttct tcaagggcaa gtgggagagg 660
ccctttgaag tgaaggacac tgaggaagag gacttccatg tggaccaagt gaccacagtg 720
aaggtgccaa tgatgaagag actggggatg ttcaatatcc agcactgcaa gaaactgagc 780
agctgggtgc tgctgatgaa gtacctgggc aatgctacag ccatattctt tctgcctgat 840
gagggcaagc tgcagcacct ggaaaatgag ctgacccatg acatcatcac caaatttctg 900
gaaaatgagg acagaagatc tgccagcctg catctgccca agctgagcat cacaggcaca 960
tatgacctga agtctgtgct gggacagctg ggaatcacca aggtgttcag caatggggca 1020
gacctgagtg gagtgacaga ggaagcccct ctgaagctgt ccaaggctgt gcacaaggca 1080
gtgctgacca ttgatgagaa gggcacagag gctgctgggg ccatgtttct ggaagccatc 1140
cccatgtcca tccccccaga agtgaagttc aacaagccct ttgtgttcct gatgattgag 1200
cagaacacca agagccccct gttcatgggc aaggttgtga accccaccca gaaatga 1257
<210> SEQ ID NO: 31
<211> 1257
<223> sohAAT completmentary strand
tacgggtcga gacacaggac cccgtaagac gacgaccgac cggacacgac agaccacgga 60
cacagggacc gactcctggg agtcccccta cgacgggtct tttgtctgtg gagggtggta 120
ctggtcctgg tggggtggaa gttgttctag tgggggttgg accgtctcaa acggaagtcg 180
gacatgtctg tcgaccgggt ggtctcgttg tcgtggttgt agaaaaagtc gggacacagg 240
taacggtgtc ggaaacggta cgactcggac ccgtggttcc gactgtgggt actactctag 300
gaccttccgg acttgaagtt ggactgtctc tagggactcc gggtctaggt actcccgaag 360
gtccttgacg actcttggga cttggtcggt ctgtcggtcg acgtcgactg ttgtccgtta 420
cccgacaagg acagactccc ggacttcgac cacctgttca aagaccttct acacttcttc 480
gacatggtga gactccggaa gtgtcacttg aaacccctgt gtcttctccg gttctttgtc 540
tagttactga tacacctttt cccgtgggtc ccgttctaac acctggaaca ctttctcgac 600
ctgtccctgt gacacaaacg ggaacacttg atgtagaaga agttcccgtt caccctctcc 660
gggaaacttc acttcctgtg actccttctc ctgaaggtac acctggttca ctggtgtcac 720
ttccacggtt actacttctc tgacccctac aagttatagg tcgtgacgtt ctttgactcg 780
tcgacccacg acgactactt catggacccg ttacgatgtc ggtataagaa agacggacta 840
ctcccgttcg acgtcgtgga ccttttactc gactgggtac tgtagtagtg gtttaaagac 900
cttttactcc tgtcttctag acggtcggac gtagacgggt tcgactcgta gtgtccgtgt 960
atactggact tcagacacga ccctgtcgac ccttagtggt tccacaagtc gttaccccgt 1020
ctggactcac ctcactgtct ccttcgggga gacttcgaca ggttccgaca cgtgttccgt 1080
cacgactggt aactactctt cccgtgtctc cgacgacccc ggtacaaaga ccttcggtag 1140
gggtacaggt aggggggtct tcacttcaag ttgttcggga aacacaagga ctactaactc 1200
gtcttgtggt tctcggggga caagtacccg ttccaacact tggggtgggt ctttact 1257
<210> SEQ ID NO: 32
<211> 419
<223> exemplary AlAT polypeptide
Ala Glu Asp Pro Gln Gly Asp Ala Ala Gln Lys Thr Asp Thr Ser His
1 5 10 15
His Asp Gln Asp His Pro Thr Phe Ala Glu Asp Pro Gln Gly Asp Ala
                        20 25 30
Ala Gln Lys Thr Asp Thr Ser His His Asp Gln Asp His Pro Thr Phe
                35 40 45
Asn Lys Ile Thr Pro Asn Leu Ala Glu Phe Ala Phe Ser Leu Tyr Arg
        50 55 60
Gln Leu Ala His Gln Ser Asn Ser Thr Asn Ile Phe Phe Ser Pro Val
65 70 75 80
Ser Ile Ala Thr Ala Phe Ala Met Leu Ser Leu Gly Thr Lys Ala Asp
                                85 90 95
Thr His Asp Glu Ile Leu Glu Gly Leu Asn Phe Asn Leu Thr Glu Ile
                        100 105 110
Pro Glu Ala Gln Ile His Glu Gly Phe Gln Glu Leu Leu Arg Thr Leu
                115 120 125
Asn Gln Pro Asp Ser Gln Leu Gln Leu Thr Thr Gly Asn Gly Leu Phe
        130 135 140
Leu Ser Glu Gly Leu Lys Leu Val Asp Lys Phe Leu Glu Asp Val Lys
145 150 155 160
Lys Leu Tyr His Ser Glu Ala Phe Thr Val Asn Phe Gly Asp Thr Glu
                                165 170 175
Glu Ala Lys Lys Gln Ile Asn Asp Tyr Val Glu Lys Gly Thr Gln Gly
                        180 185 190
Lys Ile Val Asp Leu Val Lys Glu Leu Asp Arg Asp Thr Val Phe Ala
                195 200 205
Leu Val Asn Tyr Ile Phe Phe Lys Gly Lys Trp Glu Arg Pro Phe Glu
        210 215 220
Val Lys Asp Thr Glu Glu Glu Asp Phe His Val Asp Gln Val Thr Thr
225 230 235 240
Val Lys Val Pro Met Met Lys Arg Leu Gly Met Phe Asn Ile Gln His
                                245 250 255
Cys Lys Lys Leu Ser Ser Trp Val Leu Leu Met Lys Tyr Leu Gly Asn
                        260 265 270
Ala Thr Ala Ile Phe Phe Leu Pro Asp Glu Gly Lys Leu Gln His Leu
                275 280 285
Glu Asn Glu Leu Thr His Asp Ile Ile Thr Lys Phe Leu Glu Asn Glu
        290 295 300
Asp Arg Arg Ser Ala Ser Leu His Leu Pro Lys Leu Ser Ile Thr Gly
305 310 315 320
Thr Tyr Asp Leu Lys Ser Val Leu Gly Gln Leu Gly Ile Thr Lys Val
                                325 330 335
Phe Ser Asn Gly Ala Asp Leu Ser Gly Val Thr Glu Glu Ala Pro Leu
                        340 345 350
Lys Leu Ser Lys Ala Val His Lys Ala Val Leu Thr Ile Asp Glu Lys
                355 360 365
Gly Thr Glu Ala Ala Gly Ala Met Phe Leu Glu Ala Ile Pro Met Ser
        370 375 380
Ile Pro Pro Glu Val Lys Phe Asn Lys Pro Phe Val Phe Leu Met Ile
385 390 395 400
Glu Gln Asn Thr Lys Ser Pro Leu Phe Met Gly Lys Val Val Asn Pro
                                405 410 415
Thr Gln Lys
<210> SEQ ID NO: 33
<211> 5013
<223> codon-optimised FVIII transgene (N6)
atgcagattg agctgagcac ctgcttcttc ctgtgcctgc tgaggttctg cttctctgcc 60
accaggagat actacctggg ggctgtggag ctgagctggg actacatgca gtctgacctg 120
ggggagctgc ctgtggatgc caggttcccc cccagagtgc ccaagagctt ccccttcaac 180
acctctgtgg tgtacaagaa gaccctgttt gtggagttca ctgaccacct gttcaacatt 240
gccaagccca ggcccccctg gatgggcctg ctgggcccca ccatccaggc tgaggtgtat 300
gacactgtgg tgatcaccct gaagaacatg gccagccacc ctgtgagcct gcatgctgtg 360
ggggtgagct actggaaggc ctctgagggg gctgagtatg atgaccagac cagccagagg 420
gagaaggagg atgacaaggt gttccctggg ggcagccaca cctatgtgtg gcaggtgctg 480
aaggagaatg gccccatggc ctctgacccc ctgtgcctga cctacagcta cctgagccat 540
gtggacctgg tgaaggacct gaactctggc ctgattgggg ccctgctggt gtgcagggag 600
ggcagcctgg ccaaggagaa gacccagacc ctgcacaagt tcatcctgct gtttgctgtg 660
tttgatgagg gcaagagctg gcactctgaa accaagaaca gcctgatgca ggacagggat 720
gctgcctctg ccagggcctg gcccaagatg cacactgtga atggctatgt gaacaggagc 780
ctgcctggcc tgattggctg ccacaggaag tctgtgtact ggcatgtgat tggcatgggc 840
accacccctg aggtgcacag catcttcctg gagggccaca ccttcctggt caggaaccac 900
aggcaggcca gcctggagat cagccccatc accttcctga ctgcccagac cctgctgatg 960
gacctgggcc agttcctgct gttctgccac atcagcagcc accagcatga tggcatggag 1020
gcctatgtga aggtggacag ctgccctgag gagccccagc tgaggatgaa gaacaatgag 1080
gaggctgagg actatgatga tgacctgact gactctgaga tggatgtggt gaggtttgat 1140
gatgacaaca gccccagctt catccagatc aggtctgtgg ccaagaagca ccccaagacc 1200
tgggtgcact acattgctgc tgaggaggag gactgggact atgcccccct ggtgctggcc 1260
cctgatgaca ggagctacaa gagccagtac ctgaacaatg gcccccagag gattggcagg 1320
aagtacaaga aggtcaggtt catggcctac actgatgaaa ccttcaagac cagggaggcc 1380
atccagcatg agtctggcat cctgggcccc ctgctgtatg gggaggtggg ggacaccctg 1440
ctgatcatct tcaagaacca ggccagcagg ccctacaaca tctaccccca tggcatcact 1500
gatgtgaggc ccctgtacag caggaggctg cccaaggggg tgaagcacct gaaggacttc 1560
cccatcctgc ctggggagat cttcaagtac aagtggactg tgactgtgga ggatggcccc 1620
accaagtctg accccaggtg cctgaccaga tactacagca gctttgtgaa catggagagg 1680
gacctggcct ctggcctgat tggccccctg ctgatctgct acaaggagtc tgtggaccag 1740
aggggcaacc agatcatgtc tgacaagagg aatgtgatcc tgttctctgt gtttgatgag 1800
aacaggagct ggtacctgac tgagaacatc cagaggttcc tgcccaaccc tgctggggtg 1860
cagctggagg accctgagtt ccaggccagc aacatcatgc acagcatcaa tggctatgtg 1920
tttgacagcc tgcagctgtc tgtgtgcctg catgaggtgg cctactggta catcctgagc 1980
attggggccc agactgactt cctgtctgtg ttcttctctg gctacacctt caagcacaag 2040
atggtgtatg aggacaccct gaccctgttc cccttctctg gggagactgt gttcatgagc 2100
atggagaacc ctggcctgtg gattctgggc tgccacaact ctgacttcag gaacaggggc 2160
atgactgccc tgctgaaagt ctccagctgt gacaagaaca ctggggacta ctatgaggac 2220
agctatgagg acatctctgc ctacctgctg agcaagaaca atgccattga gcccaggagc 2280
ttcagccaga acagcaggca ccccagcacc aggcagaagc agttcaatgc caccaccatc 2340
cctgagaatg acatagagaa gacagaccca tggtttgccc accggacccc catgcccaag 2400
atccagaatg tgagcagctc tgacctgctg atgctgctga ggcagagccc caccccccat 2460
ggcctgagcc tgtctgacct gcaggaggcc aagtatgaaa ccttctctga tgaccccagc 2520
cctggggcca ttgacagcaa caacagcctg tctgagatga cccacttcag gccccagctg 2580
caccactctg gggacatggt gttcacccct gagtctggcc tgcagctgag gctgaatgag 2640
aagctgggca ccactgctgc cactgagctg aagaagctgg acttcaaagt ctccagcacc 2700
agcaacaacc tgatcagcac catcccctct gacaacctgg ctgctggcac tgacaacacc 2760
agcagcctgg gcccccccag catgcctgtg cactatgaca gccagctgga caccaccctg 2820
tttggcaaga agagcagccc cctgactgag tctgggggcc ccctgagcct gtctgaggag 2880
aacaatgaca gcaagctgct ggagtctggc ctgatgaaca gccaggagag cagctggggc 2940
aagaatgtga gcagcaggga gatcaccagg accaccctgc agtctgacca ggaggagatt 3000
gactatgatg acaccatctc tgtggagatg aagaaggagg actttgacat ctacgacgag 3060
gacgagaacc agagccccag gagcttccag aagaagacca ggcactactt cattgctgct 3120
gtggagaggc tgtgggacta tggcatgagc agcagccccc atgtgctgag gaacagggcc 3180
cagtctggct ctgtgcccca gttcaagaag gtggtgttcc aggagttcac tgatggcagc 3240
ttcacccagc ccctgtacag aggggagctg aatgagcacc tgggcctgct gggcccctac 3300
atcagggctg aggtggagga caacatcatg gtgaccttca ggaaccaggc cagcaggccc 3360
tacagcttct acagcagcct gatcagctat gaggaggacc agaggcaggg ggctgagccc 3420
aggaagaact ttgtgaagcc caatgaaacc aagacctact tctggaaggt gcagcaccac 3480
atggccccca ccaaggatga gtttgactgc aaggcctggg cctacttctc tgatgtggac 3540
ctggagaagg atgtgcactc tggcctgatt ggccccctgc tggtgtgcca caccaacacc 3600
ctgaaccctg cccatggcag gcaggtgact gtgcaggagt ttgccctgtt cttcaccatc 3660
tttgatgaaa ccaagagctg gtacttcact gagaacatgg agaggaactg cagggccccc 3720
tgcaacatcc agatggagga ccccaccttc aaggagaact acaggttcca tgccatcaat 3780
ggctacatca tggacaccct gcctggcctg gtgatggccc aggaccagag gatcaggtgg 3840
tacctgctga gcatgggcag caatgagaac atccacagca tccacttctc tggccatgtg 3900
ttcactgtga ggaagaagga ggagtacaag atggccctgt acaacctgta ccctggggtg 3960
tttgagactg tggagatgct gcccagcaag gctggcatct ggagggtgga gtgcctgatt 4020
ggggagcacc tgcatgctgg catgagcacc ctgttcctgg tgtacagcaa caagtgccag 4080
acccccctgg gcatggcctc tggccacatc agggacttcc agatcactgc ctctggccag 4140
tatggccagt gggcccccaa gctggccagg ctgcactact ctggcagcat caatgcctgg 4200
agcaccaagg agcccttcag ctggatcaag gtggacctgc tggcccccat gatcatccat 4260
ggcatcaaga cccagggggc caggcagaag ttcagcagcc tgtacatcag ccagttcatc 4320
atcatgtaca gcctggatgg caagaagtgg cagacctaca ggggcaacag cactggcacc 4380
ctgatggtgt tctttggcaa tgtggacagc tctggcatca agcacaacat cttcaacccc 4440
cccatcattg ccagatacat caggctgcac cccacccact acagcatcag gagcaccctg 4500
aggatggagc tgatgggctg tgacctgaac agctgcagca tgcccctggg catggagagc 4560
aaggccatct ctgatgccca gatcactgcc agcagctact tcaccaacat gtttgccacc 4620
tggagcccca gcaaggccag gctgcacctg cagggcagga gcaatgcctg gaggccccag 4680
gtcaacaacc ccaaggagtg gctgcaggtg gacttccaga agaccatgaa ggtgactggg 4740
gtgaccaccc agggggtgaa gagcctgctg accagcatgt atgtgaagga gttcctgatc 4800
agcagcagcc aggatggcca ccagtggacc ctgttcttcc agaatggcaa ggtgaaggtg 4860
ttccagggca accaggacag cttcacccct gtggtgaaca gcctggaccc ccccctgctg 4920
accagatacc tgaggattca cccccagagc tgggtgcacc agattgccct gaggatggag 4980
gtgctgggct gtgaggccca ggacctgtac tga 5013
<210> SEQ ID NO: 34
<211> 4425
<223> codon-optimised FVIII transgene (V3)
atgcagattg agctgagcac ctgcttcttc ctgtgcctgc tgaggttctg cttctctgcc 60
accaggagat actacctggg ggctgtggag ctgagctggg actacatgca gtctgacctg 120
ggggagctgc ctgtggatgc caggttcccc cccagagtgc ccaagagctt ccccttcaac 180
acctctgtgg tgtacaagaa gaccctgttt gtggagttca ctgaccacct gttcaacatt 240
gccaagccca ggcccccctg gatgggcctg ctgggcccca ccatccaggc tgaggtgtat 300
gacactgtgg tgatcaccct gaagaacatg gccagccacc ctgtgagcct gcatgctgtg 360
ggggtgagct actggaaggc ctctgagggg gctgagtatg atgaccagac cagccagagg 420
gagaaggagg atgacaaggt gttccctggg ggcagccaca cctatgtgtg gcaggtgctg 480
aaggagaatg gccccatggc ctctgacccc ctgtgcctga cctacagcta cctgagccat 540
gtggacctgg tgaaggacct gaactctggc ctgattgggg ccctgctggt gtgcagggag 600
ggcagcctgg ccaaggagaa gacccagacc ctgcacaagt tcatcctgct gtttgctgtg 660
tttgatgagg gcaagagctg gcactctgaa accaagaaca gcctgatgca ggacagggat 720
gctgcctctg ccagggcctg gcccaagatg cacactgtga atggctatgt gaacaggagc 780
ctgcctggcc tgattggctg ccacaggaag tctgtgtact ggcatgtgat tggcatgggc 840
accacccctg aggtgcacag catcttcctg gagggccaca ccttcctggt caggaaccac 900
aggcaggcca gcctggagat cagccccatc accttcctga ctgcccagac cctgctgatg 960
gacctgggcc agttcctgct gttctgccac atcagcagcc accagcatga tggcatggag 1020
gcctatgtga aggtggacag ctgccctgag gagccccagc tgaggatgaa gaacaatgag 1080
gaggctgagg actatgatga tgacctgact gactctgaga tggatgtggt gaggtttgat 1140
gatgacaaca gccccagctt catccagatc aggtctgtgg ccaagaagca ccccaagacc 1200
tgggtgcact acattgctgc tgaggaggag gactgggact atgcccccct ggtgctggcc 1260
cctgatgaca ggagctacaa gagccagtac ctgaacaatg gcccccagag gattggcagg 1320
aagtacaaga aggtcaggtt catggcctac actgatgaaa ccttcaagac cagggaggcc 1380
atccagcatg agtctggcat cctgggcccc ctgctgtatg gggaggtggg ggacaccctg 1440
ctgatcatct tcaagaacca ggccagcagg ccctacaaca tctaccccca tggcatcact 1500
gatgtgaggc ccctgtacag caggaggctg cccaaggggg tgaagcacct gaaggacttc 1560
cccatcctgc ctggggagat cttcaagtac aagtggactg tgactgtgga ggatggcccc 1620
accaagtctg accccaggtg cctgaccaga tactacagca gctttgtgaa catggagagg 1680
gacctggcct ctggcctgat tggccccctg ctgatctgct acaaggagtc tgtggaccag 1740
aggggcaacc agatcatgtc tgacaagagg aatgtgatcc tgttctctgt gtttgatgag 1800
aacaggagct ggtacctgac tgagaacatc cagaggttcc tgcccaaccc tgctggggtg 1860
cagctggagg accctgagtt ccaggccagc aacatcatgc acagcatcaa tggctatgtg 1920
tttgacagcc tgcagctgtc tgtgtgcctg catgaggtgg cctactggta catcctgagc 1980
attggggccc agactgactt cctgtctgtg ttcttctctg gctacacctt caagcacaag 2040
atggtgtatg aggacaccct gaccctgttc cccttctctg gggagactgt gttcatgagc 2100
atggagaacc ctggcctgtg gattctgggc tgccacaact ctgacttcag gaacaggggc 2160
atgactgccc tgctgaaagt ctccagctgt gacaagaaca ctggggacta ctatgaggac 2220
agctatgagg acatctctgc ctacctgctg agcaagaaca atgccattga gcccaggagc 2280
ttcagccaga atgccactaa tgtgtctaac aacagcaaca ccagcaatga cagcaatgtg 2340
tctcccccag tgctgaagag gcaccagagg gagatcacca ggaccaccct gcagtctgac 2400
caggaggaga ttgactatga tgacaccatc tctgtggaga tgaagaagga ggactttgac 2460
atctacgacg aggacgagaa ccagagcccc aggagcttcc agaagaagac caggcactac 2520
ttcattgctg ctgtggagag gctgtgggac tatggcatga gcagcagccc ccatgtgctg 2580
aggaacaggg cccagtctgg ctctgtgccc cagttcaaga aggtggtgtt ccaggagttc 2640
actgatggca gcttcaccca gcccctgtac agaggggagc tgaatgagca cctgggcctg 2700
ctgggcccct acatcagggc tgaggtggag gacaacatca tggtgacctt caggaaccag 2760
gccagcaggc cctacagctt ctacagcagc ctgatcagct atgaggagga ccagaggcag 2820
ggggctgagc ccaggaagaa ctttgtgaag cccaatgaaa ccaagaccta cttctggaag 2880
gtgcagcacc acatggcccc caccaaggat gagtttgact gcaaggcctg ggcctacttc 2940
tctgatgtgg acctggagaa ggatgtgcac tctggcctga ttggccccct gctggtgtgc 3000
cacaccaaca ccctgaaccc tgcccatggc aggcaggtga ctgtgcagga gtttgccctg 3060
ttcttcacca tctttgatga aaccaagagc tggtacttca ctgagaacat ggagaggaac 3120
tgcagggccc cctgcaacat ccagatggag gaccccacct tcaaggagaa ctacaggttc 3180
catgccatca atggctacat catggacacc ctgcctggcc tggtgatggc ccaggaccag 3240
aggatcaggt ggtacctgct gagcatgggc agcaatgaga acatccacag catccacttc 3300
tctggccatg tgttcactgt gaggaagaag gaggagtaca agatggccct gtacaacctg 3360
taccctgggg tgtttgagac tgtggagatg ctgcccagca aggctggcat ctggagggtg 3420
gagtgcctga ttggggagca cctgcatgct ggcatgagca ccctgttcct ggtgtacagc 3480
aacaagtgcc agacccccct gggcatggcc tctggccaca tcagggactt ccagatcact 3540
gcctctggcc agtatggcca gtgggccccc aagctggcca ggctgcacta ctctggcagc 3600
atcaatgcct ggagcaccaa ggagcccttc agctggatca aggtggacct gctggccccc 3660
atgatcatcc atggcatcaa gacccagggg gccaggcaga agttcagcag cctgtacatc 3720
agccagttca tcatcatgta cagcctggat ggcaagaagt ggcagaccta caggggcaac 3780
agcactggca ccctgatggt gttctttggc aatgtggaca gctctggcat caagcacaac 3840
atcttcaacc cccccatcat tgccagatac atcaggctgc accccaccca ctacagcatc 3900
aggagcaccc tgaggatgga gctgatgggc tgtgacctga acagctgcag catgcccctg 3960
ggcatggaga gcaaggccat ctctgatgcc cagatcactg ccagcagcta cttcaccaac 4020
atgtttgcca cctggagccc cagcaaggcc aggctgcacc tgcagggcag gagcaatgcc 4080
tggaggcccc aggtcaacaa ccccaaggag tggctgcagg tggacttcca gaagaccatg 4140
aaggtgactg gggtgaccac ccagggggtg aagagcctgc tgaccagcat gtatgtgaag 4200
gagttcctga tcagcagcag ccaggatggc caccagtgga ccctgttctt ccagaatggc 4260
aaggtgaagg tgttccaggg caaccaggac agcttcaccc ctgtggtgaa cagcctggac 4320
ccccccctgc tgaccagata cctgaggatt cacccccaga gctgggtgca ccagattgcc 4380
ctgaggatgg aggtgctggg ctgtgaggcc caggacctgt actga 4425
<210> SEQ ID NO: 35
<211> 5013
<223> codon-optimised FVIII transgene (N6) complementary strand
tacgtctaac tcgactcgtg gacgaagaag gacacggacg actccaagac gaagagacgg 60
tggtcctcta tgatggaccc ccgacacctc gactcgaccc tgatgtacgt cagactggac 120
cccctcgacg gacacctacg gtccaagggg gggtctcacg ggttctcgaa ggggaagttg 180
tggagacacc acatgttctt ctgggacaaa cacctcaagt gactggtgga caagttgtaa 240
cggttcgggt ccggggggac ctacccggac gacccggggt ggtaggtccg actccacata 300
ctgtgacacc actagtggga cttcttgtac cggtcggtgg gacactcgga cgtacgacac 360
ccccactcga tgaccttccg gagactcccc cgactcatac tactggtctg gtcggtctcc 420
ctcttcctcc tactgttcca caagggaccc ccgtcggtgt ggatacacac cgtccacgac 480
ttcctcttac cggggtaccg gagactgggg gacacggact ggatgtcgat ggactcggta 540
cacctggacc acttcctgga cttgagaccg gactaacccc gggacgacca cacgtccctc 600
ccgtcggacc ggttcctctt ctgggtctgg gacgtgttca agtaggacga caaacgacac 660
aaactactcc cgttctcgac cgtgagactt tggttcttgt cggactacgt cctgtcccta 720
cgacggagac ggtcccggac cgggttctac gtgtgacact taccgataca cttgtcctcg 780
gacggaccgg actaaccgac ggtgtccttc agacacatga ccgtacacta accgtacccg 840
tggtggggac tccacgtgtc gtagaaggac ctcccggtgt ggaaggacca gtccttggtg 900
tccgtccggt cggacctcta gtcggggtag tggaaggact gacgggtctg ggacgactac 960
ctggacccgg tcaaggacga caagacggtg tagtcgtcgg tggtcgtact accgtacctc 1020
cggatacact tccacctgtc gacgggactc ctcggggtcg actcctactt cttgttactc 1080
ctccgactcc tgatactact actggactga ctgagactct acctacacca ctccaaacta 1140
ctactgttgt cggggtcgaa gtaggtctag tccagacacc ggttcttcgt ggggttctgg 1200
acccacgtga tgtaacgacg actcctcctc ctgaccctga tacgggggga ccacgaccgg 1260
ggactactgt cctcgatgtt ctcggtcatg gacttgttac cgggggtctc ctaaccgtcc 1320
ttcatgttct tccagtccaa gtaccggatg tgactacttt ggaagttctg gtccctccgg 1380
taggtcgtac tcagaccgta ggacccgggg gacgacatac ccctccaccc cctgtgggac 1440
gactagtaga agttcttggt ccggtcgtcc gggatgttgt agatgggggt accgtagtga 1500
ctacactccg gggacatgtc gtcctccgac gggttccccc acttcgtgga cttcctgaag 1560
gggtaggacg gacccctcta gaagttcatg ttcacctgac actgacacct cctaccgggg 1620
tggttcagac tggggtccac ggactggtct atgatgtcgt cgaaacactt gtacctctcc 1680
ctggaccgga gaccggacta accgggggac gactagacga tgttcctcag acacctggtc 1740
tccccgttgg tctagtacag actgttctcc ttacactagg acaagagaca caaactactc 1800
ttgtcctcga ccatggactg actcttgtag gtctccaagg acgggttggg acgaccccac 1860
gtcgacctcc tgggactcaa ggtccggtcg ttgtagtacg tgtcgtagtt accgatacac 1920
aaactgtcgg acgtcgacag acacacggac gtactccacc ggatgaccat gtaggactcg 1980
taaccccggg tctgactgaa ggacagacac aagaagagac cgatgtggaa gttcgtgttc 2040
taccacatac tcctgtggga ctgggacaag gggaagagac ccctctgaca caagtactcg 2100
tacctcttgg gaccggacac ctaagacccg acggtgttga gactgaagtc cttgtccccg 2160
tactgacggg acgactttca gaggtcgaca ctgttcttgt gacccctgat gatactcctg 2220
tcgatactcc tgtagagacg gatggacgac tcgttcttgt tacggtaact cgggtcctcg 2280
aagtcggtct tgtcgtccgt ggggtcgtgg tccgtcttcg tcaagttacg gtggtggtag 2340
ggactcttac tgtatctctt ctgtctgggt accaaacggg tggcctgggg gtacgggttc 2400
taggtcttac actcgtcgag actggacgac tacgacgact ccgtctcggg gtggggggta 2460
ccggactcgg acagactgga cgtcctccgg ttcatacttt ggaagagact actggggtcg 2520
ggaccccggt aactgtcgtt gttgtcggac agactctact gggtgaagtc cggggtcgac 2580
gtggtgagac ccctgtacca caagtgggga ctcagaccgg acgtcgactc cgacttactc 2640
ttcgacccgt ggtgacgacg gtgactcgac ttcttcgacc tgaagtttca gaggtcgtgg 2700
tcgttgttgg actagtcgtg gtaggggaga ctgttggacc gacgaccgtg actgttgtgg 2760
tcgtcggacc cgggggggtc gtacggacac gtgatactgt cggtcgacct gtggtgggac 2820
aaaccgttct tctcgtcggg ggactgactc agacccccgg gggactcgga cagactcctc 2880
ttgttactgt cgttcgacga cctcagaccg gactacttgt cggtcctctc gtcgaccccg 2940
ttcttacact cgtcgtccct ctagtggtcc tggtgggacg tcagactggt cctcctctaa 3000
ctgatactac tgtggtagag acacctctac ttcttcctcc tgaaactgta gatgctgctc 3060
ctgctcttgg tctcggggtc ctcgaaggtc ttcttctggt ccgtgatgaa gtaacgacga 3120
cacctctccg acaccctgat accgtactcg tcgtcggggg tacacgactc cttgtcccgg 3180
gtcagaccga gacacggggt caagttcttc caccacaagg tcctcaagtg actaccgtcg 3240
aagtgggtcg gggacatgtc tcccctcgac ttactcgtgg acccggacga cccggggatg 3300
tagtcccgac tccacctcct gttgtagtac cactggaagt ccttggtccg gtcgtccggg 3360
atgtcgaaga tgtcgtcgga ctagtcgata ctcctcctgg tctccgtccc ccgactcggg 3420
tccttcttga aacacttcgg gttactttgg ttctggatga agaccttcca cgtcgtggtg 3480
taccgggggt ggttcctact caaactgacg ttccggaccc ggatgaagag actacacctg 3540
gacctcttcc tacacgtgag accggactaa ccgggggacg accacacggt gtggttgtgg 3600
gacttgggac gggtaccgtc cgtccactga cacgtcctca aacgggacaa gaagtggtag 3660
aaactacttt ggttctcgac catgaagtga ctcttgtacc tctccttgac gtcccggggg 3720
acgttgtagg tctacctcct ggggtggaag ttcctcttga tgtccaaggt acggtagtta 3780
ccgatgtagt acctgtggga cggaccggac cactaccggg tcctggtctc ctagtccacc 3840
atggacgact cgtacccgtc gttactcttg taggtgtcgt aggtgaagag accggtacac 3900
aagtgacact ccttcttcct cctcatgttc taccgggaca tgttggacat gggaccccac 3960
aaactctgac acctctacga cgggtcgttc cgaccgtaga cctcccacct cacggactaa 4020
cccctcgtgg acgtacgacc gtactcgtgg gacaaggacc acatgtcgtt gttcacggtc 4080
tggggggacc cgtaccggag accggtgtag tccctgaagg tctagtgacg gagaccggtc 4140
ataccggtca cccgggggtt cgaccggtcc gacgtgatga gaccgtcgta gttacggacc 4200
tcgtggttcc tcgggaagtc gacctagttc cacctggacg accgggggta ctagtaggta 4260
ccgtagttct gggtcccccg gtccgtcttc aagtcgtcgg acatgtagtc ggtcaagtag 4320
tagtacatgt cggacctacc gttcttcacc gtctggatgt ccccgttgtc gtgaccgtgg 4380
gactaccaca agaaaccgtt acacctgtcg agaccgtagt tcgtgttgta gaagttgggg 4440
gggtagtaac ggtctatgta gtccgacgtg gggtgggtga tgtcgtagtc ctcgtgggac 4500
tcctacctcg actacccgac actggacttg tcgacgtcgt acggggaccc gtacctctcg 4560
ttccggtaga gactacgggt ctagtgacgg tcgtcgatga agtggttgta caaacggtgg 4620
acctcggggt cgttccggtc cgacgtggac gtcccgtcct cgttacggac ctccggggtc 4680
cagttgttgg ggttcctcac cgacgtccac ctgaaggtct tctggtactt ccactgaccc 4740
cactggtggg tcccccactt ctcggacgac tggtcgtaca tacacttcct caaggactag 4800
tcgtcgtcgg tcctaccggt ggtcacctgg gacaagaagg tcttaccgtt ccacttccac 4860
aaggtcccgt tggtcctgtc gaagtgggga caccacttgt cggacctggg gggggacgac 4920
tggtctatgg actcctaagt gggggtctcg acccacgtgg tctaacggga ctcctacctc 4980
cacgacccga cactccgggt cctggacatg act 5013
<210> SEQ ID NO: 36
<211> 4425
<223> codon-optimised FVIII transgene (V3) complementary strand
tacgtctaac tcgactcgtg gacgaagaag gacacggacg actccaagac gaagagacgg 60
tggtcctcta tgatggaccc ccgacacctc gactcgaccc tgatgtacgt cagactggac 120
cccctcgacg gacacctacg gtccaagggg gggtctcacg ggttctcgaa ggggaagttg 180
tggagacacc acatgttctt ctgggacaaa cacctcaagt gactggtgga caagttgtaa 240
cggttcgggt ccggggggac ctacccggac gacccggggt ggtaggtccg actccacata 300
ctgtgacacc actagtggga cttcttgtac cggtcggtgg gacactcgga cgtacgacac 360
ccccactcga tgaccttccg gagactcccc cgactcatac tactggtctg gtcggtctcc 420
ctcttcctcc tactgttcca caagggaccc ccgtcggtgt ggatacacac cgtccacgac 480
ttcctcttac cggggtaccg gagactgggg gacacggact ggatgtcgat ggactcggta 540
cacctggacc acttcctgga cttgagaccg gactaacccc gggacgacca cacgtccctc 600
ccgtcggacc ggttcctctt ctgggtctgg gacgtgttca agtaggacga caaacgacac 660
aaactactcc cgttctcgac cgtgagactt tggttcttgt cggactacgt cctgtcccta 720
cgacggagac ggtcccggac cgggttctac gtgtgacact taccgataca cttgtcctcg 780
gacggaccgg actaaccgac ggtgtccttc agacacatga ccgtacacta accgtacccg 840
tggtggggac tccacgtgtc gtagaaggac ctcccggtgt ggaaggacca gtccttggtg 900
tccgtccggt cggacctcta gtcggggtag tggaaggact gacgggtctg ggacgactac 960
ctggacccgg tcaaggacga caagacggtg tagtcgtcgg tggtcgtact accgtacctc 1020
cggatacact tccacctgtc gacgggactc ctcggggtcg actcctactt cttgttactc 1080
ctccgactcc tgatactact actggactga ctgagactct acctacacca ctccaaacta 1140
ctactgttgt cggggtcgaa gtaggtctag tccagacacc ggttcttcgt ggggttctgg 1200
acccacgtga tgtaacgacg actcctcctc ctgaccctga tacgggggga ccacgaccgg 1260
ggactactgt cctcgatgtt ctcggtcatg gacttgttac cgggggtctc ctaaccgtcc 1320
ttcatgttct tccagtccaa gtaccggatg tgactacttt ggaagttctg gtccctccgg 1380
taggtcgtac tcagaccgta ggacccgggg gacgacatac ccctccaccc cctgtgggac 1440
gactagtaga agttcttggt ccggtcgtcc gggatgttgt agatgggggt accgtagtga 1500
ctacactccg gggacatgtc gtcctccgac gggttccccc acttcgtgga cttcctgaag 1560
gggtaggacg gacccctcta gaagttcatg ttcacctgac actgacacct cctaccgggg 1620
tggttcagac tggggtccac ggactggtct atgatgtcgt cgaaacactt gtacctctcc 1680
ctggaccgga gaccggacta accgggggac gactagacga tgttcctcag acacctggtc 1740
tccccgttgg tctagtacag actgttctcc ttacactagg acaagagaca caaactactc 1800
ttgtcctcga ccatggactg actcttgtag gtctccaagg acgggttggg acgaccccac 1860
gtcgacctcc tgggactcaa ggtccggtcg ttgtagtacg tgtcgtagtt accgatacac 1920
aaactgtcgg acgtcgacag acacacggac gtactccacc ggatgaccat gtaggactcg 1980
taaccccggg tctgactgaa ggacagacac aagaagagac cgatgtggaa gttcgtgttc 2040
taccacatac tcctgtggga ctgggacaag gggaagagac ccctctgaca caagtactcg 2100
tacctcttgg gaccggacac ctaagacccg acggtgttga gactgaagtc cttgtccccg 2160
tactgacggg acgactttca gaggtcgaca ctgttcttgt gacccctgat gatactcctg 2220
tcgatactcc tgtagagacg gatggacgac tcgttcttgt tacggtaact cgggtcctcg 2280
aagtcggtct tacggtgatt acacagattg ttgtcgttgt ggtcgttact gtcgttacac 2340
agagggggtc acgacttctc cgtggtctcc ctctagtggt cctggtggga cgtcagactg 2400
gtcctcctct aactgatact actgtggtag agacacctct acttcttcct cctgaaactg 2460
tagatgctgc tcctgctctt ggtctcgggg tcctcgaagg tcttcttctg gtccgtgatg 2520
aagtaacgac gacacctctc cgacaccctg ataccgtact cgtcgtcggg ggtacacgac 2580
tccttgtccc gggtcagacc gagacacggg gtcaagttct tccaccacaa ggtcctcaag 2640
tgactaccgt cgaagtgggt cggggacatg tctcccctcg acttactcgt ggacccggac 2700
gacccgggga tgtagtcccg actccacctc ctgttgtagt accactggaa gtccttggtc 2760
cggtcgtccg ggatgtcgaa gatgtcgtcg gactagtcga tactcctcct ggtctccgtc 2820
ccccgactcg ggtccttctt gaaacacttc gggttacttt ggttctggat gaagaccttc 2880
cacgtcgtgg tgtaccgggg gtggttccta ctcaaactga cgttccggac ccggatgaag 2940
agactacacc tggacctctt cctacacgtg agaccggact aaccggggga cgaccacacg 3000
gtgtggttgt gggacttggg acgggtaccg tccgtccact gacacgtcct caaacgggac 3060
aagaagtggt agaaactact ttggttctcg accatgaagt gactcttgta cctctccttg 3120
acgtcccggg ggacgttgta ggtctacctc ctggggtgga agttcctctt gatgtccaag 3180
gtacggtagt taccgatgta gtacctgtgg gacggaccgg accactaccg ggtcctggtc 3240
tcctagtcca ccatggacga ctcgtacccg tcgttactct tgtaggtgtc gtaggtgaag 3300
agaccggtac acaagtgaca ctccttcttc ctcctcatgt tctaccggga catgttggac 3360
atgggacccc acaaactctg acacctctac gacgggtcgt tccgaccgta gacctcccac 3420
ctcacggact aacccctcgt ggacgtacga ccgtactcgt gggacaagga ccacatgtcg 3480
ttgttcacgg tctgggggga cccgtaccgg agaccggtgt agtccctgaa ggtctagtga 3540
cggagaccgg tcataccggt cacccggggg ttcgaccggt ccgacgtgat gagaccgtcg 3600
tagttacgga cctcgtggtt cctcgggaag tcgacctagt tccacctgga cgaccggggg 3660
tactagtagg taccgtagtt ctgggtcccc cggtccgtct tcaagtcgtc ggacatgtag 3720
tcggtcaagt agtagtacat gtcggaccta ccgttcttca ccgtctggat gtccccgttg 3780
tcgtgaccgt gggactacca caagaaaccg ttacacctgt cgagaccgta gttcgtgttg 3840
tagaagttgg gggggtagta acggtctatg tagtccgacg tggggtgggt gatgtcgtag 3900
tcctcgtggg actcctacct cgactacccg acactggact tgtcgacgtc gtacggggac 3960
ccgtacctct cgttccggta gagactacgg gtctagtgac ggtcgtcgat gaagtggttg 4020
tacaaacggt ggacctcggg gtcgttccgg tccgacgtgg acgtcccgtc ctcgttacgg 4080
acctccgggg tccagttgtt ggggttcctc accgacgtcc acctgaaggt cttctggtac 4140
ttccactgac cccactggtg ggtcccccac ttctcggacg actggtcgta catacacttc 4200
ctcaaggact agtcgtcgtc ggtcctaccg gtggtcacct gggacaagaa ggtcttaccg 4260
ttccacttcc acaaggtccc gttggtcctg tcgaagtggg gacaccactt gtcggacctg 4320
gggggggacg actggtctat ggactcctaa gtgggggtct cgacccacgt ggtctaacgg 4380
gactcctacc tccacgaccc gacactccgg gtcctggaca tgact 4425
<210> SEQ ID NO: 37
<211> 1670
<223> exemplary FVIII polypeptide (N6)
Met Gln Ile Glu Leu Ser Thr Cys Phe Phe Leu Cys Leu Leu Arg Phe
1 5 10 15
Cys Phe Ser Ala Thr Arg Arg Tyr Tyr Leu Gly Ala Val Glu Leu Ser
                        20 25 30
Trp Asp Tyr Met Gln Ser Asp Leu Gly Glu Leu Pro Val Asp Ala Arg
                35 40 45
Phe Pro Pro Arg Val Pro Lys Ser Phe Pro Phe Asn Thr Ser Val Val
        50 55 60
Tyr Lys Lys Thr Leu Phe Val Glu Phe Thr Asp His Leu Phe Asn Ile
65 70 75 80
Ala Lys Pro Arg Pro Pro Trp Met Gly Leu Leu Gly Pro Thr Ile Gln
                                85 90 95
Ala Glu Val Tyr Asp Thr Val Val Ile Thr Leu Lys Asn Met Ala Ser
                        100 105 110
His Pro Val Ser Leu His Ala Val Gly Val Ser Tyr Trp Lys Ala Ser
                115 120 125
Glu Gly Ala Glu Tyr Asp Asp Gln Thr Ser Gln Arg Glu Lys Glu Asp
        130 135 140
Asp Lys Val Phe Pro Gly Gly Ser His Thr Tyr Val Trp Gln Val Leu
145 150 155 160
Lys Glu Asn Gly Pro Met Ala Ser Asp Pro Leu Cys Leu Thr Tyr Ser
                                165 170 175
Tyr Leu Ser His Val Asp Leu Val Lys Asp Leu Asn Ser Gly Leu Ile
                        180 185 190
Gly Ala Leu Leu Val Cys Arg Glu Gly Ser Leu Ala Lys Glu Lys Thr
                195 200 205
Gln Thr Leu His Lys Phe Ile Leu Leu Phe Ala Val Phe Asp Glu Gly
        210 215 220
Lys Ser Trp His Ser Glu Thr Lys Asn Ser Leu Met Gln Asp Arg Asp
225 230 235 240
Ala Ala Ser Ala Arg Ala Trp Pro Lys Met His Thr Val Asn Gly Tyr
                                245 250 255
Val Asn Arg Ser Leu Pro Gly Leu Ile Gly Cys His Arg Lys Ser Val
                        260 265 270
Tyr Trp His Val Ile Gly Met Gly Thr Thr Pro Glu Val His Ser Ile
                275 280 285
Phe Leu Glu Gly His Thr Phe Leu Val Arg Asn His Arg Gln Ala Ser
        290 295 300
Leu Glu Ile Ser Pro Ile Thr Phe Leu Thr Ala Gln Thr Leu Leu Met
305 310 315 320
Asp Leu Gly Gln Phe Leu Leu Phe Cys His Ile Ser Ser His Gln His
                                325 330 335
Asp Gly Met Glu Ala Tyr Val Lys Val Asp Ser Cys Pro Glu Glu Pro
                        340 345 350
Gln Leu Arg Met Lys Asn Asn Glu Glu Ala Glu Asp Tyr Asp Asp Asp
                355 360 365
Leu Thr Asp Ser Glu Met Asp Val Val Arg Phe Asp Asp Asp Asn Ser
        370 375 380
Pro Ser Phe Ile Gln Ile Arg Ser Val Ala Lys Lys His Pro Lys Thr
385 390 395 400
Trp Val His Tyr Ile Ala Ala Glu Glu Glu Asp Trp Asp Tyr Ala Pro
                                405 410 415
Leu Val Leu Ala Pro Asp Asp Arg Ser Tyr Lys Ser Gln Tyr Leu Asn
                        420 425 430
Asn Gly Pro Gln Arg Ile Gly Arg Lys Tyr Lys Lys Val Arg Phe Met
                435 440 445
Ala Tyr Thr Asp Glu Thr Phe Lys Thr Arg Glu Ala Ile Gln His Glu
        450 455 460
Ser Gly Ile Leu Gly Pro Leu Leu Tyr Gly Glu Val Gly Asp Thr Leu
465 470 475 480
Leu Ile Ile Phe Lys Asn Gln Ala Ser Arg Pro Tyr Asn Ile Tyr Pro
                                485 490 495
His Gly Ile Thr Asp Val Arg Pro Leu Tyr Ser Arg Arg Leu Pro Lys
                        500 505 510
Gly Val Lys His Leu Lys Asp Phe Pro Ile Leu Pro Gly Glu Ile Phe
                  515 520 525
Lys Tyr Lys Trp Thr Val Thr Val Glu Asp Gly Pro Thr Lys Ser Asp
        530 535 540
Pro Arg Cys Leu Thr Arg Tyr Tyr Ser Ser Phe Val Asn Met Glu Arg
545 550 555 560
Asp Leu Ala Ser Gly Leu Ile Gly Pro Leu Leu Ile Cys Tyr Lys Glu
                                565 570 575
Ser Val Asp Gln Arg Gly Asn Gln Ile Met Ser Asp Lys Arg Asn Val
                        580 585 590
Ile Leu Phe Ser Val Phe Asp Glu Asn Arg Ser Trp Tyr Leu Thr Glu
                595 600 605
Asn Ile Gln Arg Phe Leu Pro Asn Pro Ala Gly Val Gln Leu Glu Asp
        610 615 620
Pro Glu Phe Gln Ala Ser Asn Ile Met His Ser Ile Asn Gly Tyr Val
625 630 635 640
Phe Asp Ser Leu Gln Leu Ser Val Cys Leu His Glu Val Ala Tyr Trp
                                645 650 655
Tyr Ile Leu Ser Ile Gly Ala Gln Thr Asp Phe Leu Ser Val Phe Phe
                        660 665 670
Ser Gly Tyr Thr Phe Lys His Lys Met Val Tyr Glu Asp Thr Leu Thr
                675 680 685
Leu Phe Pro Phe Ser Gly Glu Thr Val Phe Met Ser Met Glu Asn Pro
        690 695 700
Gly Leu Trp Ile Leu Gly Cys His Asn Ser Asp Phe Arg Asn Arg Gly
705 710 715 720
Met Thr Ala Leu Leu Lys Val Ser Ser Cys Asp Lys Asn Thr Gly Asp
                                725 730 735
Tyr Tyr Glu Asp Ser Tyr Glu Asp Ile Ser Ala Tyr Leu Leu Ser Lys
                        740 745 750
Asn Asn Ala Ile Glu Pro Arg Ser Phe Ser Gln Asn Ser Arg His Pro
                755 760 765
Ser Thr Arg Gln Lys Gln Phe Asn Ala Thr Thr Ile Pro Glu Asn Asp
        770 775 780
Ile Glu Lys Thr Asp Pro Trp Phe Ala His Arg Thr Pro Met Pro Lys
785 790 795 800
Ile Gln Asn Val Ser Ser Ser Asp Leu Leu Met Leu Leu Arg Gln Ser
                                805 810 815
Pro Thr Pro His Gly Leu Ser Leu Ser Asp Leu Gln Glu Ala Lys Tyr
                        820 825 830
Glu Thr Phe Ser Asp Asp Pro Ser Pro Gly Ala Ile Asp Ser Asn Asn
                835 840 845
Ser Leu Ser Glu Met Thr His Phe Arg Pro Gln Leu His His Ser Gly
        850 855 860
Asp Met Val Phe Thr Pro Glu Ser Gly Leu Gln Leu Arg Leu Asn Glu
865 870 875 880
Lys Leu Gly Thr Thr Ala Ala Thr Glu Leu Lys Lys Leu Asp Phe Lys
                                885 890 895
Val Ser Ser Thr Ser Asn Asn Leu Ile Ser Thr Ile Pro Ser Asp Asn
                        900 905 910
Leu Ala Ala Gly Thr Asp Asn Thr Ser Ser Leu Gly Pro Pro Ser Met
                915 920 925
Pro Val His Tyr Asp Ser Gln Leu Asp Thr Thr Leu Phe Gly Lys Lys
        930 935 940
Ser Ser Pro Leu Thr Glu Ser Gly Gly Pro Leu Ser Leu Ser Glu Glu
945 950 955 960
Asn Asn Asp Ser Lys Leu Leu Glu Ser Gly Leu Met Asn Ser Gln Glu
                                965 970 975
Ser Ser Trp Gly Lys Asn Val Ser Ser Arg Glu Ile Thr Arg Thr Thr
                        980 985 990
Leu Gln Ser Asp Gln Glu Glu Ile Asp Tyr Asp Asp Thr Ile Ser Val
                995 1000 1005
Glu Met Lys Lys Glu Asp Phe Asp Ile Tyr Asp Glu Asp Glu Asn
        1010 1015 1020
Gln Ser Pro Arg Ser Phe Gln Lys Lys Thr Arg His Tyr Phe Ile
        1025 1030 1035
Ala Ala Val Glu Arg Leu Trp Asp Tyr Gly Met Ser Ser Ser Pro
        1040 1045 1050
His Val Leu Arg Asn Arg Ala Gln Ser Gly Ser Val Pro Gln Phe
        1055 1060 1065
Lys Lys Val Val Phe Gln Glu Phe Thr Asp Gly Ser Phe Thr Gln
        1070 1075 1080
Pro Leu Tyr Arg Gly Glu Leu Asn Glu His Leu Gly Leu Leu Gly
        1085 1090 1095
Pro Tyr Ile Arg Ala Glu Val Glu Asp Asn Ile Met Val Thr Phe
        1100 1105 1110
Arg Asn Gln Ala Ser Arg Pro Tyr Ser Phe Tyr Ser Ser Leu Ile
        1115 1120 1125
Ser Tyr Glu Glu Asp Gln Arg Gln Gly Ala Glu Pro Arg Lys Asn
        1130 1135 1140
Phe Val Lys Pro Asn Glu Thr Lys Thr Tyr Phe Trp Lys Val Gln
        1145 1150 1155
His His Met Ala Pro Thr Lys Asp Glu Phe Asp Cys Lys Ala Trp
        1160 1165 1170
Ala Tyr Phe Ser Asp Val Asp Leu Glu Lys Asp Val His Ser Gly
        1175 1180 1185
Leu Ile Gly Pro Leu Leu Val Cys His Thr Asn Thr Leu Asn Pro
        1190 1195 1200
Ala His Gly Arg Gln Val Thr Val Gln Glu Phe Ala Leu Phe Phe
        1205 1210 1215
Thr Ile Phe Asp Glu Thr Lys Ser Trp Tyr Phe Thr Glu Asn Met
        1220 1225 1230
Glu Arg Asn Cys Arg Ala Pro Cys Asn Ile Gln Met Glu Asp Pro
        1235 1240 1245
Thr Phe Lys Glu Asn Tyr Arg Phe His Ala Ile Asn Gly Tyr Ile
        1250 1255 1260
Met Asp Thr Leu Pro Gly Leu Val Met Ala Gln Asp Gln Arg Ile
        1265 1270 1275
Arg Trp Tyr Leu Leu Ser Met Gly Ser Asn Glu Asn Ile His Ser
        1280 1285 1290
Ile His Phe Ser Gly His Val Phe Thr Val Arg Lys Lys Glu Glu
        1295 1300 1305
Tyr Lys Met Ala Leu Tyr Asn Leu Tyr Pro Gly Val Phe Glu Thr
        1310 1315 1320
Val Glu Met Leu Pro Ser Lys Ala Gly Ile Trp Arg Val Glu Cys
        1325 1330 1335
Leu Ile Gly Glu His Leu His Ala Gly Met Ser Thr Leu Phe Leu
        1340 1345 1350
Val Tyr Ser Asn Lys Cys Gln Thr Pro Leu Gly Met Ala Ser Gly
        1355 1360 1365
His Ile Arg Asp Phe Gln Ile Thr Ala Ser Gly Gln Tyr Gly Gln
        1370 1375 1380
Trp Ala Pro Lys Leu Ala Arg Leu His Tyr Ser Gly Ser Ile Asn
        1385 1390 1395
Ala Trp Ser Thr Lys Glu Pro Phe Ser Trp Ile Lys Val Asp Leu
        1400 1405 1410
Leu Ala Pro Met Ile Ile His Gly Ile Lys Thr Gln Gly Ala Arg
        1415 1420 1425
Gln Lys Phe Ser Ser Leu Tyr Ile Ser Gln Phe Ile Ile Met Tyr
        1430 1435 1440
Ser Leu Asp Gly Lys Lys Trp Gln Thr Tyr Arg Gly Asn Ser Thr
        1445 1450 1455
Gly Thr Leu Met Val Phe Phe Gly Asn Val Asp Ser Ser Gly Ile
        1460 1465 1470
Lys His Asn Ile Phe Asn Pro Pro Ile Ile Ala Arg Tyr Ile Arg
        1475 1480 1485
Leu His Pro Thr His Tyr Ser Ile Arg Ser Thr Leu Arg Met Glu
        1490 1495 1500
Leu Met Gly Cys Asp Leu Asn Ser Cys Ser Met Pro Leu Gly Met
        1505 1510 1515
Glu Ser Lys Ala Ile Ser Asp Ala Gln Ile Thr Ala Ser Ser Tyr
        1520 1525 1530
Phe Thr Asn Met Phe Ala Thr Trp Ser Pro Ser Lys Ala Arg Leu
        1535 1540 1545
His Leu Gln Gly Arg Ser Asn Ala Trp Arg Pro Gln Val Asn Asn
        1555 1560 1550
Pro Lys Glu Trp Leu Gln Val Asp Phe Gln Lys Thr Met Lys Val
        1565 1570 1575
Thr Gly Val Thr Thr Gln Gly Val Lys Ser Leu Leu Thr Ser Met
        1580 1585 1590
Tyr Val Lys Glu Phe Leu Ile Ser Ser Ser Gln Asp Gly His Gln
        1595 1600 1605
Trp Thr Leu Phe Phe Gln Asn Gly Lys Val Lys Val Phe Gln Gly
        1610 1615 1620
Asn Gln Asp Ser Phe Thr Pro Val Val Asn Ser Leu Asp Pro Pro
        1625 1630 1635
Leu Leu Thr Arg Tyr Leu Arg Ile His Pro Gln Ser Trp Val His
        1640 1645 1650
Gln Ile Ala Leu Arg Met Glu Val Leu Gly Cys Glu Ala Gln Asp
        1655 1660 1665
Leu Tyr
        1670
<210> SEQ ID NO: 38
<211> 1474
<223> exemplary FVIII polypeptide (V3)
Met Gln Ile Glu Leu Ser Thr Cys Phe Phe Leu Cys Leu Leu Arg Phe
1 5 10 15
Cys Phe Ser Ala Thr Arg Arg Tyr Tyr Leu Gly Ala Val Glu Leu Ser
                        20 25 30
Trp Asp Tyr Met Gln Ser Asp Leu Gly Glu Leu Pro Val Asp Ala Arg
                35 40 45
Phe Pro Pro Arg Val Pro Lys Ser Phe Pro Phe Asn Thr Ser Val Val
        50 55 60
Tyr Lys Lys Thr Leu Phe Val Glu Phe Thr Asp His Leu Phe Asn Ile
65 70 75 80
Ala Lys Pro Arg Pro Pro Trp Met Gly Leu Leu Gly Pro Thr Ile Gln
                                85 90 95
Ala Glu Val Tyr Asp Thr Val Val Ile Thr Leu Lys Asn Met Ala Ser
                        100 105 110
His Pro Val Ser Leu His Ala Val Gly Val Ser Tyr Trp Lys Ala Ser
                115 120 125
Glu Gly Ala Glu Tyr Asp Asp Gln Thr Ser Gln Arg Glu Lys Glu Asp
        130 135 140
Asp Lys Val Phe Pro Gly Gly Ser His Thr Tyr Val Trp Gln Val Leu
145 150 155 160
Lys Glu Asn Gly Pro Met Ala Ser Asp Pro Leu Cys Leu Thr Tyr Ser
                                165 170 175
Tyr Leu Ser His Val Asp Leu Val Lys Asp Leu Asn Ser Gly Leu Ile
                        180 185 190
Gly Ala Leu Leu Val Cys Arg Glu Gly Ser Leu Ala Lys Glu Lys Thr
                195 200 205
Gln Thr Leu His Lys Phe Ile Leu Leu Phe Ala Val Phe Asp Glu Gly
        210 215 220
Lys Ser Trp His Ser Glu Thr Lys Asn Ser Leu Met Gln Asp Arg Asp
225 230 235 240
Ala Ala Ser Ala Arg Ala Trp Pro Lys Met His Thr Val Asn Gly Tyr
                                245 250 255
Val Asn Arg Ser Leu Pro Gly Leu Ile Gly Cys His Arg Lys Ser Val
                        260 265 270
Tyr Trp His Val Ile Gly Met Gly Thr Thr Pro Glu Val His Ser Ile
                275 280 285
Phe Leu Glu Gly His Thr Phe Leu Val Arg Asn His Arg Gln Ala Ser
        290 295 300
Leu Glu Ile Ser Pro Ile Thr Phe Leu Thr Ala Gln Thr Leu Leu Met
305 310 315 320
Asp Leu Gly Gln Phe Leu Leu Phe Cys His Ile Ser Ser His Gln His
                                325 330 335
Asp Gly Met Glu Ala Tyr Val Lys Val Asp Ser Cys Pro Glu Glu Pro
                        340 345 350
Gln Leu Arg Met Lys Asn Asn Glu Glu Ala Glu Asp Tyr Asp Asp Asp
                355 360 365
Leu Thr Asp Ser Glu Met Asp Val Val Arg Phe Asp Asp Asp Asn Ser
        370 375 380
Pro Ser Phe Ile Gln Ile Arg Ser Val Ala Lys Lys His Pro Lys Thr
385 390 395 400
Trp Val His Tyr Ile Ala Ala Glu Glu Glu Asp Trp Asp Tyr Ala Pro
                                405 410 415
Leu Val Leu Ala Pro Asp Asp Arg Ser Tyr Lys Ser Gln Tyr Leu Asn
                        420 425 430
Asn Gly Pro Gln Arg Ile Gly Arg Lys Tyr Lys Lys Val Arg Phe Met
                435 440 445
Ala Tyr Thr Asp Glu Thr Phe Lys Thr Arg Glu Ala Ile Gln His Glu
        450 455 460
Ser Gly Ile Leu Gly Pro Leu Leu Tyr Gly Glu Val Gly Asp Thr Leu
465 470 475 480
Leu Ile Ile Phe Lys Asn Gln Ala Ser Arg Pro Tyr Asn Ile Tyr Pro
                                485 490 495
His Gly Ile Thr Asp Val Arg Pro Leu Tyr Ser Arg Arg Leu Pro Lys
                        500 505 510
Gly Val Lys His Leu Lys Asp Phe Pro Ile Leu Pro Gly Glu Ile Phe
                  515 520 525
Lys Tyr Lys Trp Thr Val Thr Val Glu Asp Gly Pro Thr Lys Ser Asp
        530 535 540
Pro Arg Cys Leu Thr Arg Tyr Tyr Ser Ser Phe Val Asn Met Glu Arg
545 550 555 560
Asp Leu Ala Ser Gly Leu Ile Gly Pro Leu Leu Ile Cys Tyr Lys Glu
                                565 570 575
Ser Val Asp Gln Arg Gly Asn Gln Ile Met Ser Asp Lys Arg Asn Val
                        580 585 590
Ile Leu Phe Ser Val Phe Asp Glu Asn Arg Ser Trp Tyr Leu Thr Glu
                595 600 605
Asn Ile Gln Arg Phe Leu Pro Asn Pro Ala Gly Val Gln Leu Glu Asp
        610 615 620
Pro Glu Phe Gln Ala Ser Asn Ile Met His Ser Ile Asn Gly Tyr Val
625 630 635 640
Phe Asp Ser Leu Gln Leu Ser Val Cys Leu His Glu Val Ala Tyr Trp
                                645 650 655
Tyr Ile Leu Ser Ile Gly Ala Gln Thr Asp Phe Leu Ser Val Phe Phe
                        660 665 670
Ser Gly Tyr Thr Phe Lys His Lys Met Val Tyr Glu Asp Thr Leu Thr
                675 680 685
Leu Phe Pro Phe Ser Gly Glu Thr Val Phe Met Ser Met Glu Asn Pro
        690 695 700
Gly Leu Trp Ile Leu Gly Cys His Asn Ser Asp Phe Arg Asn Arg Gly
705 710 715 720
Met Thr Ala Leu Leu Lys Val Ser Ser Cys Asp Lys Asn Thr Gly Asp
                                725 730 735
Tyr Tyr Glu Asp Ser Tyr Glu Asp Ile Ser Ala Tyr Leu Leu Ser Lys
                        740 745 750
Asn Asn Ala Ile Glu Pro Arg Ser Phe Ser Gln Asn Ala Thr Asn Val
                755 760 765
Ser Asn Asn Ser Asn Thr Ser Asn Asp Ser Asn Val Ser Pro Pro Val
        770 775 780
Leu Lys Arg His Gln Arg Glu Ile Thr Arg Thr Thr Leu Gln Ser Asp
785 790 795 800
Gln Glu Glu Ile Asp Tyr Asp Asp Thr Ile Ser Val Glu Met Lys Lys
                                805 810 815
Glu Asp Phe Asp Ile Tyr Asp Glu Asp Glu Asn Gln Ser Pro Arg Ser
                        820 825 830
Phe Gln Lys Lys Thr Arg His Tyr Phe Ile Ala Ala Val Glu Arg Leu
                835 840 845
Trp Asp Tyr Gly Met Ser Ser Ser Pro His Val Leu Arg Asn Arg Ala
        850 855 860
Gln Ser Gly Ser Val Pro Gln Phe Lys Lys Val Val Phe Gln Glu Phe
865 870 875 880
Thr Asp Gly Ser Phe Thr Gln Pro Leu Tyr Arg Gly Glu Leu Asn Glu
                                885 890 895
His Leu Gly Leu Leu Gly Pro Tyr Ile Arg Ala Glu Val Glu Asp Asn
                        900 905 910
Ile Met Val Thr Phe Arg Asn Gln Ala Ser Arg Pro Tyr Ser Phe Tyr
                915 920 925
Ser Ser Leu Ile Ser Tyr Glu Glu Asp Gln Arg Gln Gly Ala Glu Pro
        930 935 940
Arg Lys Asn Phe Val Lys Pro Asn Glu Thr Lys Thr Tyr Phe Trp Lys
945 950 955 960
Val Gln His His Met Ala Pro Thr Lys Asp Glu Phe Asp Cys Lys Ala
                                965 970 975
Trp Ala Tyr Phe Ser Asp Val Asp Leu Glu Lys Asp Val His Ser Gly
                        980 985 990
Leu Ile Gly Pro Leu Leu Val Cys His Thr Asn Thr Leu Asn Pro Ala
                995 1000 1005
His Gly Arg Gln Val Thr Val Gln Glu Phe Ala Leu Phe Phe Thr
        1010 1015 1020
Ile Phe Asp Glu Thr Lys Ser Trp Tyr Phe Thr Glu Asn Met Glu
        1025 1030 1035
Arg Asn Cys Arg Ala Pro Cys Asn Ile Gln Met Glu Asp Pro Thr
        1040 1045 1050
Phe Lys Glu Asn Tyr Arg Phe His Ala Ile Asn Gly Tyr Ile Met
        1055 1060 1065
Asp Thr Leu Pro Gly Leu Val Met Ala Gln Asp Gln Arg Ile Arg
        1070 1075 1080
Trp Tyr Leu Leu Ser Met Gly Ser Asn Glu Asn Ile His Ser Ile
        1085 1090 1095
His Phe Ser Gly His Val Phe Thr Val Arg Lys Lys Glu Glu Tyr
        1100 1105 1110
Lys Met Ala Leu Tyr Asn Leu Tyr Pro Gly Val Phe Glu Thr Val
        1115 1120 1125
Glu Met Leu Pro Ser Lys Ala Gly Ile Trp Arg Val Glu Cys Leu
        1130 1135 1140
Ile Gly Glu His Leu His Ala Gly Met Ser Thr Leu Phe Leu Val
        1145 1150 1155
Tyr Ser Asn Lys Cys Gln Thr Pro Leu Gly Met Ala Ser Gly His
        1160 1165 1170
Ile Arg Asp Phe Gln Ile Thr Ala Ser Gly Gln Tyr Gly Gln Trp
        1175 1180 1185
Ala Pro Lys Leu Ala Arg Leu His Tyr Ser Gly Ser Ile Asn Ala
        1190 1195 1200
Trp Ser Thr Lys Glu Pro Phe Ser Trp Ile Lys Val Asp Leu Leu
        1205 1210 1215
Ala Pro Met Ile Ile His Gly Ile Lys Thr Gln Gly Ala Arg Gln
        1220 1225 1230
Lys Phe Ser Ser Leu Tyr Ile Ser Gln Phe Ile Ile Met Tyr Ser
        1235 1240 1245
Leu Asp Gly Lys Lys Trp Gln Thr Tyr Arg Gly Asn Ser Thr Gly
        1250 1255 1260
Thr Leu Met Val Phe Phe Gly Asn Val Asp Ser Ser Gly Ile Lys
        1265 1270 1275
His Asn Ile Phe Asn Pro Pro Ile Ile Ala Arg Tyr Ile Arg Leu
        1280 1285 1290
His Pro Thr His Tyr Ser Ile Arg Ser Thr Leu Arg Met Glu Leu
        1295 1300 1305
Met Gly Cys Asp Leu Asn Ser Cys Ser Met Pro Leu Gly Met Glu
        1310 1315 1320
Ser Lys Ala Ile Ser Asp Ala Gln Ile Thr Ala Ser Ser Tyr Phe
        1325 1330 1335
Thr Asn Met Phe Ala Thr Trp Ser Pro Ser Lys Ala Arg Leu His
        1340 1345 1350
Leu Gln Gly Arg Ser Asn Ala Trp Arg Pro Gln Val Asn Asn Pro
        1355 1360 1365
Lys Glu Trp Leu Gln Val Asp Phe Gln Lys Thr Met Lys Val Thr
        1370 1375 1380
Gly Val Thr Thr Gln Gly Val Lys Ser Leu Leu Thr Set Met Thr
        1385 1390 1395
Val Lys Glu Phe Leu Ile Ser Ser Ser Gln Asp Gly His Gln Trp
        1400 1405 1410
Thr Leu Phe Phe Gln Asn Gly Lys Val Lys Val Phe Gln Gly Asn
        1415 1420 1425
Gln Asp Ser Phe Thr Pro Val Val Asn Ser Leu Asp Pro Pro Leu
        1430 1435 1440
Leu Thr Arg Tyr Leu Arg Ile His Pro Gln Ser Trp Val His Gln
        1445 1450 1455
Ile Ala Leu Arg Met Glu Val Leu Gly Cys Glu Ala Gln Asp Leu
        1460 1465 1470
Tyr
<210> SEQ ID NO: 39
<211> 600
<213> Woodchuck hepatitis virus mWPRE
gggcccaatc aacctctgga ttacaaaatt tgtgaaagat tgactggtat tcttaactat 60
gttgctcctt ttacgctatg tggatacgct gctttaatgc ctttgtatca tgctattgct 120
tcccgtatgg ctttcatttt ctcctccttg tataaatcct ggttgctgtc tctttatgag 180
gagttgtggc ccgttgtcag gcaacgtggc gtggtgtgca ctgtgtttgc tgacgcaacc 240
cccactggtt ggggcattgc caccacctgt cagctccttt ccgggacttt cgctttcccc 300
ctccctattg ccacggcgga actcatcgcc gcctgccttg cccgctgctg gacaggggct 360
cggctgttgg gcactgacaa ttccgtggtg ttgtcgggga aatcatcgtc ctttccttgg 420
ctgctcgcct gtgttgccac ctggattctg cgcgggacgt ccttctgcta cgtcccttcg 480
gccctcaatc cagcggacct tccttcccgc ggcctgctgc cggctctgcg gcctcttccg 540
cgtcttcgcc ttcgccctca gacgagtcgg atctcccttt gggccgcctc cccgcaagct 600
<210> SEQ ID NO: 40
<211> 7349
<223> pGM407
ggtacctcaa tattggccat tagccatatt attcattggt tatatagcat aaatcaatat 60
tggctattgg ccattgcata cgttgtatct atatcataat atgtacattt atattggctc 120
atgtccaata tgaccgccat gttggcattg attattgact agttattaat agtaatcaat 180
tacggggtca ttagttcata gcccatatat ggagttccgc gttacataac ttacggtaaa 240
tggcccgcct ggctgaccgc ccaacgaccc ccgcccattg acgtcaataa tgacgtatgt 300
tcccatagta acgccaatag ggactttcca ttgacgtcaa tgggtggagt atttacggta 360
aactgcccac ttggcagtac atcaagtgta tcatatgcca agtccgcccc ctattgacgt 420
caatgacggt aaatggcccg cctggcatta tgcccagtac atgaccttac gggactttcc 480
tacttggcag tacatctacg tattagtcat cgctattacc atggtgatgc ggttttggca 540
gtacaccaat gggcgtggat agcggtttga ctcacgggga tttccaagtc tccaccccat 600
tgacgtcaat gggagtttgt tttggcacca aaatcaacgg gactttccaa aatgtcgtaa 660
caactgcgat cgcccgcccc gttgacgcaa atgggcggta ggcgtgtacg gtgggaggtc 720
tatataagca gagctcgctg gcttgtaact cagtctctta ctaggagacc agcttgagcc 780
tgggtgttcg ctggttagcc taacctggtt ggccaccagg ggtaaggact ccttggctta 840
gaaagctaat aaacttgcct gcattagagc ttatctgagt caagtgtcct cattgacgcc 900
tcactctctt gaacgggaat cttccttact gggttctctc tctgacccag gcgagagaaa 960
ctccagcagt ggcgcccgaa cagggacttg agtgagagtg taggcacgta cagctgagaa 1020
ggcgtcggac gcgaaggaag cgcggggtgc gacgcgacca agaaggagac ttggtgagta 1080
ggcttctcga gtgccgggaa aaagctcgag cctagttaga ggactaggag aggccgtagc 1140
cgtaactact cttgggcaag tagggcaggc ggtgggtacg caatgggggc ggctacctca 1200
gcactaaata ggagacaatt agaccaattt gagaaaatac gacttcgccc gaacggaaag 1260
aaaaagtacc aaattaaaca tttaatatgg gcaggcaagg agatggagcg cttcggcctc 1320
catgagaggt tgttggagac agaggagggg tgtaaaagaa tcatagaagt cctctacccc 1380
ctagaaccaa caggatcgga gggcttaaaa agtctgttca atcttgtgtg cgtgctatat 1440
tgcttgcaca aggaacagaa agtgaaagac acagaggaag cagtagcaac agtaagacaa 1500
cactgccatc tagtggaaaa agaaaaaagt gcaacagaga catctagtgg acaaaagaaa 1560
aatgacaagg gaatagcagc gccacctggt ggcagtcaga attttccagc gcaacaacaa 1620
ggaaatgcct gggtacatgt acccttgtca ccgcgcacct taaatgcgtg ggtaaaagca 1680
gtagaggaga aaaaatttgg agcagaaata gtacccattt ttttgtttca agccctatcg 1740
aattcccgtt tgtgctaggg ttcttaggct tcttgggggc tgctggaact gcaatgggag 1800
cagcggcgac agccctgacg gtccagtctc agcatttgct tgctgggata ctgcagcagc 1860
agaagaatct gctggcggct gtggaggctc aacagcagat gttgaagctg accatttggg 1920
gtgttaaaaa cctcaatgcc cgcgtcacag cccttgagaa gtacctagag gatcaggcac 1980
gactaaactc ctgggggtgc gcatggaaac aagtatgtca taccacagtg gagtggccct 2040
ggacaaatcg gactccggat tggcaaaata tgacttggtt ggagtgggaa agacaaatag 2100
ctgatttgga aagcaacatt acgagacaat tagtgaaggc tagagaacaa gaggaaaaga 2160
atctagatgc ctatcagaag ttaactagtt ggtcagattt ctggtcttgg ttcgatttct 2220
caaaatggct taacatttta aaaatgggat ttttagtaat agtaggaata atagggttaa 2280
gattacttta cacagtatat ggatgtatag tgagggttag gcagggatat gttcctctat 2340
ctccacagat ccatatccgc ggcaatttta aaagaaaggg aggaataggg ggacagactt 2400
cagcagagag actaattaat ataataacaa cacaattaga aatacaacat ttacaaacca 2460
aaattcaaaa aattttaaat tttagagccg cggagatctg ttacataact tatggtaaat 2520
ggcctgcctg gctgactgcc caatgacccc tgcccaatga tgtcaataat gatgtatgtt 2580
cccatgtaat gccaataggg actttccatt gatgtcaatg ggtggagtat ttatggtaac 2640
tgcccacttg gcagtacatc aagtgtatca tatgccaagt atgcccccta ttgatgtcaa 2700
tgatggtaaa tggcctgcct ggcattatgc ccagtacatg accttatggg actttcctac 2760
ttggcagtac atctatgtat tagtcattgc tattaccatg ggaattcact agtggagaag 2820
agcatgcttg agggctgagt gcccctcagt gggcagagag cacatggccc acagtccctg 2880
agaagttggg gggaggggtg ggcaattgaa ctggtgccta gagaaggtgg ggcttgggta 2940
aactgggaaa gtgatgtggt gtactggctc cacctttttc cccagggtgg gggagaacca 3000
tatataagtg cagtagtctc tgtgaacatt caagcttctg ccttctccct cctgtgagtt 3060
tgctagccac catgcccagc tctgtgtcct ggggcattct gctgctggct ggcctgtgct 3120
gtctggtgcc tgtgtccctg gctgaggacc ctcaggggga tgctgcccag aaaacagaca 3180
cctcccacca tgaccaggac caccccacct tcaacaagat cacccccaac ctggcagagt 3240
ttgccttcag cctgtacaga cagctggccc accagagcaa cagcaccaac atctttttca 3300
gccctgtgtc cattgccaca gcctttgcca tgctgagcct gggcaccaag gctgacaccc 3360
atgatgagat cctggaaggc ctgaacttca acctgacaga gatccctgag gcccagatcc 3420
atgagggctt ccaggaactg ctgagaaccc tgaaccagcc agacagccag ctgcagctga 3480
caacaggcaa tgggctgttc ctgtctgagg gcctgaagct ggtggacaag tttctggaag 3540
atgtgaagaa gctgtaccac tctgaggcct tcacagtgaa ctttggggac acagaagagg 3600
ccaagaaaca gatcaatgac tatgtggaaa agggcaccca gggcaagatt gtggaccttg 3660
tgaaagagct ggacagggac actgtgtttg cccttgtgaa ctacatcttc ttcaagggca 3720
agtgggagag gccctttgaa gtgaaggaca ctgaggaaga ggacttccat gtggaccaag 3780
tgaccacagt gaaggtgcca atgatgaaga gactggggat gttcaatatc cagcactgca 3840
agaaactgag cagctgggtg ctgctgatga agtacctggg caatgctaca gccatattct 3900
ttctgcctga tgagggcaag ctgcagcacc tggaaaatga gctgacccat gacatcatca 3960
ccaaatttct ggaaaatgag gacagaagat ctgccagcct gcatctgccc aagctgagca 4020
tcacaggcac atatgacctg aagtctgtgc tgggacagct gggaatcacc aaggtgttca 4080
gcaatggggc agacctgagt ggagtgacag aggaagcccc tctgaagctg tccaaggctg 4140
tgcacaaggc agtgctgacc attgatgaga agggcacaga ggctgctggg gccatgtttc 4200
tggaagccat ccccatgtcc atccccccag aagtgaagtt caacaagccc tttgtgttcc 4260
tgatgattga gcagaacacc aagagccccc tgttcatggg caaggttgtg aaccccaccc 4320
agaaatgagg gcccaatcaa cctctggatt acaaaatttg tgaaagattg actggtattc 4380
ttaactatgt tgctcctttt acgctatgtg gatacgctgc tttaatgcct ttgtatcatg 4440
ctattgcttc ccgtatggct ttcattttct cctccttgta taaatcctgg ttgctgtctc 4500
tttatgagga gttgtggccc gttgtcaggc aacgtggcgt ggtgtgcact gtgtttgctg 4560
acgcaacccc cactggttgg ggcattgcca ccacctgtca gctcctttcc gggactttcg 4620
ctttccccct ccctattgcc acggcggaac tcatcgccgc ctgccttgcc cgctgctgga 4680
caggggctcg gctgttgggc actgacaatt ccgtggtgtt gtcggggaaa tcatcgtcct 4740
ttccttggct gctcgcctgt gttgccacct ggattctgcg cgggacgtcc ttctgctacg 4800
tcccttcggc cctcaatcca gcggaccttc cttcccgcgg cctgctgccg gctctgcggc 4860
ctcttccgcg tcttcgcctt cgccctcaga cgagtcggat ctccctttgg gccgcctccc 4920
cgcaagcttc gcacttttta aaagaaaagg gaggactgga tgggatttat tactccgata 4980
ggacgctggc ttgtaactca gtctcttact aggagaccag cttgagcctg ggtgttcgct 5040
ggttagccta acctggttgg ccaccagggg taaggactcc ttggcttaga aagctaataa 5100
acttgcctgc attagagctc ttacgcgtcc cgggctcgag atccgcatct caattagtca 5160
gcaaccatag tcccgcccct aactccgccc atcccgcccc taactccgcc cagttccgcc 5220
cattctccgc cccatggctg actaattttt tttatttatg cagaggccga ggccgcctcg 5280
gcctctgagc tattccagaa gtagtgagga ggcttttttg gaggcctagg cttttgcaaa 5340
aagctaactt gtttattgca gcttataatg gttacaaata aagcaatagc atcacaaatt 5400
tcacaaataa agcatttttt tcactgcatt ctagttgtgg tttgtccaaa ctcatcaatg 5460
tatcttatca tgtctgtccg cttcctcgct cactgactcg ctgcgctcgg tcgttcggct 5520
gcggcgagcg gtatcagctc actcaaaggc ggtaatacgg ttatccacag aatcagggga 5580
taacgcagga aagaacatgt gagcaaaagg ccagcaaaag gccaggaacc gtaaaaaggc 5640
cgcgttgctg gcgtttttcc ataggctccg cccccctgac gagcatcaca aaaatcgacg 5700
ctcaagtcag aggtggcgaa acccgacagg actataaaga taccaggcgt ttccccctgg 5760
aagctccctc gtgcgctctc ctgttccgac cctgccgctt accggatacc tgtccgcctt 5820
tctcccttcg ggaagcgtgg cgctttctca tagctcacgc tgtaggtatc tcagttcggt 5880
gtaggtcgtt cgctccaagc tgggctgtgt gcacgaaccc cccgttcagc ccgaccgctg 5940
cgccttatcc ggtaactatc gtcttgagtc caacccggta agacacgact tatcgccact 6000
ggcagcagcc actggtaaca ggattagcag agcgaggtat gtaggcggtg ctacagagtt 6060
cttgaagtgg tggcctaact acggctacac tagaagaaca gtatttggta tctgcgctct 6120
gctgaagcca gttaccttcg gaaaaagagt tggtagctct tgatccggca aacaaaccac 6180
cgctggtagc ggtggttttt ttgtttgcaa gcagcagatt acgcgcagaa aaaaaggatc 6240
tcaagaagat cctttgatct tttctacggg gtctgacgct cagtggaacg aaaactcacg 6300
ttaagggatt ttggtcatga gattatcaaa aaggatcttc acctagatcc ttttaaatta 6360
aaaatgaagt tttaaatcaa tctaaagtat atatgagtaa acttggtctg acagttagaa 6420
aaactcatcg agcatcaaat gaaactgcaa tttattcata tcaggattat caataccata 6480
tttttgaaaa agccgtttct gtaatgaagg agaaaactca ccgaggcagt tccataggat 6540
ggcaagatcc tggtatcggt ctgcgattcc gactcgtcca acatcaatac aacctattaa 6600
tttcccctcg tcaaaaataa ggttatcaag tgagaaatca ccatgagtga cgactgaatc 6660
cggtgagaat ggcaacagct tatgcatttc tttccagact tgttcaacag gccagccatt 6720
acgctcgtca tcaaaatcac tcgcatcaac caaaccgtta ttcattcgtg attgcgcctg 6780
agcgagacga aatacgcgat cgctgttaaa aggacaatta caaacaggaa tcgaatgcaa 6840
ccggcgcagg aacactgcca gcgcatcaac aatattttca cctgaatcag gatattcttc 6900
taatacctgg aatgctgttt ttccggggat cgcagtggtg agtaaccatg catcatcagg 6960
agtacggata aaatgcttga tggtcggaag aggcataaat tccgtcagcc agtttagtct 7020
gaccatctca tctgtaacat cattggcaac gctacctttg ccatgtttca gaaacaactc 7080
tggcgcatcg ggcttcccat acaatcgata gattgtcgca cctgattgcc cgacattatc 7140
gcgagcccat ttatacccat ataaatcagc atccatgttg gaatttaatc gcggcctaga 7200
gcaagacgtt tcccgttgaa tatggctcat aacacccctt gtattactgt ttatgtaagc 7260
agacagtttt attgttcatg atgatatatt tttatcttgt gcaatgtaac atcagagatt 7320
ttgagacaca acaattggtc gacggatcc 7349
<210> SEQ ID NO: 41
<211> 10812
<223> pGM411
ggtacctcaa tattggccat tagccatatt attcattggt tatatagcat aaatcaatat 60
tggctattgg ccattgcata cgttgtatct atatcataat atgtacattt atattggctc 120
atgtccaata tgaccgccat gttggcattg attattgact agttattaat agtaatcaat 180
tacggggtca ttagttcata gcccatatat ggagttccgc gttacataac ttacggtaaa 240
tggcccgcct ggctgaccgc ccaacgaccc ccgcccattg acgtcaataa tgacgtatgt 300
tcccatagta acgccaatag ggactttcca ttgacgtcaa tgggtggagt atttacggta 360
aactgcccac ttggcagtac atcaagtgta tcatatgcca agtccgcccc ctattgacgt 420
caatgacggt aaatggcccg cctggcatta tgcccagtac atgaccttac gggactttcc 480
tacttggcag tacatctacg tattagtcat cgctattacc atggtgatgc ggttttggca 540
gtacaccaat gggcgtggat agcggtttga ctcacgggga tttccaagtc tccaccccat 600
tgacgtcaat gggagtttgt tttggcacca aaatcaacgg gactttccaa aatgtcgtaa 660
caactgcgat cgcccgcccc gttgacgcaa atgggcggta ggcgtgtacg gtgggaggtc 720
tatataagca gagctcgctg gcttgtaact cagtctctta ctaggagacc agcttgagcc 780
tgggtgttcg ctggttagcc taacctggtt ggccaccagg ggtaaggact ccttggctta 840
gaaagctaat aaacttgcct gcattagagc ttatctgagt caagtgtcct cattgacgcc 900
tcactctctt gaacgggaat cttccttact gggttctctc tctgacccag gcgagagaaa 960
ctccagcagt ggcgcccgaa cagggacttg agtgagagtg taggcacgta cagctgagaa 1020
ggcgtcggac gcgaaggaag cgcggggtgc gacgcgacca agaaggagac ttggtgagta 1080
ggcttctcga gtgccgggaa aaagctcgag cctagttaga ggactaggag aggccgtagc 1140
cgtaactact ctgggcaagt agggcaggcg gtgggtacgc aatgggggcg gctacctcag 1200
cactaaatag gagacaatta gaccaatttg agaaaatacg acttcgcccg aacggaaaga 1260
aaaagtacca aattaaacat ttaatatggg caggcaagga gatggagcgc ttcggcctcc 1320
atgagaggtt gttggagaca gaggaggggt gtaaaagaat catagaagtc ctctaccccc 1380
tagaaccaac aggatcggag ggcttaaaaa gtctgttcaa tcttgtgtgc gtgctatatt 1440
gcttgcacaa ggaacagaaa gtgaaagaca cagaggaagc agtagcaaca gtaagacaac 1500
actgccatct agtggaaaaa gaaaaaagtg caacagagac atctagtgga caaaagaaaa 1560
atgacaaggg aatagcagcg ccacctggtg gcagtcagaa ttttccagcg caacaacaag 1620
gaaatgcctg ggtacatgta cccttgtcac cgcgcacctt aaatgcgtgg gtaaaagcag 1680
tagaggagaa aaaatttgga gcagaaatag tacccatgtt tcaagcccta tcgaattccc 1740
gtttgtgcta gggttcttag gcttcttggg ggctgctgga actgcaatgg gagcagcggc 1800
gacagccctg acggtccagt ctcagcattt gcttgctggg atactgcagc agcagaagaa 1860
tctgctggcg gctgtggagg ctcaacagca gatgttgaag ctgaccattt ggggtgttaa 1920
aaacctcaat gcccgcgtca cagcccttga gaagtaccta gaggatcagg cacgactaaa 1980
ctcctggggg tgcgcatgga aacaagtatg tcataccaca gtggagtggc cctggacaaa 2040
tcggactccg gattggcaaa atatgacttg gttggagtgg gaaagacaaa tagctgattt 2100
ggaaagcaac attacgagac aattagtgaa ggctagagaa caagaggaaa agaatctaga 2160
tgcctatcag aagttaacta gttggtcaga tttctggtct tggttcgatt tctcaaaatg 2220
gcttaacatt ttaaaaatgg gatttttagt aatagtagga ataatagggt taagattact 2280
ttacacagta tatggatgta tagtgagggt taggcaggga tatgttcctc tatctccaca 2340
gatccatatc cgcggcaatt ttaaaagaaa gggaggaata gggggacaga cttcagcaga 2400
gagactaatt aatataataa caacacaatt agaaatacaa catttacaaa ccaaaattca 2460
aaaaatttta aattttagag ccgcggagat ctcaatattg gccattagcc atattattca 2520
ttggttatat agcataaatc aatattggct attggccatt gcatacgttg tatctatatc 2580
ataatatgta catttatatt ggctcatgtc caatatgacc gccatgttgg cattgattat 2640
tgactagtta ttaatagtaa tcaattacgg ggtcattagt tcatagccca tatatggagt 2700
tccgcgttac ataacttacg gtaaatggcc cgcctggctg accgcccaac gacccccgcc 2760
cattgacgtc aataatgacg tatgttccca tagtaacgcc aatagggact ttccattgac 2820
gtcaatgggt ggagtattta cggtaaactg cccacttggc agtacatcaa gtgtatcata 2880
tgccaagtcc gccccctatt gacgtcaatg acggtaaatg gcccgcctgg cattatgccc 2940
agtacatgac cttacgggac tttcctactt ggcagtacat ctacgtatta gtcatcgcta 3000
ttaccatggt gatgcggttt tggcagtaca ccaatgggcg tggatagcgg tttgactcac 3060
ggggatttcc aagtctccac cccattgacg tcaatgggag tttgttttgg caccaaaatc 3120
aacgggactt tccaaaatgt cgtaataacc ccgccccgtt gacgcaaatg ggcggtaggc 3180
gtgtacggtg ggaggtctat ataagcagag ctcgtttagt gaaccgtcag atcactagaa 3240
gctttattgc ggtagtttat cacagttaaa ttgctaacgc agtcagtgct tctgacacaa 3300
cagtctcgaa cttaagctgc agaagttggt cgtgaggcac tgggcaggct agccaccaat 3360
gcagattgag ctgagcacct gcttcttcct gtgcctgctg aggttctgct tctctgccac 3420
caggagatac tacctggggg ctgtggagct gagctgggac tacatgcagt ctgacctggg 3480
ggagctgcct gtggatgcca ggttcccccc cagagtgccc aagagcttcc ccttcaacac 3540
ctctgtggtg tacaagaaga ccctgtttgt ggagttcact gaccacctgt tcaacattgc 3600
caagcccagg cccccctgga tgggcctgct gggccccacc atccaggctg aggtgtatga 3660
cactgtggtg atcaccctga agaacatggc cagccaccct gtgagcctgc atgctgtggg 3720
ggtgagctac tggaaggcct ctgagggggc tgagtatgat gaccagacca gccagaggga 3780
gaaggaggat gacaaggtgt tccctggggg cagccacacc tatgtgtggc aggtgctgaa 3840
ggagaatggc cccatggcct ctgaccccct gtgcctgacc tacagctacc tgagccatgt 3900
ggacctggtg aaggacctga actctggcct gattggggcc ctgctggtgt gcagggaggg 3960
cagcctggcc aaggagaaga cccagaccct gcacaagttc atcctgctgt ttgctgtgtt 4020
tgatgagggc aagagctggc actctgaaac caagaacagc ctgatgcagg acagggatgc 4080
tgcctctgcc agggcctggc ccaagatgca cactgtgaat ggctatgtga acaggagcct 4140
gcctggcctg attggctgcc acaggaagtc tgtgtactgg catgtgattg gcatgggcac 4200
cacccctgag gtgcacagca tcttcctgga gggccacacc ttcctggtca ggaaccacag 4260
gcaggccagc ctggagatca gccccatcac cttcctgact gcccagaccc tgctgatgga 4320
cctgggccag ttcctgctgt tctgccacat cagcagccac cagcatgatg gcatggaggc 4380
ctatgtgaag gtggacagct gccctgagga gccccagctg aggatgaaga acaatgagga 4440
ggctgaggac tatgatgatg acctgactga ctctgagatg gatgtggtga ggtttgatga 4500
tgacaacagc cccagcttca tccagatcag gtctgtggcc aagaagcacc ccaagacctg 4560
ggtgcactac attgctgctg aggaggagga ctgggactat gcccccctgg tgctggcccc 4620
tgatgacagg agctacaaga gccagtacct gaacaatggc ccccagagga ttggcaggaa 4680
gtacaagaag gtcaggttca tggcctacac tgatgaaacc ttcaagacca gggaggccat 4740
ccagcatgag tctggcatcc tgggccccct gctgtatggg gaggtggggg acaccctgct 4800
gatcatcttc aagaaccagg ccagcaggcc ctacaacatc tacccccatg gcatcactga 4860
tgtgaggccc ctgtacagca ggaggctgcc caagggggtg aagcacctga aggacttccc 4920
catcctgcct ggggagatct tcaagtacaa gtggactgtg actgtggagg atggccccac 4980
caagtctgac cccaggtgcc tgaccagata ctacagcagc tttgtgaaca tggagaggga 5040
cctggcctct ggcctgattg gccccctgct gatctgctac aaggagtctg tggaccagag 5100
gggcaaccag atcatgtctg acaagaggaa tgtgatcctg ttctctgtgt ttgatgagaa 5160
caggagctgg tacctgactg agaacatcca gaggttcctg cccaaccctg ctggggtgca 5220
gctggaggac cctgagttcc aggccagcaa catcatgcac agcatcaatg gctatgtgtt 5280
tgacagcctg cagctgtctg tgtgcctgca tgaggtggcc tactggtaca tcctgagcat 5340
tggggcccag actgacttcc tgtctgtgtt cttctctggc tacaccttca agcacaagat 5400
ggtgtatgag gacaccctga ccctgttccc cttctctggg gagactgtgt tcatgagcat 5460
ggagaaccct ggcctgtgga ttctgggctg ccacaactct gacttcagga acaggggcat 5520
gactgccctg ctgaaagtct ccagctgtga caagaacact ggggactact atgaggacag 5580
ctatgaggac atctctgcct acctgctgag caagaacaat gccattgagc ccaggagctt 5640
cagccagaat gccactaatg tgtctaacaa cagcaacacc agcaatgaca gcaatgtgtc 5700
tcccccagtg ctgaagaggc accagaggga gatcaccagg accaccctgc agtctgacca 5760
ggaggagatt gactatgatg acaccatctc tgtggagatg aagaaggagg actttgacat 5820
ctacgacgag gacgagaacc agagccccag gagcttccag aagaagacca ggcactactt 5880
cattgctgct gtggagaggc tgtgggacta tggcatgagc agcagccccc atgtgctgag 5940
gaacagggcc cagtctggct ctgtgcccca gttcaagaag gtggtgttcc aggagttcac 6000
tgatggcagc ttcacccagc ccctgtacag aggggagctg aatgagcacc tgggcctgct 6060
gggcccctac atcagggctg aggtggagga caacatcatg gtgaccttca ggaaccaggc 6120
cagcaggccc tacagcttct acagcagcct gatcagctat gaggaggacc agaggcaggg 6180
ggctgagccc aggaagaact ttgtgaagcc caatgaaacc aagacctact tctggaaggt 6240
gcagcaccac atggccccca ccaaggatga gtttgactgc aaggcctggg cctacttctc 6300
tgatgtggac ctggagaagg atgtgcactc tggcctgatt ggccccctgc tggtgtgcca 6360
caccaacacc ctgaaccctg cccatggcag gcaggtgact gtgcaggagt ttgccctgtt 6420
cttcaccatc tttgatgaaa ccaagagctg gtacttcact gagaacatgg agaggaactg 6480
cagggccccc tgcaacatcc agatggagga ccccaccttc aaggagaact acaggttcca 6540
tgccatcaat ggctacatca tggacaccct gcctggcctg gtgatggccc aggaccagag 6600
gatcaggtgg tacctgctga gcatgggcag caatgagaac atccacagca tccacttctc 6660
tggccatgtg ttcactgtga ggaagaagga ggagtacaag atggccctgt acaacctgta 6720
ccctggggtg tttgagactg tggagatgct gcccagcaag gctggcatct ggagggtgga 6780
gtgcctgatt ggggagcacc tgcatgctgg catgagcacc ctgttcctgg tgtacagcaa 6840
caagtgccag acccccctgg gcatggcctc tggccacatc agggacttcc agatcactgc 6900
ctctggccag tatggccagt gggcccccaa gctggccagg ctgcactact ctggcagcat 6960
caatgcctgg agcaccaagg agcccttcag ctggatcaag gtggacctgc tggcccccat 7020
gatcatccat ggcatcaaga cccagggggc caggcagaag ttcagcagcc tgtacatcag 7080
ccagttcatc atcatgtaca gcctggatgg caagaagtgg cagacctaca ggggcaacag 7140
cactggcacc ctgatggtgt tctttggcaa tgtggacagc tctggcatca agcacaacat 7200
cttcaacccc cccatcattg ccagatacat caggctgcac cccacccact acagcatcag 7260
gagcaccctg aggatggagc tgatgggctg tgacctgaac agctgcagca tgcccctggg 7320
catggagagc aaggccatct ctgatgccca gatcactgcc agcagctact tcaccaacat 7380
gtttgccacc tggagcccca gcaaggccag gctgcacctg cagggcagga gcaatgcctg 7440
gaggccccag gtcaacaacc ccaaggagtg gctgcaggtg gacttccaga agaccatgaa 7500
ggtgactggg gtgaccaccc agggggtgaa gagcctgctg accagcatgt atgtgaagga 7560
gttcctgatc agcagcagcc aggatggcca ccagtggacc ctgttcttcc agaatggcaa 7620
ggtgaaggtg ttccagggca accaggacag cttcacccct gtggtgaaca gcctggaccc 7680
ccccctgctg accagatacc tgaggattca cccccagagc tgggtgcacc agattgccct 7740
gaggatggag gtgctgggct gtgaggccca ggacctgtac tgagcggccg cgggcccaat 7800
caacctctgg attacaaaat ttgtgaaaga ttgactggta ttcttaacta tgttgctcct 7860
tttacgctat gtggatacgc tgctttaatg cctttgtatc atgctattgc ttcccgtatg 7920
gctttcattt tctcctcctt gtataaatcc tggttgctgt ctctttatga ggagttgtgg 7980
cccgttgtca ggcaacgtgg cgtggtgtgc actgtgtttg ctgacgcaac ccccactggt 8040
tggggcattg ccaccacctg tcagctcctt tccgggactt tcgctttccc cctccctatt 8100
gccacggcgg aactcatcgc cgcctgcctt gcccgctgct ggacaggggc tcggctgttg 8160
ggcactgaca attccgtggt gttgtcgggg aaatcatcgt cctttccttg gctgctcgcc 8220
tgtgttgcca cctggattct gcgcgggacg tccttctgct acgtcccttc ggccctcaat 8280
ccagcggacc ttccttcccg cggcctgctg ccggctctgc ggcctcttcc gcgtcttcgc 8340
cttcgccctc agacgagtcg gatctccctt tgggccgcct ccccgcaagc ttcgcacttt 8400
ttaaaagaaa agggaggact ggatgggatt tattactccg ataggacgct ggcttgtaac 8460
tcagtctctt actaggagac cagcttgagc ctgggtgttc gctggttagc ctaacctggt 8520
tggccaccag gggtaaggac tccttggctt agaaagctaa taaacttgcc tgcattagag 8580
ctcttacgcg tcccgggctc gagatccgca tctcaattag tcagcaacca tagtcccgcc 8640
cctaactccg cccatcccgc ccctaactcc gcccagttcc gcccattctc cgccccatgg 8700
ctgactaatt ttttttattt atgcagaggc cgaggccgcc tcggcctctg agctattcca 8760
gaagtagtga ggaggctttt ttggaggcct aggcttttgc aaaaagctaa cttgtttatt 8820
gcagcttata atggttacaa ataaagcaat agcatcacaa atttcacaaa taaagcattt 8880
ttttcactgc attctagttg tggtttgtcc aaactcatca atgtatctta tcatgtctgt 8940
ccgcttcctc gctcactgac tcgctgcgct cggtcgttcg gctgcggcga gcggtatcag 9000
ctcactcaaa ggcggtaata cggttatcca cagaatcagg ggataacgca ggaaagaaca 9060
tgtgagcaaa aggccagcaa aaggccagga accgtaaaaa ggccgcgttg ctggcgtttt 9120
tccataggct ccgcccccct gacgagcatc acaaaaatcg acgctcaagt cagaggtggc 9180
gaaacccgac aggactataa agataccagg cgtttccccc tggaagctcc ctcgtgcgct 9240
ctcctgttcc gaccctgccg cttaccggat acctgtccgc ctttctccct tcgggaagcg 9300
tggcgctttc tcatagctca cgctgtaggt atctcagttc ggtgtaggtc gttcgctcca 9360
agctgggctg tgtgcacgaa ccccccgttc agcccgaccg ctgcgcctta tccggtaact 9420
atcgtcttga gtccaacccg gtaagacacg acttatcgcc actggcagca gccactggta 9480
acaggattag cagagcgagg tatgtaggcg gtgctacaga gttcttgaag tggtggccta 9540
actacggcta cactagaaga acagtatttg gtatctgcgc tctgctgaag ccagttacct 9600
tcggaaaaag agttggtagc tcttgatccg gcaaacaaac caccgctggt agcggtggtt 9660
tttttgtttg caagcagcag attacgcgca gaaaaaaagg atctcaagaa gatcctttga 9720
tcttttctac ggggtctgac gctcagtgga acgaaaactc acgttaaggg attttggtca 9780
tgagattatc aaaaaggatc ttcacctaga tccttttaaa ttaaaaatga agttttaaat 9840
caatctaaag tatatatgag taaacttggt ctgacagtta gaaaaactca tcgagcatca 9900
aatgaaactg caatttattc atatcaggat tatcaatacc atatttttga aaaagccgtt 9960
tctgtaatga aggagaaaac tcaccgaggc agttccatag gatggcaaga tcctggtatc 10020
ggtctgcgat tccgactcgt ccaacatcaa tacaacctat taatttcccc tcgtcaaaaa 10080
taaggttatc aagtgagaaa tcaccatgag tgacgactga atccggtgag aatggcaaca 10140
gcttatgcat ttctttccag acttgttcaa caggccagcc attacgctcg tcatcaaaat 10200
cactcgcatc aaccaaaccg ttattcattc gtgattgcgc ctgagcgaga cgaaatacgc 10260
gatcgctgtt aaaaggacaa ttacaaacag gaatcgaatg caaccggcgc aggaacactg 10320
ccagcgcatc aacaatattt tcacctgaat caggatattc ttctaatacc tggaatgctg 10380
tttttccggg gatcgcagtg gtgagtaacc atgcatcatc aggagtacgg ataaaatgct 10440
tgatggtcgg aagaggcata aattccgtca gccagtttag tctgaccatc tcatctgtaa 10500
catcattggc aacgctacct ttgccatgtt tcagaaacaa ctctggcgca tcgggcttcc 10560
catacaatcg atagattgtc gcacctgatt gcccgacatt atcgcgagcc catttatacc 10620
catataaatc agcatccatg ttggaattta atcgcggcct agagcaagac gtttcccgtt 10680
gaatatggct cataacaccc cttgtattac tgtttatgta agcagacagt tttattgttc 10740
atgatgatat atttttatct tgtgcaatgt aacatcagag attttgagac acaacaattg 10800
gtcgacggat cc 10812
<210> SEQ ID NO: 42
<211> 10519
<223> pGM413
ggtacctcaa tattggccat tagccatatt attcattggt tatatagcat aaatcaatat 60
tggctattgg ccattgcata cgttgtatct atatcataat atgtacattt atattggctc 120
atgtccaata tgaccgccat gttggcattg attattgact agttattaat agtaatcaat 180
tacggggtca ttagttcata gcccatatat ggagttccgc gttacataac ttacggtaaa 240
tggcccgcct ggctgaccgc ccaacgaccc ccgcccattg acgtcaataa tgacgtatgt 300
tcccatagta acgccaatag ggactttcca ttgacgtcaa tgggtggagt atttacggta 360
aactgcccac ttggcagtac atcaagtgta tcatatgcca agtccgcccc ctattgacgt 420
caatgacggt aaatggcccg cctggcatta tgcccagtac atgaccttac gggactttcc 480
tacttggcag tacatctacg tattagtcat cgctattacc atggtgatgc ggttttggca 540
gtacaccaat gggcgtggat agcggtttga ctcacgggga tttccaagtc tccaccccat 600
tgacgtcaat gggagtttgt tttggcacca aaatcaacgg gactttccaa aatgtcgtaa 660
caactgcgat cgcccgcccc gttgacgcaa atgggcggta ggcgtgtacg gtgggaggtc 720
tatataagca gagctcgctg gcttgtaact cagtctctta ctaggagacc agcttgagcc 780
tgggtgttcg ctggttagcc taacctggtt ggccaccagg ggtaaggact ccttggctta 840
gaaagctaat aaacttgcct gcattagagc ttatctgagt caagtgtcct cattgacgcc 900
tcactctctt gaacgggaat cttccttact gggttctctc tctgacccag gcgagagaaa 960
ctccagcagt ggcgcccgaa cagggacttg agtgagagtg taggcacgta cagctgagaa 1020
ggcgtcggac gcgaaggaag cgcggggtgc gacgcgacca agaaggagac ttggtgagta 1080
ggcttctcga gtgccgggaa aaagctcgag cctagttaga ggactaggag aggccgtagc 1140
cgtaactact ctgggcaagt agggcaggcg gtgggtacgc aatgggggcg gctacctcag 1200
cactaaatag gagacaatta gaccaatttg agaaaatacg acttcgcccg aacggaaaga 1260
aaaagtacca aattaaacat ttaatatggg caggcaagga gatggagcgc ttcggcctcc 1320
atgagaggtt gttggagaca gaggaggggt gtaaaagaat catagaagtc ctctaccccc 1380
tagaaccaac aggatcggag ggcttaaaaa gtctgttcaa tcttgtgtgc gtgctatatt 1440
gcttgcacaa ggaacagaaa gtgaaagaca cagaggaagc agtagcaaca gtaagacaac 1500
actgccatct agtggaaaaa gaaaaaagtg caacagagac atctagtgga caaaagaaaa 1560
atgacaaggg aatagcagcg ccacctggtg gcagtcagaa ttttccagcg caacaacaag 1620
gaaatgcctg ggtacatgta cccttgtcac cgcgcacctt aaatgcgtgg gtaaaagcag 1680
tagaggagaa aaaatttgga gcagaaatag tacccatgtt tcaagcccta tcgaattccc 1740
gtttgtgcta gggttcttag gcttcttggg ggctgctgga actgcaatgg gagcagcggc 1800
gacagccctg acggtccagt ctcagcattt gcttgctggg atactgcagc agcagaagaa 1860
tctgctggcg gctgtggagg ctcaacagca gatgttgaag ctgaccattt ggggtgttaa 1920
aaacctcaat gcccgcgtca cagcccttga gaagtaccta gaggatcagg cacgactaaa 1980
ctcctggggg tgcgcatgga aacaagtatg tcataccaca gtggagtggc cctggacaaa 2040
tcggactccg gattggcaaa atatgacttg gttggagtgg gaaagacaaa tagctgattt 2100
ggaaagcaac attacgagac aattagtgaa ggctagagaa caagaggaaa agaatctaga 2160
tgcctatcag aagttaacta gttggtcaga tttctggtct tggttcgatt tctcaaaatg 2220
gcttaacatt ttaaaaatgg gatttttagt aatagtagga ataatagggt taagattact 2280
ttacacagta tatggatgta tagtgagggt taggcaggga tatgttcctc tatctccaca 2340
gatccatatc cgcggcaatt ttaaaagaaa gggaggaata gggggacaga cttcagcaga 2400
gagactaatt aatataataa caacacaatt agaaatacaa catttacaaa ccaaaattca 2460
aaaaatttta aattttagag ccgcggagat ctgttacata acttatggta aatggcctgc 2520
ctggctgact gcccaatgac ccctgcccaa tgatgtcaat aatgatgtat gttcccatgt 2580
aatgccaata gggactttcc attgatgtca atgggtggag tatttatggt aactgcccac 2640
ttggcagtac atcaagtgta tcatatgcca agtatgcccc ctattgatgt caatgatggt 2700
aaatggcctg cctggcatta tgcccagtac atgaccttat gggactttcc tacttggcag 2760
tacatctatg tattagtcat tgctattacc atgggaattc actagtggag aagagcatgc 2820
ttgagggctg agtgcccctc agtgggcaga gagcacatgg cccacagtcc ctgagaagtt 2880
ggggggaggg gtgggcaatt gaactggtgc ctagagaagg tggggcttgg gtaaactggg 2940
aaagtgatgt ggtgtactgg ctccaccttt ttccccaggg tgggggagaa ccatatataa 3000
gtgcagtagt ctctgtgaac attcaagctt ctgccttctc cctcctgtga gtttgctagc 3060
caccaatgca gattgagctg agcacctgct tcttcctgtg cctgctgagg ttctgcttct 3120
ctgccaccag gagatactac ctgggggctg tggagctgag ctgggactac atgcagtctg 3180
acctggggga gctgcctgtg gatgccaggt tcccccccag agtgcccaag agcttcccct 3240
tcaacacctc tgtggtgtac aagaagaccc tgtttgtgga gttcactgac cacctgttca 3300
acattgccaa gcccaggccc ccctggatgg gcctgctggg ccccaccatc caggctgagg 3360
tgtatgacac tgtggtgatc accctgaaga acatggccag ccaccctgtg agcctgcatg 3420
ctgtgggggt gagctactgg aaggcctctg agggggctga gtatgatgac cagaccagcc 3480
agagggagaa ggaggatgac aaggtgttcc ctgggggcag ccacacctat gtgtggcagg 3540
tgctgaagga gaatggcccc atggcctctg accccctgtg cctgacctac agctacctga 3600
gccatgtgga cctggtgaag gacctgaact ctggcctgat tggggccctg ctggtgtgca 3660
gggagggcag cctggccaag gagaagaccc agaccctgca caagttcatc ctgctgtttg 3720
ctgtgtttga tgagggcaag agctggcact ctgaaaccaa gaacagcctg atgcaggaca 3780
gggatgctgc ctctgccagg gcctggccca agatgcacac tgtgaatggc tatgtgaaca 3840
ggagcctgcc tggcctgatt ggctgccaca ggaagtctgt gtactggcat gtgattggca 3900
tgggcaccac ccctgaggtg cacagcatct tcctggaggg ccacaccttc ctggtcagga 3960
accacaggca ggccagcctg gagatcagcc ccatcacctt cctgactgcc cagaccctgc 4020
tgatggacct gggccagttc ctgctgttct gccacatcag cagccaccag catgatggca 4080
tggaggccta tgtgaaggtg gacagctgcc ctgaggagcc ccagctgagg atgaagaaca 4140
atgaggaggc tgaggactat gatgatgacc tgactgactc tgagatggat gtggtgaggt 4200
ttgatgatga caacagcccc agcttcatcc agatcaggtc tgtggccaag aagcacccca 4260
agacctgggt gcactacatt gctgctgagg aggaggactg ggactatgcc cccctggtgc 4320
tggcccctga tgacaggagc tacaagagcc agtacctgaa caatggcccc cagaggattg 4380
gcaggaagta caagaaggtc aggttcatgg cctacactga tgaaaccttc aagaccaggg 4440
aggccatcca gcatgagtct ggcatcctgg gccccctgct gtatggggag gtgggggaca 4500
ccctgctgat catcttcaag aaccaggcca gcaggcccta caacatctac ccccatggca 4560
tcactgatgt gaggcccctg tacagcagga ggctgcccaa gggggtgaag cacctgaagg 4620
acttccccat cctgcctggg gagatcttca agtacaagtg gactgtgact gtggaggatg 4680
gccccaccaa gtctgacccc aggtgcctga ccagatacta cagcagcttt gtgaacatgg 4740
agagggacct ggcctctggc ctgattggcc ccctgctgat ctgctacaag gagtctgtgg 4800
accagagggg caaccagatc atgtctgaca agaggaatgt gatcctgttc tctgtgtttg 4860
atgagaacag gagctggtac ctgactgaga acatccagag gttcctgccc aaccctgctg 4920
gggtgcagct ggaggaccct gagttccagg ccagcaacat catgcacagc atcaatggct 4980
atgtgtttga cagcctgcag ctgtctgtgt gcctgcatga ggtggcctac tggtacatcc 5040
tgagcattgg ggcccagact gacttcctgt ctgtgttctt ctctggctac accttcaagc 5100
acaagatggt gtatgaggac accctgaccc tgttcccctt ctctggggag actgtgttca 5160
tgagcatgga gaaccctggc ctgtggattc tgggctgcca caactctgac ttcaggaaca 5220
ggggcatgac tgccctgctg aaagtctcca gctgtgacaa gaacactggg gactactatg 5280
aggacagcta tgaggacatc tctgcctacc tgctgagcaa gaacaatgcc attgagccca 5340
ggagcttcag ccagaatgcc actaatgtgt ctaacaacag caacaccagc aatgacagca 5400
atgtgtctcc cccagtgctg aagaggcacc agagggagat caccaggacc accctgcagt 5460
ctgaccagga ggagattgac tatgatgaca ccatctctgt ggagatgaag aaggaggact 5520
ttgacatcta cgacgaggac gagaaccaga gccccaggag cttccagaag aagaccaggc 5580
actacttcat tgctgctgtg gagaggctgt gggactatgg catgagcagc agcccccatg 5640
tgctgaggaa cagggcccag tctggctctg tgccccagtt caagaaggtg gtgttccagg 5700
agttcactga tggcagcttc acccagcccc tgtacagagg ggagctgaat gagcacctgg 5760
gcctgctggg cccctacatc agggctgagg tggaggacaa catcatggtg accttcagga 5820
accaggccag caggccctac agcttctaca gcagcctgat cagctatgag gaggaccaga 5880
ggcagggggc tgagcccagg aagaactttg tgaagcccaa tgaaaccaag acctacttct 5940
ggaaggtgca gcaccacatg gcccccacca aggatgagtt tgactgcaag gcctgggcct 6000
acttctctga tgtggacctg gagaaggatg tgcactctgg cctgattggc cccctgctgg 6060
tgtgccacac caacaccctg aaccctgccc atggcaggca ggtgactgtg caggagtttg 6120
ccctgttctt caccatcttt gatgaaacca agagctggta cttcactgag aacatggaga 6180
ggaactgcag ggccccctgc aacatccaga tggaggaccc caccttcaag gagaactaca 6240
ggttccatgc catcaatggc tacatcatgg acaccctgcc tggcctggtg atggcccagg 6300
accagaggat caggtggtac ctgctgagca tgggcagcaa tgagaacatc cacagcatcc 6360
acttctctgg ccatgtgttc actgtgagga agaaggagga gtacaagatg gccctgtaca 6420
acctgtaccc tggggtgttt gagactgtgg agatgctgcc cagcaaggct ggcatctgga 6480
gggtggagtg cctgattggg gagcacctgc atgctggcat gagcaccctg ttcctggtgt 6540
acagcaacaa gtgccagacc cccctgggca tggcctctgg ccacatcagg gacttccaga 6600
tcactgcctc tggccagtat ggccagtggg cccccaagct ggccaggctg cactactctg 6660
gcagcatcaa tgcctggagc accaaggagc ccttcagctg gatcaaggtg gacctgctgg 6720
cccccatgat catccatggc atcaagaccc agggggccag gcagaagttc agcagcctgt 6780
acatcagcca gttcatcatc atgtacagcc tggatggcaa gaagtggcag acctacaggg 6840
gcaacagcac tggcaccctg atggtgttct ttggcaatgt ggacagctct ggcatcaagc 6900
acaacatctt caaccccccc atcattgcca gatacatcag gctgcacccc acccactaca 6960
gcatcaggag caccctgagg atggagctga tgggctgtga cctgaacagc tgcagcatgc 7020
ccctgggcat ggagagcaag gccatctctg atgcccagat cactgccagc agctacttca 7080
ccaacatgtt tgccacctgg agccccagca aggccaggct gcacctgcag ggcaggagca 7140
atgcctggag gccccaggtc aacaacccca aggagtggct gcaggtggac ttccagaaga 7200
ccatgaaggt gactggggtg accacccagg gggtgaagag cctgctgacc agcatgtatg 7260
tgaaggagtt cctgatcagc agcagccagg atggccacca gtggaccctg ttcttccaga 7320
atggcaaggt gaaggtgttc cagggcaacc aggacagctt cacccctgtg gtgaacagcc 7380
tggacccccc cctgctgacc agatacctga ggattcaccc ccagagctgg gtgcaccaga 7440
ttgccctgag gatggaggtg ctgggctgtg aggcccagga cctgtactga gcggccgcgg 7500
gcccaatcaa cctctggatt acaaaatttg tgaaagattg actggtattc ttaactatgt 7560
tgctcctttt acgctatgtg gatacgctgc tttaatgcct ttgtatcatg ctattgcttc 7620
ccgtatggct ttcattttct cctccttgta taaatcctgg ttgctgtctc tttatgagga 7680
gttgtggccc gttgtcaggc aacgtggcgt ggtgtgcact gtgtttgctg acgcaacccc 7740
cactggttgg ggcattgcca ccacctgtca gctcctttcc gggactttcg ctttccccct 7800
ccctattgcc acggcggaac tcatcgccgc ctgccttgcc cgctgctgga caggggctcg 7860
gctgttgggc actgacaatt ccgtggtgtt gtcggggaaa tcatcgtcct ttccttggct 7920
gctcgcctgt gttgccacct ggattctgcg cgggacgtcc ttctgctacg tcccttcggc 7980
cctcaatcca gcggaccttc cttcccgcgg cctgctgccg gctctgcggc ctcttccgcg 8040
tcttcgcctt cgccctcaga cgagtcggat ctccctttgg gccgcctccc cgcaagcttc 8100
gcacttttta aaagaaaagg gaggactgga tgggatttat tactccgata ggacgctggc 8160
ttgtaactca gtctcttact aggagaccag cttgagcctg ggtgttcgct ggttagccta 8220
acctggttgg ccaccagggg taaggactcc ttggcttaga aagctaataa acttgcctgc 8280
attagagctc ttacgcgtcc cgggctcgag atccgcatct caattagtca gcaaccatag 8340
tcccgcccct aactccgccc atcccgcccc taactccgcc cagttccgcc cattctccgc 8400
cccatggctg actaattttt tttatttatg cagaggccga ggccgcctcg gcctctgagc 8460
tattccagaa gtagtgagga ggcttttttg gaggcctagg cttttgcaaa aagctaactt 8520
gtttattgca gcttataatg gttacaaata aagcaatagc atcacaaatt tcacaaataa 8580
agcatttttt tcactgcatt ctagttgtgg tttgtccaaa ctcatcaatg tatcttatca 8640
tgtctgtccg cttcctcgct cactgactcg ctgcgctcgg tcgttcggct gcggcgagcg 8700
gtatcagctc actcaaaggc ggtaatacgg ttatccacag aatcagggga taacgcagga 8760
aagaacatgt gagcaaaagg ccagcaaaag gccaggaacc gtaaaaaggc cgcgttgctg 8820
gcgtttttcc ataggctccg cccccctgac gagcatcaca aaaatcgacg ctcaagtcag 8880
aggtggcgaa acccgacagg actataaaga taccaggcgt ttccccctgg aagctccctc 8940
gtgcgctctc ctgttccgac cctgccgctt accggatacc tgtccgcctt tctcccttcg 9000
ggaagcgtgg cgctttctca tagctcacgc tgtaggtatc tcagttcggt gtaggtcgtt 9060
cgctccaagc tgggctgtgt gcacgaaccc cccgttcagc ccgaccgctg cgccttatcc 9120
ggtaactatc gtcttgagtc caacccggta agacacgact tatcgccact ggcagcagcc 9180
actggtaaca ggattagcag agcgaggtat gtaggcggtg ctacagagtt cttgaagtgg 9240
tggcctaact acggctacac tagaagaaca gtatttggta tctgcgctct gctgaagcca 9300
gttaccttcg gaaaaagagt tggtagctct tgatccggca aacaaaccac cgctggtagc 9360
ggtggttttt ttgtttgcaa gcagcagatt acgcgcagaa aaaaaggatc tcaagaagat 9420
cctttgatct tttctacggg gtctgacgct cagtggaacg aaaactcacg ttaagggatt 9480
ttggtcatga gattatcaaa aaggatcttc acctagatcc ttttaaatta aaaatgaagt 9540
tttaaatcaa tctaaagtat atatgagtaa acttggtctg acagttagaa aaactcatcg 9600
agcatcaaat gaaactgcaa tttattcata tcaggattat caataccata tttttgaaaa 9660
agccgtttct gtaatgaagg agaaaactca ccgaggcagt tccataggat ggcaagatcc 9720
tggtatcggt ctgcgattcc gactcgtcca acatcaatac aacctattaa tttcccctcg 9780
tcaaaaataa ggttatcaag tgagaaatca ccatgagtga cgactgaatc cggtgagaat 9840
ggcaacagct tatgcatttc tttccagact tgttcaacag gccagccatt acgctcgtca 9900
tcaaaatcac tcgcatcaac caaaccgtta ttcattcgtg attgcgcctg agcgagacga 9960
aatacgcgat cgctgttaaa aggacaatta caaacaggaa tcgaatgcaa ccggcgcagg 10020
aacactgcca gcgcatcaac aatattttca cctgaatcag gatattcttc taatacctgg 10080
aatgctgttt ttccggggat cgcagtggtg agtaaccatg catcatcagg agtacggata 10140
aaatgcttga tggtcggaag aggcataaat tccgtcagcc agtttagtct gaccatctca 10200
tctgtaacat cattggcaac gctacctttg ccatgtttca gaaacaactc tggcgcatcg 10260
ggcttcccat acaatcgata gattgtcgca cctgattgcc cgacattatc gcgagcccat 10320
ttatacccat ataaatcagc atccatgttg gaatttaatc gcggcctaga gcaagacgtt 10380
tcccgttgaa tatggctcat aacacccctt gtattactgt ttatgtaagc agacagtttt 10440
attgttcatg atgatatatt tttatcttgt gcaatgtaac atcagagatt ttgagacaca 10500
acaattggtc gacggatcc 10519
<210> SEQ ID NO: 43
<211> 11400
<223> pGM412
ggtacctcaa tattggccat tagccatatt attcattggt tatatagcat aaatcaatat 60
tggctattgg ccattgcata cgttgtatct atatcataat atgtacattt atattggctc 120
atgtccaata tgaccgccat gttggcattg attattgact agttattaat agtaatcaat 180
tacggggtca ttagttcata gcccatatat ggagttccgc gttacataac ttacggtaaa 240
tggcccgcct ggctgaccgc ccaacgaccc ccgcccattg acgtcaataa tgacgtatgt 300
tcccatagta acgccaatag ggactttcca ttgacgtcaa tgggtggagt atttacggta 360
aactgcccac ttggcagtac atcaagtgta tcatatgcca agtccgcccc ctattgacgt 420
caatgacggt aaatggcccg cctggcatta tgcccagtac atgaccttac gggactttcc 480
tacttggcag tacatctacg tattagtcat cgctattacc atggtgatgc ggttttggca 540
gtacaccaat gggcgtggat agcggtttga ctcacgggga tttccaagtc tccaccccat 600
tgacgtcaat gggagtttgt tttggcacca aaatcaacgg gactttccaa aatgtcgtaa 660
caactgcgat cgcccgcccc gttgacgcaa atgggcggta ggcgtgtacg gtgggaggtc 720
tatataagca gagctcgctg gcttgtaact cagtctctta ctaggagacc agcttgagcc 780
tgggtgttcg ctggttagcc taacctggtt ggccaccagg ggtaaggact ccttggctta 840
gaaagctaat aaacttgcct gcattagagc ttatctgagt caagtgtcct cattgacgcc 900
tcactctctt gaacgggaat cttccttact gggttctctc tctgacccag gcgagagaaa 960
ctccagcagt ggcgcccgaa cagggacttg agtgagagtg taggcacgta cagctgagaa 1020
ggcgtcggac gcgaaggaag cgcggggtgc gacgcgacca agaaggagac ttggtgagta 1080
ggcttctcga gtgccgggaa aaagctcgag cctagttaga ggactaggag aggccgtagc 1140
cgtaactact ctgggcaagt agggcaggcg gtgggtacgc aatgggggcg gctacctcag 1200
cactaaatag gagacaatta gaccaatttg agaaaatacg acttcgcccg aacggaaaga 1260
aaaagtacca aattaaacat ttaatatggg caggcaagga gatggagcgc ttcggcctcc 1320
atgagaggtt gttggagaca gaggaggggt gtaaaagaat catagaagtc ctctaccccc 1380
tagaaccaac aggatcggag ggcttaaaaa gtctgttcaa tcttgtgtgc gtgctatatt 1440
gcttgcacaa ggaacagaaa gtgaaagaca cagaggaagc agtagcaaca gtaagacaac 1500
actgccatct agtggaaaaa gaaaaaagtg caacagagac atctagtgga caaaagaaaa 1560
atgacaaggg aatagcagcg ccacctggtg gcagtcagaa ttttccagcg caacaacaag 1620
gaaatgcctg ggtacatgta cccttgtcac cgcgcacctt aaatgcgtgg gtaaaagcag 1680
tagaggagaa aaaatttgga gcagaaatag tacccatgtt tcaagcccta tcgaattccc 1740
gtttgtgcta gggttcttag gcttcttggg ggctgctgga actgcaatgg gagcagcggc 1800
gacagccctg acggtccagt ctcagcattt gcttgctggg atactgcagc agcagaagaa 1860
tctgctggcg gctgtggagg ctcaacagca gatgttgaag ctgaccattt ggggtgttaa 1920
aaacctcaat gcccgcgtca cagcccttga gaagtaccta gaggatcagg cacgactaaa 1980
ctcctggggg tgcgcatgga aacaagtatg tcataccaca gtggagtggc cctggacaaa 2040
tcggactccg gattggcaaa atatgacttg gttggagtgg gaaagacaaa tagctgattt 2100
ggaaagcaac attacgagac aattagtgaa ggctagagaa caagaggaaa agaatctaga 2160
tgcctatcag aagttaacta gttggtcaga tttctggtct tggttcgatt tctcaaaatg 2220
gcttaacatt ttaaaaatgg gatttttagt aatagtagga ataatagggt taagattact 2280
ttacacagta tatggatgta tagtgagggt taggcaggga tatgttcctc tatctccaca 2340
gatccatatc cgcggcaatt ttaaaagaaa gggaggaata gggggacaga cttcagcaga 2400
gagactaatt aatataataa caacacaatt agaaatacaa catttacaaa ccaaaattca 2460
aaaaatttta aattttagag ccgcggagat ctcaatattg gccattagcc atattattca 2520
ttggttatat agcataaatc aatattggct attggccatt gcatacgttg tatctatatc 2580
ataatatgta catttatatt ggctcatgtc caatatgacc gccatgttgg cattgattat 2640
tgactagtta ttaatagtaa tcaattacgg ggtcattagt tcatagccca tatatggagt 2700
tccgcgttac ataacttacg gtaaatggcc cgcctggctg accgcccaac gacccccgcc 2760
cattgacgtc aataatgacg tatgttccca tagtaacgcc aatagggact ttccattgac 2820
gtcaatgggt ggagtattta cggtaaactg cccacttggc agtacatcaa gtgtatcata 2880
tgccaagtcc gccccctatt gacgtcaatg acggtaaatg gcccgcctgg cattatgccc 2940
agtacatgac cttacgggac tttcctactt ggcagtacat ctacgtatta gtcatcgcta 3000
ttaccatggt gatgcggttt tggcagtaca ccaatgggcg tggatagcgg tttgactcac 3060
ggggatttcc aagtctccac cccattgacg tcaatgggag tttgttttgg caccaaaatc 3120
aacgggactt tccaaaatgt cgtaataacc ccgccccgtt gacgcaaatg ggcggtaggc 3180
gtgtacggtg ggaggtctat ataagcagag ctcgtttagt gaaccgtcag atcactagaa 3240
gctttattgc ggtagtttat cacagttaaa ttgctaacgc agtcagtgct tctgacacaa 3300
cagtctcgaa cttaagctgc agaagttggt cgtgaggcac tgggcaggct agccaccaat 3360
gcagattgag ctgagcacct gcttcttcct gtgcctgctg aggttctgct tctctgccac 3420
caggagatac tacctggggg ctgtggagct gagctgggac tacatgcagt ctgacctggg 3480
ggagctgcct gtggatgcca ggttcccccc cagagtgccc aagagcttcc ccttcaacac 3540
ctctgtggtg tacaagaaga ccctgtttgt ggagttcact gaccacctgt tcaacattgc 3600
caagcccagg cccccctgga tgggcctgct gggccccacc atccaggctg aggtgtatga 3660
cactgtggtg atcaccctga agaacatggc cagccaccct gtgagcctgc atgctgtggg 3720
ggtgagctac tggaaggcct ctgagggggc tgagtatgat gaccagacca gccagaggga 3780
gaaggaggat gacaaggtgt tccctggggg cagccacacc tatgtgtggc aggtgctgaa 3840
ggagaatggc cccatggcct ctgaccccct gtgcctgacc tacagctacc tgagccatgt 3900
ggacctggtg aaggacctga actctggcct gattggggcc ctgctggtgt gcagggaggg 3960
cagcctggcc aaggagaaga cccagaccct gcacaagttc atcctgctgt ttgctgtgtt 4020
tgatgagggc aagagctggc actctgaaac caagaacagc ctgatgcagg acagggatgc 4080
tgcctctgcc agggcctggc ccaagatgca cactgtgaat ggctatgtga acaggagcct 4140
gcctggcctg attggctgcc acaggaagtc tgtgtactgg catgtgattg gcatgggcac 4200
cacccctgag gtgcacagca tcttcctgga gggccacacc ttcctggtca ggaaccacag 4260
gcaggccagc ctggagatca gccccatcac cttcctgact gcccagaccc tgctgatgga 4320
cctgggccag ttcctgctgt tctgccacat cagcagccac cagcatgatg gcatggaggc 4380
ctatgtgaag gtggacagct gccctgagga gccccagctg aggatgaaga acaatgagga 4440
ggctgaggac tatgatgatg acctgactga ctctgagatg gatgtggtga ggtttgatga 4500
tgacaacagc cccagcttca tccagatcag gtctgtggcc aagaagcacc ccaagacctg 4560
ggtgcactac attgctgctg aggaggagga ctgggactat gcccccctgg tgctggcccc 4620
tgatgacagg agctacaaga gccagtacct gaacaatggc ccccagagga ttggcaggaa 4680
gtacaagaag gtcaggttca tggcctacac tgatgaaacc ttcaagacca gggaggccat 4740
ccagcatgag tctggcatcc tgggccccct gctgtatggg gaggtggggg acaccctgct 4800
gatcatcttc aagaaccagg ccagcaggcc ctacaacatc tacccccatg gcatcactga 4860
tgtgaggccc ctgtacagca ggaggctgcc caagggggtg aagcacctga aggacttccc 4920
catcctgcct ggggagatct tcaagtacaa gtggactgtg actgtggagg atggccccac 4980
caagtctgac cccaggtgcc tgaccagata ctacagcagc tttgtgaaca tggagaggga 5040
cctggcctct ggcctgattg gccccctgct gatctgctac aaggagtctg tggaccagag 5100
gggcaaccag atcatgtctg acaagaggaa tgtgatcctg ttctctgtgt ttgatgagaa 5160
caggagctgg tacctgactg agaacatcca gaggttcctg cccaaccctg ctggggtgca 5220
gctggaggac cctgagttcc aggccagcaa catcatgcac agcatcaatg gctatgtgtt 5280
tgacagcctg cagctgtctg tgtgcctgca tgaggtggcc tactggtaca tcctgagcat 5340
tggggcccag actgacttcc tgtctgtgtt cttctctggc tacaccttca agcacaagat 5400
ggtgtatgag gacaccctga ccctgttccc cttctctggg gagactgtgt tcatgagcat 5460
ggagaaccct ggcctgtgga ttctgggctg ccacaactct gacttcagga acaggggcat 5520
gactgccctg ctgaaagtct ccagctgtga caagaacact ggggactact atgaggacag 5580
ctatgaggac atctctgcct acctgctgag caagaacaat gccattgagc ccaggagctt 5640
cagccagaac agcaggcacc ccagcaccag gcagaagcag ttcaatgcca ccaccatccc 5700
tgagaatgac atagagaaga cagacccatg gtttgcccac cggaccccca tgcccaagat 5760
ccagaatgtg agcagctctg acctgctgat gctgctgagg cagagcccca ccccccatgg 5820
cctgagcctg tctgacctgc aggaggccaa gtatgaaacc ttctctgatg accccagccc 5880
tggggccatt gacagcaaca acagcctgtc tgagatgacc cacttcaggc cccagctgca 5940
ccactctggg gacatggtgt tcacccctga gtctggcctg cagctgaggc tgaatgagaa 6000
gctgggcacc actgctgcca ctgagctgaa gaagctggac ttcaaagtct ccagcaccag 6060
caacaacctg atcagcacca tcccctctga caacctggct gctggcactg acaacaccag 6120
cagcctgggc ccccccagca tgcctgtgca ctatgacagc cagctggaca ccaccctgtt 6180
tggcaagaag agcagccccc tgactgagtc tgggggcccc ctgagcctgt ctgaggagaa 6240
caatgacagc aagctgctgg agtctggcct gatgaacagc caggagagca gctggggcaa 6300
gaatgtgagc agcagggaga tcaccaggac caccctgcag tctgaccagg aggagattga 6360
ctatgatgac accatctctg tggagatgaa gaaggaggac tttgacatct acgacgagga 6420
cgagaaccag agccccagga gcttccagaa gaagaccagg cactacttca ttgctgctgt 6480
ggagaggctg tgggactatg gcatgagcag cagcccccat gtgctgagga acagggccca 6540
gtctggctct gtgccccagt tcaagaaggt ggtgttccag gagttcactg atggcagctt 6600
cacccagccc ctgtacagag gggagctgaa tgagcacctg ggcctgctgg gcccctacat 6660
cagggctgag gtggaggaca acatcatggt gaccttcagg aaccaggcca gcaggcccta 6720
cagcttctac agcagcctga tcagctatga ggaggaccag aggcaggggg ctgagcccag 6780
gaagaacttt gtgaagccca atgaaaccaa gacctacttc tggaaggtgc agcaccacat 6840
ggcccccacc aaggatgagt ttgactgcaa ggcctgggcc tacttctctg atgtggacct 6900
ggagaaggat gtgcactctg gcctgattgg ccccctgctg gtgtgccaca ccaacaccct 6960
gaaccctgcc catggcaggc aggtgactgt gcaggagttt gccctgttct tcaccatctt 7020
tgatgaaacc aagagctggt acttcactga gaacatggag aggaactgca gggccccctg 7080
caacatccag atggaggacc ccaccttcaa ggagaactac aggttccatg ccatcaatgg 7140
ctacatcatg gacaccctgc ctggcctggt gatggcccag gaccagagga tcaggtggta 7200
cctgctgagc atgggcagca atgagaacat ccacagcatc cacttctctg gccatgtgtt 7260
cactgtgagg aagaaggagg agtacaagat ggccctgtac aacctgtacc ctggggtgtt 7320
tgagactgtg gagatgctgc ccagcaaggc tggcatctgg agggtggagt gcctgattgg 7380
ggagcacctg catgctggca tgagcaccct gttcctggtg tacagcaaca agtgccagac 7440
ccccctgggc atggcctctg gccacatcag ggacttccag atcactgcct ctggccagta 7500
tggccagtgg gcccccaagc tggccaggct gcactactct ggcagcatca atgcctggag 7560
caccaaggag cccttcagct ggatcaaggt ggacctgctg gcccccatga tcatccatgg 7620
catcaagacc cagggggcca ggcagaagtt cagcagcctg tacatcagcc agttcatcat 7680
catgtacagc ctggatggca agaagtggca gacctacagg ggcaacagca ctggcaccct 7740
gatggtgttc tttggcaatg tggacagctc tggcatcaag cacaacatct tcaacccccc 7800
catcattgcc agatacatca ggctgcaccc cacccactac agcatcagga gcaccctgag 7860
gatggagctg atgggctgtg acctgaacag ctgcagcatg cccctgggca tggagagcaa 7920
ggccatctct gatgcccaga tcactgccag cagctacttc accaacatgt ttgccacctg 7980
gagccccagc aaggccaggc tgcacctgca gggcaggagc aatgcctgga ggccccaggt 8040
caacaacccc aaggagtggc tgcaggtgga cttccagaag accatgaagg tgactggggt 8100
gaccacccag ggggtgaaga gcctgctgac cagcatgtat gtgaaggagt tcctgatcag 8160
cagcagccag gatggccacc agtggaccct gttcttccag aatggcaagg tgaaggtgtt 8220
ccagggcaac caggacagct tcacccctgt ggtgaacagc ctggaccccc ccctgctgac 8280
cagatacctg aggattcacc cccagagctg ggtgcaccag attgccctga ggatggaggt 8340
gctgggctgt gaggcccagg acctgtactg agcggccgcg ggcccaatca acctctggat 8400
tacaaaattt gtgaaagatt gactggtatt cttaactatg ttgctccttt tacgctatgt 8460
ggatacgctg ctttaatgcc tttgtatcat gctattgctt cccgtatggc tttcattttc 8520
tcctccttgt ataaatcctg gttgctgtct ctttatgagg agttgtggcc cgttgtcagg 8580
caacgtggcg tggtgtgcac tgtgtttgct gacgcaaccc ccactggttg gggcattgcc 8640
accacctgtc agctcctttc cgggactttc gctttccccc tccctattgc cacggcggaa 8700
ctcatcgccg cctgccttgc ccgctgctgg acaggggctc ggctgttggg cactgacaat 8760
tccgtggtgt tgtcggggaa atcatcgtcc tttccttggc tgctcgcctg tgttgccacc 8820
tggattctgc gcgggacgtc cttctgctac gtcccttcgg ccctcaatcc agcggacctt 8880
ccttcccgcg gcctgctgcc ggctctgcgg cctcttccgc gtcttcgcct tcgccctcag 8940
acgagtcgga tctccctttg ggccgcctcc ccgcaagctt cgcacttttt aaaagaaaag 9000
ggaggactgg atgggattta ttactccgat aggacgctgg cttgtaactc agtctcttac 9060
taggagacca gcttgagcct gggtgttcgc tggttagcct aacctggttg gccaccaggg 9120
gtaaggactc cttggcttag aaagctaata aacttgcctg cattagagct cttacgcgtc 9180
ccgggctcga gatccgcatc tcaattagtc agcaaccata gtcccgcccc taactccgcc 9240
catcccgccc ctaactccgc ccagttccgc ccattctccg ccccatggct gactaatttt 9300
ttttatttat gcagaggccg aggccgcctc ggcctctgag ctattccaga agtagtgagg 9360
aggctttttt ggaggcctag gcttttgcaa aaagctaact tgtttattgc agcttataat 9420
ggttacaaat aaagcaatag catcacaaat ttcacaaata aagcattttt ttcactgcat 9480
tctagttgtg gtttgtccaa actcatcaat gtatcttatc atgtctgtcc gcttcctcgc 9540
tcactgactc gctgcgctcg gtcgttcggc tgcggcgagc ggtatcagct cactcaaagg 9600
cggtaatacg gttatccaca gaatcagggg ataacgcagg aaagaacatg tgagcaaaag 9660
gccagcaaaa ggccaggaac cgtaaaaagg ccgcgttgct ggcgtttttc cataggctcc 9720
gcccccctga cgagcatcac aaaaatcgac gctcaagtca gaggtggcga aacccgacag 9780
gactataaag ataccaggcg tttccccctg gaagctccct cgtgcgctct cctgttccga 9840
ccctgccgct taccggatac ctgtccgcct ttctcccttc gggaagcgtg gcgctttctc 9900
atagctcacg ctgtaggtat ctcagttcgg tgtaggtcgt tcgctccaag ctgggctgtg 9960
tgcacgaacc ccccgttcag cccgaccgct gcgccttatc cggtaactat cgtcttgagt 10020
ccaacccggt aagacacgac ttatcgccac tggcagcagc cactggtaac aggattagca 10080
gagcgaggta tgtaggcggt gctacagagt tcttgaagtg gtggcctaac tacggctaca 10140
ctagaagaac agtatttggt atctgcgctc tgctgaagcc agttaccttc ggaaaaagag 10200
ttggtagctc ttgatccggc aaacaaacca ccgctggtag cggtggtttt tttgtttgca 10260
agcagcagat tacgcgcaga aaaaaaggat ctcaagaaga tcctttgatc ttttctacgg 10320
ggtctgacgc tcagtggaac gaaaactcac gttaagggat tttggtcatg agattatcaa 10380
aaaggatctt cacctagatc cttttaaatt aaaaatgaag ttttaaatca atctaaagta 10440
tatatgagta aacttggtct gacagttaga aaaactcatc gagcatcaaa tgaaactgca 10500
atttattcat atcaggatta tcaataccat atttttgaaa aagccgtttc tgtaatgaag 10560
gagaaaactc accgaggcag ttccatagga tggcaagatc ctggtatcgg tctgcgattc 10620
cgactcgtcc aacatcaata caacctatta atttcccctc gtcaaaaata aggttatcaa 10680
gtgagaaatc accatgagtg acgactgaat ccggtgagaa tggcaacagc ttatgcattt 10740
ctttccagac ttgttcaaca ggccagccat tacgctcgtc atcaaaatca ctcgcatcaa 10800
ccaaaccgtt attcattcgt gattgcgcct gagcgagacg aaatacgcga tcgctgttaa 10860
aaggacaatt acaaacagga atcgaatgca accggcgcag gaacactgcc agcgcatcaa 10920
caatattttc acctgaatca ggatattctt ctaatacctg gaatgctgtt tttccgggga 10980
tcgcagtggt gagtaaccat gcatcatcag gagtacggat aaaatgcttg atggtcggaa 11040
gaggcataaa ttccgtcagc cagtttagtc tgaccatctc atctgtaaca tcattggcaa 11100
cgctaccttt gccatgtttc agaaacaact ctggcgcatc gggcttccca tacaatcgat 11160
agattgtcgc acctgattgc ccgacattat cgcgagccca tttataccca tataaatcag 11220
catccatgtt ggaatttaat cgcggcctag agcaagacgt ttcccgttga atatggctca 11280
taacacccct tgtattactg tttatgtaag cagacagttt tattgttcat gatgatatat 11340
ttttatcttg tgcaatgtaa catcagagat tttgagacac aacaattggt cgacggatcc 11400
<210> SEQ ID NO: 44
<211> 11108
<223> pGM414
ggtacctcaa tattggccat tagccatatt attcattggt tatatagcat aaatcaatat 60
tggctattgg ccattgcata cgttgtatct atatcataat atgtacattt atattggctc 120
atgtccaata tgaccgccat gttggcattg attattgact agttattaat agtaatcaat 180
tacggggtca ttagttcata gcccatatat ggagttccgc gttacataac ttacggtaaa 240
tggcccgcct ggctgaccgc ccaacgaccc ccgcccattg acgtcaataa tgacgtatgt 300
tcccatagta acgccaatag ggactttcca ttgacgtcaa tgggtggagt atttacggta 360
aactgcccac ttggcagtac atcaagtgta tcatatgcca agtccgcccc ctattgacgt 420
caatgacggt aaatggcccg cctggcatta tgcccagtac atgaccttac gggactttcc 480
tacttggcag tacatctacg tattagtcat cgctattacc atggtgatgc ggttttggca 540
gtacaccaat gggcgtggat agcggtttga ctcacgggga tttccaagtc tccaccccat 600
tgacgtcaat gggagtttgt tttggcacca aaatcaacgg gactttccaa aatgtcgtaa 660
caactgcgat cgcccgcccc gttgacgcaa atgggcggta ggcgtgtacg gtgggaggtc 720
tatataagca gagctcgctg gcttgtaact cagtctctta ctaggagacc agcttgagcc 780
tgggtgttcg ctggttagcc taacctggtt ggccaccagg ggtaaggact ccttggctta 840
gaaagctaat aaacttgcct gcattagagc ttatctgagt caagtgtcct cattgacgcc 900
tcactctctt gaacgggaat cttccttact gggttctctc tctgacccag gcgagagaaa 960
ctccagcagt ggcgcccgaa cagggacttg agtgagagtg taggcacgta cagctgagaa 1020
ggcgtcggac gcgaaggaag cgcggggtgc gacgcgacca agaaggagac ttggtgagta 1080
ggcttctcga gtgccgggaa aaagctcgag cctagttaga ggactaggag aggccgtagc 1140
cgtaactact cttgggcaag tagggcaggc ggtgggtacg caatgggggc ggctacctca 1200
gcactaaata ggagacaatt agaccaattt gagaaaatac gacttcgccc gaacggaaag 1260
aaaaagtacc aaattaaaca tttaatatgg gcaggcaagg agatggagcg cttcggcctc 1320
catgagaggt tgttggagac agaggagggg tgtaaaagaa tcatagaagt cctctacccc 1380
ctagaaccaa caggatcgga gggcttaaaa agtctgttca atcttgtgtg cgtgctatat 1440
tgcttgcaca aggaacagaa agtgaaagac acagaggaag cagtagcaac agtaagacaa 1500
cactgccatc tagtggaaaa agaaaaaagt gcaacagaga catctagtgg acaaaagaaa 1560
aatgacaagg gaatagcagc gccacctggt ggcagtcaga attttccagc gcaacaacaa 1620
ggaaatgcct gggtacatgt acccttgtca ccgcgcacct taaatgcgtg ggtaaaagca 1680
gtagaggaga aaaaatttgg agcagaaata gtacccatgt ttcaagccct atcgaattcc 1740
cgtttgtgct agggttctta ggcttcttgg gggctgctgg aactgcaatg ggagcagcgg 1800
cgacagccct gacggtccag tctcagcatt tgcttgctgg gatactgcag cagcagaaga 1860
atctgctggc ggctgtggag gctcaacagc agatgttgaa gctgaccatt tggggtgtta 1920
aaaacctcaa tgcccgcgtc acagcccttg agaagtacct agaggatcag gcacgactaa 1980
actcctgggg gtgcgcatgg aaacaagtat gtcataccac agtggagtgg ccctggacaa 2040
atcggactcc ggattggcaa aatatgactt ggttggagtg ggaaagacaa atagctgatt 2100
tggaaagcaa cattacgaga caattagtga aggctagaga acaagaggaa aagaatctag 2160
atgcctatca gaagttaact agttggtcag atttctggtc ttggttcgat ttctcaaaat 2220
ggcttaacat tttaaaaatg ggatttttag taatagtagg aataataggg ttaagattac 2280
tttacacagt atatggatgt atagtgaggg ttaggcaggg atatgttcct ctatctccac 2340
agatccatat ccgcggcaat tttaaaagaa agggaggaat agggggacag acttcagcag 2400
agagactaat taatataata acaacacaat tagaaataca acatttacaa accaaaattc 2460
aaaaaatttt aaattttaga gccgcggaga tctgttacat aacttatggt aaatggcctg 2520
cctggctgac tgcccaatga cccctgccca atgatgtcaa taatgatgta tgttcccatg 2580
taatgccaat agggactttc cattgatgtc aatgggtgga gtatttatgg taactgccca 2640
cttggcagta catcaagtgt atcatatgcc aagtatgccc cctattgatg tcaatgatgg 2700
taaatggcct gcctggcatt atgcccagta catgacctta tgggactttc ctacttggca 2760
gtacatctat gtattagtca ttgctattac catgggaatt cactagtgga gaagagcatg 2820
cttgagggct gagtgcccct cagtgggcag agagcacatg gcccacagtc cctgagaagt 2880
tggggggagg ggtgggcaat tgaactggtg cctagagaag gtggggcttg ggtaaactgg 2940
gaaagtgatg tggtgtactg gctccacctt tttccccagg gtgggggaga accatatata 3000
agtgcagtag tctctgtgaa cattcaagct tctgccttct ccctcctgtg agtttgctag 3060
ccaccaatgc agattgagct gagcacctgc ttcttcctgt gcctgctgag gttctgcttc 3120
tctgccacca ggagatacta cctgggggct gtggagctga gctgggacta catgcagtct 3180
gacctggggg agctgcctgt ggatgccagg ttccccccca gagtgcccaa gagcttcccc 3240
ttcaacacct ctgtggtgta caagaagacc ctgtttgtgg agttcactga ccacctgttc 3300
aacattgcca agcccaggcc cccctggatg ggcctgctgg gccccaccat ccaggctgag 3360
gtgtatgaca ctgtggtgat caccctgaag aacatggcca gccaccctgt gagcctgcat 3420
gctgtggggg tgagctactg gaaggcctct gagggggctg agtatgatga ccagaccagc 3480
cagagggaga aggaggatga caaggtgttc cctgggggca gccacaccta tgtgtggcag 3540
gtgctgaagg agaatggccc catggcctct gaccccctgt gcctgaccta cagctacctg 3600
agccatgtgg acctggtgaa ggacctgaac tctggcctga ttggggccct gctggtgtgc 3660
agggagggca gcctggccaa ggagaagacc cagaccctgc acaagttcat cctgctgttt 3720
gctgtgtttg atgagggcaa gagctggcac tctgaaacca agaacagcct gatgcaggac 3780
agggatgctg cctctgccag ggcctggccc aagatgcaca ctgtgaatgg ctatgtgaac 3840
aggagcctgc ctggcctgat tggctgccac aggaagtctg tgtactggca tgtgattggc 3900
atgggcacca cccctgaggt gcacagcatc ttcctggagg gccacacctt cctggtcagg 3960
aaccacaggc aggccagcct ggagatcagc cccatcacct tcctgactgc ccagaccctg 4020
ctgatggacc tgggccagtt cctgctgttc tgccacatca gcagccacca gcatgatggc 4080
atggaggcct atgtgaaggt ggacagctgc cctgaggagc cccagctgag gatgaagaac 4140
aatgaggagg ctgaggacta tgatgatgac ctgactgact ctgagatgga tgtggtgagg 4200
tttgatgatg acaacagccc cagcttcatc cagatcaggt ctgtggccaa gaagcacccc 4260
aagacctggg tgcactacat tgctgctgag gaggaggact gggactatgc ccccctggtg 4320
ctggcccctg atgacaggag ctacaagagc cagtacctga acaatggccc ccagaggatt 4380
ggcaggaagt acaagaaggt caggttcatg gcctacactg atgaaacctt caagaccagg 4440
gaggccatcc agcatgagtc tggcatcctg ggccccctgc tgtatgggga ggtgggggac 4500
accctgctga tcatcttcaa gaaccaggcc agcaggccct acaacatcta cccccatggc 4560
atcactgatg tgaggcccct gtacagcagg aggctgccca agggggtgaa gcacctgaag 4620
gacttcccca tcctgcctgg ggagatcttc aagtacaagt ggactgtgac tgtggaggat 4680
ggccccacca agtctgaccc caggtgcctg accagatact acagcagctt tgtgaacatg 4740
gagagggacc tggcctctgg cctgattggc cccctgctga tctgctacaa ggagtctgtg 4800
gaccagaggg gcaaccagat catgtctgac aagaggaatg tgatcctgtt ctctgtgttt 4860
gatgagaaca ggagctggta cctgactgag aacatccaga ggttcctgcc caaccctgct 4920
ggggtgcagc tggaggaccc tgagttccag gccagcaaca tcatgcacag catcaatggc 4980
tatgtgtttg acagcctgca gctgtctgtg tgcctgcatg aggtggccta ctggtacatc 5040
ctgagcattg gggcccagac tgacttcctg tctgtgttct tctctggcta caccttcaag 5100
cacaagatgg tgtatgagga caccctgacc ctgttcccct tctctgggga gactgtgttc 5160
atgagcatgg agaaccctgg cctgtggatt ctgggctgcc acaactctga cttcaggaac 5220
aggggcatga ctgccctgct gaaagtctcc agctgtgaca agaacactgg ggactactat 5280
gaggacagct atgaggacat ctctgcctac ctgctgagca agaacaatgc cattgagccc 5340
aggagcttca gccagaacag caggcacccc agcaccaggc agaagcagtt caatgccacc 5400
accatccctg agaatgacat agagaagaca gacccatggt ttgcccaccg gacccccatg 5460
cccaagatcc agaatgtgag cagctctgac ctgctgatgc tgctgaggca gagccccacc 5520
ccccatggcc tgagcctgtc tgacctgcag gaggccaagt atgaaacctt ctctgatgac 5580
cccagccctg gggccattga cagcaacaac agcctgtctg agatgaccca cttcaggccc 5640
cagctgcacc actctgggga catggtgttc acccctgagt ctggcctgca gctgaggctg 5700
aatgagaagc tgggcaccac tgctgccact gagctgaaga agctggactt caaagtctcc 5760
agcaccagca acaacctgat cagcaccatc ccctctgaca acctggctgc tggcactgac 5820
aacaccagca gcctgggccc ccccagcatg cctgtgcact atgacagcca gctggacacc 5880
accctgtttg gcaagaagag cagccccctg actgagtctg ggggccccct gagcctgtct 5940
gaggagaaca atgacagcaa gctgctggag tctggcctga tgaacagcca ggagagcagc 6000
tggggcaaga atgtgagcag cagggagatc accaggacca ccctgcagtc tgaccaggag 6060
gagattgact atgatgacac catctctgtg gagatgaaga aggaggactt tgacatctac 6120
gacgaggacg agaaccagag ccccaggagc ttccagaaga agaccaggca ctacttcatt 6180
gctgctgtgg agaggctgtg ggactatggc atgagcagca gcccccatgt gctgaggaac 6240
agggcccagt ctggctctgt gccccagttc aagaaggtgg tgttccagga gttcactgat 6300
ggcagcttca cccagcccct gtacagaggg gagctgaatg agcacctggg cctgctgggc 6360
ccctacatca gggctgaggt ggaggacaac atcatggtga ccttcaggaa ccaggccagc 6420
aggccctaca gcttctacag cagcctgatc agctatgagg aggaccagag gcagggggct 6480
gagcccagga agaactttgt gaagcccaat gaaaccaaga cctacttctg gaaggtgcag 6540
caccacatgg cccccaccaa ggatgagttt gactgcaagg cctgggccta cttctctgat 6600
gtggacctgg agaaggatgt gcactctggc ctgattggcc ccctgctggt gtgccacacc 6660
aacaccctga accctgccca tggcaggcag gtgactgtgc aggagtttgc cctgttcttc 6720
accatctttg atgaaaccaa gagctggtac ttcactgaga acatggagag gaactgcagg 6780
gccccctgca acatccagat ggaggacccc accttcaagg agaactacag gttccatgcc 6840
atcaatggct acatcatgga caccctgcct ggcctggtga tggcccagga ccagaggatc 6900
aggtggtacc tgctgagcat gggcagcaat gagaacatcc acagcatcca cttctctggc 6960
catgtgttca ctgtgaggaa gaaggaggag tacaagatgg ccctgtacaa cctgtaccct 7020
ggggtgtttg agactgtgga gatgctgccc agcaaggctg gcatctggag ggtggagtgc 7080
ctgattgggg agcacctgca tgctggcatg agcaccctgt tcctggtgta cagcaacaag 7140
tgccagaccc ccctgggcat ggcctctggc cacatcaggg acttccagat cactgcctct 7200
ggccagtatg gccagtgggc ccccaagctg gccaggctgc actactctgg cagcatcaat 7260
gcctggagca ccaaggagcc cttcagctgg atcaaggtgg acctgctggc ccccatgatc 7320
atccatggca tcaagaccca gggggccagg cagaagttca gcagcctgta catcagccag 7380
ttcatcatca tgtacagcct ggatggcaag aagtggcaga cctacagggg caacagcact 7440
ggcaccctga tggtgttctt tggcaatgtg gacagctctg gcatcaagca caacatcttc 7500
aaccccccca tcattgccag atacatcagg ctgcacccca cccactacag catcaggagc 7560
accctgagga tggagctgat gggctgtgac ctgaacagct gcagcatgcc cctgggcatg 7620
gagagcaagg ccatctctga tgcccagatc actgccagca gctacttcac caacatgttt 7680
gccacctgga gccccagcaa ggccaggctg cacctgcagg gcaggagcaa tgcctggagg 7740
ccccaggtca acaaccccaa ggagtggctg caggtggact tccagaagac catgaaggtg 7800
actggggtga ccacccaggg ggtgaagagc ctgctgacca gcatgtatgt gaaggagttc 7860
ctgatcagca gcagccagga tggccaccag tggaccctgt tcttccagaa tggcaaggtg 7920
aaggtgttcc agggcaacca ggacagcttc acccctgtgg tgaacagcct ggaccccccc 7980
ctgctgacca gatacctgag gattcacccc cagagctggg tgcaccagat tgccctgagg 8040
atggaggtgc tgggctgtga ggcccaggac ctgtactgag cggccgcggg cccaatcaac 8100
ctctggatta caaaatttgt gaaagattga ctggtattct taactatgtt gctcctttta 8160
cgctatgtgg atacgctgct ttaatgcctt tgtatcatgc tattgcttcc cgtatggctt 8220
tcattttctc ctccttgtat aaatcctggt tgctgtctct ttatgaggag ttgtggcccg 8280
ttgtcaggca acgtggcgtg gtgtgcactg tgtttgctga cgcaaccccc actggttggg 8340
gcattgccac cacctgtcag ctcctttccg ggactttcgc tttccccctc cctattgcca 8400
cggcggaact catcgccgcc tgccttgccc gctgctggac aggggctcgg ctgttgggca 8460
ctgacaattc cgtggtgttg tcggggaaat catcgtcctt tccttggctg ctcgcctgtg 8520
ttgccacctg gattctgcgc gggacgtcct tctgctacgt cccttcggcc ctcaatccag 8580
cggaccttcc ttcccgcggc ctgctgccgg ctctgcggcc tcttccgcgt cttcgccttc 8640
gccctcagac gagtcggatc tccctttggg ccgcctcccc gcaagcttcg cactttttaa 8700
aagaaaaggg aggactggat gggatttatt actccgatag gacgctggct tgtaactcag 8760
tctcttacta ggagaccagc ttgagcctgg gtgttcgctg gttagcctaa cctggttggc 8820
caccaggggt aaggactcct tggcttagaa agctaataaa cttgcctgca ttagagctct 8880
tacgcgtccc gggctcgaga tccgcatctc aattagtcag caaccatagt cccgccccta 8940
actccgccca tcccgcccct aactccgccc agttccgccc attctccgcc ccatggctga 9000
ctaatttttt ttatttatgc agaggccgag gccgcctcgg cctctgagct attccagaag 9060
tagtgaggag gcttttttgg aggcctaggc ttttgcaaaa agctaacttg tttattgcag 9120
cttataatgg ttacaaataa agcaatagca tcacaaattt cacaaataaa gcattttttt 9180
cactgcattc tagttgtggt ttgtccaaac tcatcaatgt atcttatcat gtctgtccgc 9240
ttcctcgctc actgactcgc tgcgctcggt cgttcggctg cggcgagcgg tatcagctca 9300
ctcaaaggcg gtaatacggt tatccacaga atcaggggat aacgcaggaa agaacatgtg 9360
agcaaaaggc cagcaaaagg ccaggaaccg taaaaaggcc gcgttgctgg cgtttttcca 9420
taggctccgc ccccctgacg agcatcacaa aaatcgacgc tcaagtcaga ggtggcgaaa 9480
cccgacagga ctataaagat accaggcgtt tccccctgga agctccctcg tgcgctctcc 9540
tgttccgacc ctgccgctta ccggatacct gtccgccttt ctcccttcgg gaagcgtggc 9600
gctttctcat agctcacgct gtaggtatct cagttcggtg taggtcgttc gctccaagct 9660
gggctgtgtg cacgaacccc ccgttcagcc cgaccgctgc gccttatccg gtaactatcg 9720
tcttgagtcc aacccggtaa gacacgactt atcgccactg gcagcagcca ctggtaacag 9780
gattagcaga gcgaggtatg taggcggtgc tacagagttc ttgaagtggt ggcctaacta 9840
cggctacact agaagaacag tatttggtat ctgcgctctg ctgaagccag ttaccttcgg 9900
aaaaagagtt ggtagctctt gatccggcaa acaaaccacc gctggtagcg gtggtttttt 9960
tgtttgcaag cagcagatta cgcgcagaaa aaaaggatct caagaagatc ctttgatctt 10020
ttctacgggg tctgacgctc agtggaacga aaactcacgt taagggattt tggtcatgag 10080
attatcaaaa aggatcttca cctagatcct tttaaattaa aaatgaagtt ttaaatcaat 10140
ctaaagtata tatgagtaaa cttggtctga cagttagaaa aactcatcga gcatcaaatg 10200
aaactgcaat ttattcatat caggattatc aataccatat ttttgaaaaa gccgtttctg 10260
taatgaagga gaaaactcac cgaggcagtt ccataggatg gcaagatcct ggtatcggtc 10320
tgcgattccg actcgtccaa catcaataca acctattaat ttcccctcgt caaaaataag 10380
gttatcaagt gagaaatcac catgagtgac gactgaatcc ggtgagaatg gcaacagctt 10440
atgcatttct ttccagactt gttcaacagg ccagccatta cgctcgtcat caaaatcact 10500
cgcatcaacc aaaccgttat tcattcgtga ttgcgcctga gcgagacgaa atacgcgatc 10560
gctgttaaaa ggacaattac aaacaggaat cgaatgcaac cggcgcagga acactgccag 10620
cgcatcaaca atattttcac ctgaatcagg atattcttct aatacctgga atgctgtttt 10680
tccggggatc gcagtggtga gtaaccatgc atcatcagga gtacggataa aatgcttgat 10740
ggtcggaaga ggcataaatt ccgtcagcca gtttagtctg accatctcat ctgtaacatc 10800
attggcaacg ctacctttgc catgtttcag aaacaactct ggcgcatcgg gcttcccata 10860
caatcgatag attgtcgcac ctgattgccc gacattatcg cgagcccatt tatacccata 10920
taaatcagca tccatgttgg aatttaatcg cggcctagag caagacgttt cccgttgaat 10980
atggctcata acaccccttg tattactgtt tatgtaagca gacagtttta ttgttcatga 11040
tgatatattt ttatcttgtg caatgtaaca tcagagattt tgagacacaa caattggtcg 11100
acggatcc 11108
<210> SEQ ID NO: 45
<211> 1738
<223> CAG promoter
attgattatt gactagttat taatagtaat caattacggg gtcattagtt catagcccat 60
atatggagtt ccgcgttaca taacttacgg taaatggccc gcctggctga ccgcccaacg 120
acccccgccc attgacgtca ataatgacgt atgttcccat agtaacgcca atagggactt 180
tccattgacg tcaatgggtg gagtatttac ggtaaactgc ccacttggca gtacatcaag 240
tgtatcatat gccaagtacg ccccctattg acgtcaatga cggtaaatgg cccgcctggc 300
attatgccca gtacatgacc ttatgggact ttcctacttg gcagtacatc tacgtattag 360
tcatcgctat taccatggtc gaggtgagcc ccacgttctg cttcactctc cccatctccc 420
ccccctcccc acccccaatt ttgtatttat ttatttttta attattttgt gcagcgatgg 480
gggcgggggg gggggggggg cgcgcgccag gcggggcggg gcggggcgag gggcggggcg 540
gggcgaggcg gagaggtgcg gcggcagcca atcagagcgg cgcgctccga aagtttcctt 600
ttatggcgag gcggcggcgg cggcggccct ataaaaagcg aagcgcgcgg cgggcgggag 660
tcgctgcgcg ctgccttcgc cccgtgcccc gctccgccgc cgcctcgcgc cgcccgcccc 720
ggctctgact gaccgcgtta ctcccacagg tgagcgggcg ggacggccct tctcctccgg 780
gctgtaatta gcgcttggtt taatgacggc ttgtttcttt tctgtggctg cgtgaaagcc 840
ttgaggggct ccgggagggc cctttgtgcg gggggagcgg ctcggggggt gcgtgcgtgt 900
gtgtgtgcgt ggggagcgcc gcgtgcggct ccgcgctgcc cggcggctgt gagcgctgcg 960
ggcgcggcgc ggggctttgt gcgctccgca gtgtgcgcga ggggagcgcg gccgggggcg 1020
gtgccccgcg gtgcgggggg ggctgcgagg ggaacaaagg ctgcgtgcgg ggtgtgtgcg 1080
tgggggggtg agcagggggt gtgggcgcgt cggtcgggct gcaacccccc ctgcaccccc 1140
ctccccgagt tgctgagcac ggcccggctt cgggtgcggg gctccgtacg gggcgtggcg 1200
cggggctcgc cgtgccgggc ggggggtggc ggcaggtggg ggtgccgggc ggggcggggc 1260
cgcctcgggc cggggagggc tcgggggagg ggcgcggcgg cccccggagc gccggcggct 1320
gtcgaggcgc ggcgagccgc agccattgcc ttttatggta atcgtgcgag agggcgcagg 1380
gacttccttt gtcccaaatc tgtgcggagc cgaaatctgg gaggcgccgc cgcaccccct 1440
ctagcgggcg cggggcgaag cggtgcggcg ccggcaggaa ggaaatgggc ggggagggcc 1500
ttcgtgcgtc gccgcgccgc cgtccccttc tccctctcca gcctcggggc tgtccgcggg 1560
gggacggctg ccttcggggg ggacggggca gggcggggtt cggcttctgg cgtgtgaccg 1620
gcggctctag agcctctgct aaccatgttc atgccttctt ctttttccta cagctcctgg 1680
gcaacgtgct ggttattgtg ctgtctcatc attttggcaa agaattgctc gagccacc 1738
<210> SEQ ID NO: 46
<211> 1738
<223> Additional amino acid sequence encoded from false
transcription start site upstream of that encoding the
Fct4 of SEQ ID NO: 13
MFMPSSFSYSSWATCWLLCCLIILAKNSIA
```

1. A retroviral vector comprising a modified retroviral RNA sequence which is:
(i) codon-substitution; and
(ii) comprises a reduced number of retroviral open reading frames (ORFs) compared with a non-modified retroviral RNA sequence from which the modified retroviral RNA sequence is derived;
and wherein:
(a) the retroviral RNA sequence comprises a promoter and a transgene; and
(b) the retroviral vector is pseudotyped with hemagglutinin-neuraminidase (HN) and fusion (F) proteins from a respiratory paramyxovirus.

2. The retroviral vector of claim 1, wherein compared with the non-modified retroviral RNA sequence from which the modified retroviral RNA sequence is derived, the modified retroviral RNA sequence is lacking:
(a) one or more retroviral ORFs 5′ of the promoter:
(b) one or more retroviral ORF encoding a peptide of ≥100 amino acids in length;
(c) one or more retroviral ORF comprised in a partial RRE sequence; and/or
(d) one or more retroviral ORF encoded comprised in a partial Gag sequence.

3. The retroviral vector of claim 1, wherein the respiratory paramyxovirus is a Sendai virus.

4. The retroviral vector of claim 1, wherein the promoter is selected from the group consisting of a hybrid human CMV enhancer/EF1a (hCEF) promoter, a cytomegalovirus (CMV) promoter, and elongation factor 1a (EF1a) promoter.

5. The retroviral vector of claim 1, wherein the transgene is selected from:
a) CFTR, ABCA3, DNAH5, DNAH11, DNAI1, and DNAI2; or
b) a secreted therapeutic protein.

6. The retroviral vector of claim 1, wherein the transgene encodes:
a) CFTR;
b) A1AT; or
c) FVIII.

7. The retroviral vector of claim 1, wherein:
a) the promoter is a hCEF promoter and the transgene encodes CFTR;
b) the promoter is a hCEF promoter and the transgene encodes A1AT; or
c) the promoter is a hCEF or CMV promoter and the transgene encodes FVIII.

8. The retroviral vector of claim 1, which is a lentiviral vector.

9. The retroviral vector of claim 1, wherein the retroviral vector is an SIV vector and/or the F protein is an Fct4 protein.

10. The retroviral vector of claim 1, wherein the modified retroviral RNA sequence (i) is less than 9,000 bases in length and; (ii) comprises a nucleic acid sequence having at least 80% identity to SEQ ID NO: 1.

11. The retroviral vector of claim 10, wherein the modified retroviral RNA sequence comprises a nucleic acid sequence of SEQ ID NO: 1.

12. The retroviral vector of claim 1, wherein the vector further comprises one or more of:
(a) a p17 protein comprising an amino acid sequence having at least 80% sequence identity to SEQ ID NO: 2;
(b) a p24 protein comprising an amino acid sequence having at least 80% sequence identity to SEQ ID NO: 3;
(c) p8 protein comprising an amino acid sequence having at least 80% sequence identity to SEQ ID NO: 4;
(d) a protease comprising an amino acid sequence having at least 80% sequence identity to SEQ ID NO: 5;
(e) a p51 protein comprising an amino acid sequence having at least 80% sequence identity to SEQ ID NO: 6;
(f) a p15 protein comprising an amino acid sequence having at least 80% sequence identity to SEQ ID NO: 7; and
(g) a p31 protein comprising an amino acid sequence having at least 80% sequence identity to SEQ ID NO: 8.

13. The retroviral vector of claim 1, wherein the vector further comprises one or more of:
(a) a Gag protein comprising an amino acid sequence having at least 80% sequence identity to SEQ ID NO: 9; and/or
(b) a Pol protein comprising an amino acid sequence having at least 80% sequence identity to SEQ ID NO: 10.

14. (canceled)

15. A SIV vector pseudotyped with Sendai virus hemagglutinin-neuraminidase (HN) and fusion (F) proteins, wherein:
(a) said vector comprises a modified retroviral RNA sequence which comprises a nucleic acid sequence of SEQ ID NO: 1; and
(b) the F protein comprises a first subunit which comprises an amino acid sequence of SEQ ID NO: 14 and a second subunit which comprises an amino acid sequence of SEQ ID NO: 15.

16. The SIV vector of claim 15, wherein the vector further comprises one or more of:
(a) a p17 protein comprising an amino acid sequence of SEQ ID NO: 2;
(b) a p24 protein comprising an amino acid sequence of SEQ ID NO: 3;
(c) p8 protein comprising an amino acid sequence of SEQ ID NO: 4;
(d) a protease comprising an amino acid sequence of SEQ ID NO: 5;
(e) a p51 protein comprising an amino acid sequence of SEQ ID NO: 6;
(f) a p15 protein comprising an amino acid sequence of SEQ ID NO: 7;
(g) a p31 protein comprising an amino acid sequence of SEQ ID NO: 8;
(h) a Gag protein comprising an amino acid sequence of SEQ ID NO: 9; and/or
(i) a Pol protein comprising an amino acid sequence of SEQ ID NO: 10.

17. A method of producing a retroviral vector as defined in claim 1, said method comprising the following steps:
a) growing cells in suspension;
b) transfecting the cells with one or more plasmids;
c) adding a nuclease;
d) harvesting the lentivirus;
e) adding trypsin or an enzyme with the same cleavage specificity; and
f) purification.

18. (canceled)

19. (canceled)

20. The method of claim 17, wherein one or more of:
the addition of the nuclease is at the pre-harvest stage;
the addition of trypsin or enzyme with the same cleavage specificity is at the post-harvest stage;
the purification step comprises a chromatography step; and/or
the cells are HEK293T or 293T/17 cells.

21. (canceled)

22. (canceled)

23. A composition comprising a retroviral vector as defined in claim 1 and a pharmaceutically acceptable excipient or diluent, wherein the composition is formulated for administration to the lungs.

24. (canceled)

25. (canceled)

26. A method of treating a disease comprising administering a retroviral vector as defined in claim 1, to a subject in need thereof.

27. The method of treatment of claim 26, wherein the disease to be treated is a lung disease.


SEQUENCE LISTING (46 sequences)

SEQ ID NO: 1 - RNA, 7553 nt, synthetic construct
  misc_feature 1..7553: note = Modified SIV/CFTR RNA sequence
  source 1..7553: mol_type = other RNA
tctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactctgggcaagtagggcaggcggtgggtacgcaattgggggcggctacctcagcactaaataggagacaattagaccaatttgagaaaatacgacttcgcccgaacggaaagaaaaagtaccaaattaaacatttaatattgggcaggcaaggagattggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaattgcctgggtacatgtacccttgtcaccgcgcaccttaaatgcgtgggtaaaagcagtagaggagaaaaaatttggagcagaaatagtacccatgtttcaagccctatcgcctgcaggccgtttgtgctagggttcttaggcttcttgggggctgctggaactgcattgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaataagacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaaagggatttttagtaatagtaggaataatagggttaagattactttacacagtatatggatgtatagtgagggttaggcagggatatgttcctctatctccacagatccatataaagcggcaattttaaaagaaagggaggaatagggggacagacttcagcagagagactaattaatataataacaacacaattagaaatacaacatttacaaaccaaaattcaaaaaattttaaattttagagccgcggagatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagccaccatgcagagaagccctctggagaaggcctctgtggtgagcaagctgttcttcagctggaccaggcccatcctgaggaagggctacaggcagagactggagctgtctgacatctaccagatcccctctgtggactctgctgacaacctgtctgagaagctggagagggagtgggatagagagctggccagcaagaagaaccccaagctgatcaatgccctgaggagatgcttcttctggagattcatgttctatggcatcttcctgtacctgggggaagtgaccaaggctgtgcagcctctgctgctgggcagaatcattgccagctatgaccctgacaacaaggaggagaggagcattgccatctacctgggcattggcctgtgcctgctgttcattgtgaggaccctgctgctgcaccctgccatctttggcctgcaccacattggcatgcagatgaggattgccatgttcagcctgatctacaagaaaaccctgaagctgtccagcagagtgctggacaagatcagcattggccagctggtgagcctgctgagcaacaacctgaacaagtttgatgagggcctggccctggcccactttgtgtggattgcccctctgcaggtggccctgctgatgggcctgatttgggagctgctgcaggcctctgccttttgtggcctgggcttcctgattgtgctggccctgtttcaggctggcctgggcaggatgatgatgaagtacagggaccagagggcaggcaagatcagtgagaggctggtgatcacctctgagatgattgagaacatccagtctgtgaaggcctactgttgggaggaagctatggagaagatgattgaaaacctgaggcagacagagctgaagctgaccaggaaggctgcctatgtgagatacttcaacagctctgccttcttcttctctggcttctttgtggtgttcctgtctgtgctgccctatgccctgatcaaggggatcatcctgagaaagattttcaccaccatcagcttctgcattgtgctgaggatggctgtgaccagacagttcccctgggctgtgcagacctggtatgacagcctgggggccatcaacaagatccaggacttcctgcagaagcaggagtacaagaccctggagtacaacctgaccaccacagaagtggtgatggagaatgtgacagccttctgggaggagggctttggggagctgtttgagaaggccaagcagaacaacaacaacagaaagaccagcaatggggatgactccctgttcttctccaacttctccctgctgggcacacctgtgctgaaggacatcaacttcaagattgagagggggcagctgctggctgtggctggatctacaggggctggcaagaccagcctgctgatgatgatcatgggggagctggagccttctgagggcaagatcaagcactctggcaggatcagcttttgcagccagttcagctggatcatgcctggcaccatcaaggagaacatcatctttggagtgagctatgatgagtacagatacaggagtgtgatcaaggcctgccagctggaggaggacatcagcaagtttgctgagaaggacaacattgtgctgggggagggaggcattacactgtctgggggccagagagccagaatcagcctggccagggctgtgtacaaggatgctgacctgtacctgctggactccccctttggctacctggatgtgctgacagagaaggagatttttgagagctgtgtgtgcaagctgatggccaacaagaccagaatcctggtgaccagcaagatggagcacctgaagaaggctgacaagatcctgatcctgcatgagggcagcagctacttctatgggaccttctctgagctgcagaacctgcagcctgacttcagctctaagctgatgggctgtgacagctttgaccagttctctgctgagaggaggaacagcatcctgacagagaccctgcacagattcagcctggagggagatgcccctgtgagctggacagagaccaagaagcagagcttcaagcagacaggggagtttggggagaagaggaagaactccatcctgaaccccatcaacagcatcaggaagttcagcattgtgcagaaaacccccctgcagatgaatggcattgaggaagattctgatgagcccctggagaggagactgagcctggtgcctgattctgagcagggagaggccatcctgcctaggatctctgtgatcagcacaggccctacactgcaggccagaaggaggcagtctgtgctgaacctgatgacccactctgtgaaccagggccagaacatccacaggaaaaccacagcctccaccaggaaagtgagcctggcccctcaggccaatctgacagagctggacatctacagcaggaggctgtctcaggagacaggcctggagatttctgaggagatcaatgaggaggacctgaaagagtgcttctttgatgacatggagagcatccctgctgtgaccacctggaacacctacctgagatacatcacagtgcacaagagcctgatctttgtgctgatctggtgcctggtgatcttcctggctgaagtggctgcctctctggtggtgctgtggctgctgggaaacaccccactgcaggacaagggcaacagcacccacagcaggaacaacagctatgctgtgatcatcacctccacctccagctactatgtgttctacatctatgtgggagtggctgataccctgctggctatgggcttctttagaggcctgcccctggtgcacacactgatcacagtgagcaagatcctccaccacaagatgctgcactctgtgctgcaggctcctatgagcaccctgaataccctgaaggctgggggcatcctgaacagattctccaaggatattgccatcctggatgacctgctgcctctcaccatctttgacttcatccagctgctgctgattgtgattggggccattgctgtggtggcagtgctgcagccctacatctttgtggccacagtgcctgtgattgtggccttcatcatgctgagggcctactttctgcagacctcccagcagctgaagcagctggagtctgagggcagaagccccatcttcacccacctggtgacaagcctgaagggcctgtggaccctgagagcctttggcaggcagccctactttgagaccctgttccacaaggccctgaacctgcacacagccaactggttcctctacctgtccaccctgagatggttccagatgagaattgagatgatctttgtcatcttcttcattgctgtgaccttcatcagcattctgaccacaggagagggagagggcagagtgggcattatcctgaccctggccatgaacatcatgagcacactgcagtgggcagtgaacagcagcattgatgtggacagcctgatgaggagtgtgagcagagtgttcaagttcattgatatgcccacagagggcaagcctaccaagagcaccaagccctacaagaatggccagctgagcaaagtgatgatcattgagaacagccatgtgaagaaggatgatatctggcccagtggaggccagatgacagtgaaggacctgacagccaagtacacagaggggggcaatgctatcctggagaacatctccttcagcatctcccctggccagagagtgggactgctgggaagaacaggctctggcaagtctaccctgctgtctgccttcctgaggctgctgaacacagagggagagatccagattgatggagtgtcctgggacagcatcacactgcagcagtggaggaaggcctttggtgtgatcccccagaaagtgttcatcttcagtggcaccttcaggaagaacctggacccctatgagcagtggtctgaccaggagatttggaaagtggctgatgaagtgggcctgagaagtgtgattgagcagttccctggcaagctggactttgtcctggtggatgggggctgtgtgctgagccatggccacaagcagctgatgtgcctggccagatcagtgctgagcaaggccaagatcctgctgctggatgagccttctgcccacctggatcctgtgacctaccagatcatcaggaggaccctcaagcaggcctttgctgactgcacagtcatcctgtgtgagcacaggattgaggccatgctggagtgccagcagttcctggtgattgaggagaacaaagtgaggcagtatgacagcatccagaagctgctgaatgagaggagcctgttcaggcaggccatcagcccctctgatagagtgaagctgttcccccacaggaacagctccaagtgcaagagcaagccccagattgctgccctgaaggaggagacagaggaggaagtgcaggacaccaggctgtgagggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagct

SEQ ID NO: 2 - protein, 140 aa, Simian immunodeficiency virus
GAATSALNRRQLDQFEKIRLRPNGKKKYQIKHLIWAGKEMERFGLHERLLETEEGCKRIIEVLYPLEPTGSEGLKSLFNLVCVLYCLHKEQKVKDTEEAVATVRQHCHLVEKEKSATETSSGQKKNDKGIAAPPGGSQNF

SEQ ID NO: 3 - protein, 231 aa, Simian immunodeficiency virus
PAQQQGNAWVHVPLSPRTLNAWVKAVEEKKFGAEIVPMFQALSEGCTPYDINQMLNVLGDHQGALQIVKEIINEEAAQWDVTHPLPAGPLPAGQLRDPRGSDIAGTTSSVQEQLEWIYTANPRVDVGAIYRRWIILGLQKCVKMYNPVSVLDIRQGPKEPFKDYVDRFYKAIRAEQASGEVKQWMTESLLIQNANPDCKVILKGLGMHPTLEEMLTACQGVGGPSYKAKVM

SEQ ID NO: 4 - protein, 54 aa, Simian immunodeficiency virus
VQQGGPKRQRPPLRCYNCGKFGHMQRQCPEPRKTKCLKCGKLGHLAKDCRGQVN

SEQ ID NO: 5 - protein, 101 aa, Simian immunodeficiency virus
FELPLWRRPIKTVYIEGVPIKALLDTGADDTIIKENDLQLSGPWRPKIIGGIGGGLNVKEYNDREVKIEDKILRGTILLGATPINIIGRNLLAPAGARLVM

SEQ ID NO: 6 - protein, 441 aa, Simian immunodeficiency virus
GQLSEKIPVTPVKLKEGARGPCVRQWPLSKEKIEALQEICSQLEQEGKISRVGGENAYNTPIFCIKKKDKSQWRMLVDFRELNKATQDFFEVQLGIPHPAGLRKMRQITVLDVGDAYYSIPLDPNFRKYTAFTIPTVNNQGPGIRYQFNCLPQGWKGSPTIFQNTAASILEEIKRNLPALTIVQYMDDLWVGSQENEHTHDKLVEQLRTKLQAWGLETPEKKVQKEPPYEWMGYKLWPHKWELSRIQLEEKDEWTVNDIQKLVGKLNWAAQLYPGLRTKNICKLIRGKKNLLELVTWTPEAEAEYAENAEILKTEQEGTYYKPGIPIRAAVQKLEGGQWSYQFKQEGQVLKVGKYTKQKNTHTNELRTLAGLVQKICKEALVIWGILPVLELPIEREVWEQWWADYWQVSWIPEWDFVSTPPLLKLWYTLTKEPIPKEDVY

SEQ ID NO: 7 - protein, 120 aa, Simian immunodeficiency virus
YVDGACNRNSKEGKAGYISQYGKQRVETLENTTNQQAELTAIKMALEDSGPNVNIVTDSQYAMGILTAQPTQSDSPLVEQIIALMIQKQQIYLQWVPAHKGIGGNEEIDKLVSKGIRRVL

SEQ ID NO: 8 - protein, 291 aa, Simian immunodeficiency virus
FLEKIEEAQEEHERYHNNWKNLADTYGLPQIVAKEIVAMCPKCQIKGEPVHGQVDASPGTWQMDCTHLEGKVVIVAVHVASGFIEAEVIPRETGKETAKFLLKILSRWPITQLHTDNGPNFTSQEVAAICWWGKIEHTTGIPYNPQSQGSIESMNKQLKEIIGKIRDDCQYTETAVLMACHIHNFKRKGGIGGQTSAERLINIITTQLEIQHLQTKIQKILNFRVYYREGRDPVWKGPAQLIWKGEGAVVLKDGSDLKVVPRRKAKIIKDYEPKQRVGNEGDVEGTRGSDN

SEQ ID NO: 9 - protein, 519 aa, Simian immunodeficiency virus
MGAATSALNRRQLDQFEKIRLRPNGKKKYQIKHLIWAGKEMERFGLHERLLETEEGCKRIIEVLYPLEPTGSEGLKSLFNLVCVLYCLHKEQKVKDTEEAVATVRQHCHLVEKEKSATETSSGQKKNDKGIAAPPGGSQNFPAQQQGNAWVHVPLSPRTLNAWVKAVEEKKFGAEIVPMFQALSEGCTPYDINQMLNVLGDHQGALQIVKEIINEEAAQWDVTHPLPAGPLPAGQLRDPRGSDIAGTTSSVQEQLEWIYTANPRVDVGAIYRRWIILGLQKCVKMYNPVSVLDIRQGPKEPFKDYVDRFYKAIRAEQASGEVKQWMTESLLIQNANPDCKVILKGLGMHPTLEEMLTACQGVGGPSYKAKVMAEMMQTMQNQNMVQQGGPKRQRPPLRCYNCGKFGHMQRQCPEPRKTKCLKCGKLGHLAKDCRGQVNFLGYGRWMGAKPRNFPAATLGAEPSAPPPPSGTTPYDPAKKLLQQYAEKGKQLREQKRNPPAMNPDWTEGYSLNSLFGEDQ

SEQ ID NO: 10 - protein, 1044 aa, Simian immunodeficiency virus
MSKVWKIGTPSKRLQGTGEFFRVWTVDGGKTEKFSRRYSWSGTECASSTERHHPIRPSKEAPAAICRERETTEGAKEESTGNESGLDRGIFFELPLWRRPIKTVYIEGVPIKALLDTGADDTIIKENDLQLSGPWRPKIIGGIGGGLNVKEYNDREVKIEDKILRGTILLGATPINIIGRNLLAPAGARLVMGQLSEKIPVTPVKLKEGARGPCVRQWPLSKEKIEALQEICSQLEQEGKISRVGGENAYNTPIFCIKKKDKSQWRMLVDFRELNKATQDFFEVQLGIPHPAGLRKMRQITVLDVGDAYYSIPLDPNFRKYTAFTIPTVNNQGPGIRYQFNCLPQGWKGSPTIFQNTAASILEEIKRNLPALTIVQYMDDLWVGSQENEHTHDKLVEQLRTKLQAWGLETPEKKVQKEPPYEWMGYKLWPHKWELSRIQLEEKDEWTVNDIQKLVGKLNWAAQLYPGLRTKNICKLIRGKKNLLELVTWTPEAEAEYAENAEILKTEQEGTYYKPGIPIRAAVQKLEGGQWSYQFKQEGQVLKVGKYTKQKNTHTNELRTLAGLVQKICKEALVIWGILPVLELPIEREVWEQWWADYWQVSWIPEWDFVSTPPLLKLWYTLTKEPIPKEDVYYVDGACNRNSKEGKAGYISQYGKQRVETLENTTNQQAELTAIKMALEDSGPNVNIVTDSQYAMGILTAQPTQSDSPLVEQIIALMIQKQQIYLQWVPAHKGIGGNEEIDKLVSKGIRRVLFLEKIEEAQEEHERYHNNWKNLADTYGLPQIVAKEIVAMCPKCQIKGEPVHGQVDASPGTWQMDCTHLEGKVVIVAVHVASGFIEAEVIPRETGKETAKFLLKILSRWPITQLHTDNGPNFTSQEVAAICWWGKIEHTTGIPYNPQSQGSIESMNKQLKEIIGKIRDDCQYTETAVLMACHIHNFKRKGGIGGQTSAERLINIITTQLEIQHLQTKIQKILNFRVYYREGRDPVWKGPAQLIWKGEGAVVLKDGSDLKVVPRRKAKIIKDYEPKQRVGNEGDVEGTRGSDN

SEQ ID NO: 11 - skipped

SEQ ID NO: 12 - protein, 502 aa, synthetic construct
  REGION 1..502: note = Fct4 protein
QIPRDRLSNIGVIVDEGKSLKIAGSHESRYIVLSLVPGVDFENGCGTAQVIQYKSLLNRLLIPLRDALDLQEALITVTNDTTQNAGAPQSRFFGAVIGTIALGVATSAQITAGIALAEAREAKRDIALIKESMTKTHKSIELLQNAVGEQILALKTLQDFVNDEIKPAISELGCETAALRLGIKLTQHYSELLTAFGSNFGTIGEKSLTLQALSSLYSANITEIMTTIRTGQSNIYDVIYTEQIKGTVIDVDLERYMVTLSVKIPILSEVPGVLIHKASSISYNIDGEEWYVTVPSHILSRASFLGGADITDCVESRLTYICPRDPAQLIPDSQQKCILGDTTRCPVTKVVDSLIPKFAFVNGGVVANCIASTCTCGTGRRPISQDRSKGVVFLTHDNCGLIGVNGVELYANRRGHDATWGVQNLTVGPAIAIRPVDISLNLADATNFLQDSKAELEKARKILSEVGRWYNSRETVITIIVVMVVILVVIIVIIIVLYRLRR

SEQ ID NO: 13 - protein, 527 aa, synthetic construct
  REGION 1..527: note = Fct4 (including signal sequence)
MATYIQRVQCISTSLLVVLTTLVSCQIPRDRLSNIGVIVDEGKSLKIAGSHESRYIVLSLVPGVDFENGCGTAQVIQYKSLLNRLLIPLRDALDLQEALITVTNDTTQNAGAPQSRFFGAVIGTIALGVATSAQITAGIALAEAREAKRDIALIKESMTKTHKSIELLQNAVGEQILALKTLQDFVNDEIKPAISELGCETAALRLGIKLTQHYSELLTAFGSNFGTIGEKSLTLQALSSLYSANITEIMTTIRTGQSNIYDVIYTEQIKGTVIDVDLERYMVTLSVKIPILSEVPGVLIHKASSISYNIDGEEWYVTVPSHILSRASFLGGADITDCVESRLTYICPRDPAQLIPDSQQKCILGDTTRCPVTKVVDSLIPKFAFVNGGVVANCIASTCTCGTGRRPISQDRSKGVVFLTHDNCGLIGVNGVELYANRRGHDATWGVQNLTVGPAIAIRPVDISLNLADATNFLQDSKAELEKARKILSEVGRWYNSRETVITIIVVMVVILVVIIVIIIVLYRLRR

SEQ ID NO: 14 - protein, 411 aa, synthetic construct
  REGION 1..411: note = Fct4 (fragment 1)
FFGAVIGTIALGVATSAQITAGIALAEAREAKRDIALIKESMTKTHKSIELLQNAVGEQILALKTLQDFVNDEIKPAISELGCETAALRLGIKLTQHYSELLTAFGSNFGTIGEKSLTLQALSSLYSANITEIMTTIRTGQSNIYDVIYTEQIKGTVIDVDLERYMVTLSVKIPILSEVPGVLIHKASSISYNIDGEEWYVTVPSHILSRASFLGGADITDCVESRLTYICPRDPAQLIPDSQQKCILGDTTRCPVTKVVDSLIPKFAFVNGGVVANCIASTCTCGTGRRPISQDRSKGVVFLTHDNCGLIGVNGVELYANRRGHDATWGVQNLTVGPAIAIRPVDISLNLADATNFLQDSKAELEKARKILSEVGRWYNSRETVITIIVVMVVILVVIIVIIIVLYRLRR

SEQ ID NO: 15 - protein, 91 aa, synthetic construct
  REGION 1..91: note = Fct4 (fragment 2)
QIPRDRLSNIGVIVDEGKSLKIAGSHESRYIVLSLVPGVDFENGCGTAQVIQYKSLLNRLLIPLRDALDLQEALITVTNDTTQNAGAPQSR

SEQ ID NO: 16 - protein, 25 aa, synthetic construct
  REGION 1..25: note = Fct4 signal peptide
MATYIQRVQCISTSLLVVLTTLVSC

SEQ ID NO: 17 - DNA, 4391 bp, synthetic construct
  misc_feature 1..4391: note = codon-optimised SIV gal-pol nucleic acid sequence (from pGM691)
  source 1..4391: mol_type = other DNA
atgggagctgccacatctgccctgaatagacggcagctggaccagttcgagaagatcagactgcggcccaacggcaagaagaagtaccagatcaagcacctgatctgggccggcaaagagatggaaagattcggcctgcacgagcggctgctggaaaccgaggaaggctgcaagagaattatcgaggtgctgtaccctctggaacctaccggctctgagggcctgaagtccctgttcaatctcgtgtgcgtgctgtactgcctgcacaaagaacagaaagtgaaggacaccgaagaggccgtggccacagttagacagcactgccacctggtggaaaaagagaagtccgccacagagacaagcagcggccagaagaagaacgacaagggaattgctgcccctcctggcggcagccagaattttcctgctcagcagcagggaaacgcctgggtgcacgttccactgagccctagaacactgaatgcctgggtcaaagccgtggaagagaagaagtttggcgccgagatcgtgcccatgttccaggctctgtctgagggctgcaccccttacgacatcaaccagatgctgaacgtgctgggagatcaccagggcgctctgcagatcgtgaaagagatcatcaacgaagaggctgcccagtgggacgtgacacatccattgcctgctggacctctgccagccggacaactgagagatcctagaggctctgatatcgccggcaccaccagctctgtgcaagagcagctggaatggatctacaccgccaatcctagagtggacgtgggcgccatctacagaagatggatcatcctgggcctgcagaaatgcgtgaagatgtacaaccccgtgtccgtgctggacatcagacagggacccaaagagcccttcaaggactacgtggaccggttctataaggccattagagccgagcaggccagcggcgaagtgaagcagtggatgacagagagcctgctgatccagaacgccaatccagactgcaaagtgatcctgaaaggcctgggcatgcaccccacactggaagagatgctgacagcctgtcaaggcgttggcggcccttcttacaaagccaaagtgatggccgagatgatgcagaccatgcagaaccagaacatggtgcagcaaggcggccctaagagacagaggcctcctctgagatgctacaactgcggcaagttcggccacatgcagagacagtgtcctgagcctaggaaaacaaaatgtctaaagtgtggaaaattgggacacctagcaaaagactgcaggggacaggtgaattttttagggtatggacggtggatgggggcaaaaccgagaaattttcccgccgctactcttggagcggaaccgagtgcgcctcctccaccgagcggcaccaccccatacgacccagcaaagaagctcctgcagcaatatgcagagaaagggaaacaactgagggagcaaaagaggaatccaccggcaatgaatccggattggaccgagggatattctttgaactccctctttggagaagaccaataaagaccgtgtacatcgagggcgtgcccatcaaggctctgctggatacaggcgccgacgacaccatcatcaaagagaacgacctgcagctgagcggcccttggaggcctaagatcattggaggaatcggcggaggcctgaacgtcaaagagtacaacgaccgggaagtgaagatcgaggacaagatcctgaggggcacaatcctgctgggcgccacacctatcaacatcatcggcagaaatctgctggcccctgccggcgctagactggttatgggacagctctctgagaagatccccgtgacacccgtgaagctgaaagaaggcgctagaggaccttgtgtgcgacagtggcctctgagcaaagagaagattgaggccctgcaagaaatctgtagccagctggaacaagagggcaagatcagcagagttggcggcgagaacgcctacaatacccctatcttctgcatcaagaaaaaggacaagagccagtggcggatgctggtggactttagagagctgaacaaggctacccaggacttcttcgaggtgcagctgggaattcctcatcctgccggcctgcggaagatgagacagatcacagtgctggatgtgggcgacgcctactacagcatccctctggaccccaacttcagaaagtacaccgccttcacaatccccaccgtgaacaatcaaggccctggcatcagataccagttcaactgcctgcctcaaggctggaagggcagccccaccatttttcagaataccgccgccagcatcctggaagaaatcaagagaaacctgcctgctctgaccatcgtgcagtacatggacgatctgtgggtcggaagccaagagaatgagcacacccacgacaagctggtggaacagctgagaacaaagctgcaggcctggggcctcgaaacccctgagaagaaggtgcagaaagaacctccttacgagtggatgggctacaagctgtggcctcacaagtgggagctgagccggattcagctcgaagagaaggacgagtggaccgtgaacgacatccagaaactcgtgggcaagctgaattgggcagcccagctgtatcccggcctgaggaccaagaacatctgcaagctgatccggggaaagaagaacctgctggaactggtcacatggacacctgaggccgaggccgaatatgccgagaatgccgaaatcctgaaaaccgagcaagaggggacctactacaagcctggcattccaatcagagctgccgtgcagaaactggaaggcggccagtggtcctaccagtttaagcaagaaggccaggtcctgaaagtgggcaagtacaccaagcagaagaacacccacaccaacgagctgaggacactggctggcctggtccagaaaatctgcaaagaggccctggtcatttggggcatcctgcctgttctggaactgcccattgagcgggaagtgtgggaacagtggtgggccgattactggcaagtgtcttggatccccgagtgggacttcgtgtctacccctcctctgctgaaactgtggtacaccctgacaaaagagcccattcctaaagaggacgtctactacgttgacggcgcctgcaaccggaactccaaagaaggcaaggccggctacatcagccagtacggcaagcagagagtggaaaccctggaaaacaccaccaaccagcaggccgagctgaccgccattaagatggccctggaagatagcggccccaatgtgaacatcgtgaccgactctcagtacgccatgggaatcctgacagcccagcctacacagagcgatagccctctggttgagcagatcattgccctgatgattcagaagcagcaaatctacctgcagtgggtgcccgctcacaaaggcatcggcggaaacgaagagatcgataagctggtgtccaagggaatcagacgggtgctgttcctggaaaagattgaagaggcccaagaggaacacgagcgctaccacaacaactggaagaatctggccgacacctacggactgccccagatcgtggccaaagaaatcgtggctatgtgccccaagtgtcagatcaagggcgaacctgtgcacggccaagtggatgcttctcctggcacatggcagatggactgtacccacctggaaggcaaagtggtcatcgtggctgtgcacgtggcctccggctttattgaggccgaagtgatccccagagagacaggcaaagaaaccgccaagttcctgctgaagatcctgtccagatggcccatcacacagctgcacaccgacaacggccctaacttcacatctcaagaggtggccgccatctgttggtggggaaagattgagcacacaaccggcattccctacaatccacagagccagggcagcatcgagtccatgaacaagcagctcaaagagattatcggcaagatccgggacgactgccagtacacagaaacagccgtgctgatggcctgtcacatccacaacttcaagcggaaaggcggcatcggaggacagacatctgccgagagactgatcaatatcatcaccactcagctggaaatccagcacctccagaccaagatccagaagattctgaacttccgggtgtactaccgcgagggcagagatcctgtttggaaaggcccagcacagctgatctggaaaggcgaaggtgccgtggtgctgaaggatggctctgatctgaaggtggtgcccagacggaaggccaagattatcaaggattacgagcccaaacagcgcgtgggcaatgaaggcgacgttgagggcacaagaggcagcgacaattga

SEQ ID NO: 18 - DNA, 4391 bp, Simian immunodeficiency virus
  source 1..4391: mol_type = unassigned DNA
atgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtactatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatgtttcaagccctatcagaaggctgcacaccctatgacattaatcagatgcttaatgtgctaggagatcatcaaggggcattacaaatagtgaaagagatcattaatgaagaagcagcccagtgggatgtaacacacccactacccgcaggacccctaccagcaggacagctcagggaccctcgcggctcagatatagcagggaccaccagctcagtacaagaacagttagaatggatctatactgctaacccccgggtagatgtaggtgccatctaccggagatggattattctaggacttcaaaagtgtgtcaaaatgtacaacccagtatcagtcctagacattaggcagggacctaaagagcccttcaaggattatgtggacagattttacaaggcaattagagcagaacaagcctcaggggaagtgaaacaatggatgacagaatcattactcattcaaaatgctaatccagattgtaaggtcatcctgaagggcctaggaatgcaccccacccttgaagaaatgttaacggcttgtcagggggtaggaggcccaagctacaaagcaaaagtaatggcagaaatgatgcagaccatgcaaaatcaaaacatggtgcagcagggaggtccaaaaagacaaagacccccactaagatgttataattgtggaaaatttggccatatgcaaagacaatgtccggaaccaaggaaaacaaaatgtctaaagtgtggaaaattgggacacctagcaaaagactgcaggggacaggtgaattttttagggtatggacggtggatgggggcaaaaccgagaaattttcccgccgctactcttggagcggaaccgagtgcgcctcctccaccgagcggcaccaccccatacgacccagcaaagaagctcctgcagcaatatgcagagaaagggaaacaactgagggagcaaaagaggaatccaccggcaatgaatccggattggaccgagggatattctttgaactccctctttggagaagaccaataaagacagtgtatatagaaggggtccccattaaggcactgctagacacaggggcagatgacaccataattaaagaaaatgatttacaattatcaggtccatggagacccaaaattatagggggcataggaggaggccttaatgtaaaagaatataacgacagggaagtaaaaatagaagataaaattttgagaggaacaatattgttaggagcaactcccattaatataataggtagaaatttgctggccccggcaggtgcccggttagtaatgggacaattatcagaaaaaattcctgtcacacctgtcaaattgaaggaaggggctcggggaccctgtgtaagacaatggcctctctctaaagagaagattgaagctttacaggaaatatgttcccaattagagcaggaaggaaaaatcagtagagtaggaggagaaaatgcatacaataccccaatattttgcataaagaagaaggacaaatcccagtggaggatgctagtagactttagagagttaaataaggcaacccaagatttctttgaagtgcaattagggataccccacccagcaggattaagaaagatgagacagataacagttttagatgtaggagacgcctattattccataccattggatccaaattttaggaaatatactgcttttactattcccacagtgaataatcagggacccgggattaggtatcaattcaactgtctcccgcaagggtggaaaggatctcctacaatcttccaaaatacagcagcatccattttggaggagataaaaagaaacttgccagcactaaccattgtacaatacatggatgatttatgggtaggttctcaagaaaatgaacacacccatgacaaattagtagaacagttaagaacaaaattacaagcctggggcttagaaaccccagaaaagaaggtgcaaaaagaaccaccttatgagtggatgggatacaaactttggcctcacaaatgggaactaagcagaatacaactggaggaaaaagatgaatggactgtcaatgacatccagaagttagttgggaaactaaattgggcagcacaattgtatccaggtcttaggaccaagaatatatgcaagttaattagaggaaagaaaaatctgttagagctagtgacttggacacctgaggcagaagctgaatatgcagaaaatgcagagattcttaaaacagaacaggaaggaacctattacaaaccaggaatacctattagggcagcagtacagaaattggaaggaggacagtggagttaccaattcaaacaagaaggacaagtcttgaaagtaggaaaatacaccaagcaaaagaacacccatacaaatgaacttcgcacattagctggtttagtgcagaagatttgcaaagaagctctagttatttgggggatattaccagttctagaactcccgatagaaagagaggtatgggaacaatggtgggcggattactggcaggtaagctggattcccgaatgggattttgtcagcaccccacctttgctcaaactatggtacacattaacaaaagaacccatacccaaggaggacgtttactatgtagatggagcatgcaacagaaattcaaaagaaggaaaagcaggatacatctcacaatacggaaaacagagagtagaaacattagaaaacactaccaatcagcaagcagaattaacagctataaaaatggctttggaagacagtgggcctaatgtgaacatagtaacagactctcaatatgcaatgggaattttgacagcacaacccacacaaagtgattcaccattagtagagcaaattatagccttaatgatacaaaagcaacaaatatatttgcagtgggtaccagcacataaaggaataggaggaaatgaggagatagataaattagtgagtaaaggcattagaagagttttattcttagaaaaaatagaagaagctcaagaagagcatgaaagatatcataataattggaaaaacctagcagatacatatgggcttccacaaatagtagcaaaagagatagtggccatgtgtccaaaatgtcagataaagggagaaccagtgcatggacaagtggatgcctcacctggaacatggcagatggattgtactcatctagaaggaaaagtagtcatagttgcggtccatgtagccagtggattcatagaagcagaagtcatacctagggaaacaggaaaagaaacggcaaagtttctattaaaaatactgagtagatggcctataacacagttacacacagacaatgggcctaactttacctcccaagaagtggcagcaatatgttggtggggaaaaattgaacatacaacaggtataccatataacccccaatctcaaggatcaatagaaagcatgaacaaacaattaaaagagataattgggaaaataagagatgattgccaatatacagagacagcagtactgatggcttgccatattcacaattttaaaagaaagggaggaatagggggacagacttcagcagagagactaattaatataataacaacacaattagaaatacaacatttacaaaccaaaattcaaaaaattttaaattttagagtctactacagagaagggagagaccctgtgtggaaaggaccagcacaattaatctggaaaggggaaggagcagtggtcctcaaggacggaagtgacctaaaggttgtaccaagaaggaaagctaaaattattaaggattatgaacccaaacaaagagtgggtaatgagggtgacgtggaaggtaccaggggatctgataactaa

SEQ ID NO: 19 - DNA, 10536 bp, synthetic construct
  misc_feature 1..10536: note = pGM830
  source 1..10536: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactctgggcaagtagggcaggcggtgggtacgcaattgggggcggctacctcagcactaaataggagacaattagaccaatttgagaaaatacgacttcgcccgaacggaaagaaaaagtaccaaattaaacatttaatattgggcaggcaaggagattggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaattgcctgggtacatgtacccttgtcaccgcgcaccttaaatgcgtgggtaaaagcagtagaggagaaaaaatttggagcagaaatagtacccatgtttcaagccctatcgcctgcaggccgtttgtgctagggttcttaggcttcttgggggctgctggaactgcattgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaataagacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaaagggatttttagtaatagtaggaataatagggttaagattactttacacagtatatggatgtatagtgagggttaggcagggatatgttcctctatctccacagatccatataaagcggcaattttaaaagaaagggaggaatagggggacagacttcagcagagagactaattaatataataacaacacaattagaaatacaacatttacaaaccaaaattcaaaaaattttaaattttagagccgcggagatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagccaccatgcagagaagccctctggagaaggcctctgtggtgagcaagctgttcttcagctggaccaggcccatcctgaggaagggctacaggcagagactggagctgtctgacatctaccagatcccctctgtggactctgctgacaacctgtctgagaagctggagagggagtgggatagagagctggccagcaagaagaaccccaagctgatcaatgccctgaggagatgcttcttctggagattcatgttctatggcatcttcctgtacctgggggaagtgaccaaggctgtgcagcctctgctgctgggcagaatcattgccagctatgaccctgacaacaaggaggagaggagcattgccatctacctgggcattggcctgtgcctgctgttcattgtgaggaccctgctgctgcaccctgccatctttggcctgcaccacattggcatgcagatgaggattgccatgttcagcctgatctacaagaaaaccctgaagctgtccagcagagtgctggacaagatcagcattggccagctggtgagcctgctgagcaacaacctgaacaagtttgatgagggcctggccctggcccactttgtgtggattgcccctctgcaggtggccctgctgatgggcctgatttgggagctgctgcaggcctctgccttttgtggcctgggcttcctgattgtgctggccctgtttcaggctggcctgggcaggatgatgatgaagtacagggaccagagggcaggcaagatcagtgagaggctggtgatcacctctgagatgattgagaacatccagtctgtgaaggcctactgttgggaggaagctatggagaagatgattgaaaacctgaggcagacagagctgaagctgaccaggaaggctgcctatgtgagatacttcaacagctctgccttcttcttctctggcttctttgtggtgttcctgtctgtgctgccctatgccctgatcaaggggatcatcctgagaaagattttcaccaccatcagcttctgcattgtgctgaggatggctgtgaccagacagttcccctgggctgtgcagacctggtatgacagcctgggggccatcaacaagatccaggacttcctgcagaagcaggagtacaagaccctggagtacaacctgaccaccacagaagtggtgatggagaatgtgacagccttctgggaggagggctttggggagctgtttgagaaggccaagcagaacaacaacaacagaaagaccagcaatggggatgactccctgttcttctccaacttctccctgctgggcacacctgtgctgaaggacatcaacttcaagattgagagggggcagctgctggctgtggctggatctacaggggctggcaagaccagcctgctgatgatgatcatgggggagctggagccttctgagggcaagatcaagcactctggcaggatcagcttttgcagccagttcagctggatcatgcctggcaccatcaaggagaacatcatctttggagtgagctatgatgagtacagatacaggagtgtgatcaaggcctgccagctggaggaggacatcagcaagtttgctgagaaggacaacattgtgctgggggagggaggcattacactgtctgggggccagagagccagaatcagcctggccagggctgtgtacaaggatgctgacctgtacctgctggactccccctttggctacctggatgtgctgacagagaaggagatttttgagagctgtgtgtgcaagctgatggccaacaagaccagaatcctggtgaccagcaagatggagcacctgaagaaggctgacaagatcctgatcctgcatgagggcagcagctacttctatgggaccttctctgagctgcagaacctgcagcctgacttcagctctaagctgatgggctgtgacagctttgaccagttctctgctgagaggaggaacagcatcctgacagagaccctgcacagattcagcctggagggagatgcccctgtgagctggacagagaccaagaagcagagcttcaagcagacaggggagtttggggagaagaggaagaactccatcctgaaccccatcaacagcatcaggaagttcagcattgtgcagaaaacccccctgcagatgaatggcattgaggaagattctgatgagcccctggagaggagactgagcctggtgcctgattctgagcagggagaggccatcctgcctaggatctctgtgatcagcacaggccctacactgcaggccagaaggaggcagtctgtgctgaacctgatgacccactctgtgaaccagggccagaacatccacaggaaaaccacagcctccaccaggaaagtgagcctggcccctcaggccaatctgacagagctggacatctacagcaggaggctgtctcaggagacaggcctggagatttctgaggagatcaatgaggaggacctgaaagagtgcttctttgatgacatggagagcatccctgctgtgaccacctggaacacctacctgagatacatcacagtgcacaagagcctgatctttgtgctgatctggtgcctggtgatcttcctggctgaagtggctgcctctctggtggtgctgtggctgctgggaaacaccccactgcaggacaagggcaacagcacccacagcaggaacaacagctatgctgtgatcatcacctccacctccagctactatgtgttctacatctatgtgggagtggctgataccctgctggctatgggcttctttagaggcctgcccctggtgcacacactgatcacagtgagcaagatcctccaccacaagatgctgcactctgtgctgcaggctcctatgagcaccctgaataccctgaaggctgggggcatcctgaacagattctccaaggatattgccatcctggatgacctgctgcctctcaccatctttgacttcatccagctgctgctgattgtgattggggccattgctgtggtggcagtgctgcagccctacatctttgtggccacagtgcctgtgattgtggccttcatcatgctgagggcctactttctgcagacctcccagcagctgaagcagctggagtctgagggcagaagccccatcttcacccacctggtgacaagcctgaagggcctgtggaccctgagagcctttggcaggcagccctactttgagaccctgttccacaaggccctgaacctgcacacagccaactggttcctctacctgtccaccctgagatggttccagatgagaattgagatgatctttgtcatcttcttcattgctgtgaccttcatcagcattctgaccacaggagagggagagggcagagtgggcattatcctgaccctggccatgaacatcatgagcacactgcagtgggcagtgaacagcagcattgatgtggacagcctgatgaggagtgtgagcagagtgttcaagttcattgatatgcccacagagggcaagcctaccaagagcaccaagccctacaagaatggccagctgagcaaagtgatgatcattgagaacagccatgtgaagaaggatgatatctggcccagtggaggccagatgacagtgaaggacctgacagccaagtacacagaggggggcaatgctatcctggagaacatctccttcagcatctcccctggccagagagtgggactgctgggaagaacaggctctggcaagtctaccctgctgtctgccttcctgaggctgctgaacacagagggagagatccagattgatggagtgtcctgggacagcatcacactgcagcagtggaggaaggcctttggtgtgatcccccagaaagtgttcatcttcagtggcaccttcaggaagaacctggacccctatgagcagtggtctgaccaggagatttggaaagtggctgatgaagtgggcctgagaagtgtgattgagcagttccctggcaagctggactttgtcctggtggatgggggctgtgtgctgagccatggccacaagcagctgatgtgcctggccagatcagtgctgagcaaggccaagatcctgctgctggatgagccttctgcccacctggatcctgtgacctaccagatcatcaggaggaccctcaagcaggcctttgctgactgcacagtcatcctgtgtgagcacaggattgaggccatgctggagtgccagcagttcctggtgattgaggagaacaaagtgaggcagtatgacagcatccagaagctgctgaatgagaggagcctgttcaggcaggccatcagcccctctgatagagtgaagctgttcccccacaggaacagctccaagtgcaagagcaagccccagattgctgccctgaaggaggagacagaggaggaagtgcaggacaccaggctgtgagggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 20 - DNA, 9064 bp, synthetic construct
  misc_feature 1..9064: note = pGM691
  source 1..9064: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgctcgagccaccatgggagctgccacatctgccctgaatagacggcagctggaccagttcgagaagatcagactgcggcccaacggcaagaagaagtaccagatcaagcacctgatctgggccggcaaagagatggaaagattcggcctgcacgagcggctgctggaaaccgaggaaggctgcaagagaattatcgaggtgctgtaccctctggaacctaccggctctgagggcctgaagtccctgttcaatctcgtgtgcgtgctgtactgcctgcacaaagaacagaaagtgaaggacaccgaagaggccgtggccacagttagacagcactgccacctggtggaaaaagagaagtccgccacagagacaagcagcggccagaagaagaacgacaagggaattgctgcccctcctggcggcagccagaattttcctgctcagcagcagggaaacgcctgggtgcacgttccactgagccctagaacactgaatgcctgggtcaaagccgtggaagagaagaagtttggcgccgagatcgtgcccatgttccaggctctgtctgagggctgcaccccttacgacatcaaccagatgctgaacgtgctgggagatcaccagggcgctctgcagatcgtgaaagagatcatcaacgaagaggctgcccagtgggacgtgacacatccattgcctgctggacctctgccagccggacaactgagagatcctagaggctctgatatcgccggcaccaccagctctgtgcaagagcagctggaatggatctacaccgccaatcctagagtggacgtgggcgccatctacagaagatggatcatcctgggcctgcagaaatgcgtgaagatgtacaaccccgtgtccgtgctggacatcagacagggacccaaagagcccttcaaggactacgtggaccggttctataaggccattagagccgagcaggccagcggcgaagtgaagcagtggatgacagagagcctgctgatccagaacgccaatccagactgcaaagtgatcctgaaaggcctgggcatgcaccccacactggaagagatgctgacagcctgtcaaggcgttggcggcccttcttacaaagccaaagtgatggccgagatgatgcagaccatgcagaaccagaacatggtgcagcaaggcggccctaagagacagaggcctcctctgagatgctacaactgcggcaagttcggccacatgcagagacagtgtcctgagcctaggaaaacaaaatgtctaaagtgtggaaaattgggacacctagcaaaagactgcaggggacaggtgaattttttagggtatggacggtggatgggggcaaaaccgagaaattttcccgccgctactcttggagcggaaccgagtgcgcctcctccaccgagcggcaccaccccatacgacccagcaaagaagctcctgcagcaatatgcagagaaagggaaacaactgagggagcaaaagaggaatccaccggcaatgaatccggattggaccgagggatattctttgaactccctctttggagaagaccaataaagaccgtgtacatcgagggcgtgcccatcaaggctctgctggatacaggcgccgacgacaccatcatcaaagagaacgacctgcagctgagcggcccttggaggcctaagatcattggaggaatcggcggaggcctgaacgtcaaagagtacaacgaccgggaagtgaagatcgaggacaagatcctgaggggcacaatcctgctgggcgccacacctatcaacatcatcggcagaaatctgctggcccctgccggcgctagactggttatgggacagctctctgagaagatccccgtgacacccgtgaagctgaaagaaggcgctagaggaccttgtgtgcgacagtggcctctgagcaaagagaagattgaggccctgcaagaaatctgtagccagctggaacaagagggcaagatcagcagagttggcggcgagaacgcctacaatacccctatcttctgcatcaagaaaaaggacaagagccagtggcggatgctggtggactttagagagctgaacaaggctacccaggacttcttcgaggtgcagctgggaattcctcatcctgccggcctgcggaagatgagacagatcacagtgctggatgtgggcgacgcctactacagcatccctctggaccccaacttcagaaagtacaccgccttcacaatccccaccgtgaacaatcaaggccctggcatcagataccagttcaactgcctgcctcaaggctggaagggcagccccaccatttttcagaataccgccgccagcatcctggaagaaatcaagagaaacctgcctgctctgaccatcgtgcagtacatggacgatctgtgggtcggaagccaagagaatgagcacacccacgacaagctggtggaacagctgagaacaaagctgcaggcctggggcctcgaaacccctgagaagaaggtgcagaaagaacctccttacgagtggatgggctacaagctgtggcctcacaagtgggagctgagccggattcagctcgaagagaaggacgagtggaccgtgaacgacatccagaaactcgtgggcaagctgaattgggcagcccagctgtatcccggcctgaggaccaagaacatctgcaagctgatccggggaaagaagaacctgctggaactggtcacatggacacctgaggccgaggccgaatatgccgagaatgccgaaatcctgaaaaccgagcaagaggggacctactacaagcctggcattccaatcagagctgccgtgcagaaactggaaggcggccagtggtcctaccagtttaagcaagaaggccaggtcctgaaagtgggcaagtacaccaagcagaagaacacccacaccaacgagctgaggacactggctggcctggtccagaaaatctgcaaagaggccctggtcatttggggcatcctgcctgttctggaactgcccattgagcgggaagtgtgggaacagtggtgggccgattactggcaagtgtcttggatccccgagtgggacttcgtgtctacccctcctctgctgaaactgtggtacaccctgacaaaagagcccattcctaaagaggacgtctactacgttgacggcgcctgcaaccggaactccaaagaaggcaaggccggctacatcagccagtacggcaagcagagagtggaaaccctggaaaacaccaccaaccagcaggccgagctgaccgccattaagatggccctggaagatagcggccccaatgtgaacatcgtgaccgactctcagtacgccatgggaatcctgacagcccagcctacacagagcgatagccctctggttgagcagatcattgccctgatgattcagaagcagcaaatctacctgcagtgggtgcccgctcacaaaggcatcggcggaaacgaagagatcgataagctggtgtccaagggaatcagacgggtgctgttcctggaaaagattgaagaggcccaagaggaacacgagcgctaccacaacaactggaagaatctggccgacacctacggactgccccagatcgtggccaaagaaatcgtggctatgtgccccaagtgtcagatcaagggcgaacctgtgcacggccaagtggatgcttctcctggcacatggcagatggactgtacccacctggaaggcaaagtggtcatcgtggctgtgcacgtggcctccggctttattgaggccgaagtgatccccagagagacaggcaaagaaaccgccaagttcctgctgaagatcctgtccagatggcccatcacacagctgcacaccgacaacggccctaacttcacatctcaagaggtggccgccatctgttggtggggaaagattgagcacacaaccggcattccctacaatccacagagccagggcagcatcgagtccatgaacaagcagctcaaagagattatcggcaagatccgggacgactgccagtacacagaaacagccgtgctgatggcctgtcacatccacaacttcaagcggaaaggcggcatcggaggacagacatctgccgagagactgatcaatatcatcaccactcagctggaaatccagcacctccagaccaagatccagaagattctgaacttccgggtgtactaccgcgagggcagagatcctgtttggaaaggcccagcacagctgatctggaaaggcgaaggtgccgtggtgctgaaggatggctctgatctgaaggtggtgcccagacggaaggccaagattatcaaggattacgagcccaaacagcgcgtgggcaatgaaggcgacgttgagggcacaagaggcagcgacaattgaaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgac

SEQ ID NO: 21 - DNA, 9886 bp, synthetic construct
  misc_feature 1..9886: note = pGM297
  source 1..9886: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgctcgagactagtgacttggtgagtaggcttcgagcctagttagaggactaggagaggccgtagccgtaactactctgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtactatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatgtttcaagccctatcagaaggctgcacaccctatgacattaatcagatgcttaatgtgctaggagatcatcaaggggcattacaaatagtgaaagagatcattaatgaagaagcagcccagtgggatgtaacacacccactacccgcaggacccctaccagcaggacagctcagggaccctcgcggctcagatatagcagggaccaccagctcagtacaagaacagttagaatggatctatactgctaacccccgggtagatgtaggtgccatctaccggagatggattattctaggacttcaaaagtgtgtcaaaatgtacaacccagtatcagtcctagacattaggcagggacctaaagagcccttcaaggattatgtggacagattttacaaggcaattagagcagaacaagcctcaggggaagtgaaacaatggatgacagaatcattactcattcaaaatgctaatccagattgtaaggtcatcctgaagggcctaggaatgcaccccacccttgaagaaatgttaacggcttgtcagggggtaggaggcccaagctacaaagcaaaagtaatggcagaaatgatgcagaccatgcaaaatcaaaacatggtgcagcagggaggtccaaaaagacaaagacccccactaagatgttataattgtggaaaatttggccatatgcaaagacaatgtccggaaccaaggaaaacaaaatgtctaaagtgtggaaaattgggacacctagcaaaagactgcaggggacaggtgaattttttagggtatggacggtggatgggggcaaaaccgagaaattttcccgccgctactcttggagcggaaccgagtgcgcctcctccaccgagcggcaccaccccatacgacccagcaaagaagctcctgcagcaatatgcagagaaagggaaacaactgagggagcaaaagaggaatccaccggcaatgaatccggattggaccgagggatattctttgaactccctctttggagaagaccaataaagacagtgtatatagaaggggtccccattaaggcactgctagacacaggggcagatgacaccataattaaagaaaatgatttacaattatcaggtccatggagacccaaaattatagggggcataggaggaggccttaatgtaaaagaatataacgacagggaagtaaaaatagaagataaaattttgagaggaacaatattgttaggagcaactcccattaatataataggtagaaatttgctggccccggcaggtgcccggttagtaatgggacaattatcagaaaaaattcctgtcacacctgtcaaattgaaggaaggggctcggggaccctgtgtaagacaatggcctctctctaaagagaagattgaagctttacaggaaatatgttcccaattagagcaggaaggaaaaatcagtagagtaggaggagaaaatgcatacaataccccaatattttgcataaagaagaaggacaaatcccagtggaggatgctagtagactttagagagttaaataaggcaacccaagatttctttgaagtgcaattagggataccccacccagcaggattaagaaagatgagacagataacagttttagatgtaggagacgcctattattccataccattggatccaaattttaggaaatatactgcttttactattcccacagtgaataatcagggacccgggattaggtatcaattcaactgtctcccgcaagggtggaaaggatctcctacaatcttccaaaatacagcagcatccattttggaggagataaaaagaaacttgccagcactaaccattgtacaatacatggatgatttatgggtaggttctcaagaaaatgaacacacccatgacaaattagtagaacagttaagaacaaaattacaagcctggggcttagaaaccccagaaaagaaggtgcaaaaagaaccaccttatgagtggatgggatacaaactttggcctcacaaatgggaactaagcagaatacaactggaggaaaaagatgaatggactgtcaatgacatccagaagttagttgggaaactaaattgggcagcacaattgtatccaggtcttaggaccaagaatatatgcaagttaattagaggaaagaaaaatctgttagagctagtgacttggacacctgaggcagaagctgaatatgcagaaaatgcagagattcttaaaacagaacaggaaggaacctattacaaaccaggaatacctattagggcagcagtacagaaattggaaggaggacagtggagttaccaattcaaacaagaaggacaagtcttgaaagtaggaaaatacaccaagcaaaagaacacccatacaaatgaacttcgcacattagctggtttagtgcagaagatttgcaaagaagctctagttatttgggggatattaccagttctagaactcccgatagaaagagaggtatgggaacaatggtgggcggattactggcaggtaagctggattcccgaatgggattttgtcagcaccccacctttgctcaaactatggtacacattaacaaaagaacccatacccaaggaggacgtttactatgtagatggagcatgcaacagaaattcaaaagaaggaaaagcaggatacatctcacaatacggaaaacagagagtagaaacattagaaaacactaccaatcagcaagcagaattaacagctataaaaatggctttggaagacagtgggcctaatgtgaacatagtaacagactctcaatatgcaatgggaattttgacagcacaacccacacaaagtgattcaccattagtagagcaaattatagccttaatgatacaaaagcaacaaatatatttgcagtgggtaccagcacataaaggaataggaggaaatgaggagatagataaattagtgagtaaaggcattagaagagttttattcttagaaaaaatagaagaagctcaagaagagcatgaaagatatcataataattggaaaaacctagcagatacatatgggcttccacaaatagtagcaaaagagatagtggccatgtgtccaaaatgtcagataaagggagaaccagtgcatggacaagtggatgcctcacctggaacatggcagatggattgtactcatctagaaggaaaagtagtcatagttgcggtccatgtagccagtggattcatagaagcagaagtcatacctagggaaacaggaaaagaaacggcaaagtttctattaaaaatactgagtagatggcctataacacagttacacacagacaatgggcctaactttacctcccaagaagtggcagcaatatgttggtggggaaaaattgaacatacaacaggtataccatataacccccaatctcaaggatcaatagaaagcatgaacaaacaattaaaagagataattgggaaaataagagatgattgccaatatacagagacagcagtactgatggcttgccatattcacaattttaaaagaaagggaggaatagggggacagacttcagcagagagactaattaatataataacaacacaattagaaatacaacatttacaaaccaaaattcaaaaaattttaaattttagagtctactacagagaagggagagaccctgtgtggaaaggaccagcacaattaatctggaaaggggaaggagcagtggtcctcaaggacggaagtgacctaaaggttgtaccaagaaggaaagctaaaattattaaggattatgaacccaaacaaagagtgggtaatgagggtgacgtggaaggtaccaggggatctgataactaaatggcagggaatagtcagatattggatgagacaaagaaatttgaaatggaactattatatgcatcagctggcggccgcgaattcactagtgattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccaatcgaattcccgcggccgcaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgac

SEQ ID NO: 22 - DNA, 3384 bp, synthetic construct
  misc_feature 1..3384: note = pGM299
  source 1..3384: mol_type = other DNA
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcgattatgcccctaggaccagaagaaagaagattgcttcgcttgatttggctcctttacagcaccaatccatatccaccaagtggggaagggacggccagacaacgccgacgagccaggagaaggtggagacaacagcaggatcaaattagagtcttggtagaaagactccaagagcaggtgtatgcagttgaccgcctggctgacgaggctcaacacttggctatacaacagttgcctgaccctcctcattcagcttagaatcactagtgaattcacgcgtggtacctctagagtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatccgtcgaccaattgttgtgtctcaaaatctctgatgttacattgcacaagataaaaatatatcatcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagctgttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 23 - DNA, 6264 bp, synthetic construct
  misc_feature 1..6264: note = pGM301
  source 1..6264: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgattgccatggcaacatatatccagagagtacagtgcatctcaacatcactactggttgttctcaccacattggtctcgtgtcagattcccagggataggctctctaacataggggtcatagtcgatgaagggaaatcactgaagatagctggatcccacgaatcgaggtacatagtactgagtctagttccgggggtagactttgagaatgggtgcggaacagcccaggttatccagtacaagagcctactgaacaggctgttaatcccattgagggatgccttagatcttcaggaggctctgataactgtcaccaatgatacgacacaaaatgccggtgctccccagtcgagattcttcggtgctgtgattggtactatcgcacttggagtggcgacatcagcacaaatcaccgcagggattgcactagccgaagcgagggaggccaaaagagacatagcgctcatcaaagaatcgatgacaaaaacacacaagtctatagaactgctgcaaaacgctgtgggggaacaaattcttgctctaaagacactccaggatttcgtgaatgatgagatcaaacccgcaataagcgaattaggctgtgagactgctgccttaagactgggtataaaattgacacagcattactccgagctgttaactgcgttcggctcgaatttcggaaccatcggagagaagagcctcacgctgcaggcgctgtcttcactttactctgctaacattactgagattatgaccacaatcaggacagggcagtctaacatctatgatgtcatttatacagaacagatcaaaggaacggtgatagatgtggatctagagagatacatggtcaccctgtctgtgaagatccctattctttctgaagtcccaggtgtgctcatacacaaggcatcatctatttcttacaacatagacggggaggaatggtatgtgactgtccccagccatatactcagtcgtgcttctttcttagggggtgcagacataaccgattgtgttgagtccagattgacctatatatgccccagggatcccgcacaactgatacctgacagccagcaaaagtgtatcctgggggacacaacaaggtgtcctgtcacaaaagttgtggacagccttatccccaagtttgcttttgtgaatgggggcgttgttgctaactgcatagcatccacatgtacctgcgggacaggccgaagaccaatcagtcaggatcgctctaaaggtgtagtattcctaacccatgacaactgtggtcttataggtgtcaatggggtagaattgtatgctaaccggagagggcacgatgccacttggggggtccagaacttgacagtcggtcctgcaattgctatcagacccgttgatatttctctcaaccttgctgatgctacgaatttcttgcaagactctaaggctgagcttgagaaagcacggaaaatcctctcggaggtaggtagatggtacaactcaagagagactgtgattacgatcatagtagttatggtcgtaatattggtggtcattatagtgatcatcatcgtgctttatagactcagaaggtgaaatcactagtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgac

SEQ ID NO: 24 - DNA, 6522 bp, synthetic construct
  misc_feature 1..6522: note = pGM303
  source 1..6522: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgagcatgtggtctgagttaaaaatcaggagcaacgacggaggtgaaggaccagaggacgccaacgacccccggggaaagggggtgcaacacatccatatccagccatctctacctgtttatggacagagggttagggatggtgataggggcaaacgtgactcgtactggtctacttctcctagtggtagcaccacaaaaccagcatcaggttgggagaggtcaagtaaagccgacacatggttgctgattctctcattcacccagtgggctttgtcaattgccacagtgatcatctgtatcataatttctgctagacaagggtatagtatgaaagagtactcaatgactgtagaggcattgaacatgagcagcagggaggtgaaagagtcacttaccagtctaataaggcaagaggttatagcaagggctgtcaacattcagagctctgtgcaaaccggaatcccagtcttgttgaacaaaaacagcagggatgtcatccagatgattgataagtcgtgcagcagacaagagctcactcagcactgtgagagtacgatcgcagtccaccatgccgatggaattgccccacttgagccacatagtttctggagatgccctgtcggagaaccgtatcttagctcagatcctgaaatctcattgctgcctggtccgagcttgttatctggttctacaacgatctctggatgtgttaggctcccttcactctcaattggcgaggcaatctatgcctattcatcaaatctcattacacaaggttgtgctgacatagggaaatcatatcaggtcctgcagctagggtacatatcactcaattcagatatgttccctgatcttaaccccgtagtgtcccacacttatgacatcaacgacaatcggaaatcatgctctgtggtggcaaccgggactaggggttatcagctttgctccatgccgactgtagacgaaagaaccgactactctagtgatggtattgaggatctggtccttgatgtcctggatctcaaagggagaactaagtctcaccggtatcgcaacagcgaggtagatcttgatcacccgttctctgcactataccccagtgtaggcaacggcattgcaacagaaggctcattgatatttcttgggtatggtggactaaccacccctctgcagggtgatacaaaatgtaggacccaaggatgccaacaggtgtcgcaagacacatgcaatgaggctctgaaaattacatggctaggagggaaacaggtggtcagcgtgatcatccaggtcaatgactatctctcagagaggccaaagataagagtcacaaccattccaatcactcaaaactatctcggggcggaaggtagattattaaaattgggtgatcgggtgtacatctatacaagatcatcaggctggcactctcaactgcagataggagtacttgatgtcagccaccctttgactatcaactggacacctcatgaagccttgtctagaccaggaaataaagagtgcaattggtacaataagtgtccgaaggaatgcatatcaggcgtatacactgatgcttatccattgtcccctgatgcagctaacgtcgctaccgtcacgctatatgccaatacatcgcgtgtcaacccaacaatcatgtattctaacactactaacattataaatatgttaaggataaaggatgttcaattagaggctgcatataccacgacatcgtgtatcacgcattttggtaaaggctactgctttcacatcatcgagatcaatcagaagagcctgaataccttacagccgatgctctttaagactagcatccctaaattatgcaaggccgagtcttaagcggccgcgcatgcgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtctattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgac

SEQ ID NO: 25 - DNA, 10528 bp, synthetic construct
  misc_feature 1..10528: note = pGM326
  source 1..10528: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactctgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatgtttcaagccctatcgaattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccgcggcaattttaaaagaaagggaggaatagggggacagacttcagcagagagactaattaatataataacaacacaattagaaatacaacatttacaaaccaaaattcaaaaaattttaaattttagagccgcggagatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagccaccatgcagagaagccctctggagaaggcctctgtggtgagcaagctgttcttcagctggaccaggcccatcctgaggaagggctacaggcagagactggagctgtctgacatctaccagatcccctctgtggactctgctgacaacctgtctgagaagctggagagggagtgggatagagagctggccagcaagaagaaccccaagctgatcaatgccctgaggagatgcttcttctggagattcatgttctatggcatcttcctgtacctgggggaagtgaccaaggctgtgcagcctctgctgctgggcagaatcattgccagctatgaccctgacaacaaggaggagaggagcattgccatctacctgggcattggcctgtgcctgctgttcattgtgaggaccctgctgctgcaccctgccatctttggcctgcaccacattggcatgcagatgaggattgccatgttcagcctgatctacaagaaaaccctgaagctgtccagcagagtgctggacaagatcagcattggccagctggtgagcctgctgagcaacaacctgaacaagtttgatgagggcctggccctggcccactttgtgtggattgcccctctgcaggtggccctgctgatgggcctgatttgggagctgctgcaggcctctgccttttgtggcctgggcttcctgattgtgctggccctgtttcaggctggcctgggcaggatgatgatgaagtacagggaccagagggcaggcaagatcagtgagaggctggtgatcacctctgagatgattgagaacatccagtctgtgaaggcctactgttgggaggaagctatggagaagatgattgaaaacctgaggcagacagagctgaagctgaccaggaaggctgcctatgtgagatacttcaacagctctgccttcttcttctctggcttctttgtggtgttcctgtctgtgctgccctatgccctgatcaaggggatcatcctgagaaagattttcaccaccatcagcttctgcattgtgctgaggatggctgtgaccagacagttcccctgggctgtgcagacctggtatgacagcctgggggccatcaacaagatccaggacttcctgcagaagcaggagtacaagaccctggagtacaacctgaccaccacagaagtggtgatggagaatgtgacagccttctgggaggagggctttggggagctgtttgagaaggccaagcagaacaacaacaacagaaagaccagcaatggggatgactccctgttcttctccaacttctccctgctgggcacacctgtgctgaaggacatcaacttcaagattgagagggggcagctgctggctgtggctggatctacaggggctggcaagaccagcctgctgatgatgatcatgggggagctggagccttctgagggcaagatcaagcactctggcaggatcagcttttgcagccagttcagctggatcatgcctggcaccatcaaggagaacatcatctttggagtgagctatgatgagtacagatacaggagtgtgatcaaggcctgccagctggaggaggacatcagcaagtttgctgagaaggacaacattgtgctgggggagggaggcattacactgtctgggggccagagagccagaatcagcctggccagggctgtgtacaaggatgctgacctgtacctgctggactccccctttggctacctggatgtgctgacagagaaggagatttttgagagctgtgtgtgcaagctgatggccaacaagaccagaatcctggtgaccagcaagatggagcacctgaagaaggctgacaagatcctgatcctgcatgagggcagcagctacttctatgggaccttctctgagctgcagaacctgcagcctgacttcagctctaagctgatgggctgtgacagctttgaccagttctctgctgagaggaggaacagcatcctgacagagaccctgcacagattcagcctggagggagatgcccctgtgagctggacagagaccaagaagcagagcttcaagcagacaggggagtttggggagaagaggaagaactccatcctgaaccccatcaacagcatcaggaagttcagcattgtgcagaaaacccccctgcagatgaatggcattgaggaagattctgatgagcccctggagaggagactgagcctggtgcctgattctgagcagggagaggccatcctgcctaggatctctgtgatcagcacaggccctacactgcaggccagaaggaggcagtctgtgctgaacctgatgacccactctgtgaaccagggccagaacatccacaggaaaaccacagcctccaccaggaaagtgagcctggcccctcaggccaatctgacagagctggacatctacagcaggaggctgtctcaggagacaggcctggagatttctgaggagatcaatgaggaggacctgaaagagtgcttctttgatgacatggagagcatccctgctgtgaccacctggaacacctacctgagatacatcacagtgcacaagagcctgatctttgtgctgatctggtgcctggtgatcttcctggctgaagtggctgcctctctggtggtgctgtggctgctgggaaacaccccactgcaggacaagggcaacagcacccacagcaggaacaacagctatgctgtgatcatcacctccacctccagctactatgtgttctacatctatgtgggagtggctgataccctgctggctatgggcttctttagaggcctgcccctggtgcacacactgatcacagtgagcaagatcctccaccacaagatgctgcactctgtgctgcaggctcctatgagcaccctgaataccctgaaggctgggggcatcctgaacagattctccaaggatattgccatcctggatgacctgctgcctctcaccatctttgacttcatccagctgctgctgattgtgattggggccattgctgtggtggcagtgctgcagccctacatctttgtggccacagtgcctgtgattgtggccttcatcatgctgagggcctactttctgcagacctcccagcagctgaagcagctggagtctgagggcagaagccccatcttcacccacctggtgacaagcctgaagggcctgtggaccctgagagcctttggcaggcagccctactttgagaccctgttccacaaggccctgaacctgcacacagccaactggttcctctacctgtccaccctgagatggttccagatgagaattgagatgatctttgtcatcttcttcattgctgtgaccttcatcagcattctgaccacaggagagggagagggcagagtgggcattatcctgaccctggccatgaacatcatgagcacactgcagtgggcagtgaacagcagcattgatgtggacagcctgatgaggagtgtgagcagagtgttcaagttcattgatatgcccacagagggcaagcctaccaagagcaccaagccctacaagaatggccagctgagcaaagtgatgatcattgagaacagccatgtgaagaaggatgatatctggcccagtggaggccagatgacagtgaaggacctgacagccaagtacacagaggggggcaatgctatcctggagaacatctccttcagcatctcccctggccagagagtgggactgctgggaagaacaggctctggcaagtctaccctgctgtctgccttcctgaggctgctgaacacagagggagagatccagattgatggagtgtcctgggacagcatcacactgcagcagtggaggaaggcctttggtgtgatcccccagaaagtgttcatcttcagtggcaccttcaggaagaacctggacccctatgagcagtggtctgaccaggagatttggaaagtggctgatgaagtgggcctgagaagtgtgattgagcagttccctggcaagctggactttgtcctggtggatgggggctgtgtgctgagccatggccacaagcagctgatgtgcctggccagatcagtgctgagcaaggccaagatcctgctgctggatgagccttctgcccacctggatcctgtgacctaccagatcatcaggaggaccctcaagcaggcctttgctgactgcacagtcatcctgtgtgagcacaggattgaggccatgctggagtgccagcagttcctggtgattgaggagaacaaagtgaggcagtatgacagcatccagaagctgctgaatgagaggagcctgttcaggcaggccatcagcccctctgatagagtgaagctgttcccccacaggaacagctccaagtgcaagagcaagccccagattgctgccctgaaggaggagacagaggaggaagtgcaggacaccaggctgtgagggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 26 - DNA, 574 bp, synthetic construct
  misc_feature 1..574: note = hCEF promoter
  source 1..574: mol_type = other DNA
agatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagc

SEQ ID NO: 27 - DNA, 873 bp, synthetic construct
  misc_feature 1..873: note = CMV promoter
  source 1..873: mol_type = other DNA
ccgcggagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggctagc

SEQ ID NO: 28 - DNA, 395 bp, synthetic construct
  misc_feature 1..395: note = EF1a promoter
  source 1..395: mol_type = other DNA
agatccatatccgcggcaattttaaaagaaagggaggaatagggggacagacttcagcagagagactaattaatataataacaacacaattagaaatacaacatttacaaaccaaaattcaaaaaattttaaattttagagccgcggagatcccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctagc

SEQ ID NO: 29 - DNA, 4458 bp, synthetic construct
  misc_feature 1..4458: note = soCFTR2
  source 1..4458: mol_type = other DNA
gctagccacatgcagagaagccctctggagaaggcctctgtggtgagcaagctgttcttcagctggaccaggcccatcctgaggaagggctacaggcagagactggagctgtctgacatctaccagatcccctctgtggactctgctgacaacctgtctgagaagctggagagggagtgggatagagagctggccagcaagaagaaccccaagctgatcaatgccctgaggagatgcttcttctggagattcatgttctatggcatcttcctgtacctgggggaagtgaccaaggctgtgcagcctctgctgctgggcagaatcattgccagctatgaccctgacaacaaggaggagaggagcattgccatctacctgggcattggcctgtgcctgctgttcattgtgaggaccctgctgctgcaccctgccatctttggcctgcaccacattggcatgcagatgaggattgccatgttcagcctgatctacaagaaaaccctgaagctgtccagcagagtgctggacaagatcagcattggccagctggtgagcctgctgagcaacaacctgaacaagtttgatgagggcctggccctggcccactttgtgtggattgcccctctgcaggtggccctgctgatgggcctgatttgggagctgctgcaggcctctgccttttgtggcctgggcttcctgattgtgctggccctgtttcaggctggcctgggcaggatgatgatgaagtacagggaccagagggcaggcaagatcagtgagaggctggtgatcacctctgagatgattgagaacatccagtctgtgaaggcctactgttgggaggaagctatggagaagatgattgaaaacctgaggcagacagagctgaagctgaccaggaaggctgcctatgtgagatacttcaacagctctgccttcttcttctctggcttctttgtggtgttcctgtctgtgctgccctatgccctgatcaaggggatcatcctgagaaagattttcaccaccatcagcttctgcattgtgctgaggatggctgtgaccagacagttcccctgggctgtgcagacctggtatgacagcctgggggccatcaacaagatccaggacttcctgcagaagcaggagtacaagaccctggagtacaacctgaccaccacagaagtggtgatggagaatgtgacagccttctgggaggagggctttggggagctgtttgagaaggccaagcagaacaacaacaacagaaagaccagcaatggggatgactccctgttcttctccaacttctccctgctgggcacacctgtgctgaaggacatcaacttcaagattgagagggggcagctgctggctgtggctggatctacaggggctggcaagaccagcctgctgatgatgatcatgggggagctggagccttctgagggcaagatcaagcactctggcaggatcagcttttgcagccagttcagctggatcatgcctggcaccatcaaggagaacatcatctttggagtgagctatgatgagtacagatacaggagtgtgatcaaggcctgccagctggaggaggacatcagcaagtttgctgagaaggacaacattgtgctgggggagggaggcattacactgtctgggggccagagagccagaatcagcctggccagggctgtgtacaaggatgctgacctgtacctgctggactccccctttggctacctggatgtgctgacagagaaggagatttttgagagctgtgtgtgcaagctgatggccaacaagaccagaatcctggtgaccagcaagatggagcacctgaagaaggctgacaagatcctgatcctgcatgagggcagcagctacttctatgggaccttctctgagctgcagaacctgcagcctgacttcagctctaagctgatgggctgtgacagctttgaccagttctctgctgagaggaggaacagcatcctgacagagaccctgcacagattcagcctggagggagatgcccctgtgagctggacagagaccaagaagcagagcttcaagcagacaggggagtttggggagaagaggaagaactccatcctgaaccccatcaacagcatcaggaagttcagcattgtgcagaaaacccccctgcagatgaatggcattgaggaagattctgatgagcccctggagaggagactgagcctggtgcctgattctgagcagggagaggccatcctgcctaggatctctgtgatcagcacaggccctacactgcaggccagaaggaggcagtctgtgctgaacctgatgacccactctgtgaaccagggccagaacatccacaggaaaaccacagcctccaccaggaaagtgagcctggcccctcaggccaatctgacagagctggacatctacagcaggaggctgtctcaggagacaggcctggagatttctgaggagatcaatgaggaggacctgaaagagtgcttctttgatgacatggagagcatccctgctgtgaccacctggaacacctacctgagatacatcacagtgcacaagagcctgatctttgtgctgatctggtgcctggtgatcttcctggctgaagtggctgcctctctggtggtgctgtggctgctgggaaacaccccactgcaggacaagggcaacagcacccacagcaggaacaacagctatgctgtgatcatcacctccacctccagctactatgtgttctacatctatgtgggagtggctgataccctgctggctatgggcttctttagaggcctgcccctggtgcacacactgatcacagtgagcaagatcctccaccacaagatgctgcactctgtgctgcaggctcctatgagcaccctgaataccctgaaggctgggggcatcctgaacagattctccaaggatattgccatcctggatgacctgctgcctctcaccatctttgacttcatccagctgctgctgattgtgattggggccattgctgtggtggcagtgctgcagccctacatctttgtggccacagtgcctgtgattgtggccttcatcatgctgagggcctactttctgcagacctcccagcagctgaagcagctggagtctgagggcagaagccccatcttcacccacctggtgacaagcctgaagggcctgtggaccctgagagcctttggcaggcagccctactttgagaccctgttccacaaggccctgaacctgcacacagccaactggttcctctacctgtccaccctgagatggttccagatgagaattgagatgatctttgtcatcttcttcattgctgtgaccttcatcagcattctgaccacaggagagggagagggcagagtgggcattatcctgaccctggccatgaacatcatgagcacactgcagtgggcagtgaacagcagcattgatgtggacagcctgatgaggagtgtgagcagagtgttcaagttcattgatatgcccacagagggcaagcctaccaagagcaccaagccctacaagaatggccagctgagcaaagtgatgatcattgagaacagccatgtgaagaaggatgatatctggcccagtggaggccagatgacagtgaaggacctgacagccaagtacacagaggggggcaatgctatcctggagaacatctccttcagcatctcccctggccagagagtgggactgctgggaagaacaggctctggcaagtctaccctgctgtctgccttcctgaggctgctgaacacagagggagagatccagattgatggagtgtcctgggacagcatcacactgcagcagtggaggaaggcctttggtgtgatcccccagaaagtgttcatcttcagtggcaccttcaggaagaacctggacccctatgagcagtggtctgaccaggagatttggaaagtggctgatgaagtgggcctgagaagtgtgattgagcagttccctggcaagctggactttgtcctggtggatgggggctgtgtgctgagccatggccacaagcagctgatgtgcctggccagatcagtgctgagcaaggccaagatcctgctgctggatgagccttctgcccacctggatcctgtgacctaccagatcatcaggaggaccctcaagcaggcctttgctgactgcacagtcatcctgtgtgagcacaggattgaggccatgctggagtgccagcagttcctggtgattgaggagaacaaagtgaggcagtatgacagcatccagaagctgctgaatgagaggagcctgttcaggcaggccatcagcccctctgatagagtgaagctgttcccccacaggaacagctccaagtgcaagagcaagccccagattgctgccctgaaggaggagacagaggaggaagtgcaggacaccaggctgtgagggccc

SEQ ID NO: 30 - DNA, 1257 bp, synthetic construct
  misc_feature 1..1257: note = sohAAT
  source 1..1257: mol_type = other DNA
atgcccagctctgtgtcctggggcattctgctgctggctggcctgtgctgtctggtgcctgtgtccctggctgaggaccctcagggggatgctgcccagaaaacagacacctcccaccatgaccaggaccaccccaccttcaacaagatcacccccaacctggcagagtttgccttcagcctgtacagacagctggcccaccagagcaacagcaccaacatctttttcagccctgtgtccattgccacagcctttgccatgctgagcctgggcaccaaggctgacacccatgatgagatcctggaaggcctgaacttcaacctgacagagatccctgaggcccagatccatgagggcttccaggaactgctgagaaccctgaaccagccagacagccagctgcagctgacaacaggcaatgggctgttcctgtctgagggcctgaagctggtggacaagtttctggaagatgtgaagaagctgtaccactctgaggccttcacagtgaactttggggacacagaagaggccaagaaacagatcaatgactatgtggaaaagggcacccagggcaagattgtggaccttgtgaaagagctggacagggacactgtgtttgcccttgtgaactacatcttcttcaagggcaagtgggagaggccctttgaagtgaaggacactgaggaagaggacttccatgtggaccaagtgaccacagtgaaggtgccaatgatgaagagactggggatgttcaatatccagcactgcaagaaactgagcagctgggtgctgctgatgaagtacctgggcaatgctacagccatattctttctgcctgatgagggcaagctgcagcacctggaaaatgagctgacccatgacatcatcaccaaatttctggaaaatgaggacagaagatctgccagcctgcatctgcccaagctgagcatcacaggcacatatgacctgaagtctgtgctgggacagctgggaatcaccaaggtgttcagcaatggggcagacctgagtggagtgacagaggaagcccctctgaagctgtccaaggctgtgcacaaggcagtgctgaccattgatgagaagggcacagaggctgctggggccatgtttctggaagccatccccatgtccatccccccagaagtgaagttcaacaagccctttgtgttcctgatgattgagcagaacaccaagagccccctgttcatgggcaaggttgtgaaccccacccagaaatga

SEQ ID NO: 31 - DNA, 1257 bp, synthetic construct
  misc_feature 1..1257: note = sohAAT completmentary strand
  source 1..1257: mol_type = other DNA
tacgggtcgagacacaggaccccgtaagacgacgaccgaccggacacgacagaccacggacacagggaccgactcctgggagtccccctacgacgggtcttttgtctgtggagggtggtactggtcctggtggggtggaagttgttctagtgggggttggaccgtctcaaacggaagtcggacatgtctgtcgaccgggtggtctcgttgtcgtggttgtagaaaaagtcgggacacaggtaacggtgtcggaaacggtacgactcggacccgtggttccgactgtgggtactactctaggaccttccggacttgaagttggactgtctctagggactccgggtctaggtactcccgaaggtccttgacgactcttgggacttggtcggtctgtcggtcgacgtcgactgttgtccgttacccgacaaggacagactcccggacttcgaccacctgttcaaagaccttctacacttcttcgacatggtgagactccggaagtgtcacttgaaacccctgtgtcttctccggttctttgtctagttactgatacaccttttcccgtgggtcccgttctaacacctggaacactttctcgacctgtccctgtgacacaaacgggaacacttgatgtagaagaagttcccgttcaccctctccgggaaacttcacttcctgtgactccttctcctgaaggtacacctggttcactggtgtcacttccacggttactacttctctgacccctacaagttataggtcgtgacgttctttgactcgtcgacccacgacgactacttcatggacccgttacgatgtcggtataagaaagacggactactcccgttcgacgtcgtggaccttttactcgactgggtactgtagtagtggtttaaagaccttttactcctgtcttctagacggtcggacgtagacgggttcgactcgtagtgtccgtgtatactggacttcagacacgaccctgtcgacccttagtggttccacaagtcgttaccccgtctggactcacctcactgtctccttcggggagacttcgacaggttccgacacgtgttccgtcacgactggtaactactcttcccgtgtctccgacgaccccggtacaaagaccttcggtaggggtacaggtaggggggtcttcacttcaagttgttcgggaaacacaaggactactaactcgtcttgtggttctcgggggacaagtacccgttccaacacttggggtgggtctttact

SEQ ID NO: 32 - protein, 419 aa, Homo sapiens
AEDPQGDAAQKTDTSHHDQDHPTFAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 33 - DNA, 5013 bp, synthetic construct
  misc_feature 1..5013: note = codon-optimised FVIII transgene (N6)
  source 1..5013: mol_type = other DNA
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaacagcaggcaccccagcaccaggcagaagcagttcaatgccaccaccatccctgagaatgacatagagaagacagacccatggtttgcccaccggacccccatgcccaagatccagaatgtgagcagctctgacctgctgatgctgctgaggcagagccccaccccccatggcctgagcctgtctgacctgcaggaggccaagtatgaaaccttctctgatgaccccagccctggggccattgacagcaacaacagcctgtctgagatgacccacttcaggccccagctgcaccactctggggacatggtgttcacccctgagtctggcctgcagctgaggctgaatgagaagctgggcaccactgctgccactgagctgaagaagctggacttcaaagtctccagcaccagcaacaacctgatcagcaccatcccctctgacaacctggctgctggcactgacaacaccagcagcctgggcccccccagcatgcctgtgcactatgacagccagctggacaccaccctgtttggcaagaagagcagccccctgactgagtctgggggccccctgagcctgtctgaggagaacaatgacagcaagctgctggagtctggcctgatgaacagccaggagagcagctggggcaagaatgtgagcagcagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 34 - DNA, 4425 bp, synthetic construct
  misc_feature 1..4425: note = codon-optimised FVIII transgene (V3)
  source 1..4425: mol_type = other DNA
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatgccactaatgtgtctaacaacagcaacaccagcaatgacagcaatgtgtctcccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 35 - DNA, 5013 bp, synthetic construct
  misc_feature 1..5013: note = codon-optimised FVIII transgene (N6) complementary strand
  source 1..5013: mol_type = other DNA
tacgtctaactcgactcgtggacgaagaaggacacggacgactccaagacgaagagacggtggtcctctatgatggacccccgacacctcgactcgaccctgatgtacgtcagactggaccccctcgacggacacctacggtccaagggggggtctcacgggttctcgaaggggaagttgtggagacaccacatgttcttctgggacaaacacctcaagtgactggtggacaagttgtaacggttcgggtccggggggacctacccggacgacccggggtggtaggtccgactccacatactgtgacaccactagtgggacttcttgtaccggtcggtgggacactcggacgtacgacacccccactcgatgaccttccggagactcccccgactcatactactggtctggtcggtctccctcttcctcctactgttccacaagggacccccgtcggtgtggatacacaccgtccacgacttcctcttaccggggtaccggagactgggggacacggactggatgtcgatggactcggtacacctggaccacttcctggacttgagaccggactaaccccgggacgaccacacgtccctcccgtcggaccggttcctcttctgggtctgggacgtgttcaagtaggacgacaaacgacacaaactactcccgttctcgaccgtgagactttggttcttgtcggactacgtcctgtccctacgacggagacggtcccggaccgggttctacgtgtgacacttaccgatacacttgtcctcggacggaccggactaaccgacggtgtccttcagacacatgaccgtacactaaccgtacccgtggtggggactccacgtgtcgtagaaggacctcccggtgtggaaggaccagtccttggtgtccgtccggtcggacctctagtcggggtagtggaaggactgacgggtctgggacgactacctggacccggtcaaggacgacaagacggtgtagtcgtcggtggtcgtactaccgtacctccggatacacttccacctgtcgacgggactcctcggggtcgactcctacttcttgttactcctccgactcctgatactactactggactgactgagactctacctacaccactccaaactactactgttgtcggggtcgaagtaggtctagtccagacaccggttcttcgtggggttctggacccacgtgatgtaacgacgactcctcctcctgaccctgatacggggggaccacgaccggggactactgtcctcgatgttctcggtcatggacttgttaccgggggtctcctaaccgtccttcatgttcttccagtccaagtaccggatgtgactactttggaagttctggtccctccggtaggtcgtactcagaccgtaggacccgggggacgacatacccctccaccccctgtgggacgactagtagaagttcttggtccggtcgtccgggatgttgtagatgggggtaccgtagtgactacactccggggacatgtcgtcctccgacgggttcccccacttcgtggacttcctgaaggggtaggacggacccctctagaagttcatgttcacctgacactgacacctcctaccggggtggttcagactggggtccacggactggtctatgatgtcgtcgaaacacttgtacctctccctggaccggagaccggactaaccgggggacgactagacgatgttcctcagacacctggtctccccgttggtctagtacagactgttctccttacactaggacaagagacacaaactactcttgtcctcgaccatggactgactcttgtaggtctccaaggacgggttgggacgaccccacgtcgacctcctgggactcaaggtccggtcgttgtagtacgtgtcgtagttaccgatacacaaactgtcggacgtcgacagacacacggacgtactccaccggatgaccatgtaggactcgtaaccccgggtctgactgaaggacagacacaagaagagaccgatgtggaagttcgtgttctaccacatactcctgtgggactgggacaaggggaagagacccctctgacacaagtactcgtacctcttgggaccggacacctaagacccgacggtgttgagactgaagtccttgtccccgtactgacgggacgactttcagaggtcgacactgttcttgtgacccctgatgatactcctgtcgatactcctgtagagacggatggacgactcgttcttgttacggtaactcgggtcctcgaagtcggtcttgtcgtccgtggggtcgtggtccgtcttcgtcaagttacggtggtggtagggactcttactgtatctcttctgtctgggtaccaaacgggtggcctgggggtacgggttctaggtcttacactcgtcgagactggacgactacgacgactccgtctcggggtggggggtaccggactcggacagactggacgtcctccggttcatactttggaagagactactggggtcgggaccccggtaactgtcgttgttgtcggacagactctactgggtgaagtccggggtcgacgtggtgagacccctgtaccacaagtggggactcagaccggacgtcgactccgacttactcttcgacccgtggtgacgacggtgactcgacttcttcgacctgaagtttcagaggtcgtggtcgttgttggactagtcgtggtaggggagactgttggaccgacgaccgtgactgttgtggtcgtcggacccgggggggtcgtacggacacgtgatactgtcggtcgacctgtggtgggacaaaccgttcttctcgtcgggggactgactcagacccccgggggactcggacagactcctcttgttactgtcgttcgacgacctcagaccggactacttgtcggtcctctcgtcgaccccgttcttacactcgtcgtccctctagtggtcctggtgggacgtcagactggtcctcctctaactgatactactgtggtagagacacctctacttcttcctcctgaaactgtagatgctgctcctgctcttggtctcggggtcctcgaaggtcttcttctggtccgtgatgaagtaacgacgacacctctccgacaccctgataccgtactcgtcgtcgggggtacacgactccttgtcccgggtcagaccgagacacggggtcaagttcttccaccacaaggtcctcaagtgactaccgtcgaagtgggtcggggacatgtctcccctcgacttactcgtggacccggacgacccggggatgtagtcccgactccacctcctgttgtagtaccactggaagtccttggtccggtcgtccgggatgtcgaagatgtcgtcggactagtcgatactcctcctggtctccgtcccccgactcgggtccttcttgaaacacttcgggttactttggttctggatgaagaccttccacgtcgtggtgtaccgggggtggttcctactcaaactgacgttccggacccggatgaagagactacacctggacctcttcctacacgtgagaccggactaaccgggggacgaccacacggtgtggttgtgggacttgggacgggtaccgtccgtccactgacacgtcctcaaacgggacaagaagtggtagaaactactttggttctcgaccatgaagtgactcttgtacctctccttgacgtcccgggggacgttgtaggtctacctcctggggtggaagttcctcttgatgtccaaggtacggtagttaccgatgtagtacctgtgggacggaccggaccactaccgggtcctggtctcctagtccaccatggacgactcgtacccgtcgttactcttgtaggtgtcgtaggtgaagagaccggtacacaagtgacactccttcttcctcctcatgttctaccgggacatgttggacatgggaccccacaaactctgacacctctacgacgggtcgttccgaccgtagacctcccacctcacggactaacccctcgtggacgtacgaccgtactcgtgggacaaggaccacatgtcgttgttcacggtctggggggacccgtaccggagaccggtgtagtccctgaaggtctagtgacggagaccggtcataccggtcacccgggggttcgaccggtccgacgtgatgagaccgtcgtagttacggacctcgtggttcctcgggaagtcgacctagttccacctggacgaccgggggtactagtaggtaccgtagttctgggtcccccggtccgtcttcaagtcgtcggacatgtagtcggtcaagtagtagtacatgtcggacctaccgttcttcaccgtctggatgtccccgttgtcgtgaccgtgggactaccacaagaaaccgttacacctgtcgagaccgtagttcgtgttgtagaagttgggggggtagtaacggtctatgtagtccgacgtggggtgggtgatgtcgtagtcctcgtgggactcctacctcgactacccgacactggacttgtcgacgtcgtacggggacccgtacctctcgttccggtagagactacgggtctagtgacggtcgtcgatgaagtggttgtacaaacggtggacctcggggtcgttccggtccgacgtggacgtcccgtcctcgttacggacctccggggtccagttgttggggttcctcaccgacgtccacctgaaggtcttctggtacttccactgaccccactggtgggtcccccacttctcggacgactggtcgtacatacacttcctcaaggactagtcgtcgtcggtcctaccggtggtcacctgggacaagaaggtcttaccgttccacttccacaaggtcccgttggtcctgtcgaagtggggacaccacttgtcggacctggggggggacgactggtctatggactcctaagtgggggtctcgacccacgtggtctaacgggactcctacctccacgacccgacactccgggtcctggacatgact

SEQ ID NO: 36 - DNA, 4425 bp, synthetic construct
  misc_feature 1..4425: note = codon-optimised FVIII transgene (V3) complementary strand
  source 1..4425: mol_type = other DNA
tacgtctaactcgactcgtggacgaagaaggacacggacgactccaagacgaagagacggtggtcctctatgatggacccccgacacctcgactcgaccctgatgtacgtcagactggaccccctcgacggacacctacggtccaagggggggtctcacgggttctcgaaggggaagttgtggagacaccacatgttcttctgggacaaacacctcaagtgactggtggacaagttgtaacggttcgggtccggggggacctacccggacgacccggggtggtaggtccgactccacatactgtgacaccactagtgggacttcttgtaccggtcggtgggacactcggacgtacgacacccccactcgatgaccttccggagactcccccgactcatactactggtctggtcggtctccctcttcctcctactgttccacaagggacccccgtcggtgtggatacacaccgtccacgacttcctcttaccggggtaccggagactgggggacacggactggatgtcgatggactcggtacacctggaccacttcctggacttgagaccggactaaccccgggacgaccacacgtccctcccgtcggaccggttcctcttctgggtctgggacgtgttcaagtaggacgacaaacgacacaaactactcccgttctcgaccgtgagactttggttcttgtcggactacgtcctgtccctacgacggagacggtcccggaccgggttctacgtgtgacacttaccgatacacttgtcctcggacggaccggactaaccgacggtgtccttcagacacatgaccgtacactaaccgtacccgtggtggggactccacgtgtcgtagaaggacctcccggtgtggaaggaccagtccttggtgtccgtccggtcggacctctagtcggggtagtggaaggactgacgggtctgggacgactacctggacccggtcaaggacgacaagacggtgtagtcgtcggtggtcgtactaccgtacctccggatacacttccacctgtcgacgggactcctcggggtcgactcctacttcttgttactcctccgactcctgatactactactggactgactgagactctacctacaccactccaaactactactgttgtcggggtcgaagtaggtctagtccagacaccggttcttcgtggggttctggacccacgtgatgtaacgacgactcctcctcctgaccctgatacggggggaccacgaccggggactactgtcctcgatgttctcggtcatggacttgttaccgggggtctcctaaccgtccttcatgttcttccagtccaagtaccggatgtgactactttggaagttctggtccctccggtaggtcgtactcagaccgtaggacccgggggacgacatacccctccaccccctgtgggacgactagtagaagttcttggtccggtcgtccgggatgttgtagatgggggtaccgtagtgactacactccggggacatgtcgtcctccgacgggttcccccacttcgtggacttcctgaaggggtaggacggacccctctagaagttcatgttcacctgacactgacacctcctaccggggtggttcagactggggtccacggactggtctatgatgtcgtcgaaacacttgtacctctccctggaccggagaccggactaaccgggggacgactagacgatgttcctcagacacctggtctccccgttggtctagtacagactgttctccttacactaggacaagagacacaaactactcttgtcctcgaccatggactgactcttgtaggtctccaaggacgggttgggacgaccccacgtcgacctcctgggactcaaggtccggtcgttgtagtacgtgtcgtagttaccgatacacaaactgtcggacgtcgacagacacacggacgtactccaccggatgaccatgtaggactcgtaaccccgggtctgactgaaggacagacacaagaagagaccgatgtggaagttcgtgttctaccacatactcctgtgggactgggacaaggggaagagacccctctgacacaagtactcgtacctcttgggaccggacacctaagacccgacggtgttgagactgaagtccttgtccccgtactgacgggacgactttcagaggtcgacactgttcttgtgacccctgatgatactcctgtcgatactcctgtagagacggatggacgactcgttcttgttacggtaactcgggtcctcgaagtcggtcttacggtgattacacagattgttgtcgttgtggtcgttactgtcgttacacagagggggtcacgacttctccgtggtctccctctagtggtcctggtgggacgtcagactggtcctcctctaactgatactactgtggtagagacacctctacttcttcctcctgaaactgtagatgctgctcctgctcttggtctcggggtcctcgaaggtcttcttctggtccgtgatgaagtaacgacgacacctctccgacaccctgataccgtactcgtcgtcgggggtacacgactccttgtcccgggtcagaccgagacacggggtcaagttcttccaccacaaggtcctcaagtgactaccgtcgaagtgggtcggggacatgtctcccctcgacttactcgtggacccggacgacccggggatgtagtcccgactccacctcctgttgtagtaccactggaagtccttggtccggtcgtccgggatgtcgaagatgtcgtcggactagtcgatactcctcctggtctccgtcccccgactcgggtccttcttgaaacacttcgggttactttggttctggatgaagaccttccacgtcgtggtgtaccgggggtggttcctactcaaactgacgttccggacccggatgaagagactacacctggacctcttcctacacgtgagaccggactaaccgggggacgaccacacggtgtggttgtgggacttgggacgggtaccgtccgtccactgacacgtcctcaaacgggacaagaagtggtagaaactactttggttctcgaccatgaagtgactcttgtacctctccttgacgtcccgggggacgttgtaggtctacctcctggggtggaagttcctcttgatgtccaaggtacggtagttaccgatgtagtacctgtgggacggaccggaccactaccgggtcctggtctcctagtccaccatggacgactcgtacccgtcgttactcttgtaggtgtcgtaggtgaagagaccggtacacaagtgacactccttcttcctcctcatgttctaccgggacatgttggacatgggaccccacaaactctgacacctctacgacgggtcgttccgaccgtagacctcccacctcacggactaacccctcgtggacgtacgaccgtactcgtgggacaaggaccacatgtcgttgttcacggtctggggggacccgtaccggagaccggtgtagtccctgaaggtctagtgacggagaccggtcataccggtcacccgggggttcgaccggtccgacgtgatgagaccgtcgtagttacggacctcgtggttcctcgggaagtcgacctagttccacctggacgaccgggggtactagtaggtaccgtagttctgggtcccccggtccgtcttcaagtcgtcggacatgtagtcggtcaagtagtagtacatgtcggacctaccgttcttcaccgtctggatgtccccgttgtcgtgaccgtgggactaccacaagaaaccgttacacctgtcgagaccgtagttcgtgttgtagaagttgggggggtagtaacggtctatgtagtccgacgtggggtgggtgatgtcgtagtcctcgtgggactcctacctcgactacccgacactggacttgtcgacgtcgtacggggacccgtacctctcgttccggtagagactacgggtctagtgacggtcgtcgatgaagtggttgtacaaacggtggacctcggggtcgttccggtccgacgtggacgtcccgtcctcgttacggacctccggggtccagttgttggggttcctcaccgacgtccacctgaaggtcttctggtacttccactgaccccactggtgggtcccccacttctcggacgactggtcgtacatacacttcctcaaggactagtcgtcgtcggtcctaccggtggtcacctgggacaagaaggtcttaccgttccacttccacaaggtcccgttggtcctgtcgaagtggggacaccacttgtcggacctggggggggacgactggtctatggactcctaagtgggggtctcgacccacgtggtctaacgggactcctacctccacgacccgacactccgggtcctggacatgact

SEQ ID NO: 37 - protein, 1670 aa, synthetic construct
  REGION 1..1670: note = exemplary FVIII polypeptide (N6)
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 38 - protein, 1474 aa, synthetic construct [sequence omitted]

SEQ ID NO: 39 - DNA, 600 bp, synthetic construct
  source 1..600: mol_type = other DNA
gggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagct

SEQ ID NO: 40 - DNA, 7349 bp, synthetic construct
  misc_feature 1..7349: note = pGM407
  source 1..7349: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactcttgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatttttttgtttcaagccctatcgaattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccgcggcaattttaaaagaaagggaggaatagggggacagacttcagcagagagactaattaatataataacaacacaattagaaatacaacatttacaaaccaaaattcaaaaaattttaaattttagagccgcggagatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagccaccatgcccagctctgtgtcctggggcattctgctgctggctggcctgtgctgtctggtgcctgtgtccctggctgaggaccctcagggggatgctgcccagaaaacagacacctcccaccatgaccaggaccaccccaccttcaacaagatcacccccaacctggcagagtttgccttcagcctgtacagacagctggcccaccagagcaacagcaccaacatctttttcagccctgtgtccattgccacagcctttgccatgctgagcctgggcaccaaggctgacacccatgatgagatcctggaaggcctgaacttcaacctgacagagatccctgaggcccagatccatgagggcttccaggaactgctgagaaccctgaaccagccagacagccagctgcagctgacaacaggcaatgggctgttcctgtctgagggcctgaagctggtggacaagtttctggaagatgtgaagaagctgtaccactctgaggccttcacagtgaactttggggacacagaagaggccaagaaacagatcaatgactatgtggaaaagggcacccagggcaagattgtggaccttgtgaaagagctggacagggacactgtgtttgcccttgtgaactacatcttcttcaagggcaagtgggagaggccctttgaagtgaaggacactgaggaagaggacttccatgtggaccaagtgaccacagtgaaggtgccaatgatgaagagactggggatgttcaatatccagcactgcaagaaactgagcagctgggtgctgctgatgaagtacctgggcaatgctacagccatattctttctgcctgatgagggcaagctgcagcacctggaaaatgagctgacccatgacatcatcaccaaatttctggaaaatgaggacagaagatctgccagcctgcatctgcccaagctgagcatcacaggcacatatgacctgaagtctgtgctgggacagctgggaatcaccaaggtgttcagcaatggggcagacctgagtggagtgacagaggaagcccctctgaagctgtccaaggctgtgcacaaggcagtgctgaccattgatgagaagggcacagaggctgctggggccatgtttctggaagccatccccatgtccatccccccagaagtgaagttcaacaagccctttgtgttcctgatgattgagcagaacaccaagagccccctgttcatgggcaaggttgtgaaccccacccagaaatgagggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 41 - DNA, 10812 bp, synthetic construct
  misc_feature 1..10812: note = pGM411
  source 1..10812: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactctgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatgtttcaagccctatcgaattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccgcggcaattttaaaagaaagggaggaatagggggacagacttcagcagagagactaattaatataataacaacacaattagaaatacaacatttacaaaccaaaattcaaaaaattttaaattttagagccgcggagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggctagccaccaatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatgccactaatgtgtctaacaacagcaacaccagcaatgacagcaatgtgtctcccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgagcggccgcgggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 42 - DNA, 10519 bp, synthetic construct
  misc_feature 1..10519: note = pGM413
  source 1..10519: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactctgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatgtttcaagccctatcgaattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccgcggcaattttaaaagaaagggaggaatagggggacagacttcagcagagagactaattaatataataacaacacaattagaaatacaacatttacaaaccaaaattcaaaaaattttaaattttagagccgcggagatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagccaccaatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatgccactaatgtgtctaacaacagcaacaccagcaatgacagcaatgtgtctcccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgagcggccgcgggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 43 - DNA, 11400 bp, synthetic construct
  misc_feature 1..11400: note = pGM412
  source 1..11400: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactctgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatgtttcaagccctatcgaattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccgcggcaattttaaaagaaagggaggaatagggggacagacttcagcagagagactaattaatataataacaacacaattagaaatacaacatttacaaaccaaaattcaaaaaattttaaattttagagccgcggagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggctagccaccaatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaacagcaggcaccccagcaccaggcagaagcagttcaatgccaccaccatccctgagaatgacatagagaagacagacccatggtttgcccaccggacccccatgcccaagatccagaatgtgagcagctctgacctgctgatgctgctgaggcagagccccaccccccatggcctgagcctgtctgacctgcaggaggccaagtatgaaaccttctctgatgaccccagccctggggccattgacagcaacaacagcctgtctgagatgacccacttcaggccccagctgcaccactctggggacatggtgttcacccctgagtctggcctgcagctgaggctgaatgagaagctgggcaccactgctgccactgagctgaagaagctggacttcaaagtctccagcaccagcaacaacctgatcagcaccatcccctctgacaacctggctgctggcactgacaacaccagcagcctgggcccccccagcatgcctgtgcactatgacagccagctggacaccaccctgtttggcaagaagagcagccccctgactgagtctgggggccccctgagcctgtctgaggagaacaatgacagcaagctgctggagtctggcctgatgaacagccaggagagcagctggggcaagaatgtgagcagcagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgagcggccgcgggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 44 - DNA, 11108 bp, synthetic construct
  misc_feature 1..11108: note = pGM414
  source 1..11108: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactcttgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatgtttcaagccctatcgaattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccgcggcaattttaaaagaaagggaggaatagggggacagacttcagcagagagactaattaatataataacaacacaattagaaatacaacatttacaaaccaaaattcaaaaaattttaaattttagagccgcggagatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagccaccaatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaacagcaggcaccccagcaccaggcagaagcagttcaatgccaccaccatccctgagaatgacatagagaagacagacccatggtttgcccaccggacccccatgcccaagatccagaatgtgagcagctctgacctgctgatgctgctgaggcagagccccaccccccatggcctgagcctgtctgacctgcaggaggccaagtatgaaaccttctctgatgaccccagccctggggccattgacagcaacaacagcctgtctgagatgacccacttcaggccccagctgcaccactctggggacatggtgttcacccctgagtctggcctgcagctgaggctgaatgagaagctgggcaccactgctgccactgagctgaagaagctggacttcaaagtctccagcaccagcaacaacctgatcagcaccatcccctctgacaacctggctgctggcactgacaacaccagcagcctgggcccccccagcatgcctgtgcactatgacagccagctggacaccaccctgtttggcaagaagagcagccccctgactgagtctgggggccccctgagcctgtctgaggagaacaatgacagcaagctgctggagtctggcctgatgaacagccaggagagcagctggggcaagaatgtgagcagcagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgagcggccgcgggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 45 - DNA, 1738 bp, synthetic construct
  misc_feature 1..1738: note = CAG promoter
  source 1..1738: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgctcgagccacc

SEQ ID NO: 46 - protein, 30 aa, synthetic construct
  REGION 1..30: note = Additional amino acid sequence encoded from false transcriptionstart site upstream of that encoding the Fct4 of SEQ ID NO: 13
MFMPSSFSYSSWATCWLLCCLIILAKNSIA